(19) United States
(12) Patent Application Publication
(10) Pub. No.: US 2018/0243435 A1
(43) Pub. Date: Aug. 30, 2018

(54) ANTI-DLL3 ANTIBODY DRUG CONJUGATES AND METHODS OF USE

(71) Applicant: ABBVIE STEMCENTRX LLC, NORTH CHICAGO, IL (US)

(72) Inventors: SCOTT J. DYLLA, EMERALD HILLS, CA (US); BRIAN SLINGERLAND, SOUTH SAN FRANCISCO, CA (US); TAE HAN, WOODINVILLE, WA (US); JOHN PEATTIE, SAN FRANCISCO, CA (US); HOLGER KARSUNKY, REDWOOD CITY, CA (US); WADE C. ANDERSON, FAIRFIELD, CA (US)

(73) Assignee: ABBVIE STEMCENTRX LLC, NORTH CHICAGO, IL (US)

(21) Appl. No.: 15/753,509

(22) PCT Filed: Aug. 19, 2016
(86) PCT No.: PCT/US16/47870

§ 371 Date: Feb. 19, 2018

Related U.S. Application Data
(60) Provisional application No. 62/207,830, filed on Aug. 20, 2015.
(60) Provisional application No. 62/323,998, filed on Apr. 18, 2016.
(60) Provisional application No. 62/373,906, filed on Aug. 11, 2016.

(51) Int. Cl. A61K 47/68; C07K 16/18; A61P 35/00

(52) U.S. Cl. CPC A61K 47/6849; C07K 16/18; C07K 2317/34; A61P 35/00; C07K 2317/24; A61K 47/6803

(57) ABSTRACT

Provided are novel anti-DLL3 antibodies and antibody drug conjugates, and methods of using such anti-DLL3 antibodies and antibody drug conjugates to treat cancer.

### CROSS REFERENCED APPLICATIONS

This application claims the benefit of U.S. Provisional Application No. 62/207,830 filed on 20 Aug. 2015, U.S. Provisional Application No. 62/323,998 filed on 18 Apr. 2016 and U.S. Provisional Application No. 62/373,906 filed on 11 Aug. 2016, each of which is incorporated herein by reference in its entirety.

### SEQUENCE LISTING

This application contains a sequence listing which has been submitted in ASCII format via EFS-Web and is hereby incorporated by reference in its entirety. Said ASCII copy, created on Aug. 8, 2016, is named sc1607_p3_Sequence_Listing_08082016 and is 591 KB (605,521 bytes) in size.

### FIELD OF THE INVENTION

This application generally relates to novel compounds and compositions and methods of administering anti-DLL3 antibodies or immunoreactive fragments thereof, including antibody drug conjugates (ADCs), comprising the same for the treatment, diagnosis or prophylaxis of cancer and any recurrence or metastasis thereof. Selected embodiments of the invention provide for the administration of such anti-DLL3 antibodies or antibody drug conjugates for the treatment of cancer comprising a reduction in tumorigenic cell frequency.

### BACKGROUND OF THE INVENTION

Differentiation and proliferation of stem cells and progenitor cells are normal ongoing processes that act in concert to support tissue growth during organogenesis, cell repair and cell replacement. The system is tightly regulated to ensure that only appropriate signals are generated based on the needs of the organism. Cell proliferation and differentiation normally occur only as necessary for the replacement of damaged or dying cells or for growth. However, disruption of these processes can be triggered by many factors including the under- or overabundance of various signaling chemicals, the presence of altered microenvironments, genetic mutations or a combination thereof. Disruption of normal cellular proliferation and/or differentiation can lead to various disorders including proliferative diseases such as cancer.

Conventional therapeutic treatments for cancer include chemotherapy, radiotherapy and immunotherapy. Often these treatments are ineffective and surgical resection may not provide a viable clinical alternative. Limitations in the current standard of care are particularly evident in those cases where patients undergo first line treatments and subsequently relapse. In such cases refractory tumors, often aggressive and incurable, frequently arise. The overall survival rate for many solid tumors have remained largely unchanged over the years due, at least in part, to the failure of existing therapies to prevent relapse, tumor recurrence and metastasis. There remains therefore a great need to develop more targeted and potent therapies for proliferative disorders. The current invention addresses this need.

### SUMMARY OF THE INVENTION

In a broad aspect the present invention provides novel compounds and compositions comprising isolated antibodies, and/or corresponding antibody drug conjugates, which specifically bind to human DLL3 determinant. In certain embodiments the DLL3 determinant is a DLL3 protein expressed on tumor cells while in other embodiments the DLL3 determinant is expressed on tumor initiating cells. In selected aspects the DLL3 ADC compositions will comprise lyophilized compositions. In other aspects the invention provides novel methods of administering the disclosed compounds or compositions including particularly effective dosing regimens and the dosing of certain patient subpopulations identified as set forth herein. As discussed in more detail below, the disclosed methodology relating to patient selection and patient dosing can prove particularly efficacious due, at least in part, to the resulting favorable therapeutic index and treatment susceptible population. In yet other embodiments the present invention provides for certain therapeutic regimens comprising a combination of DLL3 ADCs and immunotherapeutic compounds, including checkpoint inhibitors, which appear to be particularly efficacious.

In selected aspects the antibodies of the invention bind to a DLL3 protein and compete for binding with a reference antibody that binds to an epitope on human DLL3 protein. In certain embodiments the present invention comprises DLL3 antibodies or ADCs wherein the antibody or ADC binding domain binds specifically to human DLL3 (hDLL3) and comprises or competes for binding with an antibody comprising: a light chain variable region (VL) of SEQ ID NO: 21 and a heavy chain variable region (VH) of SEQ ID NO: 23; or a VL of SEQ ID NO: 25 and a VH of SEQ ID NO: 27; or a VL of SEQ ID NO: 29 and a VH of SEQ ID NO: 31; or a VL of SEQ ID NO: 33 and a VH of SEQ ID NO: 35; or a VL of SEQ ID NO: 37 and a VH of SEQ ID NO: 39; or a VL of SEQ ID NO: 41 and a VH of SEQ ID NO: 43; or a VL of SEQ ID NO: 45 and a VH of SEQ ID NO: 47; or a VL of SEQ ID NO: 49 and a VH of SEQ ID NO: 51; or a VL of SEQ ID NO: 53 and a VH of SEQ ID NO: 55; or a VL of SEQ ID NO: 57 and a VH of SEQ ID NO: 59; or a VL of SEQ ID NO: 61 and a VH of SEQ ID NO: 63; or a VL of SEQ ID NO: 65 and a VH of SEQ ID NO: 67; or a VL of SEQ ID NO: 69 and a VH of SEQ ID NO: 71; or a VL of SEQ ID NO: 73 and a VH of SEQ ID NO: 75; or a VL of SEQ ID NO: 77 and a VH of SEQ ID NO: 79; or a VL of SEQ ID NO: 81 and a VH of SEQ ID NO: 83; or a VL of SEQ ID NO: 85 and a VH of SEQ ID NO: 87; or a VL of SEQ ID NO: 89 and a VH of SEQ ID NO: 91; or a VL of SEQ ID NO: 93 and a VH of SEQ ID NO: 95; or a VL of SEQ ID NO: 97 and a VH of SEQ ID NO: 99; or a VL of SEQ ID NO: 101 and a VH of SEQ ID NO: 103; or a VL of SEQ ID NO: 105 and a VH of SEQ ID NO: 107; or a VL of SEQ ID NO: 109 and a VH of SEQ ID NO: 111; or a VL of SEQ ID NO: 113 and a VH of SEQ ID NO: 115; or a VL of SEQ ID NO: 117 and a VH of SEQ ID NO: 119; or a VL of SEQ ID NO: 121 and a VH of SEQ ID NO: 123; or a VL of SEQ ID NO: 125 and a VH of SEQ ID NO: 127; or a VL of SEQ ID NO: 129 and a VH of SEQ ID NO: 131; or a VL of SEQ ID NO: 133 and a VH of SEQ ID NO: 135; or a VL of SEQ ID NO: 137 and a VH of SEQ ID NO: 139; or a VL of SEQ ID NO: 141 and a VH of SEQ ID NO: 143; or a VL of SEQ ID NO: 145 and a VH of SEQ ID NO: 147; or a VL of SEQ ID NO: 149 and a VH of SEQ ID NO: 151; or a VL of SEQ ID NO: 153 and a VH of SEQ ID NO: 155; or a VL of SEQ ID NO: 157 and a VH of SEQ ID NO: 159; or a VL of SEQ ID NO: 161 and a VH of SEQ ID NO: 163; or a VL of SEQ ID NO: 165 and a VH of SEQ ID NO: 167; or a VL of SEQ ID NO: 169 and a VH of SEQ ID NO: 171; or a VL of SEQ ID NO: 173 and a VH of SEQ ID NO: 175; or a VL of SEQ ID NO: 177 and a VH of SEQ ID NO: 179; or a VL of SEQ ID NO: 181 and a VH of SEQ ID NO: 183; or a VL of SEQ ID NO: 185 and a VH of SEQ ID NO: 187; or a VL of SEQ ID NO: 189 and a VH of SEQ ID NO: 191; or a VL of SEQ ID NO: 193 and a VH of SEQ ID NO: 195; or a VL of SEQ ID NO: 197 and a VH of SEQ ID NO: 199; or a VL of SEQ ID NO: 201 and a VH of SEQ ID NO: 203; or a VL of SEQ ID NO: 205 and a VH of SEQ ID NO: 207; or a VL of SEQ ID NO: 209 and a VH of SEQ ID NO: 211; or a VL of SEQ ID NO: 213 and a VH of SEQ ID NO: 215; or a VL of SEQ ID NO: 217 and a VH of SEQ ID NO: 219; or a VL of SEQ ID NO: 221 and a VH of SEQ ID NO: 223; or a VL of SEQ ID NO: 225 and a VH of SEQ ID NO: 227; or a VL of SEQ ID NO: 229 and a VH of SEQ ID NO: 231; or a VL of SEQ ID NO: 233 and a VH of SEQ ID NO: 235; or a VL of SEQ ID NO: 237 and a VH of SEQ ID NO: 239; or a VL of SEQ ID NO: 241 and a VH of SEQ ID NO: 243; or a VL of SEQ ID NO: 245 and a VH of SEQ ID NO: 247; or a VL of SEQ ID NO: 249 and a VH of SEQ ID NO: 251; or a VL of SEQ ID NO: 253 and a VH of SEQ ID NO: 255; or a VL of SEQ ID NO: 257 and a VH of SEQ ID NO: 259; or a VL of SEQ ID NO: 261 and a VH of SEQ ID NO: 263; or a VL of SEQ ID NO: 265 and a VH of SEQ ID NO: 267; or a VL of SEQ ID NO: 269 and a VH of SEQ ID NO: 271; or a VL of SEQ ID NO: 273 and a VH of SEQ ID NO: 275; or a VL of SEQ ID NO: 277 and a VH of SEQ ID NO: 279; or a VL of SEQ ID NO: 281 and a VH of SEQ ID NO: 283; or a VL of SEQ ID NO: 285 and a VH of SEQ ID NO: 287; or a VL of SEQ ID NO: 289 and a VH of SEQ ID NO: 291; or a VL of SEQ ID NO: 293 and a VH of SEQ ID NO: 295; or a VL of SEQ ID NO: 297 and a VH of SEQ ID NO: 299; or a VL of SEQ ID NO: 301 and a VH of SEQ ID NO: 303; or a VL of SEQ ID NO: 305 and a VH of SEQ ID NO: 307; or a VL of SEQ ID NO: 309 and a VH of SEQ ID NO: 311; or a VL of SEQ ID NO: 313 and a VH of SEQ ID NO: 315; or a VL of SEQ ID NO: 317 and a VH of SEQ ID NO: 319; or a VL of SEQ ID NO: 321 and a VH of SEQ ID NO: 323; or a VL of SEQ ID NO: 325 and a VH of SEQ ID NO: 327; or a VL of SEQ ID NO: 329 and a VH of SEQ ID NO: 331; or a VL of SEQ ID NO: 333 and a VH of SEQ ID NO: 335; or a VL of SEQ ID NO: 337 and a VH of SEQ ID NO: 339; or a VL of SEQ ID NO: 341 and a VH of SEQ ID NO: 343; or a VL of SEQ ID NO: 345 and a VH of SEQ ID NO: 347; or a VL of SEQ ID NO: 349 and a VH of SEQ ID NO: 351; or a VL of SEQ ID NO: 353 and a VH of SEQ ID NO: 355; or a VL of SEQ ID NO: 357 and a VH of SEQ ID NO: 359; or a VL of SEQ ID NO: 361 and a VH of SEQ ID NO: 363; or a VL of SEQ ID NO: 365 and a VH of SEQ ID NO: 367; or a VL of SEQ ID NO: 369 and a VH of SEQ ID NO: 371; or a VL of SEQ ID NO: 373 and a VH of SEQ ID NO: 375; or a VL of SEQ ID NO: 377 and a VH of SEQ ID NO: 379; or a VL of SEQ ID NO: 381 and a VH of SEQ ID NO: 383; or a VL of SEQ ID NO: 385 and a VH of SEQ ID NO: 387; or a VL of SEQ ID NO: 389 and a VH of SEQ ID NO: 391; or a VL of SEQ ID NO: 393 and a VH of SEQ ID NO: 395; or a VL of SEQ ID NO: 397 and a VH of SEQ ID NO: 399; or a VL of SEQ ID NO: 401 and a VH of SEQ ID NO: 403; or a VL of SEQ ID NO: 405 and a VH of SEQ ID NO: 407. In one aspect the invention comprises a nucleic acid encoding an anti-DLL3 antibody of the invention or an immunoreactive fragment thereof. In other embodiments the invention comprises a vector comprising one or more of the above described nucleic acids and/or a host cell comprising said vector.

In some aspects of the invention the antibody comprises a chimeric, CDR grafted, humanized or human antibody or an immunoreactive fragment thereof. In other aspects of the invention the antibody, preferably comprising all or part of the aforementioned sequences, is an internalizing antibody. In yet other embodiments the antibodies will comprise site-specific antibodies. In other selected embodiments the invention comprises antibody drug conjugates incorporating any of the aforementioned antibodies.

In certain embodiments the invention comprises an antibody drug conjugate of the formula Ab-[L-D]n or a pharmaceutically acceptable salt thereof wherein: Ab comprises an anti-DLL3 antibody; L comprises an optional linker; D comprises a drug; and n is an integer from 1 to 20. In one aspect the ADC of the invention comprises an anti-DLL3 antibody such as those described above or an immunoreactive fragment thereof. In other embodiments the ADCs of the invention comprise a cytotoxic compound selected from calicheamicins, pyrrolobenzodiazepines, auristatins, duocarmycins, maytansinoids or any one of the compatible therapeutic moieties as described herein. In certain embodiments the invention comprises a pharmaceutical composition comprising an ADC as described above.

In yet other selected embodiments the ADCs of the instant invention have improved pharmacokinetic and pharmacodynamic properties that provide for an enhanced therapeutic index and allow for optimization of dosing regimens. In this regard the disclosed ADCs will have a terminal half-life exceeding six days, a terminal half-life of greater than seven days or a terminal half-life of greater than eight days when measured as set forth herein. Still other aspects of the invention will comprise ADCs having a terminal half-life of greater than nine days, a terminal half-life of greater than ten days, a terminal half-life of greater than eleven days, a terminal half-life of greater than twelve days (each as measured in human subjects). In still other embodiments the disclosed ADCs will have a terminal half-life of greater than thirteen days, a terminal half-life of greater than about fourteen days, a terminal half-life of greater than fifteen days, a terminal half-life of greater than about sixteen days, a terminal half-life of greater than about seventeen days, a terminal half-life of greater than eighteen days, a terminal half-life of greater than about nineteen days, a terminal half-life of greater than about twenty days or a terminal half-life of greater than three weeks in human subjects. In yet other embodiments the ADCs of the invention will exhibit a terminal half-life of about six days, a terminal half-life of about seven days, a terminal half-life of about eight days, a terminal half-life of about nine days, a terminal half-life of about ten days, a terminal half-life of about eleven days, a terminal half-life of about twelve days, a terminal half-life of about thirteen days, a terminal half-live of about fourteen days, a terminal half-life of about fifteen days, a terminal half-life of about sixteen days, a terminal half-life of about seventeen days, a terminal half-life of about eighteen days, a terminal half-life of about nineteen days, a terminal half-life of about twenty days or a terminal half-live of about three weeks. Those of skill in the art will appreciate that such protracted half-lives will allow for less frequent dosing of the disclosed ADCs thereby providing the desired efficacy while exhibiting similar or reduced toxicity.

To this end other aspects of the invention are directed to methods of treating cancer comprising administering a pharmaceutical composition such as those described herein to a subject in need thereof. In selected embodiments the methods will comprise administering an ADC having a terminal half-life of greater than about six days, a terminal half-life of greater than about seven days, a terminal half-life of greater than about eight days, a terminal half-life of greater than about nine days, a terminal half-life of greater than about ten days, a terminal half-life of greater than about eleven days, a terminal half-life of greater than about twelve days, a terminal half-life of greater than about thirteen days, a terminal half-live of greater than about fourteen days, a terminal half-life of greater than about fifteen days, a terminal half-life of greater than about sixteen days, a terminal half-life of greater than about seventeen days, a terminal half-life of greater than about eighteen days, a terminal half-life of greater than about nineteen days, a terminal half-life of greater than about twenty days or a terminal half-life of greater than about three weeks.

In other certain embodiments the cancer will comprise a solid tumor including, but not limited to, adrenal, liver, kidney, bladder, breast, gastric, ovarian, cervical, uterine, esophageal, colorectal, prostate, pancreatic, lung (both small cell and non-small cell), thyroid, carcinomas, sarcomas, glioblastomas and various head and neck tumors. In yet other selected aspects the cancer is selected from the group comprising lung cancer (including small cell lung cancer or large cell neuroendocrine carcinoma), prostate cancer, colorectal cancer and skin cancer such as melanoma (e.g. skin cancer expressing wild type or mutated BRAF). In some embodiments the methods of treating cancer described above comprise administering to the subject at least one additional therapeutic moiety in addition to the disclosed pharmaceutical compositions. In preferred aspects the additional therapeutic moiety will comprise an anti-PD-1 antibody or an anti-PD-L1 antibody.

As to the aforementioned methods in certain embodiments the present invention provides anti-DLL3 antibody drug conjugates for use in the treatment of cancer wherein the treatment may comprise administering an effective amount of an anti-DLL3 antibody drug conjugate (DLL3 ADC) at least once every week (QW), at least once every two weeks (Q2W), at least once every three weeks (Q3W), at least once every four weeks (Q4W), at least once every five weeks (Q5W), at least once every six weeks (Q6W), at least once every seven weeks (Q7W), at least once every eight weeks (08W), at least once every nine weeks (Q9W), at least once every ten weeks (Q10W), at least once every eleven weeks (Q11W) or at least once every twelve weeks (Q12W). In selected embodiments the DLL3 ADC will be administered at least every three weeks (Q3W), at least once every four weeks (Q4W), at least once every five weeks (Q5W) or at least once every six weeks (Q6W). In other selected embodiments the DLL3 ADC will be administered at a dose of about 0.01 mg/kg, 0.025 mg/kg, 0.05 mg/kg, 0.075 mg/kg, 0.1 mg/kg, 0.15 mg/kg, 0.2 mg/kg, 0.25 mg/kg, 0.3 mg/kg, 0.4 mg/kg, 0.5 mg/kg, 0.6 mg/kg, 0.7 mg/kg, 0.8 mg/kg, 0.9 mg/kg or 1.0 mg/kg. Selected embodiments will comprise treating the patient with a single administration of the DLL3 ADC. Certain other embodiments will comprise treating the patient at specified intervals (i.e. Q2W, 03W, Q4W, Q5W, Q6W, etc) for two cycles (×2), for three cycles (×3), for four cycles (×4), for five cycles (×5), for six cycles (×6), for seven cycles (×7), for eight cycles (×8), for nine cycles (×9), or for ten cycles (×10). In other embodiments the initial DLL3 ADC treatment (of x cycles) may be completed and no further DLL3 ADC treatment is undertaken until the cancer shows signs of progressing (treatment at progression). In yet other embodiments the initial DLL3 ADC treatment (of x cycles) may be completed and then the patient is put on maintenance therapy (e.g., 0.1 mg/kg DLL3 ADC Q6W indefinitely). In such maintenance settings the DLL3 ADC may be administered at relatively low levels as a continuous or periodic (e.g., every hour) infusion via a peristaltic pump. In still other embodiments the patient will receive the initial DLL3 ADC treatment (of x cycles) and then not be treated (with the disclosed ADCs or with another agent) for the same or related cancer again until progression (i.e., treat at progression). In such cases the second treatment cycle (e.g., with DLL3 ADCs) will not be effected until after four, five, six, seven, eight, nine, ten, eleven, twelve, thirteen, fourteen, fifteen or sixteen or more weeks after conclusion of the first treatment cycle.

In one embodiment the invention comprises a method of reducing tumor initiating cells in a tumor cell population, wherein the method comprises contacting (e.g. in vitro or in vivo) a tumor initiating cell population with an ADCs as described herein whereby the frequency of the tumor initiating cells is reduced.

In one aspect, the invention comprises a method of delivering a cytotoxin to a cell comprising contacting the cell with any of the above described ADCs.

In another aspect, the invention comprises a method of detecting, diagnosing, or monitoring cancer (e.g., lung cancer, prostate cancer or melanoma) in a subject, the method comprising the steps of contacting (e.g. in vitro or in vivo) tumor cells with an DLL3 detection agent and detecting the DLL3 detection agent associated with the tumor cells. In selected embodiments the detection agent shall comprise an anti-DLL3 antibody or a nucleic acid probe that associates with a DLL3 genotypic determinant. In related embodiments the diagnostic method will comprise immunohistochemistry (IHC) or in situ hybridization (ISH).

To this end it will be appreciated that certain aspects of the instant invention comprise the use of DLL3 antibodies for immunohistochemistry. More particularly DLL3 IHC may be used as a diagnostic tool to aid in the diagnosis of various proliferative disorders and to monitor the potential response to treatments including DLL3 antibody therapy. In this respect and as shown in the Examples below immunohistochemistry techniques may be used to derive an H-score as known in the art. Such H-scores (i.e., those 90 and above on a 300 point scale) may be used to indicate which patients may be amenable to treatment with compositions of the instant invention as well as being used to guide treatment decisions and determine dosing regimens and timing. In other embodiments the percentage of positively stained DLL3 cells in the tumor may be used to indicate which patients may be susceptible to treatment with the disclosed DLL3 ADCs.

In a similar vein the present invention also provides kits or devices and associated methods that are useful in the diagnosis, monitoring or treatment of DLL3 associated disorders such as cancer. To this end the present invention preferably provides an article of manufacture useful for detecting, diagnosing or treating DLL3 associated disorders comprising a receptacle containing a DLL3 ADC and instructional materials for using said DLL3 ADC to treat, monitor or diagnose the DLL3 associated disorder. In selected embodiments the devices and associated methods will comprise the step of contacting at least one circulating tumor cell. In other embodiments the disclosed kits will comprise instructions, labels, inserts, readers or the like indicating that the kit or device is used for the diagnosis, monitoring or treatment of a DLL3 associated cancer.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description.

### BRIEF DESCRIPTION OF THE FIGURES

FIGS. 1A and 1B provide, in a tabular form, contiguous amino acid sequences (SEQ ID NOS: 21-407, odd numbers) of light and heavy chain variable regions of a number of murine and humanized exemplary DLL3 antibodies compatible with the disclosed DLL3 ADCs isolated, cloned and engineered as described in the Examples herein;

FIG. 2 depicts, in schematic form, the results of domain level mapping analysis of exemplary DLL3 antibodies isolated, cloned and engineered as described in the Examples herein;

FIGS. 3A and 3B provide immunohistochemistry data showing that DLL3 expression does not correlate with clinical outcome of standard of care therapies (FIG. 3A) and that DLL3 expression is elevated in both naïve and chemorefractory SCLC patients (FIG. 3B);

FIGS. 4A-4F provide various data regarding a Phase I study where FIG. 4A summarizes the study design and results, FIG. 4B depicts clinical pharmacokinetics of the DLL3 ADCs, FIG. 4C shows durations of response provided by the DLL3 ADCs, FIGS. 4D and 4E show responses of all treated patients and DLL3+ positive patients and FIG. 4F provides a tabular summary of reported adverse events;

FIGS. 5A-5C provide data in a tabular form demonstrating that lyophilized embodiments of the disclosed DLL3 ADCs are stable at three different temperatures; and

FIGS. 6A and 6B illustrate that, when administered in combination, DLL3 ADCs and anti-PD-1 antibodies act to inhibit tumor growth in immunocompetent mice.

### DETAILED DESCRIPTION OF THE INVENTION

The invention may be embodied in many different forms. Disclosed herein are non-limiting, illustrative embodiments of the invention that exemplify the principles thereof. Any section headings used herein are for organizational purposes only and are not to be construed as limiting the subject matter described. For the purposes of the instant disclosure all identifying sequence accession numbers may be found in the NCBI Reference Sequence (RefSeq) database and/or the NCBI GenBank® archival sequence database unless otherwise noted.

DLL3 expression has surprisingly been found to correlate with a number of tumor types and, as a determinant, may be exploited in the treatment of such tumors. It has also unexpectedly been found that DLL3 expression is associated with tumorigenic cells and, as such, may be effectively exploited to inhibit or eliminate such cells. Tumorigenic cells, which will be described in more detail below, are known to exhibit resistance to many conventional treatments. In contrast to the teachings of the prior art, the disclosed compounds and methods effectively overcome this inherent resistance.

The invention provides anti-DLL3 antibodies (including antibody drug conjugates) and their use in the prognosis, diagnosis, theragnosis, treatment and/or prevention of a variety of DLL3-associated cancers regardless of any particular mechanism of action or specifically targeted cellular or molecular component. Surprisingly, certain disclosed DLL3 ADCs have been found to have a relatively long half-life in vivo allowing for novel dosing regimens that provide an unexpectedly robust therapeutic index. Moreover, patients suffering from tumors expressing certain levels of DLL3 are particularly receptive to treatment with the instant antibodies and ADCs as evidenced by clinical trial data appended hereto. Yet other embodiments comprise the use of the disclosed DLL3 ADCs in combination with certain immunotherapeutic compounds, including antibodies to PD-1 that appear surprisingly effective in inhibiting tumor growth. In still other embodiments the disclosed ADCs have been found to exhibit unusual stability in a lyophilized form. It will be appreciated that such advances generally provides for more effective administration of the disclosed compositions and, ultimately, better patient outcomes than provided by standard of care therapies.

### I. DLL3 PHYSIOLOGY

It has been found that DLL3 phenotypic determinants are clinically associated with various proliferative disorders, including neoplasia exhibiting neuroendocrine features, and that DLL3 protein and variants or isoforms thereof provide useful tumor markers which may be exploited in the treatment of related diseases. In this regard the present invention provides a number of antibody drug conjugates comprising an anti-DLL3 antibody targeting agent and a payload (e.g., a payload comprising a PBD warhead). As discussed in more detail below and set forth in the appended Examples, the disclosed anti-DLL3 ADCs are particularly effective at eliminating tumorigenic cells and therefore useful for the treatment and prophylaxis of certain proliferative disorders or the progression or recurrence thereof. In addition, certain disclosed ADC compositions (e.g., site-specific constructs) may exhibit a relatively high DAR=2 percentage and unexpected stability that may provide for an improved therapeutic index when compared with conventional ADC compositions comprising the same components. Additionally, the disclosed ADCs may exhibit protracted terminal half-lives allowing for the use of novel dosing regimens that may further increase the therapeutic index.

Moreover, it has been found that DLL3 markers or determinants such as cell surface DLL3 protein are therapeutically associated with cancer stem cells (also known as tumor perpetuating cells) and may be effectively exploited to eliminate or silence the same. The ability to selectively reduce or eliminate cancer stem cells through the use of anti-DLL3 conjugates as disclosed herein is surprising in that such cells are known to generally be resistant to many conventional treatments. That is, the effectiveness of traditional, as well as more recent targeted treatment methods, is often limited by the existence and/or emergence of resistant cancer stem cells that are capable of perpetuating tumor growth even in face of these diverse treatment methods. Further, determinants associated with cancer stem cells often make poor therapeutic targets due to low or inconsistent expression, failure to remain associated with the tumorigenic cell or failure to present at the cell surface. In sharp contrast to the teachings of the prior art, the instantly disclosed ADCs and methods effectively overcome this inherent resistance and to specifically eliminate, deplete, silence or promote the differentiation of such cancer stem cells thereby negating their ability to sustain or re-induce the underlying tumor growth. Moreover, as indicated herein the unexpected stability provided by the disclosed ADCs and relatively homogeneous DAR preparations allow for novel dosing regimens that may be particularly efficacious.

Thus DLL3 conjugates such as those disclosed herein may advantageously be used in the treatment and/or prevention of selected proliferative (e.g., neoplastic) disorders or progression or recurrence thereof. It will be appreciated that, while preferred embodiments of the invention will be discussed extensively below, particularly in terms of particular domains, regions or epitopes or in the context of cancer stem cells or tumors comprising neuroendocrine features and their interactions with the disclosed antibody drug conjugates, those skilled in the art will appreciate that the scope of the instant invention is not limited by such exemplary embodiments. Rather, the most expansive embodiments of the present invention and the appended claims are broadly and expressly directed to the disclosed anti-DLL3 conjugates and their use in the treatment and/or prevention of a variety of DLL3 associated or mediated disorders, including neoplastic or cell proliferative disorders, regardless of any particular mechanism of action or specifically targeted tumor, cellular or molecular component.

In Drosophila, Notch signaling is mediated primarily by one Notch receptor gene and two ligand genes, known as Serrate and Delta (Wharton et al, 1985; Rebay et al., 1991). In humans, there are four known Notch receptors and five DSL (Delta-Serrate LAG2) ligands—two homologs of Serrate, known as Jagged1 and Jagged 2, and three homologs of Delta, termed delta-like ligands or DLL1, DLL3 and DLL4. In general, Notch receptors on the surface of the signal-receiving cell are activated by interactions with ligands expressed on the surface of an opposing, signal-sending cell (termed a trans-interaction). These trans-interactions lead to a sequence of protease mediated cleavages of the Notch receptor. In consequence, the Notch receptor intracellular domain is free to translocate from the membrane to the nucleus, where it partners with the CSL family of transcription factors (RBPJ in humans) and converts them from transcriptional repressors into activators of Notch responsive genes.

Of the human Notch ligands, DLL3 is different in that it seems incapable of activating the Notch receptor via trans-interactions (Ladi et al., 2005). Notch ligands may also interact with Notch receptors in cis (on the same cell) leading to inhibition of the Notch signal, although the exact mechanisms of cis-inhibition remain unclear and may vary depending upon the ligand (for instance, see Klein et al., 1997; Ladi et al., 2005; Glittenberg et al., 2006). Two hypothesized modes of inhibition include modulating Notch signaling at the cell surface by preventing trans-interactions, or by reducing the amount of Notch receptor on the surface of the cell by perturbing the processing of the receptor or by physically causing retention of the receptor in the endoplasmic reticulum or Golgi (Sakamoto et al., 2002; Dunwoodie, 2009). It is clear, however, that stochastic differences in expression of Notch receptors and ligands on neighboring cells can be amplified through both transcriptional and non-transcriptional processes, and subtle balances of cis- and trans-interactions can result in a fine tuning of the Notch mediated delineation of divergent cell fates in neighboring tissues (Sprinzak et al., 2010).

DLL3 is a member of the Delta-like family of Notch DSL ligands. Representative DLL3 protein orthologs include, but are not limited to, human (Accession Nos. NP_058637 and NP_982353), chimpanzee (Accession No. XP_003316395), mouse (Accession No. NP_031892), and rat (Accession No. NP_446118). In humans, the DLL3 gene consists of 8 exons spanning 9.5 kBp located on chromosome 19q13. Alternate splicing within the last exon gives rise to two processed transcripts, one of 2389 bases (Accession No. NM_016941) and one of 2052 bases (Accession No. NM_203486). The former transcript encodes a 618 amino acid protein (Accession No. NP_058637; SEQ ID NO: 1), whereas the latter encodes a 587 amino acid protein (Accession No. NP_982353; SEQ ID NO: 2). These two protein isoforms of DLL3 share overall 100% identity across their extracellular domains and their transmembrane domains, differing only in that the longer isoform contains an extended cytoplasmic tail containing 32 additional residues at the carboxy terminus of the protein. The biological relevance of the isoforms is unclear, although both isoforms can be detected in tumor cells.

The extracellular region of the DLL3 protein comprises six EGF-like domains, the single DSL domain and the N-terminal domain. Generally, the EGF domains are recognized as occurring at about amino acid residues 216-249 (domain 1), 274-310 (domain 2), 312-351 (domain 3), 353-389 (domain 4), 391-427 (domain 5) and 429-465 (domain 6), with the DSL domain at about amino acid residues 176-215 and the N-terminal domain at about amino acid residues 27-175 of hDLL3 (SEQ ID NOS: 1 and 2). As discussed in more detail herein and shown in the Examples below, each of the EGF-like domains, the DSL domain and the N-terminal domain comprise part of the DLL3 protein as defined by a distinct amino acid sequence. Note that, for the purposes of the instant disclosure the respective EGF-like domains may be termed EGF1 to EGF6 with EGF1 being closest to the N-terminal portion of the protein. In regard to the structural composition of the protein one significant aspect of the instant invention is that the disclosed DLL3 modulators may be generated, fabricated, engineered or selected so as to react with a selected domain, motif or epitope. In certain cases such antibodies or ADCs may provide enhanced reactivity and/or efficacy depending on their primary mode of action. In particularly preferred embodiments the anti-DLL3 ADCs will bind to the DSL domain and, in even more preferred embodiments, will bind to an epitope comprising G203, R205, P206 (SEQ ID NO: 4) within the DSL domain.

### II. CANCER STEM CELLS

According to the current models, a tumor comprises non-tumorigenic cells and tumorigenic cells. Non-tumorigenic cells do not have the capacity to self-renew and are incapable of reproducibly forming tumors, even when transplanted into immunocompromised mice in excess cell numbers. Tumorigenic cells, also referred to herein as “tumor initiating cells” (TICs), which make up 0.1-40% (more typically 0.1-10% or 0.01-1.0%) of a tumor's cell population, have the ability to form tumors. Tumorigenic cells encompass both tumor perpetuating cells (TPCs), referred to interchangeably as cancer stem cells (CSCs) and tumor progenitor cells (TProgs).

CSCs, like normal stem cells that support cellular hierarchies in normal tissue, are able to self-replicate indefinitely while maintaining the capacity for multilineage differentiation. In this regard CSCs are able to generate both tumorigenic progeny and non-tumorigenic progeny and are able to completely recapitulate the heterogeneous cellular composition of the parental tumor as demonstrated by serial isolation and transplantation of low numbers of isolated CSCs into immunocompromised mice. Evidence indicates that unless these “seed cells” are eliminated tumors are much more likely to metastasize or reoccur leading to relapse and ultimate progression of the disease.

TProgs, like CSCs have the ability to fuel tumor growth in a primary transplant. However, unlike CSCs, they are not able to recapitulate the cellular heterogeneity of the parental tumor and are less efficient at reinitiating tumorigenesis in subsequent transplants because TProgs are typically only capable of a finite number of cell divisions as demonstrated by serial transplantation of low numbers of highly purified TProg into immunocompromised mice. TProgs may further be divided into early TProgs and late TProgs, which may be distinguished by phenotype (e.g., cell surface markers) and their different capacities to recapitulate tumor cell architecture. While neither can recapitulate a tumor to the same extent as CSCs, early TProgs have a greater capacity to recapitulate the parental tumor's characteristics than late TProgs. Notwithstanding the foregoing distinctions, it has been shown that some TProg populations can, on rare occasion, gain self-renewal capabilities normally attributed to CSCs and can themselves become CSCs.

CSCs exhibit higher tumorigenicity and are relatively more quiescent than: (i) TProgs (both early and late TProgs); and (ii) non-tumorigenic cells such as tumor-infiltrating cells, for example, fibroblasts/stroma, endothelial and hematopoietic cells that may be derived from CSCs and typically comprise the bulk of a tumor. Given that conventional therapies and regimens have, in large part, been designed to debulk tumors and attack rapidly proliferating cells, CSCs are more resistant to conventional therapies and regimens than the faster proliferating TProgs and other bulk tumor cell populations such as non-tumorigenic cells. Other characteristics that may make CSCs relatively chemoresistant to conventional therapies are increased expression of multi-drug resistance transporters, enhanced DNA repair mechanisms and anti-apoptotic gene expression. Such CSC properties have been implicated in the failure of standard treatment regimens to provide a lasting response in patients with advanced stage neoplasia as standard chemotherapy does not effectively target the CSCs that actually fuel continued tumor growth and recurrence. By inhibiting or eliminating the ability of these seed cells to propagate tumor growth and spread the disclosed compounds and compositions

It has surprisingly been discovered that DLL3 expression is associated with various tumorigenic cell subpopulations in a manner which renders them susceptible to treatment as set forth herein. The invention provides anti-DLL3 antibodies that may be particularly useful for targeting tumorigenic cells and may be used to silence, sensitize, neutralize, reduce the frequency, block, abrogate, interfere with, decrease, hinder, restrain, control, deplete, moderate, mediate, diminish, reprogram, eliminate, kill or otherwise inhibit (collectively, “inhibit”) tumorigenic cells, thereby facilitating the treatment, management and/or prevention of proliferative disorders (e.g. cancer). Advantageously, the novel anti-DLL3 antibodies of the invention may be selected so they preferably reduce the frequency or tumorigenicity of tumorigenic cells upon administration to a subject regardless of the form of the DLL3 determinant (e.g., phenotypic or genotypic). The reduction in tumorigenic cell frequency may occur as a result of (i) inhibition or eradication of tumorigenic cells; (ii) controlling the growth, expansion or recurrence of tumorigenic cells; (iii) interrupting the initiation, propagation, maintenance, or proliferation of tumorigenic cells; or (iv) by otherwise hindering the survival, regeneration and/or metastasis of the tumorigenic cells. In some embodiments, the inhibition of tumorigenic cells may occur as a result of a change in one or more physiological pathways. The change in the pathway, whether by inhibition of the tumorigenic cells, modification of their potential (for example, by induced differentiation or niche disruption) or otherwise interfering with the ability of tumorigenic cells to influence the tumor environment or other cells, allows for the more effective treatment of DLL3 associated disorders by inhibiting tumorigenesis, tumor maintenance and/or metastasis and recurrence. It will further be appreciated that the same characteristics of the disclosed antibodies make them particularly effective at treating recurrent tumors which have proved resistant or refractory to standard treatment regimens.

Methods that can be used to assess the reduction in the frequency of tumorigenic cells, include but are not limited to, cytometric or immunohistochemical analysis, preferably by in vitro or in vivo limiting dilution analysis (Dylla et al. 2008, PMID: PMC2413402 and Hoey et al. 2009, PMID: 19664991).

In vitro limiting dilution analysis may be performed by culturing fractionated or unfractionated tumor cells (e.g. from treated and untreated tumors, respectively) on solid medium that fosters colony formation and counting and characterizing the colonies that grow. Alternatively, the tumor cells can be serially diluted onto plates with wells containing liquid medium and each well can be scored as either positive or negative for colony formation at any time after inoculation but preferably more than 10 days after inoculation.

In vivo limiting dilution is performed by transplanting tumor cells, from either untreated controls or from tumors exposed to selected therapeutic agents, into immunocompromised mice in serial dilutions and subsequently scoring each mouse as either positive or negative for tumor formation. The scoring may occur at any time after the implanted tumors are detectable but is preferably done 60 or more days after the transplant. The analysis of the results of limiting dilution experiments to determine the frequency of tumorigenic cells is preferably done using Poisson distribution statistics or assessing the frequency of predefined definitive events such as the ability to generate tumors in vivo or not (Fazekas et al., 1982, PMID: 7040548).

Flow cytometry and immunohistochemistry may also be used to determine tumorigenic cell frequency. Both techniques employ one or more antibodies or reagents that bind art recognized cell surface proteins or markers known to enrich for tumorigenic cells (see WO 2012/031280). As known in the art, flow cytometry (e.g. florescence activated cell sorting (FACS)) can also be used to characterize, isolate, purify, enrich or sort for various cell populations including tumorigenic cells. Flow cytometry measures tumorigenic cell levels by passing a stream of fluid, in which a mixed population of cells is suspended, through an electronic detection apparatus which is able to measure the physical and/or chemical characteristics of up to thousands of particles per second. Immunohistochemistry provides additional information in that it enables visualization of tumorigenic cells in situ (e.g., in a tissue section) by staining the tissue sample with labeled antibodies or reagents which bind to tumorigenic cell markers.

As such, the antibodies of the invention may be useful for identifying, characterizing, monitoring, isolating, sectioning or enriching populations or subpopulations of tumorigenic cells through methods such as, for example, flow cytometry, magnetic activated cell sorting (MACS), laser mediated sectioning or FACS. FACS is a reliable method used to isolate cell subpopulations at more than 99.5% purity based on specific cell surface markers. Other compatible techniques for the characterization and manipulation of tumorigenic cells including CSCs can be seen, for example, in U.S. Ser. Nos. 12/686,359, 12/669,136 and 12/757,649.

Listed below are markers that have been associated with CSC populations and have been used to isolate or characterize CSCs: ABCA1, ABCA3, ABCG2, ADAMS, ADCY9, ADORA2A, AFP, AXIN1, B7H3, BCL9, Bmi-1, BMP-4, C20orf52, C4.4A, carboxypeptidase M, CAV1, CAV2, CD105, CD133, CD14, CD16, CD166, CD16a, CD16b, CD2, CD20, CD24, CD29, CD3, CD31, CD324, CD325, CD34, CD38, CD44, CD45, CD46, CD49b, CD49f, CD56, CD64, CD74, CD9, CD90, CEACAM6, CELSR1, CPD, CRIM1, CX3CL1, CXCR4, DAF, decorin, easyh1, easyh2, EDG3, eed, EGFR, ENPP1, EPCAM, EPHA1, EPHA2, FLJ10052, FLVCR, FZD1, FZD10, FZD2, FZD3, FZD4, FZD6, FZD7, FZD8, FZD9, GD2, GJA1, GLI1, GLI2, GPNMB, GPR54, GPRC5B, IL1R1, ID1RAP, JAM3, Lgr5, Lgr6, LRP3, LY6E, MCP, mf2, mllt3, MPZL1, MUC1, MUC16, MYC, N33, Nanog, NB84, nestin, NID2, NMA, NPC1, oncostatin M, OCT4, OPN3, PCDH7, PCDHA10, PCDHB2, PPAP2C, PTPN3, PATIENTS, RARRES1, SEMA4B, SLC19A2, SLC1A1, SLC39A1, SLC4A11, SLC6A14, SLC7A8, smarcA3, smarcD3, smarcE1, smarcA5, Sox1, STAT3, STEAP, TCF4, TEM8, TGFBR3, TMEPAI, TMPRSS4, transferrin receptor, TrkA, WNT10B, WNT16, WNT2, WNT2B, WNT3, WNT5A, YY1 and β-catenin. See, for example, Schulenburg et al., 2010, PMID: 20185329, U.S. Pat. No. 7,632,678 and U.S.P.N.s. 2007/0292414, 2008/0175870, 2010/0275280, 2010/0162416 and 2011/0020221.

Similarly, non-limiting examples of cell surface phenotypes associated with CSCs of certain tumor types include CD44^{hi}CD24^{low}, ALDH⁺, CD133⁺, CD123⁺, CD34⁺CD38⁻, CD44⁺CD24⁻, CD46^{hi}CD324⁺CD66c⁻, CD133⁺CD34⁺CD10⁻CD19⁻, CD138⁻CD34⁻CD19⁺, CD133⁺RC2⁺, CD44⁺α₂β₁^{hi}CD133⁺, CD44⁺CD24⁺ESA⁺, CD271⁺, ABCB5⁺ as well as other CSC surface phenotypes that are known in the art. See, for example, Schulenburg et al., 2010, supra, Visvader et al., 2008, PMID: 18784658 and U.S.P.N. 2008/0138313. Of particular interest with respect to the instant invention are CSC preparations comprising CD46^{hi}CD324⁺ phenotypes.

“Positive,” “low” and “negative” expression levels as they apply to markers or marker phenotypes are defined as follows. Cells with negative expression (i.e. “−”) are herein defined as those cells expressing less than, or equal to, the 95th percentile of expression observed with an isotype control antibody in the channel of fluorescence in the presence of the complete antibody staining cocktail labeling for other proteins of interest in additional channels of fluorescence emission. Those skilled in the art will appreciate that this procedure for defining negative events is referred to as “fluorescence minus one”, or “FMO”, staining. Cells with expression greater than the 95th percentile of expression observed with an isotype control antibody using the FMO staining procedure described above are herein defined as “positive” (i.e. “+”). As defined herein there are various populations of cells broadly defined as “positive.” A cell is defined as positive if the mean observed expression of the antigen is above the 95th percentile determined using FMO staining with an isotype control antibody as described above. The positive cells may be termed cells with low expression (i.e. “lo”) if the mean observed expression is above the 95^{th} percentile determined by FMO staining and is within one standard deviation of the 95^{th} percentile. Alternatively, the positive cells may be termed cells with high expression (i.e. “hi”) if the mean observed expression is above the 95^{th} percentile determined by FMO staining and greater than one standard deviation above the 95^{th} percentile. In other embodiments the 99th percentile may preferably be used as a demarcation point between negative and positive FMO staining and in some embodiments the percentile may be greater than 99%.

The CD46^{hi}CD324⁺ marker phenotype and those exemplified immediately above may be used in conjunction with standard flow cytometric analysis and cell sorting techniques to characterize, isolate, purify or enrich TIC and/or TPC cells or cell populations for further analysis.

The ability of the antibodies of the current invention to reduce the frequency of tumorigenic cells can therefore be determined using the techniques and markers described above. In some instances, the anti-DLL3 antibodies may reduce the frequency of tumorigenic cells by 10%, 15%, 20%, 25%, 30% or even by 35%. In other embodiments, the reduction in frequency of tumorigenic cells may be in the order of 40%, 45%, 50%, 55%, 60% or 65%. In certain embodiments, the disclosed compounds my reduce the frequency of tumorigenic cells by 70%, 75%, 80%, 85%, 90% or even 95%. It will be appreciated that any reduction of the frequency of tumorigenic cells is likely to result in a corresponding reduction in the tumorigenicity, persistence, recurrence and aggressiveness of the neoplasia.

### III. ANTIBODIES

A. Antibody Structure

Antibodies and variants and derivatives thereof, including accepted nomenclature and numbering systems, have been extensively described, for example, in Abbas et al. (2010), Cellular and Molecular Immunology (6^{th} Ed.), W.B. Saunders Company; or Murphey et al. (2011), Janeway's Immunobiology (8^{th} Ed.), Garland Science.

An “antibody” or “intact antibody” typically refers to a Y-shaped tetrameric protein comprising two heavy (H) and two light (L) polypeptide chains held together by covalent disulfide bonds and non-covalent interactions. Each light chain is composed of one variable domain (VL) and one constant domain (CL). Each heavy chain comprises one variable domain (VH) and a constant region, which in the case of IgG, IgA, and IgD antibodies, comprises three domains termed CH1, CH2, and CH3 (IgM and IgE have a fourth domain, CH4). In IgG, IgA, and IgD classes the CH1 and CH2 domains are separated by a flexible hinge region, which is a proline and cysteine rich segment of variable length (from about 10 to about 60 amino acids in various IgG subclasses). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues.

As used herein the term “antibody” includes polyclonal antibodies, multiclonal antibodies, monoclonal antibodies, chimeric antibodies, humanized and primatized antibodies, CDR grafted antibodies, human antibodies (including recombinantly produced human antibodies), recombinantly produced antibodies, intrabodies, multispecific antibodies, bispecific antibodies, monovalent antibodies, multivalent antibodies, anti-idiotypic antibodies, synthetic antibodies, including muteins and variants thereof, immunospecific antibody fragments such as Fd, Fab, F(ab′)₂, F(ab′) fragments, single-chain fragments (e.g. ScFv and ScFvFc); and derivatives thereof including Fc fusions and other modifications, and any other immunoreactive molecule so long as it exhibits preferential association or binding with a determinant. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2). Heavy-chain constant domains that correspond to the different classes of antibodies are typically denoted by the corresponding lower case Greek letter α, δ, ε, γ, and μ, respectively. Light chains of the antibodies from any vertebrate species can be assigned to one of two clearly distinct types, called kappa (κ) and lambda (λ), based on the amino acid sequences of their constant domains.

The variable domains of antibodies show considerable variation in amino acid composition from one antibody to another and are primarily responsible for antigen recognition and binding. Variable regions of each light/heavy chain pair form the antibody binding site such that an intact IgG antibody has two binding sites (i.e. it is bivalent). VH and VL domains comprise three regions of extreme variability, which are termed hypervariable regions, or more commonly, complementarity-determining regions (CDRs), framed and separated by four less variable regions known as framework regions (FRs). The non-covalent association between the VH and the VL region forms the Fv fragment (for “fragment variable”) which contains one of the two antigen-binding sites of the antibody. ScFv fragments (for single chain fragment variable), which can be obtained by genetic engineering, associates in a single polypeptide chain, the VH and the VL region of an antibody, separated by a peptide linker.

As used herein, the assignment of amino acids to each domain, framework region and CDR may be in accordance with one of the schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co or AbM (Oxford Molecular/MSI Pharmacopia) unless otherwise noted. As is well known in the art variable region residue numbering is typically as set forth in Chothia or Kabat. Amino acid residues which comprise CDRs as defined by Kabat, Chothia, MacCallum (also known as Contact) and AbM as obtained from the Abysis website database (infra.) are set out below in Table 1. Note that MacCallum uses the Chothia numbering system.

TABLE 1

| | Kabat | Chothia | MacCallum | AbM |
|---|---|---|---|---|
| VH CDR1 | 31-35 | 26-32 | 30-35 | 26-35 |
| VH CDR2 | 50-65 | 52-56 | 47-58 | 50-58 |
| VH CDR3 | 95-102 | 95-102 | 93-101 | 95-102 |
| VL CDR1 | 24-34 | 24-34 | 30-36 | 24-34 |
| VL CDR2 | 50-56 | 50-56 | 46-55 | 50-56 |
| VL CDR3 | 89-97 | 89-97 | 89-96 | 89-97 |

Variable regions and CDRs in an antibody sequence can be identified according to general rules that have been developed in the art (as set out above, such as, for example, the Kabat nomenclature system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably the sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, all CDRs set forth herein are derived according to the Abysis database website as per Kabat et al.

For heavy chain constant region amino acid positions discussed in the invention, numbering is according to the Eu index first described in Edelman et al., 1969, Proc. Natl. Acad. Sci. USA 63(1): 78-85 describing the amino acid sequence of the myeloma protein Eu, which reportedly was the first human IgG1 sequenced. The Eu index of Edelman is also set forth in Kabat et al., 1991 (supra.). Thus, the terms “Eu index as set forth in Kabat” or “Eu index of Kabat” or “Eu index” or “Eu numbering” in the context of the heavy chain refers to the residue numbering system based on the human IgG1 Eu antibody of Edelman et al. as set forth in Kabat et al., 1991 (supra.). The numbering system used for the light chain constant region amino acid sequence is similarly set forth in Kabat et al., (supra.). An exemplary kappa light chain constant region amino acid sequence compatible with the present invention is set forth immediately below:

(SEQ ID NO: 5)
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSS
PVTKSFNRGEC.

Similarly, an exemplary IgG1 heavy chain constant region amino acid sequence compatible with the present invention is set forth immediately below:

(SEQ ID NO: 6)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFF
LYSKLTVDKSRWQQGNVFSCSVMHEALHNHYTQKSLSLSPG.

The disclosed constant region sequences, or art-known variations or derivatives thereof, may be operably associated with the disclosed heavy and light chain variable regions using standard molecular biology techniques to provide full-length antibodies that may be used as such or incorporated in the anti-DLL3 ADCs of the invention. In this regard, and for the purposes of illustration, the full length heavy (SEQ ID NO: 7) and light (SEQ ID NO: 8) chain amino acid sequences for the exemplary antibody hSC16.56 are set forth immediately below.

(SEQ ID NO: 7)
QVQLVQSGAEVKKPGASVKVSCKASGYTFTNYGMNWVRQAPGQGLEWM
GWINTYTGEPTYADDFKGRVTMTTDTSTSTAYMELRSLRSDDTAVYYC
ARIGDSSPSDYWGQGTLVTVSSASTKGPSVFPLAPSSKSTSGGTAALG
CLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWES
NGQPENNYKTIPPVLDSDGSFFLYSKLTVDKSRWQQGNVFSCSVMHEA
LHNHYTQKSLSLSPG.
(SEQ ID NO: 8)
EIVMTQSPATLSVSPGERATLSCKASQSVSNDVVWYQQKPGQAPRLLI
YYASNRYTGIPARFSGSGSGTEFTLTISSLQSEDFAVYYCQQDYTSPW
TFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC.

It will be appreciated that the antibodies or immunoglobulins (and ADCs) of the invention may be generated from an antibody that specifically recognizes or associates with any DLL3 determinant. As used herein “determinant” or “target” means any detectable trait, property, marker or factor that is identifiably associated with, or specifically found in or on a particular cell, cell population or tissue. Determinants or targets may be morphological, functional or biochemical in nature and are preferably phenotypic. In some embodiments a determinant is a protein that is differentially expressed (over- or under-expressed) by specific cell types or by cells under certain conditions (e.g., during specific points of the cell cycle or cells in a particular niche). For the purposes of the instant invention a determinant preferably is differentially expressed on aberrant cancer cells and may comprise a DLL3 protein, or any of its splice variants, isoforms, homologs or family members, or specific domains, regions or epitopes thereof. An “antigen”, “immunogenic determinant”, “antigenic determinant” or “immunogen” means any protein or any fragment, region or domain thereof that can stimulate an immune response when introduced into an immunocompetent animal and is recognized by the antibodies produced from the immune response. The presence or absence of the DLL3 determinants contemplated herein may be used to identify a cell, cell subpopulation or tissue (e.g., tumors, tumorigenic cells or CSCs).

There are two types of disulfide bridges or bonds in immunoglobulin molecules: interchain and intrachain disulfide bonds. As is well known in the art the location and number of interchain disulfide bonds vary according to the immunoglobulin class and species. While the invention is not limited to any particular class or subclass of antibody, the IgG1 immunoglobulin shall be used throughout the instant disclosure for illustrative purposes. In wild-type IgG1 molecules there are twelve intrachain disulfide bonds (four on each heavy chain and two on each light chain) and four interchain disulfide bonds. Intrachain disulfide bonds are generally somewhat protected and relatively less susceptible to reduction than interchain bonds. Conversely, interchain disulfide bonds are located on the surface of the immunoglobulin, are accessible to solvent and are usually relatively easy to reduce. Two interchain disulfide bonds exist between the heavy chains and one from each heavy chain to its respective light chain. It has been demonstrated that interchain disulfide bonds are not essential for chain association. The IgG1 hinge region contain the cysteines in the heavy chain that form the interchain disulfide bonds, which provide structural support along with the flexibility that facilitates Fab movement. The heavy/heavy IgG1 interchain disulfide bonds are located at residues C226 and C229 (Eu numbering) while the IgG1 interchain disulfide bond between the light and heavy chain of IgG1 (heavy/light) are formed between C214 of the kappa or lambda light chain and C220 in the upper hinge region of the heavy chain.

B. Antibody Generation and Production

Antibodies of the invention can be produced using a variety of methods known in the art.

1. Generation of Polyclonal Antibodies in Host Animals

The production of polyclonal antibodies in various host animals is well known in the art (see for example, Harlow and Lane (Eds.) (1988) Antibodies: A Laboratory Manual, CSH Press; and Harlow et al. (1989) Antibodies, NY, Cold Spring Harbor Press). In order to generate polyclonal antibodies, an immunocompetent animal (e.g., mouse, rat, rabbit, goat, non-human primate, etc.) is immunized with an antigenic protein or cells or preparations comprising an antigenic protein. After a period of time, polyclonal antibody-containing serum is obtained by bleeding or sacrificing the animal. The serum may be used in the form obtained from the animal or the antibodies may be partially or fully purified to provide immunoglobulin fractions or isolated antibody preparations.

Any form of antigen, or cells or preparations containing the antigen, can be used to generate an antibody that is specific for a determinant. The term “antigen” is used in a broad sense and may comprise any immunogenic fragment or determinant of the selected target including a single epitope, multiple epitopes, single or multiple domains or the entire extracellular domain (ECD). The antigen may be an isolated full-length protein, a cell surface protein (e.g., immunizing with cells expressing at least a portion of the antigen on their surface), or a soluble protein (e.g., immunizing with only the ECD portion of the protein). The antigen may be produced in a genetically modified cell. Any of the aforementioned antigens may be used alone or in combination with one or more immunogenicity enhancing adjuvants known in the art. The DNA encoding the antigen may be genomic or non-genomic (e.g., cDNA) and may encode at least a portion of the ECD, sufficient to elicit an immunogenic response. Any vector may be employed to transform the cells in which the antigen is expressed, including but not limited to adenoviral vectors, lentiviral vectors, plasmids, and non-viral vectors, such as cationic lipids.

2. Monoclonal Antibodies

In selected embodiments, the invention contemplates use of monoclonal antibodies. As known in the art, the term “monoclonal antibody” or “mAb” refers to an antibody obtained from a population of substantially homogeneous antibodies, i.e., the individual antibodies comprising the population are identical except for possible mutations (e.g., naturally occurring mutations), that may be present in minor amounts.

Monoclonal antibodies can be prepared using a wide variety of techniques known in the art including hybridoma techniques, recombinant techniques, phage display technologies, transgenic animals (e.g., a XenoMouse®) or some combination thereof. For example, monoclonal antibodies can be produced using hybridoma and biochemical and genetic engineering techniques such as described in more detail in An, Zhigiang (ed.) Therapeutic Monoclonal Antibodies: From Bench to Clinic, John Wiley and Sons, 1^{st} ed. 2009; Shire et. al. (eds.) Current Trends in Monoclonal Antibody Development and Manufacturing, Springer Science+Business Media LLC, 1^{st} ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N.Y., 1981). Following production of multiple monoclonal antibodies that bind specifically to a determinant, particularly effective antibodies may be selected through various screening processes, based on, for example, its affinity for the determinant or rate of internalization. Antibodies produced as described herein may be used as “source” antibodies and further modified to, for example, improve affinity for the target, improve its production in cell culture, reduce immunogenicity in vivo, create multispecific constructs, etc. A more detailed description of monoclonal antibody production and screening is set out below and in the appended Examples.

3. Human Antibodies

In another embodiment, the antibodies may comprise fully human antibodies. The term “human antibody” refers to an antibody which possesses an amino acid sequence that corresponds to that of an antibody produced by a human and/or has been made using any of the techniques for making human antibodies described below.

Human antibodies can be produced using various techniques known in the art. In one embodiment, recombinant human antibodies may be isolated by screening a recombinant combinatorial antibody library prepared using phage display. In one embodiment, the library is a scFv phage or yeast display library, generated using human VL and VH cDNAs prepared from mRNA isolated from B-cells. Such techniques advantageously allow for the screening of large numbers of candidate antibodies and provide for relatively easy manipulation of candidate sequences (e.g., by affinity maturation or recombinant shuffling).

Human antibodies can also be made by introducing human immunoglobulin loci into transgenic animals, e.g., mice in which the endogenous immunoglobulin genes have been partially or completely inactivated and human immunoglobulin genes have been introduced. Upon challenge, human antibody production is observed, which closely resembles that seen in humans in all respects, including gene rearrangement, assembly and fully human antibody repertoire. This approach is described, for example, in U.S. Pat. Nos. 5,545,807; 5,545,806; 5,569,825; 5,625,126; 5,633,425; 5,661,016, and U.S. Pat. Nos. 6,075,181 and 6,150,584 regarding XenoMouse® technology; and Lonberg and Huszar, 1995, PMID: 7494109). Alternatively, a human antibody may be prepared via immortalization of human B lymphocytes producing an antibody directed against a target antigen (such B lymphocytes may be recovered from an individual suffering from a neoplastic disorder or may have been immunized in vitro). See, e.g., Cole et al., Monoclonal Antibodies and Cancer Therapy, Alan R. Liss, p. 77 (1985); Boerner et al., 1991, PMID: 2051030; and U.S. Pat. No. 5,750,373. As with other monoclonal antibodies such human antibodies may be used as source antibodies.

Moreover, such antibodies may be produced recombinantly after selected host cells have been transformed or transfected with the genetic material (from any source including those described immediately above) encoding the desired human heavy and light chains. Such non-naturally occurring recombinantly manufactured fully human antibody compositions are explicitly within the scope of the instant invention and may be used to provide the disclose anti-DLL3 ADCs.

4. Derived Antibodies:

Once source antibodies have been generated, selected and isolated as described above they may be further altered to provide anti-DLL3 antibodies having improved pharmaceutical characteristics. Preferably the source antibodies are modified or altered using known molecular engineering techniques to provide derived antibodies having the desired therapeutic properties.

4.1. Chimeric and Humanized Antibodies

Selected embodiments of the invention comprise murine monoclonal antibodies that immunospecifically bind to DLL3 and which can be considered “source” antibodies. In certain embodiments, antibodies of the invention can be derived from such “source” antibodies through optional modification of the constant region and/or the epitope-binding amino acid sequences of the source antibody. In various embodiments an antibody is “derived” from a source antibody if selected amino acids in the source antibody are altered through deletion, mutation, substitution, integration or combination. In another embodiment, a “derived” antibody is one in which fragments of the source antibody (e.g., one or more CDRs or the entire heavy and light chain variable regions) are combined with or incorporated into an acceptor antibody sequence to provide the derivative antibody (e.g. chimeric or humanized antibodies). These “derived” antibodies can be generated using standard molecular biological techniques as described below, such as, for example, to improve affinity for the determinant; to improve antibody stability; to improve production and yield in cell culture; to reduce immunogenicity in vivo; to reduce toxicity; to facilitate conjugation of an active moiety; or to create a multispecific antibody. Such antibodies may also be derived from source antibodies through modification of the mature molecule (e.g., glycosylation patterns or pegylation) by chemical means or post-translational modification.

In one embodiment, the antibodies of the invention comprise chimeric antibodies that are derived from protein segments from at least two different species or class of antibodies that have been covalently joined. The term “chimeric” antibody is directed to constructs in which a portion of the heavy and/or light chain is identical or homologous to corresponding sequences in antibodies from a particular species or belonging to a particular antibody class or subclass, while the remainder of the chain(s) is identical or homologous to corresponding sequences in antibodies from another species or belonging to another antibody class or subclass, as well as fragments of such antibodies (U.S. Pat. No. 4,816,567; Morrison et al., 1984, PMID: 6436822). In some embodiments chimeric antibodies of the instant invention may comprise all or most of the selected murine heavy and light chain variable regions operably linked to human light and heavy chain constant regions. In other selected embodiments, chimeric anti-DLL3 antibodies may be “derived” from the mouse antibodies disclosed herein.

In other embodiments, chimeric antibodies of the invention are “CDR-grafted” antibodies, where the CDRs (as defined using Kabat, Chothia, McCallum, etc.) are derived from a particular species or belonging to a particular antibody class or subclass, while the remainder of the antibody is largely derived from an antibody from another species or belonging to another antibody class or subclass. For use in humans, one or more selected rodent CDRs (e.g., mouse CDRs) may be grafted into a human acceptor antibody, replacing one or more of the naturally occurring CDRs of the human antibody. These constructs generally have the advantages of providing full strength human antibody functions, e.g., complement dependent cytotoxicity (CDC) and antibody-dependent cell-mediated cytotoxicity (ADCC) while reducing unwanted immune responses to the antibody by the subject. In one embodiment the CDR grafted antibodies will comprise one or more CDRs obtained from a mouse incorporated in a human framework sequence.

Similar to the CDR-grafted antibody is a “humanized” antibody. As used herein, a “humanized” antibody is a human antibody (acceptor antibody) comprising one or more amino acid sequences (e.g. CDR sequences) derived from one or more non-human antibodies (donor or source antibody). In certain embodiments, “back mutations” can be introduced into the humanized antibody, in which residues in one or more framework domains (FRs) of the variable region of the recipient human antibody are replaced by corresponding residues from the non-human species donor antibody. Such back mutations may to help maintain the appropriate three-dimensional configuration of the grafted CDR(s) and thereby improve affinity and antibody stability. Antibodies from various donor species may be used including, without limitation, mouse, rat, rabbit, or non-human primate. Furthermore, humanized antibodies may comprise new residues that are not found in the recipient antibody or in the donor antibody to, for example, further refine antibody performance. CDR grafted and humanized antibodies compatible with the instant invention comprising murine components from source antibodies and human components from acceptor antibodies are provided as set forth in the Examples below.

Various art-recognized techniques can be used to determine which human sequences to use as acceptor antibodies to provide humanized constructs in accordance with the instant invention. Compilations of compatible human germline sequences and methods of determining their suitability as acceptor sequences are disclosed, for example, in Dubel and Reichert (Eds.) (2014) Handbook of Therapeutic Antibodies, 2^{nd} Edition, Wiley-Blackwell GmbH; Tomlinson, I. A. et al. (1992) J. Mol. Biol. 227:776-798; Cook, G. P. et al. (1995) Immunol. Today 16: 237-242; Chothia, D. et al. (1992) J. Mol. Biol. 227:799-817; and Tomlinson et al. (1995) EMBO J 14:4628-4638). The V-BASE directory (VBASE2—Retter et al., Nucleic Acid Res. 33; 671-674, 2005) which provides a comprehensive directory of human immunoglobulin variable region sequences (compiled by Tomlinson, I. A. et al. MRC Centre for Protein Engineering, Cambridge, UK) may also be used to identify compatible acceptor sequences. Additionally, consensus human framework sequences described, for example, in U.S. Pat. No. 6,300,064 may also prove to be compatible acceptor sequences are can be used in accordance with the instant teachings. In general, human framework acceptor sequences are selected based on homology with the murine source framework sequences along with an analysis of the CDR canonical structures of the source and acceptor antibodies. The derived sequences of the heavy and light chain variable regions of the derived antibody may then be synthesized using art recognized techniques.

By way of example CDR grafted and humanized antibodies, and associated methods, are described in U.S. Pat. Nos. 6,180,370 and 5,693,762. For further details, see, e.g., Jones et al., 1986, (PMID: 3713831); and U.S. Pat. Nos. 6,982,321 and 7,087,409.

The sequence identity or homology of the CDR grafted or humanized antibody variable region to the human acceptor variable region may be determined as discussed herein and, when measured as such, will preferably share at least 60% or 65% sequence identity, more preferably at least 70%, 75%, 80%, 85%, or 90% sequence identity, even more preferably at least 93%, 95%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution.

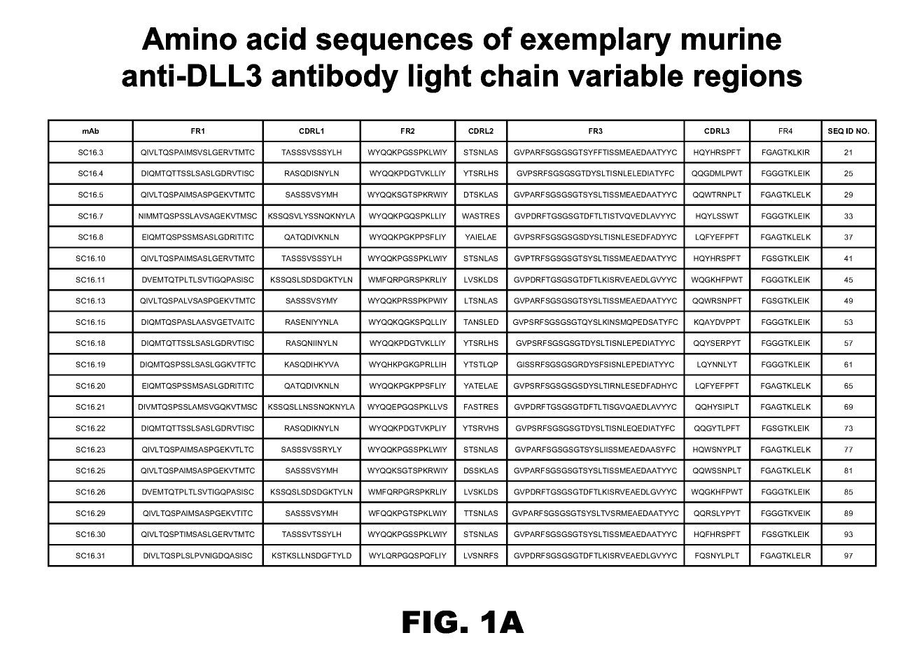

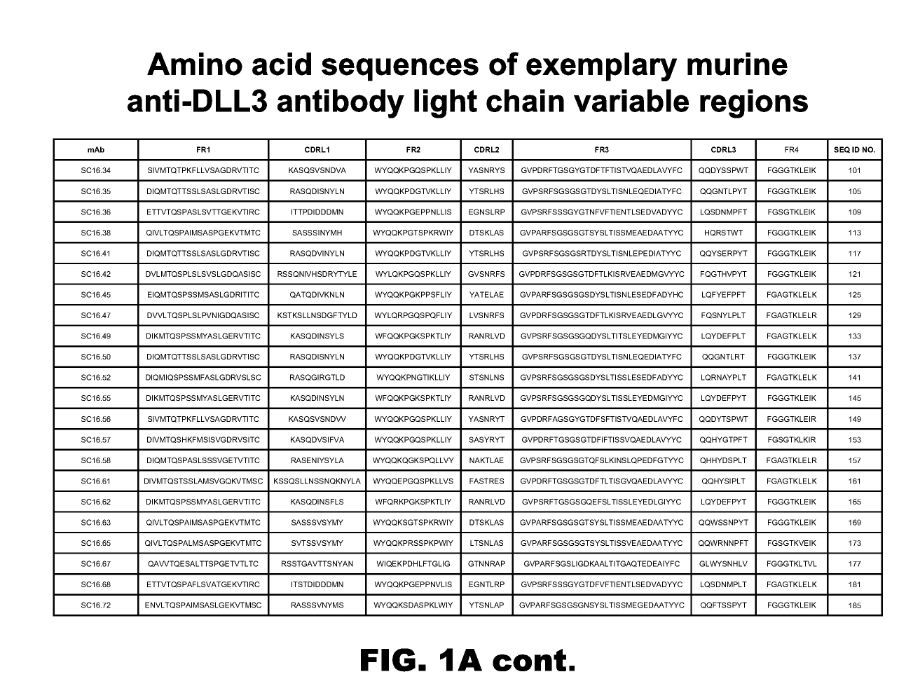

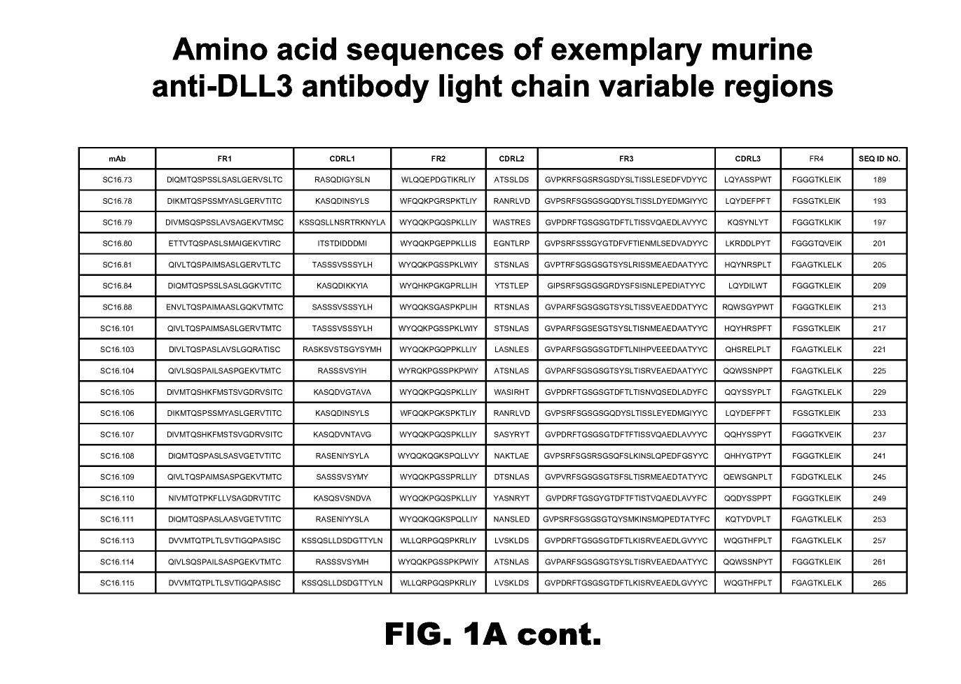

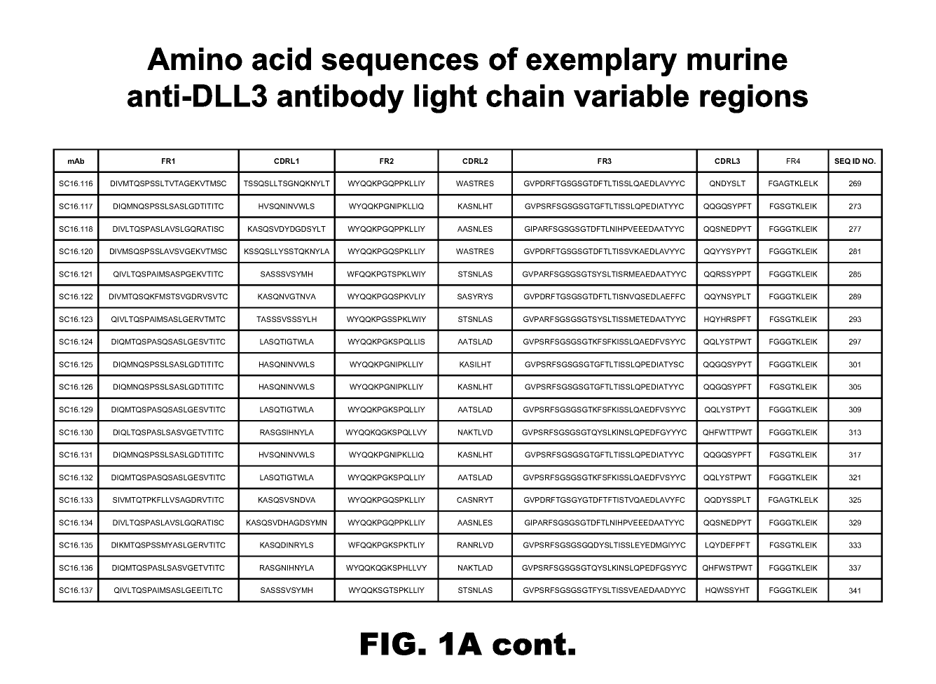

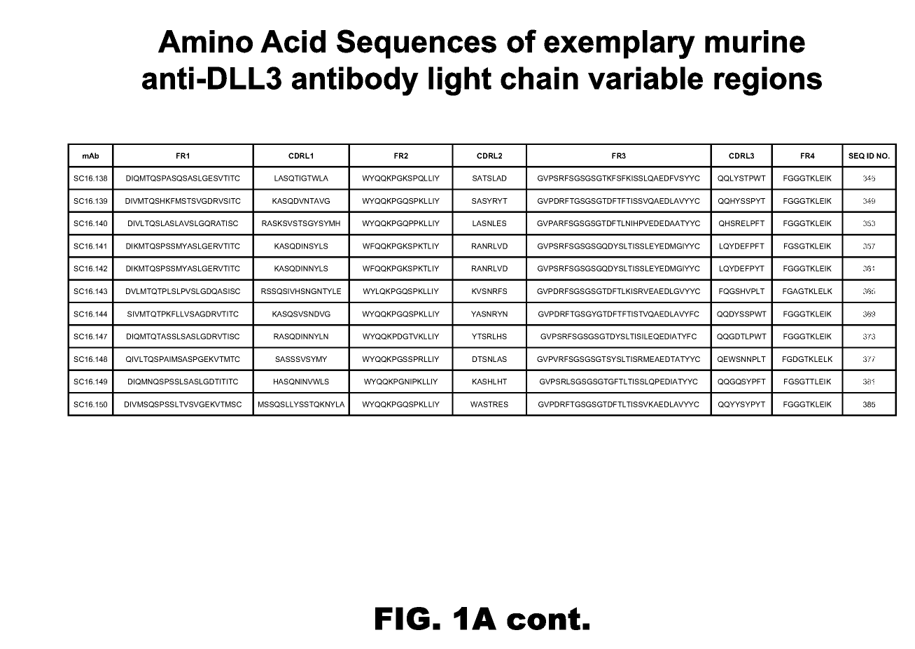

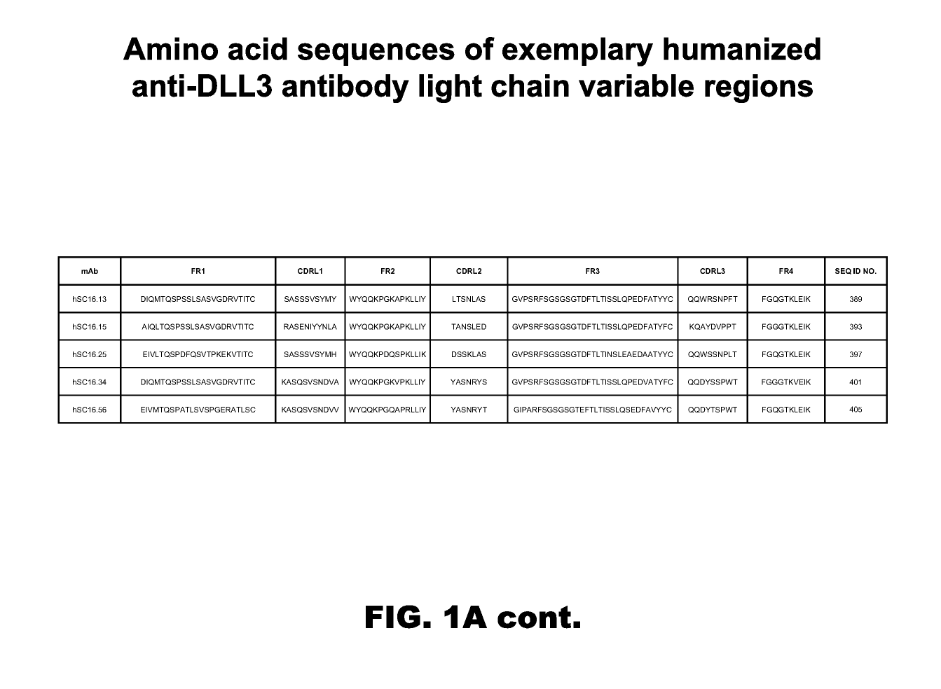

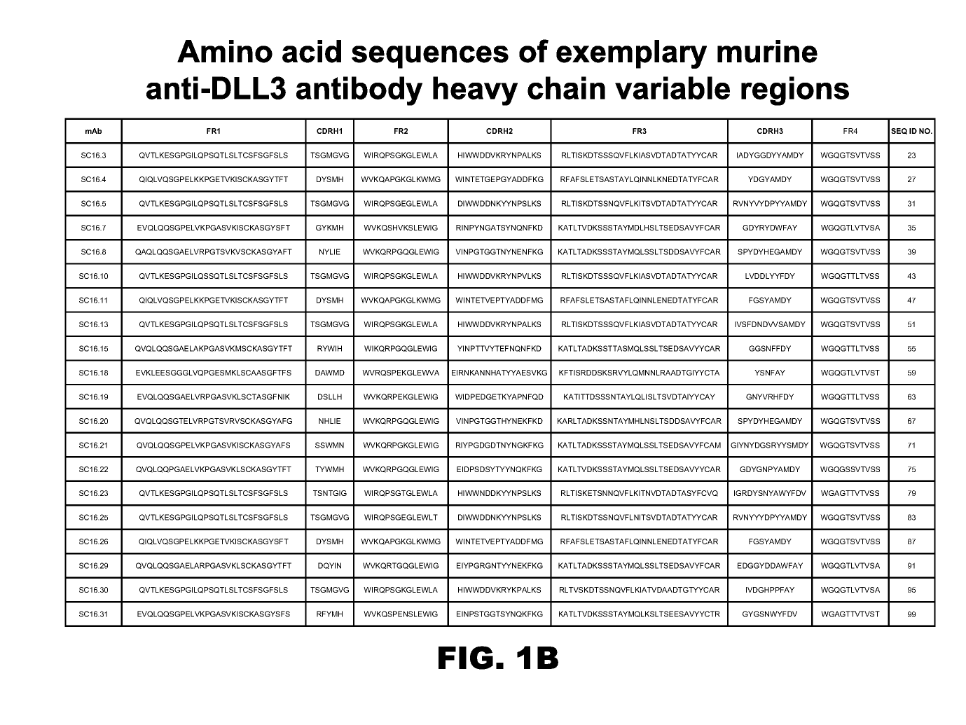

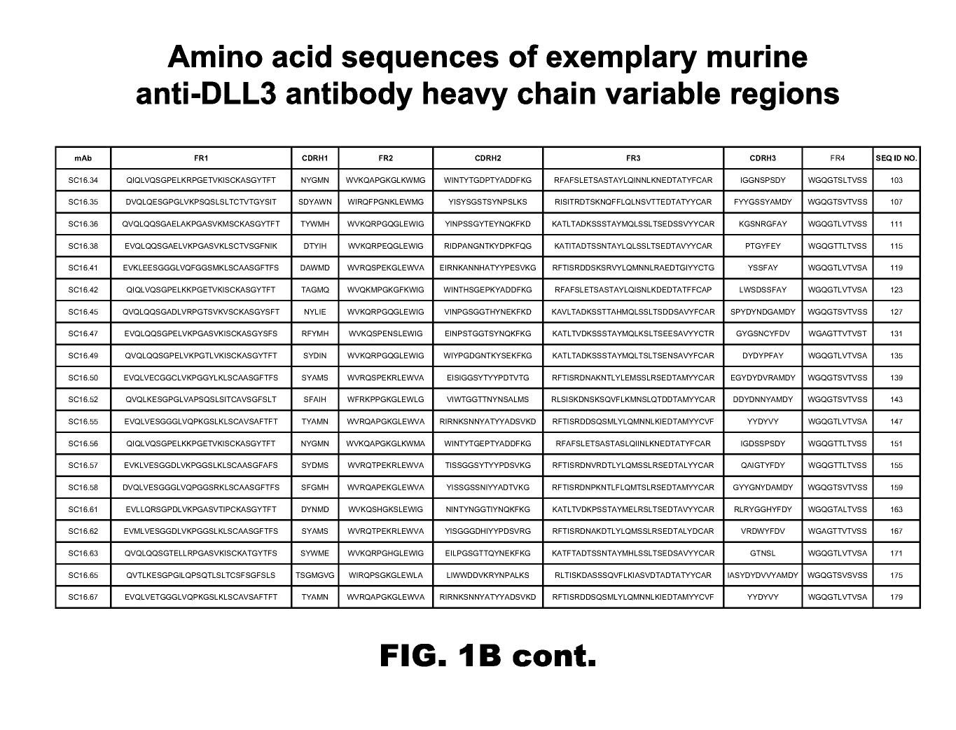

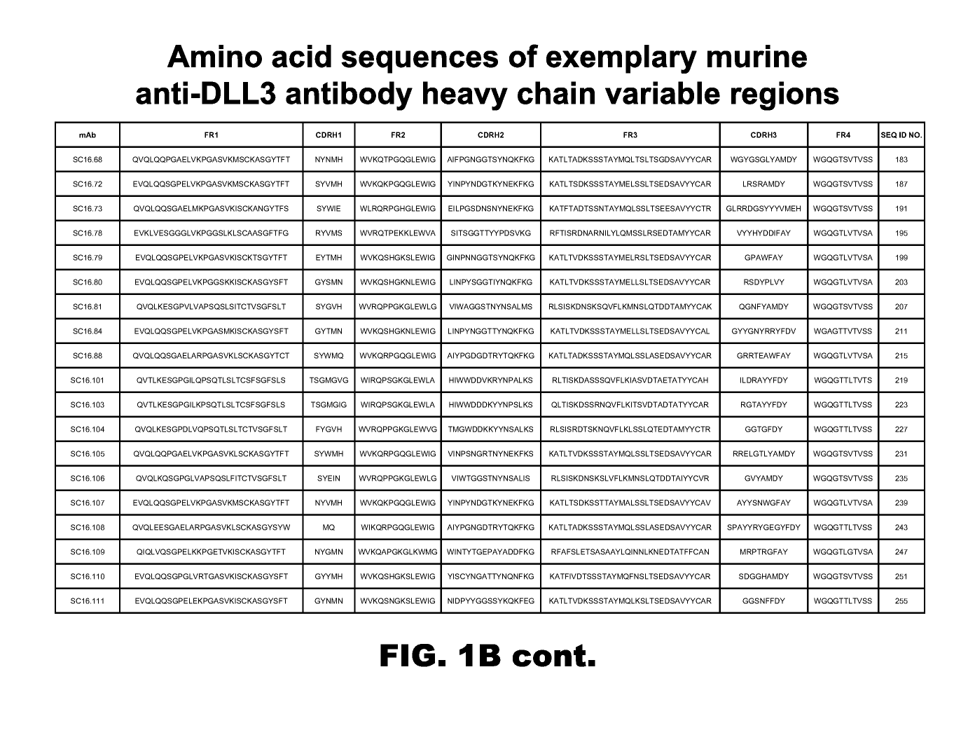

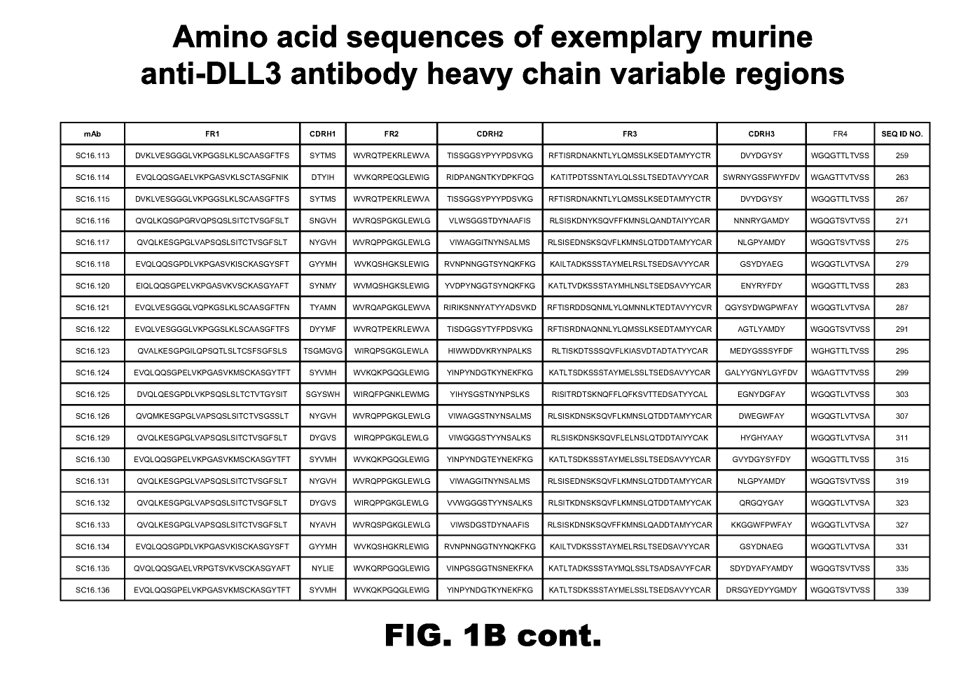

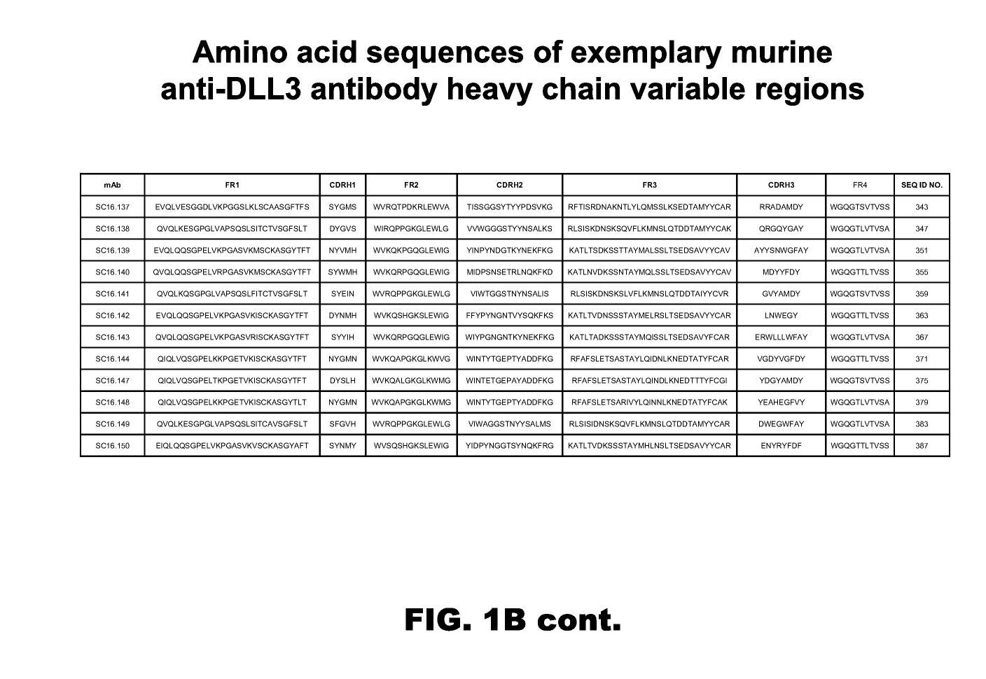

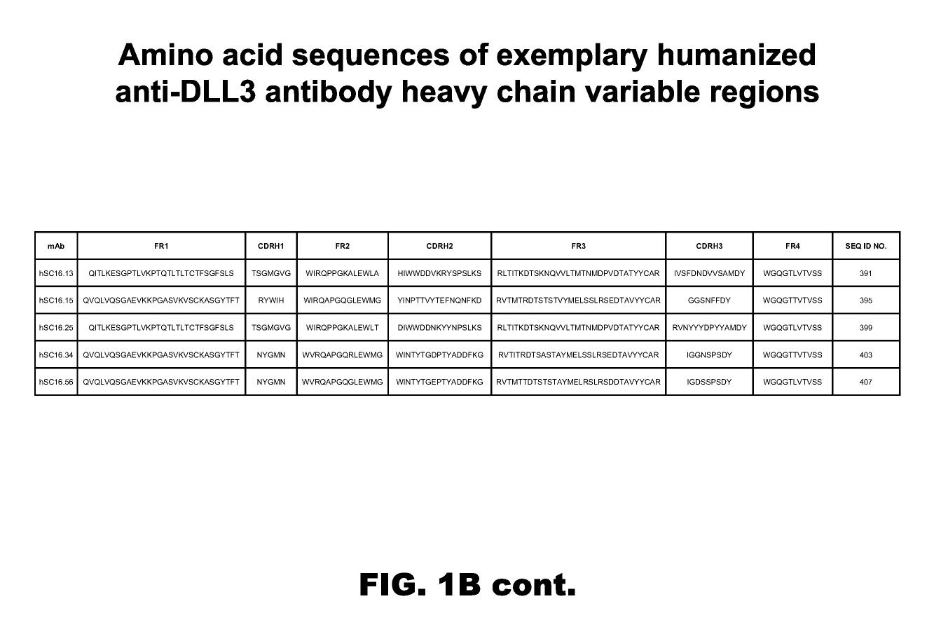

It will be appreciated that the annotated CDRs and framework sequences as provided in the appended FIGS. 1A and 1B are defined as per Kabat et al. using a proprietary Abysis database. However, as discussed herein one skilled in the art could readily identify CDRs in accordance with definitions provided by Chothia et al., ABM or MacCallum et al as well as Kabat et al. As such, anti-DLL3 humanized antibodies comprising one or more CDRs derived according to any of the aforementioned systems are explicitly held to be within the scope of the instant invention.

4.2. Site-Specific Antibodies

The antibodies of the instant invention may be engineered to facilitate conjugation to a cytotoxin or other anti-cancer agent (as discussed in more detail below). It is advantageous for the antibody drug conjugate preparation to comprise a homogenous population of ADC molecules in terms of the position of the cytotoxin on the antibody and the drug to antibody ratio (DAR). Based on the instant disclosure one skilled in the art could readily fabricate site-specific engineered constructs as described herein. As used herein a “site-specific antibody” or “site-specific construct” means an antibody, or immunoreactive fragment thereof, wherein at least one amino acid in either the heavy or light chain is deleted, altered or substituted (preferably with another amino acid) to provide at least one free cysteine. Similarly, a “site-specific conjugate” shall be held to mean an ADC comprising a site-specific antibody and at least one cytotoxin or other compound conjugated to the unpaired or free cysteine(s). In certain embodiments the unpaired cysteine residue will comprise an unpaired intrachain residue. In other embodiments the free cysteine residue will comprise an unpaired interchain cysteine residue. In still other embodiments the free cysteine may be engineered into the amino acid sequence of the antibody (e.g., in the CH3 domain). In any event the site-specific antibody can be of various isotypes, for example, IgG, IgE, IgA or IgD; and within those classes the antibody can be of various subclasses, for example, IgG1, IgG2, IgG3 or IgG4. For IgG constructs the light chain of the antibody can comprise either a kappa or lambda isotype each incorporating a C214 that, in selected embodiments, may be unpaired due to a lack of a C220 residue in the IgG1 heavy chain.

Thus, as used herein, the terms “free cysteine” or “unpaired cysteine” may be used interchangeably unless otherwise dictated by context and shall mean any cysteine (or thiol containing) constituent of an antibody, whether naturally present or specifically incorporated in a selected residue position using molecular engineering techniques. In certain selected embodiments the free cysteine may comprise a naturally occurring cysteine whose native interchain or intrachain disulfide bridge partner has been substituted, eliminated or otherwise altered to disrupt the naturally occurring disulfide bridge under physiological conditions thereby rendering the unpaired cysteine suitable for site-specific conjugation. In other preferred embodiments the free or unpaired cysteine will comprise a cysteine residue that is selectively placed at a predetermined site within the antibody heavy or light chain amino acid sequences. It will be appreciated that, prior to conjugation, free or unpaired cysteines may be present as a thiol (reduced cysteine), as a capped cysteine (oxidized) or as a non-natural intramolecular disulfide bond (oxidized) with another free cysteine on the same antibody depending on the oxidation state of the system. As discussed in more detail below, mild reduction of this antibody construct will provide thiols available for site-specific conjugation. In particularly preferred embodiments the free or unpaired cysteines (whether naturally occurring or incorporated) will be subject to selective reduction and subsequent conjugation to provide homogenous DAR compositions.

It will be appreciated that the favorable properties exhibited by the disclosed engineered conjugate preparations is predicated, at least in part, on the ability to specifically direct the conjugation and largely limit the fabricated conjugates in terms of conjugation position and absolute DAR of the composition. Unlike most conventional ADC preparations the present invention need not rely entirely on partial or total reduction of the antibody to provide random conjugation sites and relatively uncontrolled generation of DAR species. Rather, in certain aspects the present invention preferably provides one or more predetermined unpaired (or free) cysteine sites by engineering the targeting antibody to disrupt one or more of the naturally occurring (i.e., “native”) interchain or intrachain disulfide bridges or to introduce a cysteine residue at any position. To this end it will be appreciated that, in selected embodiments, a cysteine residue may be incorporated anywhere along the antibody (or immunoreactive fragment thereof) heavy or light chain or appended thereto using standard molecular engineering techniques. In other preferred embodiments disruption of native disulfide bonds may be effected in combination with the introduction of a non-native cysteine (which will then comprise a free cysteine) that may then be used as a conjugation site.

In one embodiment the engineered antibody comprises at least one amino acid deletion or substitution of an intrachain or interchain cysteine residue. As used herein “interchain cysteine residue” means a cysteine residue that is involved in a native disulfide bond either between the light and heavy chain of an antibody or between the two heavy chains of an antibody while an “intrachain cysteine residue” is one naturally paired with another cysteine in the same heavy or light chain. In one embodiment the deleted or substituted interchain cysteine residue is involved in the formation of a disulfide bond between the light and heavy chain. In another embodiment the deleted or substituted cysteine residue is involved in a disulfide bond between the two heavy chains. In a typical embodiment, due to the complementary structure of an antibody, in which the light chain is paired with the VH and CH1 domains of the heavy chain and wherein the CH2 and CH3 domains of one heavy chain are paired with the CH2 and CH3 domains of the complementary heavy chain, a mutation or deletion of a single cysteine in either the light chain or in the heavy chain would result in two unpaired cysteine residues in the engineered antibody.

In some embodiments an interchain cysteine residue is deleted. In other embodiments an interchain cysteine is substituted for another amino acid (e.g., a naturally occurring amino acid). For example, the amino acid substitution can result in the replacement of an interchain cysteine with a neutral (e.g. serine, threonine or glycine) or hydrophilic (e.g. methionine, alanine, valine, leucine or isoleucine) residue. In one embodiment an interchain cysteine is replaced with a serine.

In some embodiments contemplated by the invention the deleted or substituted cysteine residue is on the light chain (either kappa or lambda) thereby leaving a free cysteine on the heavy chain. In other embodiments the deleted or substituted cysteine residue is on the heavy chain leaving the free cysteine on the light chain constant region. Upon assembly it will be appreciated that deletion or substitution of a single cysteine in either the light or heavy chain of an intact antibody results in a site-specific antibody having two unpaired cysteine residues.

In one embodiment the cysteine at position 214 (C214) of the IgG light chain (kappa or lambda) is deleted or substituted. In another embodiment the cysteine at position 220 (C220) on the IgG heavy chain is deleted or substituted. In further embodiments the cysteine at position 226 or position 229 on the heavy chain is deleted or substituted. In one embodiment C220 on the heavy chain is substituted with serine (C220S) to provide the desired free cysteine in the light chain. In another embodiment C214 in the light chain is substituted with serine (C214S) to provide the desired free cysteine in the heavy chain. Such site-specific constructs provided in Example 15. A summary of these constructs is shown in Table 2 immediately below where numbering is generally according to the Eu index as set forth in Kabat and WT stands for “wild-type” or native constant region sequences without alterations and delta (Δ) designates the deletion of an amino acid residue (e.g., C214Δ indicates that the cysteine at position 214 has been deleted).

TABLE 2

| | Antibody | |
|---|---|---|
| Designation | Component | Alteration |
| ss1 | Heavy Chain | C220S |
| | Light Chain | WT |
| ss2 | Heavy Chain | C220Δ |
| | Light Chain | WT |
| ss3 | Heavy Chain | WT |
| | Light Chain | C214Δ |
| ss4 | Heavy Chain | WT |
| | Light Chain | C214S |

In this regard and for the purposes of illustration the full length heavy (SEQ ID NO: 9) and light (SEQ ID NO: 8) chain amino acid sequences for the exemplary antibody hSC16.56ss1 are set forth immediately below. Note that the kappa light chain is identical to the light chain found in the hSC16.56 antibody while the hSC16.56ss1 heavy chain (SEQ ID NO: 9) is different from the heavy chain in the hSC16.56 antibody (SEQ ID NO: 7) in that it contains the C220S mutation which is underlined and bolded in the sequence below.

(SEQ ID NO: 9)
QVQLVQSGAEVKKPGASVKVSCKASGYTFTNYGMNWVRQAPGQGLEWM
GWINTYTGEPTYADDFKGRVTMTTDTSTSTAYMELRSLRSDDTAVYYC
ARIGDSSPSDYWGQGTLVTVSSASTKGPSVFPLAPSSKSTSGGTAALG
CLVKDYFPEPVTVSWNSGALTSGVHTFPAVLQSSGLYSLSSVVTVPSS
SLGTQTYICNVNHKPSNTKVDKKVEPKSSDKTHTCPPCPAPELLGGPS
VFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEA
LHNHYTQKSLSLSPG.
(SEQ ID NO: 8)
EIVMTQSPATLSVSPGERATLSCKASQSVSNDVVWYQQKPGQAPRLLI
YYASNRYTGIPARFSGSGSGTEFTLTISSLQSEDFAVYYCQQDYTSPW
TFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC.

With regard to the introduction or addition of a cysteine residue or residues to provide a free cysteine (as opposed to disrupting a native disulfide bond) compatible position(s) on the antibody or antibody fragment may readily be discerned by one skilled in the art. Accordingly, in selected embodiments the cysteine(s) may be introduced in the CH1 domain, the CH2 domain or the CH3 domain or any combination thereof depending on the desired DAR, the antibody construct, the selected payload and the antibody target. In other preferred embodiments the cysteines may be introduced into a kappa or lambda CL domain and, in particularly preferred embodiments, in the c-terminal region of the CL domain. In each case other amino acid residues proximal to the site of cysteine insertion may be altered, removed or substituted to facilitate molecular stability, conjugation efficiency or provide a protective environment for the payload once it is attached. In particular embodiments, the substituted residues occur at any accessible sites of the antibody. By substituting such surface residues with cysteine, reactive thiol groups are thereby positioned at readily accessible sites on the antibody and may be selectively reduced as described further herein. In particular embodiments, the substituted residues occur at accessible sites of the antibody. By substituting those residues with cysteine, reactive thiol groups are thereby positioned at accessible sites of the antibody and may be used to selectively conjugate the antibody. In certain embodiments, any one or more of the following residues may be substituted with cysteine: V205 (Kabat numbering) of the light chain; A118 (Eu numbering) of the heavy chain; and S400 (Eu numbering) of the heavy chain Fc region. Additional substitution positions and methods of fabricating compatible site-specific antibodies are set forth in U.S. Pat. No. 7,521,541 which is incorporated herein in its entirety.

The strategy for generating antibody-drug conjugates with defined sites and stoichiometries of drug loading, as disclosed herein, is broadly applicable to all anti-DLL3 antibodies as it primarily involves engineering of the conserved constant domains of the antibody. As the amino acid sequences and native disulfide bridges of each class and subclass of antibody are well documented, one skilled in the art could readily fabricate engineered constructs of various DLL3 antibodies without undue experimentation and, accordingly, such constructs are expressly contemplated as being within the scope of the instant invention. This is particularly true of site-specific constructs comprising heavy and light chain variable region amino acid sequences as set forth in the instant disclosure.

4.3. Constant Region Modifications and Altered Glycosylation

Selected embodiments of the present invention may also comprise substitutions or modifications of the constant region (i.e. the Fc region), including without limitation, amino acid residue substitutions, mutations and/or modifications, which result in a compound with characteristics including, but not limited to: altered pharmacokinetics, increased serum half-life, increase binding affinity, reduced immunogenicity, increased production, altered Fc ligand binding to an Fc receptor (FcR), enhanced or reduced ADCC or CDC, altered glycosylation and/or disulfide bonds and modified binding specificity.

Compounds with improved Fc effector functions can be generated, for example, through changes in amino acid residues involved in the interaction between the Fc domain and an Fc receptor (e.g., FcγRI, FcγRIIA and B, FcγRIII and FcRn), which may lead to increased cytotoxicity and/or altered pharmacokinetics, such as increased serum half-life (see, for example, Ravetch and Kinet, Annu. Rev. Immunol 9:457-92 (1991); Capel et al., Immunomethods 4:25-34 (1994); and de Haas et al., J. Lab. Clin. Med. 126:330-41 (1995).

In selected embodiments, antibodies with increased in vivo half-lives can be generated by modifying (e.g., substituting, deleting or adding) amino acid residues identified as involved in the interaction between the Fc domain and the FcRn receptor (see, e.g., International Publication Nos. WO 97/34631; WO 04/029207; U.S. Pat. No. 6,737,056 and U.S.P.N. 2003/0190311). With regard to such embodiments, Fc variants may provide half-lives in a mammal, preferably a human, of greater than 5 days, greater than 10 days, greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-life results in a higher serum titer which thus reduces the frequency of the administration of the antibodies and/or reduces the concentration of the antibodies to be administered. Binding to human FcRn in vivo and serum half-life of human FcRn high affinity binding polypeptides can be assayed, e.g., in transgenic mice or transfected human cell lines expressing human FcRn, or in primates to which the polypeptides with a variant Fc region are administered. WO 2000/42072 describes antibody variants with improved or diminished binding to FcRns. See also, e.g., Shields et al. J. Biol. Chem. 9(2):6591-6604 (2001). Surprisingly, certain ADCs of the instant invention exhibit protracted terminal half-lives (e.g., on the order of two weeks) without any antibody constant region modifications other than those used to provide optional site-specific conjugates.

In other embodiments, Fc alterations may lead to enhanced or reduced ADCC or CDC activity. As in known in the art, CDC refers to the lysing of a target cell in the presence of complement, and ADCC refers to a form of cytotoxicity in which secreted Ig bound onto FcRs present on certain cytotoxic cells (e.g., Natural Killer cells, neutrophils, and macrophages) enables these cytotoxic effector cells to bind specifically to an antigen-bearing target cell and subsequently kill the target cell with cytotoxins. In the context of the instant invention antibody variants are provided with “altered” FcR binding affinity, which is either enhanced or diminished binding as compared to a parent or unmodified antibody or to an antibody comprising a native sequence FcR. Such variants which display decreased binding may possess little or no appreciable binding, e.g., 0-20% binding to the FcR compared to a native sequence, e.g. as determined by techniques well known in the art. In other embodiments the variant will exhibit enhanced binding as compared to the native immunoglobulin Fc domain. It will be appreciated that these types of Fc variants may advantageously be used to enhance the effective anti-neoplastic properties of the disclosed antibodies. In yet other embodiments, such alterations lead to increased binding affinity, reduced immunogenicity, increased production, altered glycosylation and/or disulfide bonds (e.g., for conjugation sites), modified binding specificity, increased phagocytosis; and/or down regulation of cell surface receptors (e.g. B cell receptor; BCR), etc.

Still other embodiments comprise one or more engineered glycoforms, e.g., a site-specific antibody comprising an altered glycosylation pattern or altered carbohydrate composition that is covalently attached to the protein (e.g., in the Fc domain). See, for example, Shields, R. L. et al. (2002) J. Biol. Chem. 277:26733-26740. Engineered glycoforms may be useful for a variety of purposes, including but not limited to enhancing or reducing effector function, increasing the affinity of the antibody for a target or facilitating production of the antibody. In certain embodiments where reduced effector function is desired, the molecule may be engineered to express an aglycosylated form. Substitutions that may result in elimination of one or more variable region framework glycosylation sites to thereby eliminate glycosylation at that site are well known (see e.g. U.S. Pat. Nos. 5,714,350 and 6,350,861). Conversely, enhanced effector functions or improved binding may be imparted to the Fc containing molecule by engineering in one or more additional glycosylation sites.

Other embodiments include an Fc variant that has an altered glycosylation composition, such as a hypofucosylated antibody having reduced amounts of fucosyl residues or an antibody having increased bisecting GlcNAc structures. Such altered glycosylation patterns have been demonstrated to increase the ADCC ability of antibodies. Engineered glycoforms may be generated by any method known to one skilled in the art, for example by using engineered or variant expression strains, by co-expression with one or more enzymes (for example N-acetylglucosaminyltransferase III (GnTIII)), by expressing a molecule comprising an Fc region in various organisms or cell lines from various organisms or by modifying carbohydrate(s) after the molecule comprising Fc region has been expressed (see, for example, WO 2012/117002).

4.4. Fragments

Regardless of which form of antibody (e.g. chimeric, humanized, etc.) is selected to practice the invention it will be appreciated that immunoreactive fragments, either by themselves or as part of an antibody drug conjugate, of the same may be used in accordance with the teachings herein. An “antibody fragment” comprises at least a portion of an intact antibody. As used herein, the term “fragment” of an antibody molecule includes antigen-binding fragments of antibodies, and the term “antigen-binding fragment” refers to a polypeptide fragment of an immunoglobulin or antibody that immunospecifically binds or reacts with a selected antigen or immunogenic determinant thereof or competes with the intact antibody from which the fragments were derived for specific antigen binding.

Exemplary site-specific fragments include: variable light chain fragments (VL), an variable heavy chain fragments (VH), scFv, F(ab′)2 fragment, Fab fragment, Fd fragment, Fv fragment, single domain antibody fragments, diabodies, linear antibodies, single-chain antibody molecules and multispecific antibodies formed from antibody fragments. In addition, an active site-specific fragment comprises a portion of the antibody that retains its ability to interact with the antigen/substrates or receptors and modify them in a manner similar to that of an intact antibody (though maybe with somewhat less efficiency). Such antibody fragments may further be engineered to comprise one or more free cysteines as described herein.

In other embodiments, an antibody fragment is one that comprises the Fc region and that retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may comprise an antigen binding arm linked to an Fc sequence comprising at least one free cysteine capable of conferring in vivo stability to the fragment.

As would be well recognized by those skilled in the art, fragments can be obtained by molecular engineering or via chemical or enzymatic treatment (such as papain or pepsin) of an intact or complete antibody or antibody chain or by recombinant means. See, e.g., Fundamental Immunology, W. E. Paul, ed., Raven Press, N.Y. (1999), for a more detailed description of antibody fragments.

4.5. Multivalent Constructs

In other embodiments, the antibodies and conjugates of the invention may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen). See, for example, U.S.P.N. 2009/0130105.

In one embodiment, the antibodies are bispecific antibodies in which the two chains have different specificities, as described in Millstein et al., 1983, Nature, 305:537-539. Other embodiments include antibodies with additional specificities such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

Multivalent antibodies may immunospecifically bind to different epitopes of the desired target molecule or may immunospecifically bind to both the target molecule as well as a heterologous epitope, such as a heterologous polypeptide or solid support material. While selected embodiments may only bind two antigens (i.e. bispecific antibodies), antibodies with additional specificities such as trispecific antibodies are also encompassed by the instant invention. Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

5. Recombinant Production of Antibodies

Antibodies and fragments thereof may be produced or modified using genetic material obtained from antibody producing cells and recombinant technology (see, for example; Dubel and Reichert (Eds.) (2014) Handbook of Therapeutic Antibodies, 2^{nd} Edition, Wiley-Blackwell GmbH; Sambrook and Russell (Eds.) (2000) Molecular Cloning: A Laboratory Manual (3^{rd} Ed.), NY, Cold Spring Harbor Laboratory Press; Ausubel et al. (2002) Short Protocols in Molecular Biology: A Compendium of Methods from Current Protocols in Molecular Biology, Wiley, John & Sons, Inc.; and U.S. Pat. No. 7,709,611).

Another aspect of the invention pertains to nucleic acid molecules that encode the antibodies of the invention. The nucleic acids may be present in whole cells, in a cell lysate, or in a partially purified or substantially pure form. A nucleic acid is “isolated” or rendered substantially pure when separated from other cellular components or other contaminants, e.g., other cellular nucleic acids or proteins, by standard techniques, including alkaline/SDS treatment, CsCl banding, column chromatography, agarose gel electrophoresis and others well known in the art. A nucleic acid of the invention can be, for example, DNA (e.g. genomic DNA, cDNA), RNA and artificial variants thereof (e.g., peptide nucleic acids), whether single-stranded or double-stranded or RNA, RNA and may or may not contain introns. In selected embodiments the nucleic acid is a cDNA molecule.

Nucleic acids of the invention can be obtained using standard molecular biology techniques. For antibodies expressed by hybridomas (e.g., hybridomas prepared as described in the Examples below), cDNAs encoding the light and heavy chains of the antibody can be obtained by standard PCR amplification or cDNA cloning techniques. For antibodies obtained from an immunoglobulin gene library (e.g., using phage display techniques), nucleic acid encoding the antibody can be recovered from the library.

DNA fragments encoding VH and VL segments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein or protein fragment, such as an antibody constant region or a flexible linker. The term “operatively linked”, as used in this context, means that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3 in the case of IgG1). The sequences of human heavy chain constant region genes are known in the art (see e.g., Kabat, et al. (1991) (supra)) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. An exemplary IgG1 constant region is set forth in SEQ ID NO: 2. For a Fab fragment heavy chain gene, the VH-encoding DNA can be operatively linked to another DNA molecule encoding only the heavy chain CH1 constant region.

Isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, et al. (1991) (supra)) and DNA fragments encompassing these regions can be obtained by standard PCR amplification. The light chain constant region can be a kappa or lambda constant region, but most preferably is a kappa constant region. An exemplary compatible kappa light chain constant region is set forth in SEQ ID NO: 5.

Contemplated herein are certain polypeptides (e.g. antigens or antibodies) that exhibit “sequence identity”, sequence similarity” or “sequence homology” to the polypeptides of the invention. For example, a derived humanized antibody VH or VL domain may exhibit a sequence similarity with the source (e.g., murine) or acceptor (e.g., human) VH or VL domain. A “homologous” polypeptide may exhibit 65%, 70%, 75%, 80%, 85%, or 90% sequence identity. In other embodiments a “homologous” polypeptides may exhibit 93%, 95% or 98% sequence identity. As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

Residue positions which are not identical may differ by conservative amino acid substitutions or by non-conservative amino acid substitutions. A “conservative amino acid substitution” is one in which an amino acid residue is substituted by another amino acid residue having a side chain with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. In cases where there is a substitution with a non-conservative amino acid, in embodiments the polypeptide exhibiting sequence identity will retain the desired function or activity of the polypeptide of the invention (e.g., antibody.)

Also contemplated herein are nucleic acids that that exhibit “sequence identity”, sequence similarity” or “sequence homology” to the nucleic acids of the invention. A “homologous sequence” means a sequence of nucleic acid molecules exhibiting at least about 65%, 70%, 75%, 80%, 85%, or 90% sequence identity. In other embodiments, a “homologous sequence” of nucleic acids may exhibit 93%, 95% or 98% sequence identity to the reference nucleic acid.

The instant invention also provides vectors comprising such nucleic acids described above, which may be operably linked to a promoter (see, e.g., WO 86/05807; WO 89/01036; and U.S. Pat. No. 5,122,464); and other transcriptional regulatory and processing control elements of the eukaryotic secretory pathway. The invention also provides host cells harboring those vectors and host-expression systems.

As used herein, the term “host-expression system” includes any type of cellular system that can be engineered to generate either the nucleic acids or the polypeptides and antibodies of the invention. Such host-expression systems include, but are not limited to microorganisms (e.g., E. coli or B. subtilis) transformed or transfected with recombinant bacteriophage DNA or plasmid DNA; yeast (e.g., Saccharomyces) transfected with recombinant yeast expression vectors; or mammalian cells (e.g., COS, CHO-S, HEK293T, 3T3 cells) harboring recombinant expression constructs containing promoters derived from the genome of mammalian cells or viruses (e.g., the adenovirus late promoter). The host cell may be co-transfected with two expression vectors, for example, the first vector encoding a heavy chain derived polypeptide and the second vector encoding a light chain derived polypeptide. In particularly preferred aspects of the invention the disclosed antibodies will be produced using engineered CHO cells.

Methods of transforming mammalian cells are well known in the art. See, for example, U.S. Pat. Nos. 4,399,216, 4,912,040, 4,740,461, and 4,959,455. The host cell may also be engineered to allow the production of an antigen binding molecule with various characteristics (e.g. modified glycoforms or proteins having GnTIII activity).

For long-term, high-yield production of recombinant proteins stable expression is preferred. Accordingly, cell lines that stably express the selected antibody may be engineered using standard art recognized techniques and form part of the invention. Rather than using expression vectors that contain viral origins of replication, host cells can be transformed with DNA controlled by appropriate expression control elements (e.g., promoter or enhancer sequences, transcription terminators, polyadenylation sites, etc.), and a selectable marker. Any of the selection systems well known in the art may be used, including the glutamine synthetase gene expression system (the GS system) which provides an efficient approach for enhancing expression under selected conditions. The GS system is discussed in whole or part in connection with EP 0 216 846, EP 0 256 055, EP 0 323 997 and EP 0 338 841 and U.S. Pat. Nos. 5,591,639 and 5,879,936. Another compatible expression system for the development of stable cell lines is the Freedom™ CHO-S Kit (Life Technologies).

Once an antibody of the invention has been produced by recombinant expression or any other of the disclosed techniques, it may be purified or isolated by methods known in the art in that it is identified and separated and/or recovered from its natural environment and separated from contaminants that would interfere with diagnostic or therapeutic uses for the antibody or related ADC. Isolated antibodies include antibodies in situ within recombinant cells.

These isolated preparations may be purified using various art-recognized techniques, such as, for example, ion exchange and size exclusion chromatography, dialysis, diafiltration, and affinity chromatography, particularly Protein A or Protein G affinity chromatography. Compatible methods are discussed more fully in the Examples below.

6. Post-Production Selection

No matter how obtained, antibody-producing cells (e.g., hybridomas, yeast colonies, etc.) may be selected, cloned and further screened for desirable characteristics including, for example, robust growth, high antibody production and desirable antibody characteristics such as high affinity for the antigen of interest. Hybridomas can be expanded in vitro in cell culture or in vivo in syngeneic immunocompromised animals. Methods of selecting, cloning and expanding hybridomas and/or colonies are well known to those of ordinary skill in the art. Once the desired antibodies are identified the relevant genetic material may be isolated, manipulated and expressed using common, art-recognized molecular biology and biochemical techniques.

The antibodies produced by naïve libraries (either natural or synthetic) may be of moderate affinity (Kₐ of about 10⁶ to 10⁷ M⁻¹). To enhance affinity, affinity maturation may be mimicked in vitro by constructing antibody libraries (e.g., by introducing random mutations in vitro by using error-prone polymerase) and reselecting antibodies with high affinity for the antigen from those secondary libraries (e.g. by using phage or yeast display). WO 9607754 describes a method for inducing mutagenesis in a CDR of an immunoglobulin light chain to create a library of light chain genes.

Various techniques can be used to select antibodies, including but not limited to, phage or yeast display in which a library of human combinatorial antibodies or scFv fragments is synthesized on phages or yeast, the library is screened with the antigen of interest or an antibody-binding portion thereof, and the phage or yeast that binds the antigen is isolated, from which one may obtain the antibodies or immunoreactive fragments (Vaughan et al., 1996, PMID: 9630891; Sheets et al., 1998, PMID: 9600934; Boder et al., 1997, PMID: 9181578; Pepper et al., 2008, PMID: 18336206). Kits for generating phage or yeast display libraries are commercially available. There also are other methods and reagents that can be used in generating and screening antibody display libraries (see U.S. Pat. No. 5,223,409; WO 92/18619, WO 91/17271, WO 92/20791, WO 92/15679, WO 93/01288, WO 92/01047, WO 92/09690; and Barbas et al., 1991, PMID: 1896445). Such techniques advantageously allow for the screening of large numbers of candidate antibodies and provide for relatively easy manipulation of sequences (e.g., by recombinant shuffling).

### IV. CHARACTERISTICS OF ANTIBODIES

In certain embodiments, antibody-producing cells (e.g., hybridomas or yeast colonies) may be selected, cloned and further screened for favorable properties including, for example, robust growth, high antibody production and, as discussed in more detail below, desirable site-specific antibody characteristics. In other cases characteristics of the antibody may be imparted by selecting a particular antigen (e.g., a specific DLL3 isoform) or immunoreactive fragment of the target antigen for inoculation of the animal. In still other embodiments the selected antibodies may be engineered as described above to enhance or refine immunochemical characteristics such as affinity or pharmacokinetics.

A. Neutralizing Antibodies

In certain embodiments, the conjugates will comprise “neutralizing” antibodies or derivatives or fragments thereof. That is, the present invention may comprise antibody molecules that bind specific domains, motifs or epitopes and are capable of blocking, reducing or inhibiting the biological activity of DLL3. More generally the term “neutralizing antibody” refers to an antibody that binds to or interacts with a target molecule or ligand and prevents binding or association of the target molecule to a binding partner such as a receptor or substrate, thereby interrupting a biological response that otherwise would result from the interaction of the molecules.

It will be appreciated that competitive binding assays known in the art may be used to assess the binding and specificity of an antibody or immunologically functional fragment or derivative thereof. With regard to the instant invention an antibody or fragment will be held to inhibit or reduce binding of DLL3 to a binding partner or substrate when an excess of antibody reduces the quantity of binding partner bound to DLL3 by at least about 20%, 30%, 40%, 50%, 60%, 70%, 80%, 85%, 90%, 95%, 97%, 99% or more as measured, for example, by Notch receptor activity or in an in vitro competitive binding assay. In the case of antibodies to DLL3 for example, a neutralizing antibody or antagonist will preferably alter Notch receptor activity by at least about 20%, 30%, 40%, 50%, 60%, 70%, 80%, 85%, 90%, 95%, 97%, 99% or more. It will be appreciated that this modified activity may be measured directly using art-recognized techniques or may be measured by the impact the altered activity has downstream (e.g., oncogenesis, cell survival or activation or suppression of Notch responsive genes). Preferably, the ability of an antibody to neutralize DLL3 activity is assessed by inhibition of DLL3 binding to a Notch receptor or by assessing its ability to relieve DLL3 mediated repression of Notch signaling.

B. Internalizing Antibodies

In certain embodiments the antibodies may comprise internalizing antibodies such that the antibody will bind to a determinant and will be internalized (along with any conjugated pharmaceutically active moiety) into a selected target cell including tumorigenic cells. The number of antibody molecules internalized may be sufficient to kill an antigen-expressing cell, especially an antigen-expressing tumorigenic cell. Depending on the potency of the antibody or, in some instances, antibody drug conjugate, the uptake of a single antibody molecule into the cell may be sufficient to kill the target cell to which the antibody binds. With regard to the instant invention there is evidence that a substantial portion of expressed DLL3 protein remains associated with the tumorigenic cell surface, thereby allowing for localization and internalization of the disclosed antibodies or ADCs. In selected embodiments such antibodies will be associated with, or conjugated to, one or more drugs that kill the cell upon internalization. In some embodiments the ADCs of the instant invention will comprise an internalizing site-specific ADC.

As used herein, an antibody that “internalizes” is one that is taken up (along with any conjugated cytotoxin) by a target cell upon binding to an associated determinant. The number of such ADCs internalized will preferably be sufficient to kill the determinant-expressing cell, especially a determinant-expressing cancer stem cell. Depending on the potency of the cytotoxin or ADC as a whole, in some instances the uptake of a few antibody molecules into the cell is sufficient to kill the target cell to which the antibody binds. For example, certain drugs such as PBDs or calicheamicin are so potent that the internalization of a few molecules of the toxin conjugated to the antibody is sufficient to kill the target cell. Whether an antibody internalizes upon binding to a mammalian cell can be determined by various art-recognized assays (e.g., saporin assays such as Mab-Zap and Fab-Zap; Advanced Targeting Systems). Methods of detecting whether an antibody internalizes into a cell are also described in U.S. Pat. No. 7,619,068.

C. Depleting Antibodies

In other embodiments the antibodies of the invention are depleting antibodies. The term “depleting” antibody refers to an antibody that preferably binds to an antigen on or near the cell surface and induces, promotes or causes the death of the cell (e.g., by CDC, ADCC or introduction of a cytotoxic agent). In embodiments, the selected depleting antibodies will be conjugated to a cytotoxin.

Preferably a depleting antibody will be able to kill at least 20%, 30%, 40%, 50%, 60%, 70%, 80%, 85%, 90%, 95%, 97%, or 99% of DLL3-expressing cells in a defined cell population. In some embodiments the cell population may comprise enriched, sectioned, purified or isolated tumorigenic cells, including cancer stem cells. In other embodiments the cell population may comprise whole tumor samples or heterogeneous tumor extracts that comprise cancer stem cells. Standard biochemical techniques may be used to monitor and quantify the depletion of tumorigenic cells in accordance with the teachings herein.

D. Binding Affinity

Disclosed herein are antibodies that have a high binding affinity for a specific determinant e.g. DLL3. The term “K_{D}” refers to the dissociation constant or apparent affinity of a particular antibody-antigen interaction. An antibody of the invention can immunospecifically bind its target antigen when the dissociation constant K_{D} (k_{off}/kₒₙ) is ≤10⁻⁷ M. The antibody specifically binds antigen with high affinity when the K_{D} is 5×10⁻⁹ M, and with very high affinity when the K_{D} is ≤5×10⁻¹⁰ M. In one embodiment of the invention, the antibody has a K_{D} of ≤10⁻⁹ M and an off-rate of about 1×10⁻⁴/sec. In one embodiment of the invention, the off-rate is <1×10⁻⁵/sec. In other embodiments of the invention, the antibodies will bind to a determinant with a K_{D} of between about 10⁻⁷ M and 10⁻¹⁰ M, and in yet another embodiment it will bind with a K_{D}≤2×10⁻¹⁰ M. Still other selected embodiments of the invention comprise antibodies that have a K_{D} (k_{off}/kₒₙ) of less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴M, less than 5×10⁻¹⁴M, less than 10⁻¹⁵ M or less than 5×10⁻¹⁵ M.

In certain embodiments, an antibody of the invention that immunospecifically binds to a determinant e.g. DLL3 may have an association rate constant or kₒₙ (or kₐ) rate (antibody+antigen (Ag)^{k}ₒₙ←antibody-Ag) of at least 10⁵M⁻¹s⁻¹, at least 2×10⁵M⁻¹s⁻¹, at least 5×10⁵M⁻¹s⁻¹, at least 10⁶M⁻¹s⁻¹, at least 5×10⁶M⁻¹s⁻¹, at least 10⁷M⁻¹s⁻¹, at least 5×10⁷M⁻¹s⁻¹, or at least 10⁸M⁻¹s⁻¹.

In another embodiment, an antibody of the invention that immunospecifically binds to a determinant e.g. DLL3 may have a disassociation rate constant or k_{off} (or k_{d}) rate (antibody+antigen (Ag)^{k}_{off}←antibody-Ag) of less than 10⁻¹s⁻¹, less than 5×10⁻¹s⁻¹, less than 10⁻²s⁻¹, less than 5×10⁻² s^{-′}, less than 10⁻³s⁻¹, less than 5×10⁻³s⁻¹, less than 10⁻⁴ s⁻¹, less than 5×10⁴ s⁻¹, less than 10⁻⁵s⁻¹, less than 5×10⁻⁵s⁻¹, less than 10⁻⁶s⁻¹, less than 5×10⁻⁶ s⁻¹ less than 10⁻⁷s⁻¹, less than 5×10⁻⁷s⁻¹, less than 10⁻⁸ s⁻¹, less than 5×10⁻⁸s⁻¹, less than 10⁻⁸s⁻¹, less than 5×10⁻⁸s⁻¹ or less than 10⁻¹⁰s⁻¹.

Binding affinity may be determined using various techniques known in the art, for example, surface plasmon resonance, bio-layer interferometry, dual polarization interferometry, static light scattering, dynamic light scattering, isothermal titration calorimetry, ELISA, analytical ultracentrifugation, and flow cytometry.

E. Binning and Epitope Mapping

Antibodies disclosed herein may be characterized in terms of the discrete epitope with which they associate. An “epitope” is the portion(s) of a determinant to which the antibody or immunoreactive fragment specifically binds. Immunospecific binding can be confirmed and defined based on binding affinity, as described above, or by the preferential recognition by the antibody of its target antigen in a complex mixture of proteins and/or macromolecules (e.g. in competition assays). A “linear epitope”, is formed by contiguous amino acids in the antigen that allow for immunospecific binding of the antibody. The ability to preferentially bind linear epitopes is typically maintained even when the antigen is denatured. Conversely, a “conformational epitope”, usually comprises non-contiguous amino acids in the antigen's amino acid sequence but, in the context of the antigen's secondary, tertiary or quaternary structure, are sufficiently proximate to be bound concomitantly by a single antibody. When antigens with conformational epitopes are denatured, the antibody will typically no longer recognize the antigen. An epitope (contiguous or non-contiguous) typically includes at least 3, and more usually, at least 5 or 8-10 or 12-20 amino acids in a unique spatial conformation.

It is also possible to characterize the antibodies of the invention in terms of the group or “bin” to which they belong. “Binning” refers to the use of competitive antibody binding assays to identify pairs of antibodies that are incapable of binding an immunogenic determinant simultaneously, thereby identifying antibodies that “compete” for binding. Competing antibodies may be determined by an assay in which the antibody or immunologically functional fragment being tested prevents or inhibits specific binding of a reference antibody to a common antigen. Typically, such an assay involves the use of purified antigen (e.g., DLL3 or a domain or fragment thereof) bound to a solid surface or cells, an unlabeled test antibody and a labeled reference antibody. Competitive inhibition is measured by determining the amount of label bound to the solid surface or cells in the presence of the test antibody. Additional details regarding methods for determining competitive binding are provided in the Examples herein. Usually, when a competing antibody is present in excess, it will inhibit specific binding of a reference antibody to a common antigen by at least 30%, 40%, 45%, 50%, 55%, 60%, 65%, 70% or 75%. In some instance, binding is inhibited by at least 80%, 85%, 90%, 95%, or 97% or more. Conversely, when the reference antibody is bound it will preferably inhibit binding of a subsequently added test antibody (i.e., a DLL3 antibody) by at least 30%, 40%, 45%, 50%, 55%, 60%, 65%, 70% or 75%. In some instance, binding of the test antibody is inhibited by at least 80%, 85%, 90%, 95%, or 97% or more.

Generally binning or competitive binding may be determined using various art-recognized techniques, such as, for example, immunoassays such as western blots, radioimmunoassays, enzyme linked immunosorbent assay (ELISA), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such immunoassays are routine and well known in the art (see, Ausubel et al, eds, (1994) Current Protocols in Molecular Biology, Vol. 1, John Wiley & Sons, Inc., New York). Additionally, cross-blocking assays may be used (see, for example, WO 2003/48731; and Harlow et al. (1988) Antibodies, A Laboratory Manual, Cold Spring Harbor Laboratory, Ed Harlow and David Lane).

Other technologies used to determine competitive inhibition (and hence “bins”), include: surface plasmon resonance using, for example, the BIAcore™ 2000 system (GE Healthcare); bio-layer interferometry using, for example, a ForteBio® Octet RED (ForteBio); or flow cytometry bead arrays using, for example, a FACSCanto II (BD Biosciences) or a multiplex LUMINEX™ detection assay (Luminex).

Luminex is a bead-based immunoassay platform that enables large scale multiplexed antibody pairing. The assay compares the simultaneous binding patterns of antibody pairs to the target antigen. One antibody of the pair (capture mAb) is bound to Luminex beads, wherein each capture mAb is bound to a bead of a different color. The other antibody (detector mAb) is bound to a fluorescent signal (e.g. phycoerythrin (PE)). The assay analyzes the simultaneous binding (pairing) of antibodies to an antigen and groups together antibodies with similar pairing profiles. Similar profiles of a detector mAb and a capture mAb indicates that the two antibodies bind to the same or closely related epitopes. In one embodiment, pairing profiles can be determined using Pearson correlation coefficients to identify the antibodies which most closely correlate to any particular antibody on the panel of antibodies that are tested. In embodiments a test/detector mAb will be determined to be in the same bin as a reference/capture mAb if the Pearson's correlation coefficient of the antibody pair is at least 0.9. In other embodiments the Pearson's correlation coefficient is at least 0.8, 0.85, 0.87 or 0.89. In further embodiments, the Pearson's correlation coefficient is at least 0.91, 0.92, 0.93, 0.94, 0.95, 0.96, 0.97, 0.98, 0.99 or 1. Other methods of analyzing the data obtained from the Luminex assay are described in U.S. Pat. No. 8,568,992. The ability of Luminex to analyze 100 different types of beads (or more) simultaneously provides almost unlimited antigen and/or antibody surfaces, resulting in improved throughput and resolution in antibody epitope profiling over a biosensor assay (Miller, et al., 2011, PMID: 21223970).

Similarly binning techniques comprising surface plasmon resonance are compatible with the instant invention. As used herein “surface plasmon resonance,” refers to an optical phenomenon that allows for the analysis of real-time specific interactions by detection of alterations in protein concentrations within a biosensor matrix. Using commercially available equipment such as the BIAcore™ 2000 system it may readily be determined if selected antibodies compete with each other for binding to a defined antigen.

In other embodiments, a technique that can be used to determine whether a test antibody “competes” for binding with a reference antibody is “bio-layer interferometry”, an optical analytical technique that analyzes the interference pattern of white light reflected from two surfaces: a layer of immobilized protein on a biosensor tip, and an internal reference layer. Any change in the number of molecules bound to the biosensor tip causes a shift in the interference pattern that can be measured in real-time. Such biolayer interferometry assays may be conducted using a ForteBio® Octet RED machine as follows. A reference antibody (Ab1) is captured onto an anti-mouse capture chip, a high concentration of non-binding antibody is then used to block the chip and a baseline is collected. Monomeric, recombinant target protein is then captured by the specific antibody (Ab1) and the tip is dipped into a well with either the same antibody (Ab1) as a control or into a well with a different test antibody (Ab2). If no further binding occurs, as determined by comparing binding levels with the control Ab1, then Ab1 and Ab2 are determined to be “competing” antibodies. If additional binding is observed with Ab2, then Ab1 and Ab2 are determined not to compete with each other. This process can be expanded to screen large libraries of unique antibodies using a full row of antibodies in a 96-well plate representing unique bins. In embodiments a test antibody will compete with a reference antibody if the reference antibody inhibits specific binding of the test antibody to a common antigen by at least 40%, 45%, 50%, 55%, 60%, 65%, 70% or 75%. In other embodiments, binding is inhibited by at least 80%, 85%, 90%, 95%, or 97% or more.

Once a bin, encompassing a group of competing antibodies, has been defined further characterization can be carried out to determine the specific domain or epitope on the antigen to which that group of antibodies binds. Domain-level epitope mapping may be performed using a modification of the protocol described by Cochran et al., 2004, PMID: 15099763. Fine epitope mapping is the process of determining the specific amino acids on the antigen that comprise the epitope of a determinant to which the antibody binds.

In certain embodiments fine epitope mapping can be performed using phage or yeast display. Other compatible epitope mapping techniques include alanine scanning mutants, peptide blots (Reineke, 2004, PMID: 14970513), or peptide cleavage analysis. In addition, methods such as epitope excision, epitope extraction and chemical modification of antigens can be employed (Tomer, 2000, PMID: 10752610) using enzymes such as proteolytic enzymes (e.g., trypsin, endoproteinase Glu-C, endoproteinase Asp-N, chymotrypsin, etc.); chemical agents such as succinimidyl esters and their derivatives, primary amine-containing compounds, hydrazines and carbohydrazines, free amino acids, etc. In another embodiment Modification-Assisted Profiling, also known as Antigen Structure-based Antibody Profiling (ASAP) can be used to categorize large numbers of monoclonal antibodies directed against the same antigen according to the similarities of the binding profile of each antibody to chemically or enzymatically modified antigen surfaces (U.S.P.N. 2004/0101920).

Once a desired epitope on an antigen is determined, it is possible to generate additional antibodies to that epitope, e.g., by immunizing with a peptide comprising the selected epitope using techniques described herein.

### V. ANTIBODY CONJUGATES

In some embodiments the antibodies of the invention may be conjugated with pharmaceutically active or diagnostic moieties to form an “antibody drug conjugate” (ADC) or “antibody conjugate”. The term “conjugate” is used broadly and means the covalent or non-covalent association of any pharmaceutically active or diagnostic moiety with an antibody of the instant invention regardless of the method of association. In certain embodiments the association is effected through a lysine or cysteine residue of the antibody. In some embodiments the pharmaceutically active or diagnostic moieties may be conjugated to the antibody via one or more site-specific free cysteine(s). The disclosed ADCs may be used for therapeutic and diagnostic purposes.

The ADCs of the instant invention may be used to deliver cytotoxins or other payloads to the target location (e.g., tumorigenic cells and/or cells expressing DLL3). As used herein the terms “drug” or “warhead” may be used interchangeably and will mean a biologically active or detectable molecule or drug, including anti-cancer agents and cytotoxins as described below. A “payload” may comprise a drug or “warhead” in combination with an optional linker compound. The “warhead” on the conjugate may comprise peptides, proteins or prodrugs which are metabolized to an active agent in vivo, polymers, nucleic acid molecules, small molecules, binding agents, mimetic agents, synthetic drugs, inorganic molecules, organic molecules and radioisotopes. In an advantageous embodiment, the disclosed ADCs will direct the bound payload to the target site in a relatively unreactive, non-toxic state before releasing and activating the warhead. This targeted release of the warhead is preferably achieved through stable conjugation of the payloads (e.g., via one or more cysteines on the antibody) and the relatively homogeneous composition of the ADC preparations which minimize over-conjugated toxic ADC species. Coupled with drug linkers that are designed to largely release the warhead once it has been delivered to the tumor site, the conjugates of the instant invention can substantially reduce undesirable non-specific toxicity. This advantageously provides for relatively high levels of the active cytotoxin at the tumor site while minimizing exposure of non-targeted cells and tissue thereby providing an enhanced therapeutic index.

It will be appreciated that, while some embodiments of the invention comprise payloads incorporating therapeutic moieties (e.g., cytotoxins), other payloads incorporating diagnostic agents and biocompatible modifiers may benefit from the targeted release provided by the disclosed conjugates. Accordingly, any disclosure directed to exemplary therapeutic payloads is also applicable to payloads comprising diagnostic agents or biocompatible modifiers as discussed herein unless otherwise dictated by context. The selected payload may be covalently or non-covalently linked to, the antibody and exhibit various stoichiometric molar ratios depending, at least in part, on the method used to effect the conjugation. Conjugates of the instant invention may be generally represented by the formula:

Ab-[L-D]n or a pharmaceutically acceptable salt thereof wherein:
a) Ab comprises an anti-DLL3 antibody;
b) L comprises an optional linker;
c) D comprises a drug; and
d) n is an integer from about 1 to about 20.

Those of skill in the art will appreciate that conjugates according to the aforementioned formula may be fabricated using a number of different linkers and drugs and that conjugation methodology will vary depending on the selection of components. As such, any drug or drug linker compound that associates with a reactive residue (e.g., cysteine or lysine) of the disclosed antibodies are compatible with the teachings herein. Similarly, any reaction conditions that allow for conjugation (including site-specific conjugation) of the selected drug to an antibody are within the scope of the present invention. Notwithstanding the foregoing, some embodiments of the instant invention comprise selective conjugation of the drug or drug linker to free cysteines using stabilization agents in combination with mild reducing agents as described herein. Such reaction conditions tend to provide more homogeneous preparations with less non-specific conjugation and contaminants and correspondingly less toxicity.

A. Payloads and Warheads

1. Therapeutic Agents

The antibodies of the invention may be conjugated, linked or fused to or otherwise associated with a pharmaceutically active moiety which is a therapeutic moiety or a drug such as an anti-cancer agent including, but not limited to, cytotoxic agents (or cytotoxins), cytostatic agents, anti-angiogenic agents, debulking agents, chemotherapeutic agents, radiotherapeutic agents, targeted anti-cancer agents, biological response modifiers, cancer vaccines, cytokines, hormone therapies, anti-metastatic agents and immunotherapeutic agents.

Exemplary anti-cancer agents or cytotoxins (including homologs and derivatives thereof) comprise 1-dehydrotestosterone, anthramycins, actinomycin D, bleomycin, calicheamicins (including n-acetyl calicheamicin), colchicin, cyclophosphamide, cytochalasin B, dactinomycin (formerly actinomycin), dihydroxy anthracin, dione, duocarmycin, emetine, epirubicin, ethidium bromide, etoposide, glucocorticoids, gramicidin D, lidocaine, maytansinoids such as DM-1 and DM-4 (Immunogen), mithramycin, mitomycin, mitoxantrone, paclitaxel, procaine, propranolol, puromycin, tenoposide, tetracaine and pharmaceutically acceptable salts or solvates, acids or derivatives of any of the above.

Additional compatible cytotoxins comprise dolastatins and auristatins, including monomethyl auristatin E (MMAE) and monomethyl auristatin F (MMAF) (Seattle Genetics), amanitins such as alpha-amanitin, beta-amanitin, gamma-amanitin or epsilon-amanitin (Heidelberg Pharma), DNA minor groove binding agents such as duocarmycin derivatives (Syntarga), alkylating agents such as modified or dimeric pyrrolobenzodiazepines (PBD), mechlorethamine, thioepa, chlorambucil, melphalan, carmustine (BCNU), lomustine (CCNU), cyclothosphamide, busulfan, dibromomannitol, streptozotocin, mitomycin C and cisdichlorodiamine platinum (II) (DDP) cisplatin, splicing inhibitors such as meayamycin analogs or derivatives (e.g., FR901464 as set forth in U.S. Pat. No. 7,825,267), tubular binding agents such as epothilone analogs and tubulysins, paclitaxel and DNA damaging agents such as calicheamicins and esperamicins, antimetabolites such as methotrexate, 6-mercaptopurine, 6-thioguanine, cytarabine, and 5-fluorouracil decarbazine, anti-mitotic agents such as vinblastine and vincristine and anthracyclines such as daunorubicin (formerly daunomycin) and doxorubicin and pharmaceutically acceptable salts or solvates, acids or derivatives of any of the above.

In selected embodiments the antibodies of the instant invention may be associated with anti-CD3 binding molecules to recruit cytotoxic T-cells and have them target tumorigenic cells (BiTE technology; see e.g., Fuhrmann et. al. (2010) Annual Meeting of AACR Abstract No. 5625).

In further embodiments ADCs of the invention may comprise cytotoxins comprising therapeutic radioisotopes conjugated using appropriate linkers. Exemplary radioisotopes that may be compatible with such embodiments include, but are not limited to, iodine (¹³¹I, ¹²⁵I, ¹²³I, ¹²¹I), carbon (¹⁴C), copper (⁶²Cu, ⁶⁴Cu, ⁶⁷Cu), sulfur (³⁵S), radium (²²³R), tritium (³H), indium (¹¹⁵In, ¹¹³In, ¹¹²In, ¹¹¹In), bismuth (²¹²Bi, ²¹³Bi), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P ¹⁵³Gd, ¹⁶⁹Yb, ⁵¹Cr ⁵⁴Mn, ⁷⁵Se, ¹¹³Sn, ¹¹⁷Sn, ²²⁵Ac, ⁷⁶Br, and ²¹¹At. Other radionuclides are also available as diagnostic and therapeutic agents, especially those in the energy range of 60 to 4,000 keV.

In other selected embodiments the ADCs of the instant invention will be conjugated to a cytotoxic benzodiazepine derivative warhead. Compatible benzodiazepine derivatives (and optional linkers) that may be conjugated to the disclosed antibodies are described, for example, in U.S. Pat. No. 8,426,402 and PCT filings WO2012/128868 and WO2014/031566. As with the PBDs discussed below, compatible benzodiazepine derivatives are believed to bind in the minor grove of DNA and inhibit nucleic acid synthesis. Such compounds reportedly have potent antitumor properties and, as such, are particularly suitable for use in the ADCs of the instant invention.

In certain embodiments, the ADCs of the invention may comprise PBDs, and pharmaceutically acceptable salts or solvates, acids or derivatives thereof, as warheads. PBDs are alkylating agents that exert antitumor activity by covalently binding to DNA in the minor groove and inhibiting nucleic acid synthesis. PBDs have been shown to have potent antitumor properties while exhibiting minimal bone marrow depression. PBDs compatible with the invention may be linked to an antibody using several types of linkers (e.g., a peptidyl linker comprising a maleimido moiety with a free sulfhydryl), and in certain embodiments are dimeric in form (i.e., PBD dimers). Compatible PBDs (and optional linkers) that may be conjugated to the disclosed antibodies are described, for example, in U.S. Pat. Nos. 6,362,331, 7,049,311, 7,189,710, 7,429,658, 7,407,951, 7,741,319, 7,557,099, 8,034,808, 8,163,736, 2011/0256157 and PCT filings WO2011/130613, WO2011/128650, WO2011/130616, WO2014/057073 and WO2014/057074. Examples of PBD compounds compatible with the instant invention are shown below.

In this respect PBDs have been shown to have potent antitumor properties while exhibiting minimal bone marrow depression. PBDs compatible with the present invention may be linked to the DLL3 modulator using any one of several types of linker (e.g., a peptidyl linker comprising a maleimido moiety with a free sulfhydryl) and, in certain embodiments are dimeric in form (i.e., PBD dimers). PBDs are of the general structure:

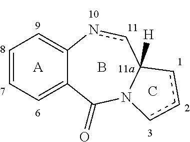

They differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position which is the electrophilic center responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). Their ability to form an adduct in the minor groove, enables them to interfere with DNA processing, hence their use as cytotoxic agents. As alluded to above, in order to increase their potency PBDs are often used in a dimeric form which may be conjugated to anti-DLL3 antibodies as described herein.

In particularly preferred embodiments compatible PBDs that may be conjugated to the disclosed modulators are described, in U.S.P.N. 2011/0256157. In this disclosure, PBD dimers, i.e. those comprising two PBD moieties may be preferred. Thus, preferred conjugates of the present invention are those having the formula (AB) or (AC):

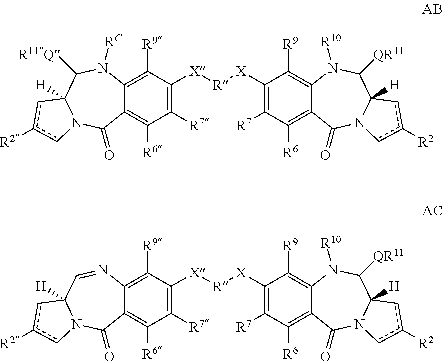

wherein:
the dotted lines indicate the optional presence of a double bond between C1 and C2 or C2 and C3;
R² is independently selected from H, OH, ═O, ═CH₂, CN, R, OR, ═CH—R^{D}, ═C(R^{D})₂, O—SO₂—R, CO₂R and COR, and optionally further selected from halo or dihalo;
where R^{D} is independently selected from R, CO₂R, COR, CHO, CO₂H, and halo;
R⁶ and R⁹ are independently selected from H, R, OH, OR, SH, SR, NH₂, NHR, NRR′, NO₂, Me₃Sn and halo;
R⁷ is independently selected from H, R, OH, OR, SH, SR, NH₂, NHR, NRR′, NO₂, Me₃Sn and halo;
R¹⁰ is a linker connected to a DLL3 antibody or fragment or derivative thereof, as described herein;
Q is independently selected from O, S and NH;
R¹¹ is either H, or R or, where Q is O, R¹¹ may be SO₃M, where M is a metal cation;
X is selected from O, S, or N(H) and in selected embodiments comprises O;
R″ is a C₃₋₁₂ alkylene group, which chain may be interrupted by one or more heteroatoms (e.g., O, S, N(H), NMe and/or aromatic rings, e.g. benzene or pyridine, which rings are optionally substituted);
R and R′ are each independently selected from optionally substituted C₁₋₁₂ alkyl, C₃₋₂₀ heterocyclyl and C₅₋₂₀ aryl groups, and optionally in relation to the group NRR′, R and R′ together with the nitrogen atom to which they are attached form an optionally substituted 4-, 5-, 6- or 7-membered heterocyclic ring; and
wherein R^{2″}, R^{6″}, R^{7″}, R^{9″}, X″, Q″ and R^{11″} (where present) are as defined according to R², R⁶, R⁷, R⁹, X, Q and R¹¹ respectively, and R^{C} is a capping group.

Selected embodiments comprising the aforementioned structures are described in more detail immediately below.

Double Bond

In one embodiment, there is no double bond present between C1 and C2, and C2 and C3.

In one embodiment, the dotted lines indicate the optional presence of a double bond between C2 and C3, as shown below:

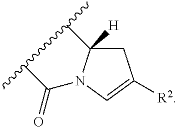

In one embodiment, a double bond is present between C2 and C3 when R² is C₅₋₂₀ aryl or C₁₋₁₂ alkyl.

In one embodiment, the dotted lines indicate the optional presence of a double bond between C1 and C2, as shown below:

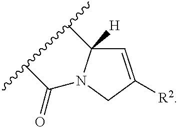

In one embodiment, a double bond is present between C1 and C2 when R² is C₅₋₂₀ aryl or C₁₋₁₂ alkyl.

R²

In one embodiment, R² is independently selected from H, OH, ═O, ═CH₂, CN, R, OR, ═CH—R^{D}, ═C(R^{D})₂, O—SO₂—R, CO₂R and COR, and optionally further selected from halo or dihalo.

In one embodiment, R² is independently selected from H, OH, ═O, ═CH₂, CN, R, OR, ═CH—R^{D}, ═C(R^{D})₂, O—SO₂—R, CO₂R and COR.

In one embodiment, R² is independently selected from H, ═O, ═CH₂, R, ═CH—R^{D}, and ═C(R^{D})₂.

In one embodiment, R² is independently H.

In one embodiment R² is independently R wherein R comprises CH₃.

In one embodiment, R² is independently ═O.

In one embodiment, R² is independently ═CH₂.

In one embodiment, R² is independently ═CH—R^{D}. Within the PBD compound, the group ═CH—R^{D} may have either configuration shown below:

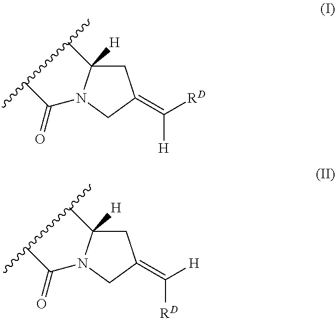

In one embodiment, the configuration is configuration (I).

In one embodiment, R² is independently ═C(R^{D})₂.

In one embodiment, R² is independently ═CF₂.

In one embodiment, R² is independently R.

In one embodiment, R² is independently optionally substituted C₅₋₂₀ aryl.

In one embodiment, R² is independently optionally substituted C₁₋₁₂ alkyl.

In one embodiment, R² is independently optionally substituted C₅₋₂₀ aryl.

In one embodiment, R² is independently optionally substituted C₅₋₇ aryl.

In one embodiment, R² is independently optionally substituted C₈₋₁₀ aryl.

In one embodiment, R² is independently optionally substituted phenyl.

In one embodiment, R² is independently optionally substituted napthyl.

In one embodiment, R² is independently optionally substituted pyridyl.

In one embodiment, R² is independently optionally substituted quinolinyl or isoquinolinyl.

In one embodiment, R² bears one to three substituent groups, with 1 and 2 being more preferred, and singly substituted groups being most preferred. The substituents may be any position.

Where R² is a C₅₋₇ aryl group, a single substituent is preferably on a ring atom that is not adjacent the bond to the remainder of the compound, i.e. it is preferably β or γ to the bond to the remainder of the compound. Therefore, where the C₅₋₇ aryl group is phenyl, the substituent is preferably in the meta- or para-positions, and more preferably is in the para-position.

In one embodiment, R² is selected from:

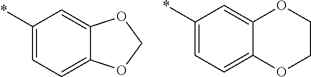

where the asterisk indicates the point of attachment.

Where R² is a C₈₋₁₀ aryl group, for example quinolinyl or isoquinolinyl, it may bear any number of substituents at any position of the quinoline or isoquinoline rings. In some embodiments, it bears one, two or three substituents, and these may be on either the proximal and distal rings or both (if more than one substituent).

In one embodiment, where R² is optionally substituted, the substituents are selected from those substituents given in the substituent section below.

Where R is optionally substituted, the substituents are preferably selected from:

Halo, Hydroxyl, Ether, Formyl, Acyl, Carboxy, Ester, Acyloxy, Amino, Amido, Acylamido, Aminocarbonyloxy, Ureido, Nitro, Cyano and Thioether.

In one embodiment, where R or R² is optionally substituted, the substituents are selected from the group consisting of R, OR, SR, NRR′, NO₂, halo, CO₂R, COR, CONH₂, CONHR, and CONRR′.

Where R² is C₁₋₁₂ alkyl, the optional substituent may additionally include C₃₋₂₀ heterocyclyl and C₅₋₂₀ aryl groups.

Where R² is C₃₋₂₀ heterocyclyl, the optional substituent may additionally include C₁₋₁₂ alkyl and C₅₋₂₀ aryl groups.

Where R² is C₅₋₂₀ aryl groups, the optional substituent may additionally include C₃₋₂₀ heterocyclyl and C₁₋₁₂ alkyl groups.

It is understood that the term “alkyl” encompasses the sub-classes alkenyl and alkynyl as well as cycloalkyl. Thus, where R² is optionally substituted C₁₋₁₂ alkyl, it is understood that the alkyl group optionally contains one or more carbon-carbon double or triple bonds, which may form part of a conjugated system. In one embodiment, the optionally substituted C₁₋₁₂ alkyl group contains at least one carbon-carbon double or triple bond, and this bond is conjugated with a double bond present between C1 and C2, or C2 and C3. In one embodiment, the C₁₋₁₂ alkyl group is a group selected from saturated C₁₋₁₂ alkyl, C₂₋₁₂ alkenyl, C₂₋₁₂ alkynyl and C₃₋₁₂ cycloalkyl.

If a substituent on R² is halo, it is preferably F or Cl, more preferably Cl.

If a substituent on R² is ether, it may in some embodiments be an alkoxy group, for example, a C₁₋₇ alkoxy group (e.g. methoxy, ethoxy) or it may in some embodiments be a C₅₋₇ aryloxy group (e.g phenoxy, pyridyloxy, furanyloxy).

If a substituent on R² is C₁₋₇ alkyl, it may preferably be a C₁₋₄ alkyl group (e.g. methyl, ethyl, propyl, butyl).

If a substituent on R² is C₃₋₇ heterocyclyl, it may in some embodiments be C₆ nitrogen containing heterocyclyl group, e.g. morpholino, thiomorpholino, piperidinyl, piperazinyl. These groups may be bound to the rest of the PBD moiety via the nitrogen atom. These groups may be further substituted, for example, by C₁₋₄ alkyl groups.

If a substituent on R² is bis-oxy-C₁₋₃ alkylene, this is preferably bis-oxy-methylene or bis-oxy-ethylene.

Particularly preferred substituents for R² include methoxy, ethoxy, fluoro, chloro, cyano, bis-oxy-methylene, methyl-piperazinyl, morpholino and methyl-thienyl.

Particularly preferred substituted R² groups include, but are not limited to, 4-methoxy-phenyl, 3-methoxyphenyl, 4-ethoxy-phenyl, 3-ethoxy-phenyl, 4-fluoro-phenyl, 4-chloro-phenyl, 3,4-bisoxymethylene-phenyl, 4-methylthienyl, 4-cyanophenyl, 4-phenoxyphenyl, quinolin-3-yl and quinolin-6-yl, isoquinolin-3-yl and isoquinolin-6-yl, 2-thienyl, 2-furanyl, methoxynaphthyl, and naphthyl.

In one embodiment, R² is halo or dihalo. In one embodiment, R² is —F or —F₂, which substituents are illustrated below as (III) and (IV) respectively:

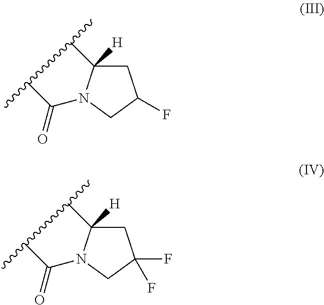

R^{D}

In one embodiment, R^{D} is independently selected from R, CO₂R, COR, CHO, CO₂H, and halo.

In one embodiment, R^{D} is independently R.

In one embodiment, R^{D} is independently halo.

R⁶

In one embodiment, R⁶ is independently selected from H, R, OH, OR, SH, SR, NH₂, NHR, NRR′, NO₂, Me₃Sn— and Halo.

In one embodiment, R⁶ is independently selected from H, OH, OR, SH, NH₂, NO₂ and Halo.

In one embodiment, R⁶ is independently selected from H and Halo.

In one embodiment, R⁶ is independently H.

In one embodiment, R⁶ and R⁷ together form a group —O—(CH₂)ₚ—O—, where p is 1 or 2.

R⁷

R⁷ is independently selected from H, R, OH, OR, SH, SR, NH₂, NHR, NRR′, NO₂, Me₃Sn and halo.

In one embodiment, R⁷ is independently OR.

In one embodiment, R⁷ is independently OR^{7A}, where R^{7A} is independently optionally substituted C₁₋₆ alkyl.

In one embodiment, R^{7A} is independently optionally substituted saturated C₁₋₆ alkyl.

In one embodiment, R^{7A} is independently CH₃.

In one embodiment, R^{7A} is independently optionally substituted C₂₋₄ alkenyl.

In one embodiment, R^{7A} is independently Me.

In one embodiment, R^{7A} is independently CH₂Ph.

In one embodiment, R^{7A} is independently allyl.

In one embodiment, the compound is a dimer where the R⁷ groups of each monomer form together a dimer bridge having the formula X—R″—X linking the monomers.

R⁹

In one embodiment, R⁹ is independently selected from H, R, OH, OR, SH, SR, NH₂, NHR, NRR′, NO₂, Me₃Sn— and Halo.

In one embodiment, R⁹ is independently H.

In one embodiment, R⁹ is independently R or OR.

R¹⁰

Preferably compatible linkers such as those described herein attach the DLL3 antibody to the PBD drug moiety through covalent bond(s) at the R¹⁰ position (i.e., N10).

Q

In certain embodiments Q is independently selected from O, S and NH.

In one embodiment, Q is independently 0.

In one embodiment, Q is independently S.

In one embodiment, Q is independently NH.

R¹¹

In selected embodiments R¹¹ is either H, or R or, where Q is O, R¹¹ may be SO₃M where M is a metal cation. The cation may be Nat.

In certain embodiments R¹¹ is H.

In certain embodiments R¹¹ is R.

In certain embodiments, where Q is O, R¹¹ is SO₃M where M is a metal cation. The cation may be Nat.

In certain embodiments where Q is O, R¹¹ is H.

In certain embodiments where Q is O, R¹¹ is R.

X

In one embodiment, X is selected from O, S, or N(H).

Preferably, X is O.

R″

R″ is a C₃₋₁₂ alkylene group, which chain may be interrupted by one or more heteroatoms, e.g. O, S, N(H), NMe and/or aromatic rings, e.g. benzene or pyridine, which rings are optionally substituted.

In one embodiment, R″ is a C₃₋₁₂ alkylene group, which chain may be interrupted by one or more heteroatoms and/or aromatic rings, e.g. benzene or pyridine.

In one embodiment, the alkylene group is optionally interrupted by one or more heteroatoms selected from O, S, and NMe and/or aromatic rings, which rings are optionally substituted.

In one embodiment, the aromatic ring is a C₅₋₂₀ arylene group, where arylene pertains to a divalent moiety obtained by removing two hydrogen atoms from two aromatic ring atoms of an aromatic compound, which moiety has from 5 to 20 ring atoms.

In one embodiment, R″ is a C₃₋₁₂ alkylene group, which chain may be interrupted by one or more heteroatoms, e.g. O, S, N(H), NMe and/or aromatic rings, e.g. benzene or pyridine, which rings are optionally substituted by NH₂.

In one embodiment, R″ is a C₃₋₁₂ alkylene group.

In one embodiment, R″ is selected from a C₃, C₅, C₇, C₉ and a C₁₁ alkylene group.

In one embodiment, R″ is selected from a C₃, C₅ and a C₇ alkylene group.

In one embodiment, R″ is selected from a C₃ and a C₅ alkylene group.

In one embodiment, R″ is a C₃ alkylene group.

In one embodiment, R″ is a C₅ alkylene group.

The alkylene groups listed above may be optionally interrupted by one or more heteroatoms and/or aromatic rings, e.g. benzene or pyridine, which rings are optionally substituted.

The alkylene groups listed above may be optionally interrupted by one or more heteroatoms and/or aromatic rings, e.g. benzene or pyridine.

The alkylene groups listed above may be unsubstituted linear aliphatic alkylene groups.

R and R′

In one embodiment, R is independently selected from optionally substituted C₁₋₁₂ alkyl, C₃₋₂₀ heterocyclyl and C₅₋₂₀ aryl groups. These groups are each defined in the substituents section below.

In one embodiment, R is independently optionally substituted C₁₋₁₂ alkyl.

In one embodiment, R is independently optionally substituted C₃₋₂₀ heterocyclyl.

In one embodiment, R is independently optionally substituted C₅₋₂₀ aryl.

In one embodiment, R is independently optionally substituted C₁₋₁₂ alkyl.

Described above in relation to R² are various embodiments relating to preferred alkyl and aryl groups and the identity and number of optional substituents. The preferences set out for R² as it applies to R are applicable, where appropriate, to all other groups R, for examples where R⁶, R⁷, R⁸ or R⁹ is R.

The preferences for R apply also to R′.

In some embodiments of the invention there is provided a compound having a substituent group —NRR′. In one embodiment, R and R′ together with the nitrogen atom to which they are attached form an optionally substituted 4-, 5-, 6- or 7-membered heterocyclic ring. The ring may contain a further heteroatom, for example N, O or S.

In one embodiment, the heterocyclic ring is itself substituted with a group R. Where a further N heteroatom is present, the substituent may be on the N heteroatom.

In addition to the aforementioned PBDs certain dimeric PBDs have been shown to be particularly active and may be used in conjunction with the instant invention. To this end antibody drug conjugates (i.e., ADCs 1-6 as disclosed herein) of the instant invention may comprise a PBD compound as set forth immediately below as PBD 1-5. The synthesis of each of PBD 1-5 as a component of drug-linker compounds is presented in great detail in WO 2014/130879 which is hereby incorporated by reference as to such synthesis. In view of WO 2014/130879 cytotoxic compounds that may comprise selected warheads of the ADCs of the present invention could readily be generated and employed as set forth herein. Thus selected PBD compounds that may be released from the disclosed ADCs upon cleavage of a linker are set forth immediately below:

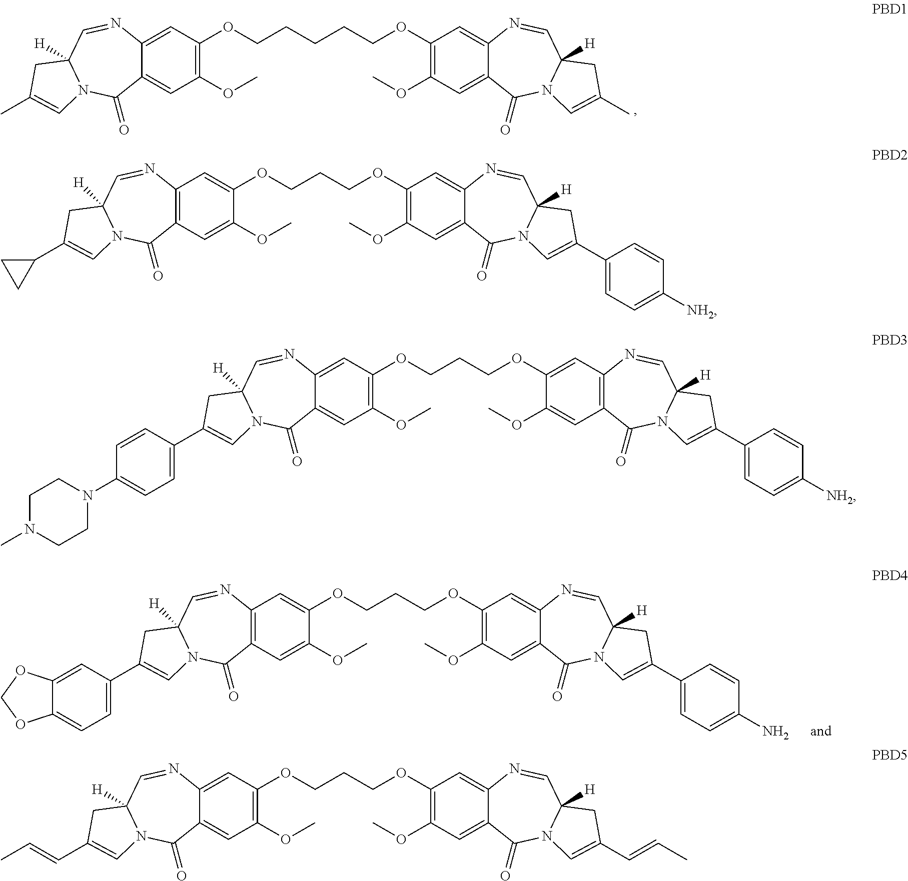

It will be appreciated that each of the aforementioned dimeric PBD warheads would be preferably be released upon internalization by the target cell and destruction of the linker. As described in more detail below, preferable linkers will comprise cleavable linkers incorporating a self-immolation moiety that allows release of the active PBD warhead without retention of any part of the linker. Upon release the PBD warhead will then bind and cross-link with the target cell's DNA. Such binding apparently blocks division of the target cancer cell without distorting its DNA helix, thus potentially avoiding the common phenomenon of emergent drug resistance.

Delivery and release of such compounds at the tumor site(s) may prove clinically effective in treating or managing proliferative disorders in accordance with the instant disclosure. With regard to the compounds it will be appreciated that each of the disclosed PBDs have two sp² centers in each C-ring, which may allow for stronger binding in the minor groove of DNA (and hence greater toxicity), than for compounds with only one sp² center in each C-ring. Thus, when used in DLL3 ADCs as set forth herein the disclosed PBDs may prove to be particularly effective for the treatment of proliferative disorders.

The foregoing provides exemplary PBD compounds that are compatible with the instant invention and is in no way meant to be limiting as to other PBDs that may be successfully incorporated in anti-DLL3 conjugates according to the teachings herein. Rather, any PBD that may be conjugated to an antibody as described herein and set forth in the Examples below is compatible with the disclosed conjugates and expressly within the metes and bounds of the invention.

In addition to the aforementioned agents the antibodies of the present invention may also be conjugated to biological response modifiers. For example, in some embodiments the drug moiety can be a polypeptide possessing a desired biological activity. Such proteins may include, for example, a toxin such as abrin, ricin A, Onconase (or another cytotoxic RNase), pseudomonas exotoxin, cholera toxin, diphtheria toxin; an apoptotic agent such as tumor necrosis factor e.g. TNF-α or TNF-β, α-interferon, β-interferon, nerve growth factor, platelet derived growth factor, tissue plasminogen activator, AIM I (WO 97/33899), AIM II (WO 97/34911), Fas Ligand (Takahashi et al., 1994, PMID: 7826947), and VEGI (WO 99/23105), a thrombotic agent, an anti-angiogenic agent, e.g., angiostatin or endostatin, a lymphokine, for example, interleukin-1 (IL-1), interleukin-2 (IL-2), interleukin-6 (IL-6), granulocyte macrophage colony stimulating factor (GM-CSF), and granulocyte colony stimulating factor (G-CSF), or a growth factor e.g., growth hormone (GH).

2. Diagnostic or Detection Agents

In other embodiments, the antibodies of the invention, or fragments or derivatives thereof, are conjugated to a diagnostic or detectable agent, marker or reporter which may be, for example, a biological molecule (e.g., a peptide or nucleotide), a small molecule, fluorophore, or radioisotope. Labeled antibodies can be useful for monitoring the development or progression of a hyperproliferative disorder or as part of a clinical testing procedure to determine the efficacy of a particular therapy including the disclosed antibodies (i.e. theragnostics) or to determine a future course of treatment. Such markers or reporters may also be useful in purifying the selected antibody, for use in antibody analytics (e.g., epitope binding or antibody binning), separating or isolating tumorigenic cells or in preclinical procedures or toxicology studies.

Such diagnosis, analysis and/or detection can be accomplished by coupling the antibody to detectable substances including, but not limited to, various enzymes comprising for example horseradish peroxidase, alkaline phosphatase, beta-galactosidase, or acetylcholinesterase; prosthetic groups, such as but not limited to streptavidinlbiotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to, iodine (¹³¹I, ¹²⁵I, ¹²³I, ¹²¹I), carbon (¹⁴C), sulfur (³⁵S), tritium (³H), indium (¹¹⁵In, ¹¹³In, ¹¹²In, ¹¹¹In), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ⁸⁹Zr, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies, non-radioactive paramagnetic metal ions, and molecules that are radiolabeled or conjugated to specific radioisotopes. In such embodiments appropriate detection methodology is well known in the art and readily available from numerous commercial sources.

In other embodiments the antibodies or fragments thereof can be fused or conjugated to marker sequences or compounds, such as a peptide or fluorophore to facilitate purification or diagnostic or analytic procedures such as immunohistochemistry, bio-layer interferometry, surface plasmon resonance, flow cytometry, competitive ELISA, FACs, etc. In some embodiments, the marker comprises a histidine tag such as that provided by the pQE vector (Qiagen), among others, many of which are commercially available. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag (U.S. Pat. No. 4,703,004).

3. Biocompatible Modifiers

In selected embodiments the antibodies of the invention may be conjugated with biocompatible modifiers that may be used to adjust, alter, improve or moderate antibody characteristics as desired. For example, antibodies or fusion constructs with increased in vivo half-lives can be generated by attaching relatively high molecular weight polymer molecules such as commercially available polyethylene glycol (PEG) or similar biocompatible polymers. Those skilled in the art will appreciate that PEG may be obtained in many different molecular weights and molecular configurations that can be selected to impart specific properties to the antibody (e.g. the half-life may be tailored). PEG can be attached to antibodies or antibody fragments or derivatives with or without a multifunctional linker either through conjugation of the PEG to the N- or C-terminus of said antibodies or antibody fragments or via epsilon-amino groups present on lysine residues. Linear or branched polymer derivatization that results in minimal loss of biological activity may be used. The degree of conjugation can be closely monitored by SDS-PAGE and mass spectrometry to ensure optimal conjugation of PEG molecules to antibody molecules. Unreacted PEG can be separated from antibody-PEG conjugates by, e.g., size exclusion or ion-exchange chromatography. In a similar manner, the disclosed antibodies can be conjugated to albumin in order to make the antibody or antibody fragment more stable in vivo or have a longer half-life in vivo. The techniques are well known in the art, see e.g., WO 93/15199, WO 93/15200, and WO 01/77137; and EP 0 413, 622. Other biocompatible conjugates are evident to those of ordinary skill and may readily be identified in accordance with the teachings herein.

B. Linker Compounds

Numerous linker compounds can be used to conjugate the antibodies of the invention to the relevant warhead. The linkers merely need to covalently bind with the reactive residue on the antibody (preferably a cysteine or lysine) and the selected drug compound. Accordingly, any linker that reacts with the selected antibody residue and may be used to provide the relatively stable conjugates (site-specific or otherwise) of the instant invention is compatible with the teachings herein.

Compatible linkers can advantageously bind to reduced cysteines and lysines, which are nucleophilic. Conjugation reactions involving reduced cysteines and lysines include, but are not limited to, thiol-maleimide, thiol-halogeno (acyl halide), thiol-ene, thiol-yne, thiol-vinylsulf one, thiol-bisulfone, thiol-thiosulfonate, thiol-pyridyl disulfide and thiol-parafluoro reactions. As further discussed herein, thiol-maleimide bioconjugation is one of the most widely used approaches due to its fast reaction rates and mild conjugation conditions. One issue with this approach is the possibility of the retro-Michael reaction and loss or transfer of the maleimido-linked payload from the antibody to other proteins in the plasma, such as, for example, human serum albumin. However, in some embodiments the use of selective reduction and site-specific antibodies as set forth herein may be used to stabilize the conjugate and reduce this undesired transfer. Thiol-acyl halide reactions provide bioconjugates that cannot undergo retro-Michael reaction and therefore are more stable. However, the thiol-halide reactions in general have slower reaction rates compared to maleimide-based conjugations and are thus not as efficient in providing undesired drug to antibody ratios. Thiol-pyridyl disulfide reaction is another popular bioconjugation route. The pyridyl disulfide undergoes fast exchange with free thiol resulting in the mixed disulfide and release of pyridine-2-thione. Mixed disulfides can be cleaved in the reductive cell environment releasing the payload. Other approaches gaining more attention in bioconjugation are thiol-vinylsulf one and thiol-bisulfone reactions, each of which are compatible with the teachings herein and expressly included within the scope of the invention.

In some embodiments compatible linkers will confer stability on the ADCs in the extracellular environment, prevent aggregation of the ADC molecules and keep the ADC freely soluble in aqueous media and in a monomeric state. Before transport or delivery into a cell, the ADC is preferably stable and remains intact, i.e. the antibody remains linked to the drug moiety. While the linkers are stable outside the target cell they are designed to be cleaved or degraded at some efficacious rate inside the cell. Accordingly an effective linker will: (i) maintain the specific binding properties of the antibody; (ii) allow intracellular delivery of the conjugate or drug moiety; (iii) remain stable and intact, i.e. not cleaved or degraded, until the conjugate has been delivered or transported to its targeted site; and (iv) maintain a cytotoxic, cell-killing effect or a cytostatic effect of the drug moiety (including, in some cases, any bystander effects). The stability of the ADC may be measured by standard analytical techniques such as HPLC/UPLC, mass spectroscopy, HPLC, and the separation/analysis techniques LC/MS and LC/MS/MS. As set forth above covalent attachment of the antibody and the drug moiety requires the linker to have two reactive functional groups, i.e. bivalency in a reactive sense. Bivalent linker reagents which are useful to attach two or more functional or biologically active moieties, such as MMAE and antibodies are known, and methods have been described to provide their resulting conjugates.

Linkers compatible with the present invention may broadly be classified as cleavable and non-cleavable linkers. Cleavable linkers, which may include acid-labile linkers (e.g., oximes and hydrozones), protease cleavable linkers and disulfide linkers, are internalized into the target cell and are cleaved in the endosomal-lysosomal pathway inside the cell. Release and activation of the cytotoxin relies on endosome/lysosome acidic compartments that facilitate cleavage of acid-labile chemical linkages such as hydrazone or oxime. If a lysosomal-specific protease cleavage site is engineered into the linker the cytotoxins will be released in proximity to their intracellular targets. Alternatively, linkers containing mixed disulfides provide an approach by which cytotoxic payloads are released intracellularly as they are selectively cleaved in the reducing environment of the cell, but not in the oxygen-rich environment in the bloodstream. By way of contrast, compatible non-cleavable linkers containing amide linked polyethyleneglycol or alkyl spacers liberate toxic payloads during lysosomal degradation of the ADC within the target cell. In some respects the selection of linker will depend on the particular drug used in the conjugate, the particular indication and the antibody target.

Accordingly, certain embodiments of the invention comprise a linker that is cleavable by a cleaving agent that is present in the intracellular environment (e.g., within a lysosome or endosome or caveolae). The linker can be, for example, a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include Cathepsins B and D and plasmin, each of which is known to hydrolyze dipeptide drug derivatives resulting in the release of active drug inside target cells. Exemplary peptidyl linkers that are cleavable by the thiol-dependent protease Cathepsin-B are peptides comprising Phe-Leu since cathepsin-B has been found to be highly expressed in cancerous tissue. Other examples of such linkers are described, for example, in U.S. Pat. No. 6,214,345. In specific embodiments, the peptidyl linker cleavable by an intracellular protease is a Val-Cit linker, a Val-Ala linker or a Phe-Lys linker. One advantage of using intracellular proteolytic release of the therapeutic agent is that the agent is typically attenuated when conjugated and the serum stabilities of the conjugates are relatively high.

In other embodiments, the cleavable linker is pH-sensitive. Typically, the pH-sensitive linker will be hydrolyzable under acidic conditions. For example, an acid-labile linker that is hydrolyzable in the lysosome (e.g., a hydrazone, oxime, semicarbazone, thiosemicarbazone, cis-aconitic amide, orthoester, acetal, ketal, or the like) can be used (See, e.g., U.S. Pat. Nos. 5,122,368; 5,824,805; 5,622,929). Such linkers are relatively stable under neutral pH conditions, such as those in the blood, but are unstable (e.g., cleavable) at below pH 5.5 or 5.0 which is the approximate pH of the lysosome.

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio) butyrate) and SMPT (N-succinimidyl-oxycarbonyl-alpha-methyl-alpha-(2-pyridyl-dithio)toluene). In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In selected aspects the selected linker will comprise a compound of the formula:

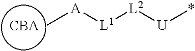

wherein the asterisk indicates the point of attachment to the drug, CBA (i.e. cell binding agent) comprises the anti-DLL3 antibody, L¹ comprises a linker and optionally a cleavable linker, A is a connecting group (optionally comprising a spacer) connecting L¹ to a reactive residue on the antibody, L² is preferably a covalent bond and U, which may or may not be present, can comprise all or part of a self-immolative unit that facilitates a clean separation of the linker from the warhead at the tumor site.

In some embodiments (such as those set forth in U.S.P.N. 2011/0256157) compatible linkers may comprise:

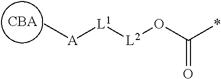

where the asterisk indicates the point of attachment to the drug, CBA (i.e. cell binding agent) comprises an anti-DLL3 antibody, L¹ comprises a linker and optionally a cleavable linker, A is a connecting group (optionally comprising a spacer) connecting L¹ to a reactive residue on the antibody and L² is a covalent bond or together with —OC(═O)— forms a self-immolative moiety.

It will be appreciated that the nature of L¹ and L², where present, can vary widely. These groups are chosen on the basis of their cleavage characteristics, which may be dictated by the conditions at the site to which the conjugate is delivered. Those linkers that are cleaved by the action of enzymes are preferred, although linkers that are cleavable by changes in pH (e.g. acid or base labile), temperature or upon irradiation (e.g. photolabile) may also be used. Linkers that are cleavable under reducing or oxidizing conditions may also find use in the present invention.

In certain embodiments L¹ may comprise a contiguous sequence of amino acids. The amino acid sequence may be the target substrate for enzymatic cleavage, thereby allowing release of the drug.

In one embodiment, L¹ is cleavable by the action of an enzyme. In one embodiment, the enzyme is an esterase or a peptidase.

In another embodiment L¹ is as a Cathepsin labile linker.

In one embodiment, L¹ comprises a dipeptide. The dipeptide may be represented as —NH—X₁—X₂—CO—, where —NH— and —CO— represent the N- and C-terminals of the amino acid groups X₁ and X₂ respectively. The amino acids in the dipeptide may be any combination of natural amino acids. Where the linker is a Cathepsin labile linker, the dipeptide may be the site of action for Cathepsin-mediated cleavage.

Additionally, for those amino acids groups having carboxyl or amino side chain functionality, for example Glu and Lys respectively, CO and NH may represent that side chain functionality.

In one embodiment, the group —X₁—X₂— in dipeptide, —NH—X₁—X₂—CO—, is selected from: -Phe-Lys-, -Val-Ala-, -Val-Lys-, -Ala-Lys-, -Val-Cit-, -Phe-Cit-, -Leu-Cit-, -Ile-Cit-, -Phe-Arg- and -Trp-Cit- where Cit is citrulline.

Preferably, the group —X₁—X₂— in dipeptide, —NH—X₁—X₂—CO—, is selected from: -Phe-Lys-, -Val-Ala-, -Val-Lys-, -Ala-Lys-, and -Val-Cit-.

Most preferably, the group —X₁—X₂— in dipeptide, —NH—X₁—X₂—CO—, is -Phe-Lys- or -Val-Ala- or Val-Cit. In certain selected embodiments the dipeptide will comprise -Val-Ala-.

In one embodiment, L² is present and together with —C(═O)O— forms a self-immolative linker.

In one embodiment, L² is a substrate for enzymatic activity, thereby allowing release of the warhead.

In one embodiment, where L¹ is cleavable by the action of an enzyme and L² is present, the enzyme cleaves the bond between L¹ and L².

L¹ and L², where present, may be connected by a bond selected from: —C(═O)NH—, —C(═O)O—, —NHC(═O)—, —OC(═O)—, —OC(═O)O—, —NHC(═O)O—, —OC(═O)NH—, and —NHC(═O)NH—.

An amino group of L¹ that connects to L² may be the N-terminus of an amino acid or may be derived from an amino group of an amino acid side chain, for example a lysine amino acid side chain.

A carboxyl group of L¹ that connects to L² may be the C-terminus of an amino acid or may be derived from a carboxyl group of an amino acid side chain, for example a glutamic acid amino acid side chain.

A hydroxyl group of L¹ that connects to L² may be derived from a hydroxyl group of an amino acid side chain, for example a serine amino acid side chain.

The term “amino acid side chain” includes those groups found in: (i) naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (ii) minor amino acids such as ornithine and citrulline; (iii) unnatural amino acids, beta-amino acids, synthetic analogs and derivatives of naturally occurring amino acids; and (iv) all enantiomers, diastereomers, isomerically enriched, isotopically labelled (e.g. ²H, ³H, ¹⁴C, ¹⁵N), protected forms, and racemic mixtures thereof.

In one embodiment, —C(═O)O— and L² together form the group:

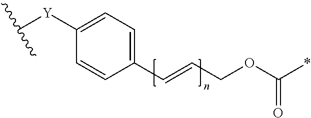

where the asterisk indicates the point of attachment to the drug or cytotoxic agent position, the wavy line indicates the point of attachment to the linker L¹, Y is —N(H)—, —O—, —C(═O)N(H)— or —C(═O)O—, and n is 0 to 3. The phenylene ring is optionally substituted with one, two or three substituents. In one embodiment, the phenylene group is optionally substituted with halo, NO₂, alkyl or hydroxyalkyl.

In one embodiment, Y is NH.

In one embodiment, n is 0 or 1. Preferably, n is 0.

Where Y is NH and n is 0, the self-immolative linker may be referred to as a p-aminobenzylcarbonyl linker (PABC).

In other embodiments the linker may include a self-immolative linker and the dipeptide together form the group —NH-Val-Cit-CO—NH-PABC-. In other selected embodiments the linker may comprise the group —NH-Val-Ala-CO—NH-PABC-, which is illustrated below:

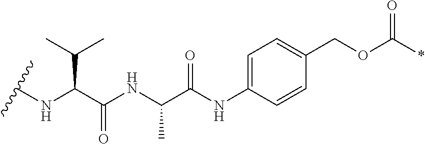 [SMILES: *N[C@H](C(=O)N[C@@H](C)C(=O)Nc1ccc(COC(*)=O)cc1)C(C)C]

where the asterisk indicates the point of attachment to the selected cytotoxic moiety, and the wavy line indicates the point of attachment to the remaining portion of the linker (e.g., the spacer-antibody binding segments) which may be conjugated to the antibody. Upon enzymatic cleavage of the dipeptide, the self-immolative linker will allow for clean release of the protected compound (i.e., the cytotoxin) when a remote site is activated, proceeding along the lines shown below:

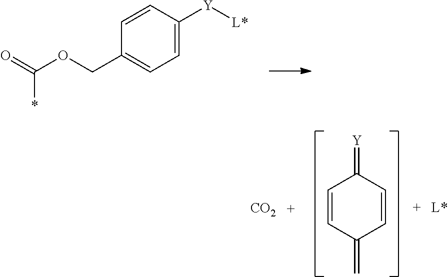

where the asterisk indicates the point of attachment to the selected cytotoxic moiety and where L* is the activated form of the remaining portion of the linker comprising the now cleaved peptidyl unit. The clean release of the warhead ensures it will maintain the desired toxic activity.

In one embodiment, A is a covalent bond. Thus, L¹ and the antibody are directly connected. For example, where L¹ comprises a contiguous amino acid sequence, the N-terminus of the sequence may connect directly to the antibody residue.

In another embodiment, A is a spacer group. Thus, L¹ and the antibody are indirectly connected.

In certain embodiments L¹ and A may be connected by a bond selected from: —C(═O)NH—, —C(═O)O—, —NHC(═O)—, —OC(═O)—, —OC(═O)O—, —NHC(═O)O—, —OC(═O)NH—, and —NHC(═O)NH—.

As will be discussed in more detail below the drug linkers of the instant invention will preferably be linked to reactive thiol nucleophiles on cysteines, including free cysteines. To this end the cysteines of the antibodies may be made reactive for conjugation with linker reagents by treatment with various reducing agent such as DTT or TCEP or mild reducing agents as set forth herein. In other embodiments the drug linkers of the instant invention will preferably be linked to a lysine.

Preferably, the linker contains an electrophilic functional group for reaction with a nucleophilic functional group on the antibody. Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) maleimide groups (ii) activated disulfides, (iii) active esters such as NHS (N-hydroxysuccinimide) esters, HOBt (N-hydroxybenzotriazole) esters, haloformates, and acid halides; (iv) alkyl and benzyl halides such as haloacetamides; and (v) aldehydes, ketones and carboxyl groups.

Exemplary functional groups compatible with the invention are illustrated immediately below:

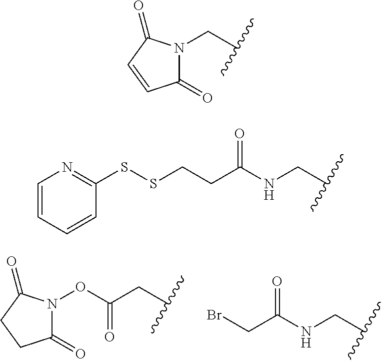 [SMILES: *CN1C(=O)C=CC1=O ; *CNC(=O)CCSSc1ccccn1 ; *CC(=O)ON1C(=O)CCC1=O ; *CNC(=O)CBr]

In some embodiments the connection between a cysteine (including a free cysteine of a site-specific antibody) and the drug-linker moiety is through a thiol residue and a terminal maleimide group of present on the linker. In such embodiments, the connection between the antibody and the drug-linker may be:

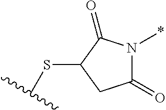 [SMILES: *SC1CC(=O)N(*)C1=O]

where the asterisk indicates the point of attachment to the remaining portion of drug-linker and the wavy line indicates the point of attachment to the remaining portion of the antibody. In this embodiment, the S atom is preferably derived from a site-specific free cysteine.

With regard to other compatible linkers the binding moiety may comprise a terminal iodoacetamide that may be reacted with activated residues on the antibody to provide the desired conjugate. In any event one skilled in the art could readily conjugate each of the disclosed drug-linker compounds with a compatible anti-DLL3 antibody (including site-specific antibodies) in view of the instant disclosure.

In accordance with the instant disclosure the invention provides methods of making compatible antibody drug conjugates comprising conjugating an anti-DLL3 antibody with a drug-linker compound selected from the group consisting of:

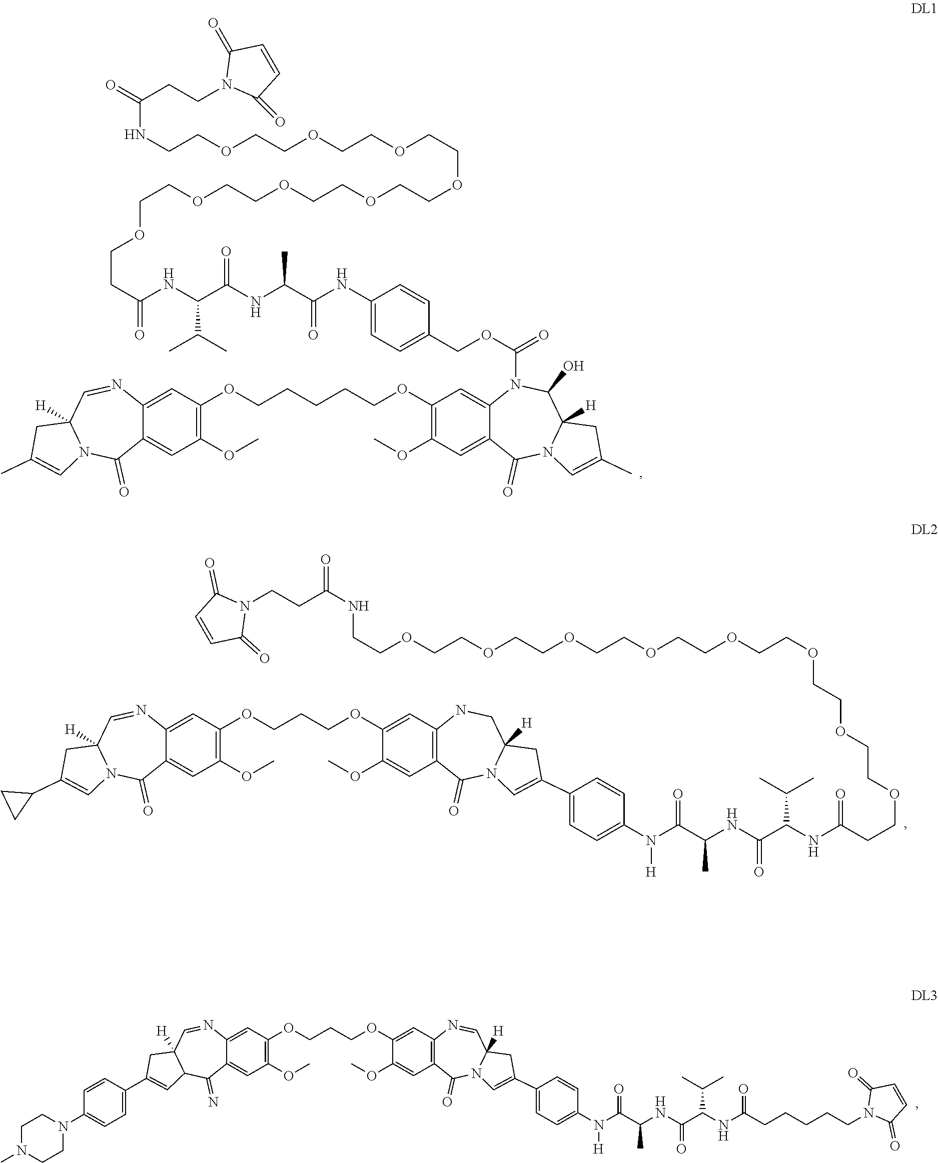

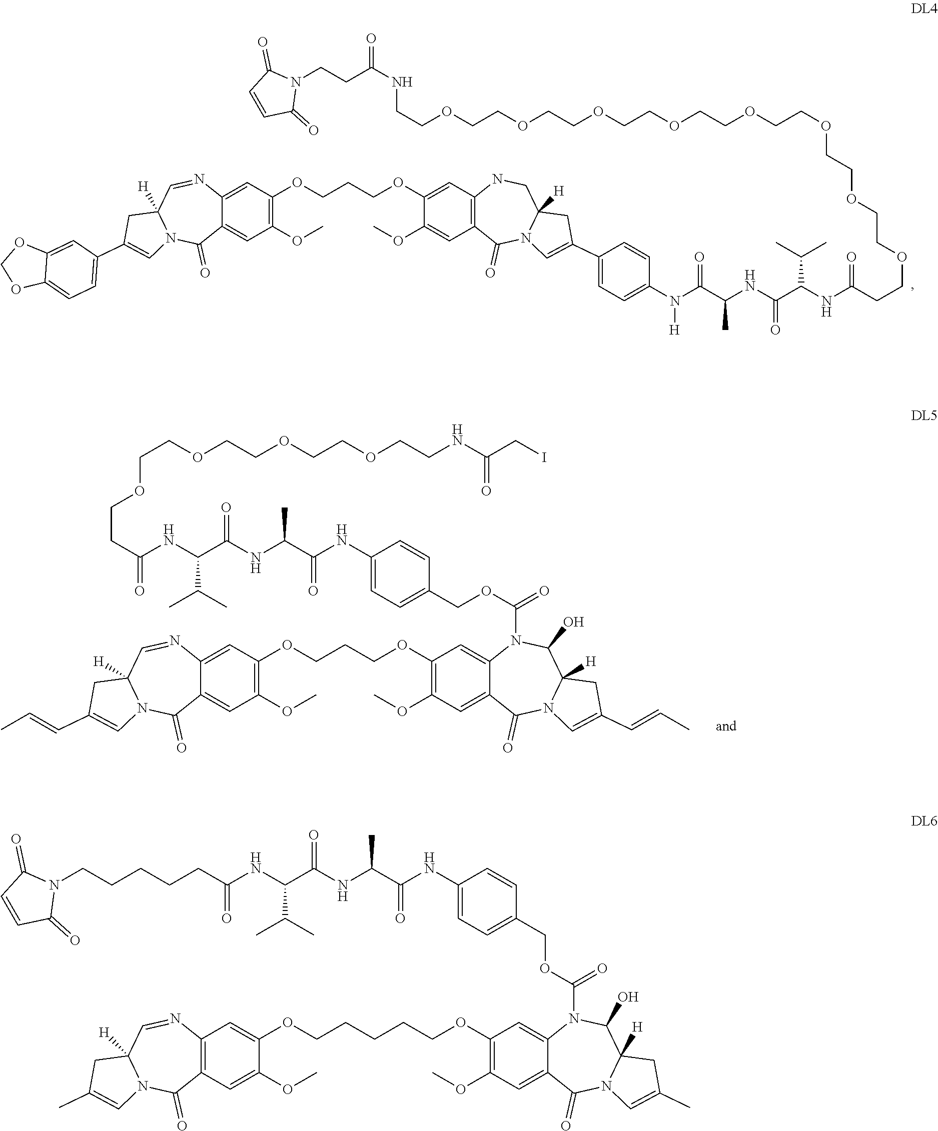

For the purposes of then instant application DL will be used as an abbreviation for “drug-linker” and will comprise drug linkers 1-6 (i.e., DL1, DL2, DL3, DL4 DL5, and DL6) as set forth above. Note that DL1 and DL6 comprise the same warhead and same dipeptide subunit but differ in the connecting group spacer. Accordingly, upon cleavage of the linker both DL1 and DL6 will release PBD1.

It will be appreciated that the linker appended terminal maleimido moiety (DL1-DL4 and DL6) or iodoacetamide moiety (DL5) may be conjugated to free sulfhydryl(s) on the selected DLL3 antibody using art-recognized techniques. Synthetic routes for the aforementioned compounds are set forth in WO2014/130879 which is incorporated herein by reference while specific methods of conjugating such PBDs are set forth in the Examples below.

Thus, in selected aspects the present invention relates to DLL3 antibodies conjugated to the disclosed pyrrolobenzodiazepines to provide DLL3 immunoconjugates substantially set forth in ADCs 1-6 immediately below. Accordingly, in certain aspects the invention is directed to an antibody drug conjugate selected from the group consisting of

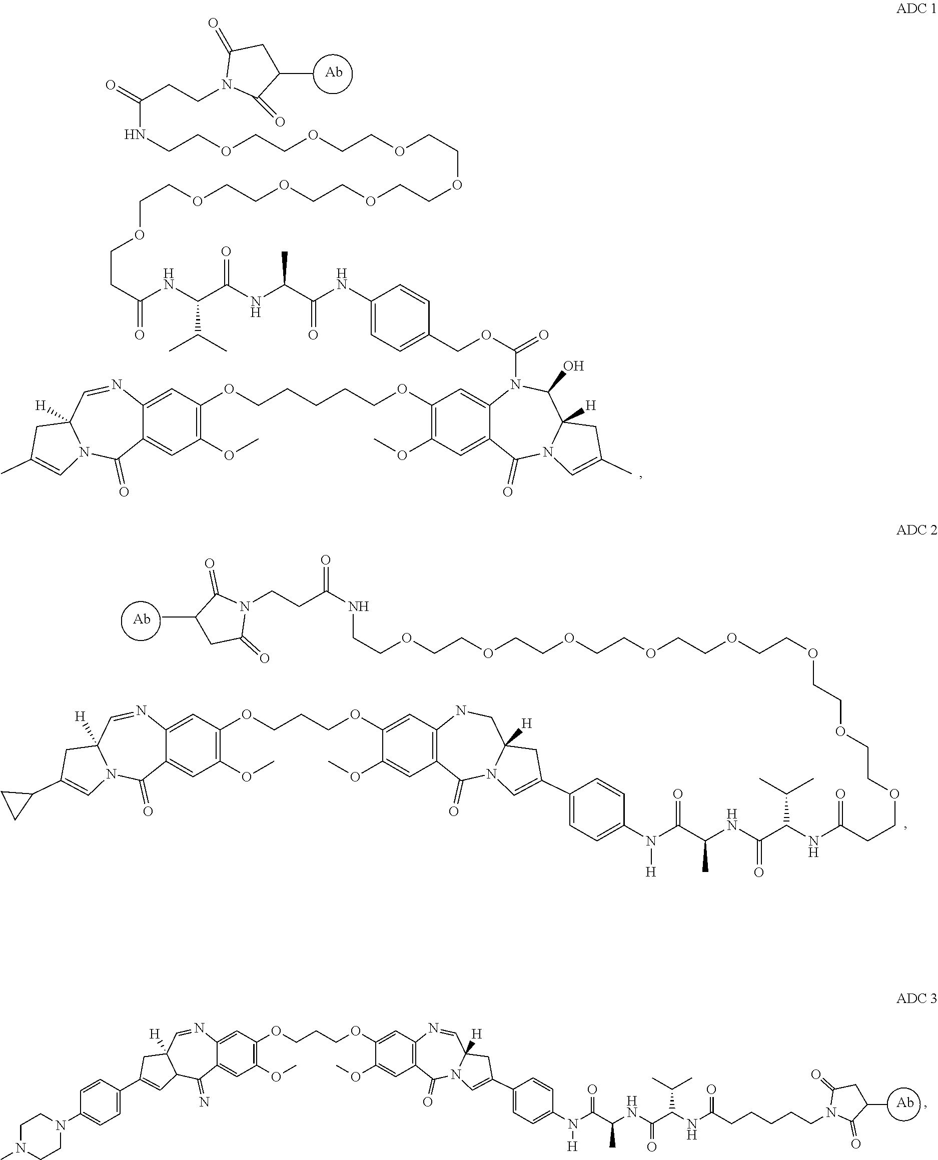

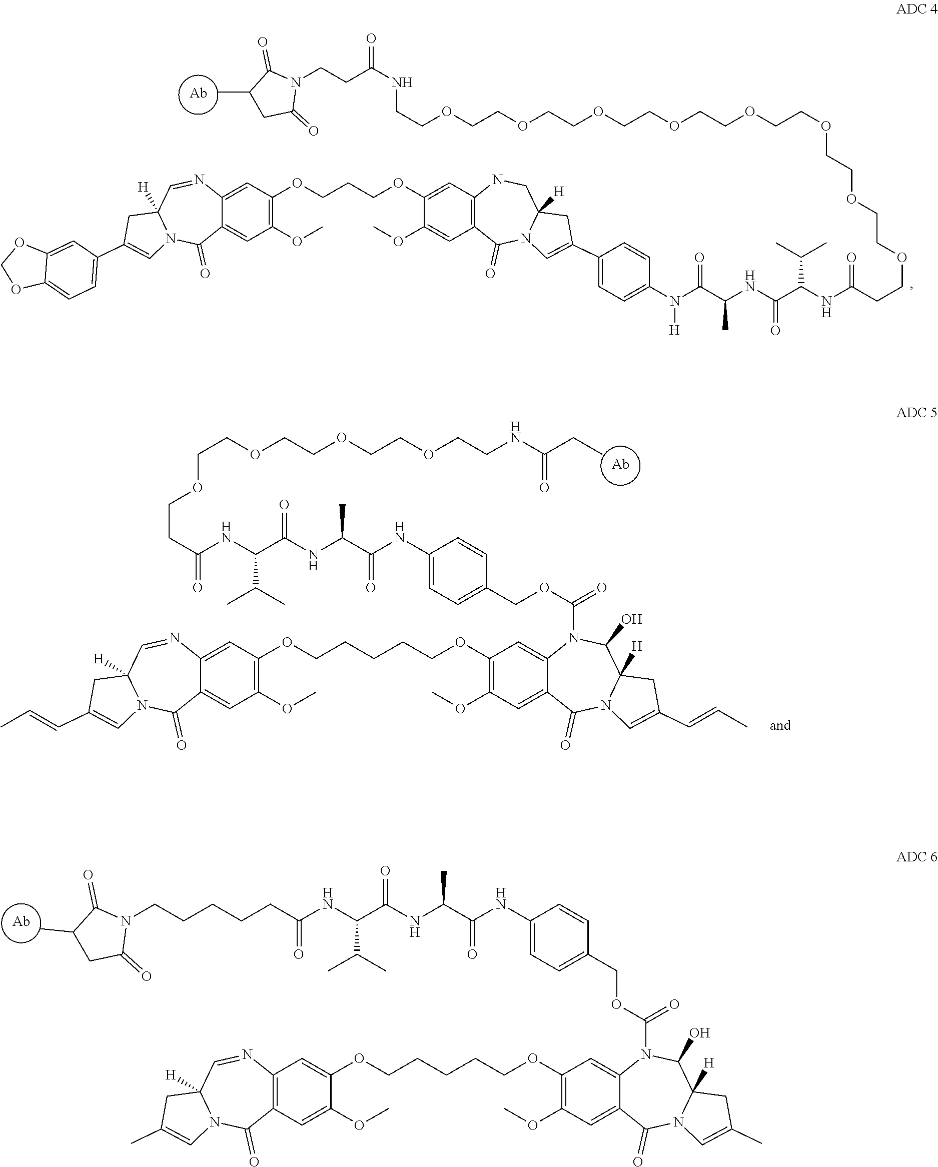

wherein Ab comprises an anti-DLL3 antibody or immunoreactive fragment thereof. Note that ADC1, when it comprises the hSC16.56 antibody (SEQ ID NOS: 7 and 8), may also be referred to as SC16LD6.5 or hSC16.56PBD1 (which may comprise DL1 or DL6) or hSC16.56DL1 or rovalpituzumab tesirine (Rova-T) for the purposes of the instant disclosure. Similarly, ADC6, when it comprises the hSC16.56ss1 antibody (SEQ ID NOS: 8 and 9), may be referred to hSC16.56ss1PBD1 (which may comprise DL1 or DL6) or hSC16.56ss1 DL6 for the purposes of the instant disclosure.

C. Conjugation

It will be appreciated that a number of well-known different reactions may be used to attach the drug moiety and/or linker to the selected antibody. For example, various reactions exploiting sulfhydryl groups of cysteines may be employed to conjugate the desired moiety. Some embodiments will comprise conjugation of antibodies comprising one or more free cysteines as discussed in detail below. In other embodiments ADCs of the instant invention may be generated through conjugation of drugs to solvent-exposed amino groups of lysine residues present in the selected antibody. Still other embodiments comprise activation of N-terminal threonine and serine residues which may then be used to attach the disclosed payloads to the antibody. The selected conjugation methodology will preferably be tailored to optimize the number of drugs attached to the antibody and provide a relatively high therapeutic index.

Various methods are known in the art for conjugating a therapeutic compound to a cysteine residue and will be apparent to the skilled artisan. Under basic conditions the cysteine residues will be deprotonated to generate a thiolate nucleophile which may be reacted with soft electrophiles such as maleimides and iodoacetamides. Generally reagents for such conjugations may react directly with a cysteine thiol to form the conjugated protein or with a linker-drug to form a linker-drug intermediate. In the case of a linker, several routes, employing organic chemistry reactions, conditions, and reagents are known to those skilled in the art, including: (1) reaction of a cysteine group of the protein of the invention with a linker reagent, to form a protein-linker intermediate, via a covalent bond, followed by reaction with an activated compound; and (2) reaction of a nucleophilic group of a compound with a linker reagent, to form a drug-linker intermediate, via a covalent bond, followed by reaction with a cysteine group of a protein of the invention. As will be apparent to the skilled artisan from the foregoing, bifunctional (or bivalent) linkers are useful in the present invention. For example, the bifunctional linker may comprise a thiol modification group for covalent linkage to the cysteine residue(s) and at least one attachment moiety (e.g., a second thiol modification moiety) for covalent or non-covalent linkage to the compound.

Prior to conjugation, antibodies may be made reactive for conjugation with linker reagents by treatment with a reducing agent such as dithiothreitol (DTT) or (tris(2-carboxyethyl)phosphine (TCEP). In other embodiments additional nucleophilic groups can be introduced into antibodies through the reaction of lysines with reagents, including but not limited to, 2-iminothiolane (Traut's reagent), SATA, SATP or SAT(PEG)4, resulting in conversion of an amine into a thiol.

With regard to such conjugations cysteine thiol or lysine amino groups are nucleophilic and capable of reacting to form covalent bonds with electrophilic groups on linker reagents or compound-linker intermediates or drugs including: (i) active esters such as NHS esters, HOBt esters, haloformates, and acid halides; (ii) alkyl and benzyl halides, such as haloacetamides; (iii) aldehydes, ketones, carboxyl, and maleimide groups; and (iv) disulfides, including pyridyl disulfides, via sulfide exchange. Nucleophilic groups on a compound or linker include, but are not limited to amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents.

Conjugation reagents commonly include maleimide, haloacetyl, iodoacetamide succinimidyl ester, isothiocyanate, sulfonyl chloride, 2,6-dichlorotriazinyl, pentafluorophenyl ester, and phosphoramidite, although other functional groups can also be used. In certain embodiments methods include, for example, the use of maleimides, iodoacetimides or haloacetyl/alkyl halides, aziridne, acryloyl derivatives to react with the thiol of a cysteine to produce a thioether that is reactive with a compound. Disulphide exchange of a free thiol with an activated piridyldisulphide is also useful for producing a conjugate (e.g., use of 5-thio-2-nitrobenzoic (TNB) acid). Preferably, a maleimide is used.

As indicated above, lysine may also be used as a reactive residue to effect conjugation as set forth herein. The nucleophilic lysine residue is commonly targeted through amine-reactive succinimidylesters. To obtain an optimal number of deprotonated lysine residues, the pH of the aqueous solution must be below the pKa of the lysine ammonium group, which is around 10.5, so the typical pH of the reaction is about 8 and 9. The common reagent for the coupling reaction is NHS-ester which reacts with nucleophilic lysine through a lysine acylation mechanism. Other compatible reagents that undergo similar reactions comprise isocyanates and isothiocyanates, which also may be used in conjunction with the teachings herein to provide ADCs. Once the lysines have been activated, many of the aforementioned linking groups may be used to covalently bind the warhead to the antibody.

Methods are also known in the art for conjugating a compound to a threonine or serine residue (preferably a N-terminal residue). For example methods have been described in which carbonyl precursors are derived from the 1,2-aminoalcohols of serine or threonine, which can be selectively and rapidly converted to aldehyde form by periodate oxidation. Reaction of the aldehyde with a 1,2-aminothiol of cysteine in a compound to be attached to a protein of the invention forms a stable thiazolidine product. This method is particularly useful for labeling proteins at N-terminal serine or threonine residues.

In some embodiments reactive thiol groups may be introduced into the selected antibody (or fragment thereof) by introducing one, two, three, four, or more free cysteine residues (e.g., preparing antibodies comprising one or more free non-native cysteine amino acid residues). Such site-specific antibodies or engineered antibodies, allow for conjugate preparations that exhibit enhanced stability and substantial homogeneity due, at least in part, to the provision of engineered free cysteine site(s) and/or the novel conjugation procedures set forth herein. Unlike conventional conjugation methodology that fully or partially reduces each of the intrachain or interchain antibody disulfide bonds to provide conjugation sites (and is fully compatible with the instant invention), the present invention additionally provides for the selective reduction of certain prepared free cysteine sites and direction of the drug-linker to the same.

In this regard it will be appreciated that the conjugation specificity promoted by the engineered sites and the selective reduction allows for a high percentage of site directed conjugation at the desired positions. Significantly some of these conjugation sites, such as those present in the terminal region of the light chain constant region, are typically difficult to conjugate effectively as they tend to cross-react with other free cysteines. However, through molecular engineering and selective reduction of the resulting free cysteines, efficient conjugation rates may be obtained which considerably reduces unwanted high-DAR contaminants and non-specific toxicity. More generally the engineered constructs and disclosed novel conjugation methods comprising selective reduction provide ADC preparations having improved pharmacokinetics and/or pharmacodynamics and, potentially, an improved therapeutic index.

In certain embodiments site-specific constructs present free cysteine(s) which, when reduced, comprise thiol groups that are nucleophilic and capable of reacting to form covalent bonds with electrophilic groups on linker moieties such as those disclosed above. As discussed above antibodies of the instant invention may have reducible unpaired interchain or intrachain cysteines or introduced non-native cysteines, i.e. cysteines providing such nucleophilic groups. Thus, in certain embodiments the reaction of free sulfhydryl groups of the reduced free cysteines and the terminal maleimido or haloacetamide groups of the disclosed drug-linkers will provide the desired conjugation. In such cases free cysteines of the antibodies may be made reactive for conjugation with linker reagents by treatment with a reducing agent such as dithiothreitol (DTT) or (tris (2-carboxyethyl)phosphine (TCEP). Each free cysteine will thus present, theoretically, a reactive thiol nucleophile. While such reagents are compatible it will be appreciated that conjugation of site-specific antibodies may be effected using various reactions, conditions and reagents generally known to those skilled in the art.

In addition it has been found that the free cysteines of engineered antibodies may be selectively reduced to provide enhanced site-directed conjugation and a reduction in unwanted, potentially toxic contaminants. More specifically “stabilizing agents” such as arginine have been found to modulate intra- and inter-molecular interactions in proteins and may be used, in conjunction with selected reducing agents (preferably relatively mild), to selectively reduce the free cysteines and to facilitate site-specific conjugation as set forth herein. As used herein the terms “selective reduction” or “selectively reducing” may be used interchangeably and shall mean the reduction of free cysteine(s) without substantially disrupting native disulfide bonds present in the engineered antibody. In selected embodiments this reduction may be effected solely by certain reducing agents. In other embodiments selective reduction of an engineered construct will comprise the use of stabilization agents in combination with reducing agents (including mild reducing agents). It will be appreciated that the term “selective conjugation” shall mean the conjugation of an engineered antibody that has been selectively reduced, with a cytotoxin as described herein. In this respect the use of such stabilizing agents in combination with selected reducing agents can markedly improve the efficiency of site-specific conjugation as determined by extent of conjugation at selected sites on the heavy and/or light antibody chains and DAR distribution of the preparation. Compatible antibody constructs and selective conjugation techniques and reagents are extensively disclosed in WO2015/031698 which is incorporated herein in its entirety as to such methodology and constructs.

While not wishing to be bound by any particular theory, such stabilizing agents may act to modulate the electrostatic microenvironment and/or modulate conformational changes at the desired conjugation site, thereby allowing relatively mild reducing agents (which do not materially reduce intact native disulfide bonds) to facilitate conjugation at the desired free cysteine site(s). Such agents (e.g., certain amino acids) are known to form salt bridges (via hydrogen bonding and electrostatic interactions) and can modulate protein-protein interactions in such a way as to impart a stabilizing effect that may cause favorable conformation changes and/or reduce unfavorable protein-protein interactions. Moreover, such agents may act to inhibit the formation of undesired intramolecular (and intermolecular) cysteine-cysteine bonds after reduction thus facilitating the desired conjugation reaction wherein the engineered site-specific cysteine is bound to the drug (preferably via a linker). Since selective reduction conditions do not provide for the significant reduction of intact native disulfide bonds, the subsequent conjugation reaction is naturally driven to the relatively few reactive thiols on the free cysteines (e.g., preferably 2 free thiols per antibody). As previously alluded to, such techniques may be used to considerably reduce levels of non-specific conjugation and corresponding impurities in conjugate preparations fabricated in accordance with the instant disclosure. Consequently these site-specific ADC compositions may exhibit reduced toxicity.

In selected embodiments stabilizing agents compatible with the present invention will generally comprise compounds with at least one moiety having a basic pKa. In certain embodiments the moiety will comprise a primary amine while in other embodiments the amine moiety will comprise a secondary amine. In still other embodiments the amine moiety will comprise a tertiary amine or a guanidinium group. In other selected embodiments the amine moiety will comprise an amino acid while in other compatible embodiments the amine moiety will comprise an amino acid side chain. In yet other embodiments the amine moiety will comprise a proteinogenic amino acid. In still other embodiments the amine moiety comprises a non-proteinogenic amino acid. In some embodiments, compatible stabilizing agents may comprise arginine, lysine, proline and cysteine. In addition compatible stabilizing agents may include guanidine and nitrogen containing heterocycles with basic pKa.

In certain embodiments compatible stabilizing agents comprise compounds with at least one amine moiety having a pKa of greater than about 7.5, in other embodiments the subject amine moiety will have a pKa of greater than about 8.0, in yet other embodiments the amine moiety will have a pKa greater than about 8.5 and in still other embodiments the stabilizing agent will comprise an amine moiety having a pKa of greater than about 9.0. Other embodiments will comprise stabilizing agents where the amine moiety will have a pKa of greater than about 9.5 while certain other embodiments will comprise stabilizing agents exhibiting at least one amine moiety having a pKa of greater than about 10.0. In still other embodiments the stabilizing agent will comprise a compound having the amine moiety with a pKa of greater than about 10.5, in other embodiments the stabilizing agent will comprise a compound having a amine moiety with a pKa greater than about 11.0, while in still other embodiments the stabilizing agent will comprise a amine moiety with a pKa greater than about 11.5. In yet other embodiments the stabilizing agent will comprise a compound having an amine moiety with a pKa greater than about 12.0, while in still other embodiments the stabilizing agent will comprise an amine moiety with a pKa greater than about 12.5. Those of skill in the art will understand that relevant pKa's may readily be calculated or determined using standard techniques and used to determine the applicability of using a selected compound as a stabilizing agent.

The disclosed stabilizing agents are shown to be particularly effective at targeting conjugation to free site-specific cysteines when combined with certain reducing agents. For the purposes of the instant invention, compatible reducing agents may include any compound that produces a reduced free site-specific cysteine for conjugation without significantly disrupting the native disulfide bonds of the engineered antibody. Under such conditions, preferably provided by the combination of selected stabilizing and reducing agents, the activated drug linker is largely limited to binding to the desired free site-specific cysteine site(s). Relatively mild reducing agents or reducing agents used at relatively low concentrations to provide mild conditions are particularly preferred. As used herein the terms “mild reducing agent” or “mild reducing conditions” shall be held to mean any agent or state brought about by a reducing agent (optionally in the presence of stabilizing agents) that provides thiols at the free cysteine site(s) without substantially disrupting native disulfide bonds present in the engineered antibody. That is, mild reducing agents or conditions (preferably in combination with a stabilizing agent) are able to effectively reduce free cysteine(s) (provide a thiol) without significantly disrupting the protein's native disulfide bonds. The desired reducing conditions may be provided by a number of sulfhydryl-based compounds that establish the appropriate environment for selective conjugation. In embodiments mild reducing agents may comprise compounds having one or more free thiols while in some embodiments mild reducing agents will comprise compounds having a single free thiol. Non-limiting examples of reducing agents compatible with the selective reduction techniques of the instant invention comprise glutathione, n-acetyl cysteine, cysteine, 2-aminoethane-1-thiol and 2-hydroxyethane-1-thiol.

It will be appreciated that selective reduction process set forth above is particularly effective at targeted conjugation to the free cysteine. In this respect the extent of conjugation to the desired target site (defined here as “conjugation efficiency”) in site-specific antibodies may be determined by various art-accepted techniques. The efficiency of the site-specific conjugation of a drug to an antibody may be determined by assessing the percentage of conjugation on the target conjugation site(s) (e.g. free cysteines on the c-terminus of each light chain) relative to all other conjugated sites. In certain embodiments, the method herein provides for efficiently conjugating a drug to an antibody comprising free cysteines. In some embodiments, the conjugation efficiency is at least 5%, at least 10%, at least 15%, at least 20%, at least 25%, at least 30%, at least 35%, at least 40%, at least 45%, at least 50%, at least 55%, at least 60%, at least 70%, at least 75%, at least 80%, at least 85%, at least 90%, at least 95%, at least 98% or more as measured by the percentage of target conjugation relative to all other conjugation sites.

It will further be appreciated that engineered antibodies capable of conjugation may contain free cysteine residues that comprise sulfhydryl groups that are blocked or capped as the antibody is produced or stored. Such caps include small molecules, proteins, peptides, ions and other materials that interact with the sulfhydryl group and prevent or inhibit conjugate formation. In some cases the unconjugated engineered antibody may comprise free cysteines that bind other free cysteines on the same or different antibodies. As discussed herein such cross-reactivity may lead to various contaminants during the fabrication procedure. In some embodiments, the engineered antibodies may require uncapping prior to a conjugation reaction. In specific embodiments, antibodies herein are uncapped and display a free sulfhydryl group capable of conjugation. In other specific embodiments, antibodies herein are subjected to an uncapping reaction that does not disturb or rearrange the naturally occurring disulfide bonds. It will be appreciated that in most cases the uncapping reactions will occur during the normal reduction reactions (reduction or selective reduction).

D. DAR Distribution and Purification

In selected embodiments conjugation and purification methodology compatible with the present invention advantageously provides the ability to generate relatively homogeneous ADC preparations comprising a narrow DAR distribution. In this regard the disclosed constructs (e.g., site specific constructs) and/or selective conjugation provides for homogeneity of the ADC species within a sample in terms of the stoichiometric ratio between the drug and the engineered antibody and with respect to the toxin location. As briefly discussed above the term “drug to antibody ratio” or “DAR” refers to the molar ratio of drug to antibody. In certain embodiments a conjugate preparation may be substantially homogeneous with respect to its DAR distribution, meaning that within the ADC preparation is a predominant species of site-specific ADC with a particular DAR (e.g., a DAR of 2 or 4) that is also uniform with respect to the site of loading (i.e., on the free cysteines). In other certain embodiments of the invention it is possible to achieve the desired homogeneity through the use of site-specific antibodies and/or selective reduction and conjugation. In other embodiments the desired homogeneity may be achieved through the use of site-specific constructs in combination with selective reduction. In yet other embodiments compatible preparations may be further purified using analytical or preparative chromatography techniques to provide the desired homogeneity. In each of these embodiments the homogeneity of the ADC sample can be analyzed using various techniques known in the art including but not limited to mass spectrometry, HPLC (e.g. size exclusion HPLC, RP-HPLC, HIC-HPLC etc.) or capillary electrophoresis.

With regard to the purification of ADC preparations it will be appreciated that standard pharmaceutical preparative methods may be employed to obtain the desired purity. As discussed herein liquid chromatography methods such as reverse phase (RP) and hydrophobic interaction chromatography (HIC) may separate compounds in the mixture by drug loading value. In some cases, ion-exchange (IEC) or mixed-mode chromatography (MMC) may also be used to isolate species with a specific drug load.

The disclosed ADCs and preparations thereof may comprise drug and antibody moieties in various stoichiometric molar ratios depending on the configuration of the antibody and, at least in part, on the method used to effect conjugation. In certain embodiments the drug loading per ADC may comprise from 1-20 warheads (i.e., n is 1-20). Other selected embodiments may comprise ADCs with a drug loading of from 1 to 15 warheads. In still other embodiments the ADCs may comprise from 1-12 warheads or, more preferably, from 1-10 warheads. In some embodiments the ADCs will comprise from 1 to 8 warheads.

While theoretical drug loading may be relatively high, practical limitations such as free cysteine cross reactivity and warhead hydrophobicity tend to limit the generation of homogeneous preparations comprising such DAR due to aggregates and other contaminants. That is, higher drug loading, e.g. >6 or 8, may cause aggregation, insolubility, toxicity, or loss of cellular permeability of certain antibody-drug conjugates depending on the payload. In view of such concerns practical drug loading provided by the instant invention preferably ranges from 1 to 8 drugs per conjugate, i.e. where 1, 2, 3, 4, 5, 6, 7, or 8 drugs are covalently attached to each antibody (e.g., for IgG1, other antibodies may have different loading capacity depending the number of disulfide bonds). Preferably the DAR of compositions of the instant invention will be approximately 2, 4 or 6 and in some embodiments the DAR will comprise approximately 2.

Despite the relatively high level of homogeneity provided by the instant invention the disclosed compositions actually comprise a mixture of conjugates with a range of drugs compounds (e.g., potentially from 1 to 8 in the case of an IgG1). As such, the disclosed ADC compositions include mixtures of conjugates where most of the constituent antibodies are covalently linked to one or more drug moieties and (despite the relative conjugate specificity provided by engineered constructs and selective reduction) where the drug moieties may be attached to the antibody by various thiol groups. That is, following conjugation ADC compositions of the invention will comprise a mixture of conjugates with different drug loads (e.g., from 1 to 8 drugs per IgG1 antibody) at various concentrations (along with certain reaction contaminants primarily caused by free cysteine cross reactivity). However using selective reduction and post-fabrication purification the conjugate compositions may be driven to the point where they largely contain a single predominant desired ADC species (e.g., with a drug loading of 2) with relatively low levels of other ADC species (e.g., with a drug loading of 1, 4, 6, etc.). The average DAR value represents the weighted average of drug loading for the composition as a whole (i.e., all the ADC species taken together). Due to inherent uncertainty in the quantification methodology employed and the difficulty in completely removing the non-predominant ADC species in a commercial setting, acceptable DAR values or specifications are often presented as an average, a range or distribution (i.e., an average DAR of 2+/−0.5). Preferably compositions comprising a measured average DAR within the range (i.e., 1.5 to 2.5) would be used in a pharmaceutical setting.

Thus, in some embodiments the present invention will comprise compositions having an average DAR of 1, 2, 3, 4, 5, 6, 7 or 8 each +/−0.5. In other embodiments the present invention will comprise an average DAR of 2, 4, 6 or 8+/−0.5. Finally, in selected embodiments the present invention will comprise an average DAR of 2+/−0.5 or 4+/−0.5. It will be appreciated that the range or deviation may be less than 0.4 in some embodiments. Thus, in other embodiments the compositions will comprise an average DAR of 1, 2, 3, 4, 5, 6, 7 or 8 each +/−0.3, an average DAR of 2, 4, 6 or 8+/−0.3, even more preferably an average DAR of 2 or 4+/−0.3 or even an average DAR of 2+/−0.3. In other embodiments IgG1 conjugate compositions will preferably comprise a composition with an average DAR of 1, 2, 3, 4, 5, 6, 7 or 8 each +/−0.4 and relatively low levels (i.e., less than 30%) of non-predominant ADC species. In other embodiments the ADC composition will comprise an average DAR of 2, 4, 6 or 8 each +/−0.4 with relatively low levels (<30%) of non-predominant ADC species. In some embodiments the ADC composition will comprise an average DAR of 2+/−0.4 with relatively low levels (<30%) of non-predominant ADC species. In yet other embodiments the predominant ADC species (e.g., DAR of 2 or DAR of 4) will be present at a concentration of greater than 65%, at a concentration of greater than 70%, at a concentration of greater than 75%, at a concentration of greater that 80%, at a concentration of greater than 85%, at a concentration of greater than 90%, at a concentration of greater than 93%, at a concentration of greater than 95% or even at a concentration of greater than 97% when measured against other DAR species.

Those of skill in the art will appreciate that the distribution of drugs per antibody in preparations of ADC from conjugation reactions may be characterized by conventional means such as UV-Vis spectrophotometry, reverse phase HPLC, HIC, mass spectroscopy, ELISA, and electrophoresis. By way of example the quantitative distribution of ADC in terms of drugs per antibody may also be determined using a combination of HIC and RP chromatography. Similarly the averaged value of the drugs per antibody in a particular preparation of ADC may be determined using ELISA although the distribution of drug per antibody value is not readily discernible due to antibody-antigen binding and detection limitations. Also, ELISA assays do not provide information as to where the drug moieties are attached on the antibody. However, as alluded to above such data is readily obtainable using various chromatography and electrophoresis techniques well known in the art.

### VI. DIAGNOSTICS AND SCREENING

A. Diagnostics

The invention provides in vitro and in vivo methods for detecting, diagnosing or monitoring proliferative disorders and methods of screening cells from a patient to identify tumor cells including tumorigenic cells. Such methods include identifying an individual having cancer for treatment or monitoring progression of a cancer, comprising contacting the patient or a sample obtained from a patient (either in vivo or in vitro) with a detection agent (e.g., an antibody or nucleic acid probe) capable of specifically recognizing and associating with a DLL3 determinant and detecting the presence or absence, or level of association of the detection agent in the sample. In selected embodiments the detection agent will comprise an antibody associated with a detectable label or reporter molecule as described herein. In certain other embodiments the DLL3 antibody will be administered and detected using a secondary labelled antibody (e.g., an anti-murine antibody). In yet other embodiments (e.g., In situ hybridization or ISH) a nucleic acid probe that reacts with a genomic DLL3 determinant will be used in the detection, diagnosis or monitoring of the proliferative disorder.

More generally the presence and/or levels of DLL3 determinants may be measured using any of a number of techniques available to the person of ordinary skill in the art for protein or nucleic acid analysis, e.g., direct physical measurements (e.g., mass spectrometry), binding assays (e.g., immunoassays, agglutination assays, and immunochromatographic assays), Polymerase Chain Reaction (PCR, RT-PCR; RT-qPCR) technology, branched oligonucleotide technology, Northern blot technology, oligonucleotide hybridization technology and in situ hybridization technology. The method may also comprise measuring a signal that results from a chemical reaction, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques may detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques may be used that do not require the use of labels, e.g., techniques based on measuring mass (e.g., surface acoustic wave measurements), refractive index (e.g., surface plasmon resonance measurements), or the inherent luminescence of an analyte.

In some embodiments, the association of the detection agent with particular cells or cellular components in the sample indicates that the sample may contain tumorigenic cells, thereby denoting that the individual having cancer may be effectively treated with an antibody or ADC as described herein.

In certain preferred embodiments the assays may comprise immunohistochemistry (IHC) assays or variants thereof (e.g., fluorescent, chromogenic, standard ABC, standard LSAB, etc.), immunocytochemistry or variants thereof (e.g., direct, indirect, fluorescent, chromogenic, etc.) or In situ hybridization (ISH) or variants thereof (e.g., chromogenic in situ hybridization (CISH) or fluorescence in situ hybridization (DNA-FISH or RNA-FISH]))

In this regard certain aspects of the instant invention comprise the use of labeled DLL3 antibodies for immunohistochemistry (IHC). In other embodiments the DLL3 antibody will be detected using a secondary labelled antibody. More particularly DLL3 IHC may be used as a diagnostic tool to aid in the diagnosis of various proliferative disorders and to monitor the potential response to treatments including DLL3 antibody therapy. As discussed herein and shown in the Examples below compatible diagnostic assays may be performed on tissues that have been chemically fixed (compatible techniques include, but are not limited to: formaldehyde, gluteraldehyde, osmium tetroxide, potassium dichromate, acetic acid, alcohols, zinc salts, mercuric chloride, chromium tetroxide and picric acid) and embedded (compatible methods include but are not limited to: glycol methacrylate, paraffin and resins) or preserved via freezing. Such assays can be used to guide treatment decisions and determine dosing regimens and timing.

As shown in the Examples below immunohistochemistry techniques may be used to derive an H-score as known in the art. Such H-scores may be used to indicate which patients may be amenable to treatment with the compositions of the instant invention. Based on the Examples below H-scores of approximately 90, approximately 100, approximately 110, approximately 120, approximately 130, approximately 140, approximately 150, approximately 160, approximately 170, approximately 180, approximately 190 or approximately 200 or above on a 300 point scale may be used in selected embodiments to indicate which patients may respond favorably to the treatment methods of the instant invention. Accordingly in one embodiment a patient to be treated with the DLL3 ADCs of the instant invention will have an H-score of at least 90 (i.e., the tumor is DLL3+) on a 300 point scale. In other embodiments a patient to be treated with the DLL3 ADCs of the instant invention will have an H-score of at least 120. In yet other embodiments a patient to be treated with the DLL3 ADCs of the instant invention will have an H-score of at least 180. For the purposes of the instant disclosure any tumor exhibiting an H-score of 90 or above on a 300 point scale will be considered a DLL3+ tumor and subject to treatment with the disclosed antibodies or ADCs.

In other embodiments patient selection may be based on the more simple measurement of percent of positively stained DLL3 cells in a tumor sample. In this regard patients exhibiting a certain percentage of positively stained cells in an IHC sample when interrogated with an anti-DLL3 antibody would be considered DLL3+ and would be selected for treatment in accordance with the teachings herein. In such embodiments tumor samples exhibiting greater than 10%, greater than 20%, greater than 30%, greater than 40% or greater than 50% positive cell staining may be classified as DLL3+. In other embodiments tumor samples exhibiting greater than 60%, greater than 70%, greater than 80%, greater than 90% or greater than 95% positive cell staining may be classified as DLL3+. In certain preferred aspects the DLL3+ tumor will express DLL3 in 50% of the constituent cells. In each of the forgoing embodiments patients suffering from DLL3+ positive tumors may be treated with the disclosed compositions as set forth herein.

Other particularly compatible aspects of the invention involve the use of in situ hybridization to detect or monitor DLL3 determinants. In situ hybridization technology or ISH is well known to those of skill in the art. Briefly, cells are fixed and detectable probes which contain a specific nucleotide sequence are added to the fixed cells. If the cells contain complementary nucleotide sequences, the probes, which can be detected, will hybridize to them. Using the sequence information set forth herein, probes can be designed to identify cells that express genotypic DLL3 determinants. Probes preferably hybridize to a nucleotide sequence that corresponds to such determinants. Hybridization conditions can be routinely optimized to minimize background signal by non-fully complementary hybridization though preferably the probes are preferably fully complementary to the selected DLL3 determinant. In selected embodiments the probes are labeled with fluorescent dye attached to the probes that is readily detectable by standard fluorescent methodology.

Compatible in vivo theragnostics or diagnostic assays may comprise art-recognized imaging or monitoring techniques such as magnetic resonance imaging, computerized tomography (e.g. CAT scan), positron tomography (e.g., PET scan) radiography, ultrasound, etc., as would be known by those skilled in the art.

In certain embodiments the antibodies of the instant invention may be used to detect and quantify levels of a particular determinant (e.g., DLL3 protein) in a patient sample (e.g., plasma or blood) which may, in turn, be used to detect, diagnose or monitor proliferative disorders that are associated with the relevant determinant. In related embodiments the antibodies of the instant invention may be used to detect, monitor and/or quantify circulating tumor cells either in vivo or in vitro (WO 2012/0128801). In still other embodiments the circulating tumor cells may comprise tumorigenic cells.

In certain embodiments of the invention, the tumorigenic cells in a subject or a sample from a subject may be assessed or characterized using the disclosed antibodies prior to therapy or regimen to establish a baseline. In other examples, the tumorigenic cells can be assessed from a sample that is derived from a subject that was treated.

In another embodiment, the invention provides a method of analyzing cancer progression and/or pathogenesis in vivo. In another embodiment, analysis of cancer progression and/or pathogenesis in vivo comprises determining the extent of tumor progression. In another embodiment, analysis comprises the identification of the tumor. In another embodiment, analysis of tumor progression is performed on the primary tumor. In another embodiment, analysis is performed over time depending on the type of cancer as known to one skilled in the art. In another embodiment, further analysis of secondary tumors originating from metastasizing cells of the primary tumor is conducted in vivo. In another embodiment, the size and shape of secondary tumors are analyzed. In some embodiments, further ex vivo analysis is performed.

In another embodiment, the invention provides a method of analyzing cancer progression and/or pathogenesis in vivo including determining cell metastasis or detecting and quantifying the level of circulating tumor cells. In yet another embodiment, analysis of cell metastasis comprises determination of progressive growth of cells at a site that is discontinuous from the primary tumor. In some embodiments, procedures may be undertaken to monitor tumor cells that disperse via blood vasculature, lymphatics, within body cavities or combinations thereof. In another embodiment, cell metastasis analysis is performed in view of cell migration, dissemination, extravasation, proliferation or combinations thereof.

In certain examples, the tumorigenic cells in a subject or a sample from a subject may be assessed or characterized using the disclosed antibodies prior to therapy to establish a baseline. In other examples the sample is derived from a subject that was treated. In some examples the sample is taken from the subject at least about 1, 2, 4, 6, 7, 8, 10, 12, 14, 15, 16, 18, 20, 30, 60, 90 days, 6 months, 9 months, 12 months, or >12 months after the subject begins or terminates treatment. In certain examples, the tumorigenic cells are assessed or characterized after a certain number of doses (e.g., after 2, 5, 10, 20, 30 or more doses of a therapy). In other examples, the tumorigenic cells are characterized or assessed after 1 week, 2 weeks, 1 month, 2 months, 1 year, 2 years, 3 years, 4 years or more after receiving one or more therapies.

B. Screening

In certain embodiments, antibodies of the instant invention can be used to screen samples in order to identify compounds or agents (e.g., antibodies or ADCs) that alter a function or activity of tumor cells by interacting with a determinant. In one embodiment, tumor cells are put in contact with an antibody or ADC and the antibody or ADC can be used to screen the tumor for cells expressing a certain target (e.g. DLL3) in order to identify such cells for purposes, including but not limited to, diagnostic purposes, to monitor such cells to determine treatment efficacy or to enrich a cell population for such target-expressing cells.

In yet another embodiment, a method includes contacting, directly or indirectly, tumor cells with a test agent or compound and determining if the test agent or compound modulates an activity or function of the determinant-associated tumor cells for example, changes in cell morphology or viability, expression of a marker, differentiation or de-differentiation, cell respiration, mitochondrial activity, membrane integrity, maturation, proliferation, viability, apoptosis or cell death. One example of a direct interaction is physical interaction, while an indirect interaction includes, for example, the action of a composition upon an intermediary molecule that, in turn, acts upon the referenced entity (e.g., cell or cell culture).

Screening methods include high throughput screening, which can include arrays of cells (e.g., microarrays) positioned or placed, optionally at pre-determined locations, for example, on a culture dish, tube, flask, roller bottle or plate. High-throughput robotic or manual handling methods can probe chemical interactions and determine levels of expression of many genes in a short period of time. Techniques have been developed that utilize molecular signals, for example via fluorophores or microarrays (Mocellin and Rossi, 2007, PMID: 17265713) and automated analyses that process information at a very rapid rate (see, e.g., Pinhasov et al., 2004, PMID: 15032660). Libraries that can be screened include, for example, small molecule libraries, phage display libraries, fully human antibody yeast display libraries (Adimab), siRNA libraries, and adenoviral transfection vectors.

### VII. PHARMACEUTICAL PREPARATIONS AND THERAPEUTIC USES

A. Formulations and Routes of Administration

The antibodies or ADCs of the invention can be formulated in various ways using art recognized techniques. In some embodiments, the therapeutic compositions of the invention can be administered neat or with a minimum of additional components while others may optionally be formulated to contain suitable pharmaceutically acceptable carriers. As used herein, “pharmaceutically acceptable carriers” comprise excipients, vehicles, adjuvants and diluents that are well known in the art and can be available from commercial sources for use in pharmaceutical preparation (see, e.g., Gennaro (2003) Remington: The Science and Practice of Pharmacy with Facts and Comparisons: Drugfacts Plus, 20th ed., Mack Publishing; Ansel et al. (2004) Pharmaceutical Dosage Forms and Drug Delivery Systems, 7^{th} ed., Lippencott Williams and Wilkins; Kibbe et al. (2000) Handbook of Pharmaceutical Excipients, 3^{rd} ed., Pharmaceutical Press.)

Suitable pharmaceutically acceptable carriers comprise substances that are relatively inert and can facilitate administration of the antibody or ADC or can aid processing of the active compounds into preparations that are pharmaceutically optimized for delivery to the site of action.

Such pharmaceutically acceptable carriers include agents that can alter the form, consistency, viscosity, pH, tonicity, stability, osmolarity, pharmacokinetics, protein aggregation or solubility of the formulation and include buffering agents, wetting agents, emulsifying agents, diluents, encapsulating agents and skin penetration enhancers. Certain non-limiting examples of carriers include saline, buffered saline, dextrose, arginine, sucrose, water, glycerol, ethanol, sorbitol, dextran, sodium carboxymethyl cellulose and combinations thereof. Antibodies for systemic administration may be formulated for enteral, parenteral or topical administration. In certain embodiments the disclosed compositions will be formulated for intravenous administration and will preferably be infused using an IV container (e.g. an IV drip bag). Indeed, all three types of formulation may be used simultaneously to achieve systemic administration of the active ingredient. Excipients as well as formulations for parenteral and nonparenteral drug delivery are set forth in Remington: The Science and Practice of Pharmacy (2000) 20th Ed. Mack Publishing.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection or infusion), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additionally contain other pharmaceutically acceptable carriers, such as anti-oxidants, buffers, preservatives, stabilizers, bacteriostats, suspending agents, thickening agents, and solutes that render the formulation isotonic with the blood (or other relevant bodily fluid) of the intended recipient. Examples of excipients include, for example, water, alcohols, polyols, glycerol, vegetable oils, and the like. Examples of suitable isotonic pharmaceutically acceptable carriers for use in such formulations include Sodium Chloride Injection, Ringer's Solution, or Lactated Ringer's Injection.

In particularly preferred embodiments formulated compositions of the present invention may be lyophilized to provide a powdered form of the antibody or ADC which may then be reconstituted prior to administration. Sterile powders for the preparation of injectable solutions may be generated by lyophilizing a solution comprising the disclosed antibodies or ADCs to yield a powder comprising the active ingredient along with any optional co-solubilized biocompatible ingredients. Generally, dispersions or solutions are prepared by incorporating the active compound into a sterile vehicle that contains a basic dispersion medium or solvent (e.g., a diluent) and, optionally, other biocompatible ingredients. A compatible diluent is one which is pharmaceutically acceptable (safe and non-toxic for administration to a human) and is useful for the preparation of a liquid formulation, such as a formulation reconstituted after lyophilization. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

In certain preferred embodiments the anti-DLL3 antibodies or ADCs will be lyophilized in combination with a pharmaceutically acceptable sugar. A “pharmaceutically acceptable sugar” is a molecule which, when combined with a protein of interest, significantly prevents or reduces chemical and/or physical instability of the protein upon storage. Such sugars are particularly compatible when the formulation is intended to be lyophilized and then reconstituted. As used herein pharmaceutically acceptable sugars may also be referred to as a “lyoprotectant”. Exemplary sugars and their corresponding sugar alcohols include: an amino acid such as monosodium glutamate or histidine; a methylamine such as betaine; a lyotropic salt such as magnesium sulfate; a polyol such as trihydric or higher molecular weight sugar alcohols, e.g. glycerin, dextran, erythritol, glycerol, arabitol, xylitol, sorbitol, and mannitol; propylene glycol; polyethylene glycol; PLURONICS®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred pharmaceutically-acceptable sugars are the non-reducing sugars trehalose or sucrose. Pharmaceutically acceptable sugars are added to the formulation in a “protecting amount” (e.g. pre-lyophilization) which means that the protein essentially retains its physical and chemical stability and integrity during storage (e.g., after reconstitution and storage).

Those skilled in the art will appreciate that compatible lyoprotectant may be added to the liquid or lyophilized formulation at concentrations ranging from about 1 mM to about 1000 mM, from about 25 mM to about 750 mM, from about 50 mM to about 500 mM, from about 100 mM to about 300 mM, from about 125 mM to about 250 mM, from about 150 mM to about 200 mM or from about 165 mM to about 185 mM. In certain embodiments the lyoprotectant(s) may be added to provide a concentration of about 10 mM, about 25 mM, about 50 mM, about 75 mM, about 100 mM, about 125 mM, about 130 mM, about 140 mM, about 150 mM, about 160 mM, about 165 mM, about 170 mM, about 175 mM, about 180 mM, about 185 mM about 190 mM, about 200 mM, about 225 mM, about 250 mM, about 300 mM, about 400 mM, about 500 mM, about 600 mM, about 700 mM, about 800 mM about 900 mM, or about 1000 mM. In certain preferred embodiments the lyoprotectant(s) may comprise pharmaceutically acceptable sugars. In particularly preferred aspects the pharmaceutically acceptable sugars will comprise trehalose or sucrose.

In other selected embodiments liquid and lyophilized formulations of the instant invention may comprise certain compounds, including amino acids or pharmaceutically acceptable salts thereof, to act as stabilizing or buffering agents. Such compounds may be added at concentrations ranging from about 1 mM to about 100 mM, from about 5 mM to about 75 mM, from about 5 mM to about 50 mM, from about 10 mM to about 30 mM or from about 15 mM to about 25 mM. In certain embodiments the buffering agent(s) may be added to provide a concentration of about 1 mM, about 5 mM, about 10 mM, about 15 mM, about 20 mM, about 25 mM, about 30 mM, about 35 mM, about 40 mM, about 50 mM, about 60 mM, about 70 mM, about 80 mM, about 90 mM or about 100 mM. In other selected embodiments the buffering agent may be added to provide a concentration of about 5 mM, about 10 mM, about 15 mM, about 20 mM, about 25 mM, about 30 mM, about 35 mM, about 40 mM, about 50 mM, about 60 mM, about 70 mM, about 80 mM, about 90 mM or about 100 mM. In certain preferred embodiments the buffering agent will comprise histidine hydrochloride (e.g., L-histidine HCL).

In yet other selected embodiments liquid and lyophilized formulations of the instant invention may comprise nonionic surfactants such as polysorbate 20, polysorbate 40, polysorbate 60 or polysorbate 80 as stabilizing agents. Such compounds may be added at concentrations ranging from about 0.1 mgiml to about 2.0 mg/ml, from about 0.1 mg/ml to about 1.0 mg/ml, from about 0.2 mg/ml to about 0.8 mg/ml, from about 0.2 mg/ml to about 0.6 mg/ml or from about 0.3 mg/ml to about 0.5 mg/ml. In certain embodiments the surfactant may be added to provide a concentration of about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml or about 1.0 mg/ml. In other selected embodiments the surfactant may be added to provide a concentration of about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml or about 2.0 mg/ml. In certain preferred embodiments the surfactant will comprise polysorbate 20 or polysorbate 40. In particularly preferred aspects the surfactant will comprise polysorbate 20.

Whether reconstituted from a lyophilized powder or a native solution, compatible formulations of the disclosed antibodies or ADCs for parenteral administration (e.g., intravenous injection) may comprise ADC or antibody concentrations of from about 10 μg/mL to about 100 mg/mL. In certain selected embodiments antibody or ADC concentrations will comprise 20 μg/mL, 40 μg/mL, 60 μg/mL, 80 μg/mL, 100 μg/mL, 200 μg/mL, 300, μg/mL, 400 μg/mL, 500 μg/mL, 600 μg/mL, 700 μg/mL, 800 μg/mL, 900 μg/mL or 1 mg/mL. In other embodiments ADC concentrations will comprise 2 mg/mL, 3 mg/mL, 4 mg/mL, 5 mg/mL, 6 mg/mL, 8 mg/mL, 10 mg/mL, 12 mg/mL, 14 mg/mL, 16 mg/mL, 18 mg/mL, 20 mg/mL, 25 mg/mL, 30 mg/mL, 35 mg/mL, 40 mg/mL, 45 mg/mL, 50 mg/mL, 60 mg/mL, 70 mg/mL, 80 mg/mL, 90 mg/mL or 100 mg/mL.

In certain preferred aspects compositions of the present invention will comprise a liquid formulation comprising 10 mg/ml DLL3 ADC (e.g., hSC16.56DL1, hSC16.56ss1DL6, etc.), 20 mM histidine hydrochloride, 0.175M sucrose, 0.4 mg/mL polysorbate 20 at pH 6.0. In one aspect compositions of the instant invention comprise 10 mg/ml hSC16.56DL1 (i.e., Rova-T), 20 mM histidine hydrochloride, 0.175M sucrose, 0.4 mg/mL polysorbate 20 at pH 6.0. In another aspect compositions of the instant invention comprise 10 mg/ml hSC16.56ss1DL6, 20 mM histidine hydrochloride, 0.175M sucrose, 0.4 mg/mL polysorbate 20 at pH 6.0. As discussed herein, and shown in the Examples below, such liquid formulations may be lyophilized to provide powdered compositions that may be reconstituted with a pharmaceutically compatible (e.g., aqueous) carrier prior to use. When in a liquid solution such compositions should preferably be stored at -70° C. and protected from light. When lyophilized the DLL3 ADC powdered formulations should preferably be stored at 2-8° C. and protected from light. Each of the aforementioned solutions or powders is preferably contained in a sterile glass vial (e.g., USP Type 110 ml) and may be configured to consistently provide a set volume (e.g., 3 or 5 mL) of 10 mg/mL DLL3 ADC (in a native or reconstituted solution).

Whether reconstituted from lyophilized powder or not, the liquid DLL3 ADC formulations (e.g., as set forth immediately above) may be further diluted (preferably in an aqueous carrier) prior to administration. For example the aforementioned liquid formulations may further be diluted into an infusion bag containing 0.9% Sodium Chloride Injection, USP, or equivalent (mutatis mutandis), to achieve the desired dose level for administration. In certain aspects the fully diluted DLL3 ADO solution will be administered via intravenous infusion using an IV apparatus. Preferably the administered DLL3 ADO drug solution (whether by intravenous (IV) infusion or injection) is clear, colorless and free from visible particulates.

More generally the compounds and compositions of the invention may be administered in vivo, to a subject in need thereof, by various routes, including, but not limited to, oral, intravenous, intra-arterial, subcutaneous, parenteral, intranasal, intramuscular, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

B. Dosages and Dosing Regimens

The particular dosage regimen, i.e., dose, timing and repetition, will depend on the individual subject, as well as empirical considerations such as pharmacokinetics (e.g., half-life, clearance rate, etc.). Determination of the frequency of administration may be made by persons skilled in the art, such as an attending physician based on considerations of the condition and severity of the condition being treated, age and general state of health of the subject being treated and the like. Frequency of administration may be adjusted over the course of therapy based on assessment of the efficacy of the selected composition and the dosing regimen. Such assessment can be made on the basis of markers of the specific disease, disorder or condition or assessments of the individuals wellbeing (as measured using quality of life assessments, activities of daily living, etc.). In embodiments where the individual has cancer, these include direct measurements of tumor size via palpation or visual observation; indirect measurement of tumor size by x-ray or other imaging techniques; an improvement as assessed by direct tumor biopsy and microscopic examination of a tumor sample; the measurement of an indirect tumor marker (e.g., PSA for prostate cancer) or an antigen identified according to the methods described herein; reduction in the number of proliferative or tumorigenic cells, maintenance of the reduction of such neoplastic cells; reduction of the proliferation of neoplastic cells; or delay in the development of metastasis.

The DLL3 ADCs of the invention may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In selected embodiments the DLL3 antibodies or ADCs will be administered (preferably intravenously) at approximately 5, 10, 20, 30, 40, 50, 60, 70, 80, 90 or 100 μg/kg body weight per dose. Other embodiments may comprise the administration of antibodies or ADCs at about 200, 250, 300, 350, 400, 450, 500, 550, 600, 650, 700, 750, 800, 850, 900, 950, 1000, 1100, 1200, 1300, 1400, 1500, 1600, 1700, 1800, 1900 or 2000 μg/kg body weight per dose. In other embodiments the disclosed conjugates will be administered at 2.5, 3, 3.5, 4, 4.5, 5, 5.5, 6, 6.5, 7, 7.5, 8, 9 or 10 mg/kg. In still other embodiments the conjugates may be administered at 12, 14, 16, 18 or 20 mg/kg body weight per dose. In yet other embodiments the conjugates may be administered at 25, 30, 35, 40, 45, 50, 55, 60, 65, 70, 75, 80, 90 or 100 mg/kg body weight per dose. With the teachings herein one of skill in the art could readily determine appropriate dosages for various DLL3 antibodies or ADCs based on preclinical animal studies, clinical observations and standard medical and biochemical techniques and measurements.

Other dosing regimens may be predicated on Body Surface Area (BSA) calculations as disclosed in U.S. Pat. No. 7,744,877. As is well known, the BSA is calculated using the patient's height and weight and provides a measure of a subject's size as represented by the surface area of his or her body. In certain embodiments, the conjugates may be administered in dosages from 1 mg/m² to 800 mg/m², from 50 mg/m² to 500 mg/m² and at dosages of 100 mg/m², 150 mg/m², 200 mg/m², 250 mg/m², 300 mg/m², 350 mg/m², 400 mg/m² or 450 mg/m². It will also be appreciated that art recognized and empirical techniques may be used to determine appropriate dosage.

In other embodiments the anti-DLL3 antibodies or ADCs may be administered on a specific schedule. Generally, an effective dose of the DLL3 conjugate is administered to a subject one or more times. More particularly, an effective dose of the antibody or ADC is administered to the subject once a week, once every two weeks, once every three weeks, once a month or less than once a month. In certain embodiments, the effective dose of the DLL3 antibody or ADC may be administered multiple times, including for periods of at least a month, at least six months, at least a year, at least two years or a period of several years. In yet other embodiments, several days (2, 3, 4, 5, 6 or 7), several weeks (1, 2, 3, 4, 5, 6, 7 or 8) or several months (1, 2, 3, 4, 5, 6, 7 or 8) or even a year or several years may lapse between administration of the disclosed antibodies or ADCs.

In some embodiments the course of treatment involving conjugated antibodies will comprise multiple doses (cycles) of the selected drug product over a period of weeks or months. More specifically, antibodies or ADCs of the instant invention may be administered once every day, every two days, every four days, every week, every ten days, every two weeks, every three weeks, every four weeks, every five weeks, every six weeks, every eight weeks, every ten weeks every or every twelve weeks. In this regard it will be appreciated that the dosages may be altered or the interval may be adjusted based on patient response and clinical practices. The invention also contemplates discontinuous administration or daily doses divided into several partial administrations. The compositions of the instant invention and anti-cancer agent may be administered interchangeably, on alternate days or weeks; or a sequence of antibody treatments may be given, followed by one or more treatments of anti-cancer agent therapy. In any event, as will be understood by those of ordinary skill in the art, the appropriate doses of chemotherapeutic agents will be generally around those already employed in clinical therapies wherein the chemotherapeutics are administered alone or in combination with other chemotherapeutics.

In certain embodiments the present invention provides anti-DLL3 antibody drug conjugates for use in the treatment of cancer wherein the treatment may comprise administering an effective amount of an anti-DLL3 antibody drug conjugate (DLL3 ADC) at least once every week (QW), at least once every two weeks (Q2W), at least once every three weeks (Q3W), at least once every four weeks (Q4W), at least once every five weeks (Q5W), at least once every six weeks (Q6W), at least once every seven weeks (Q7W), at least once every eight weeks (08W), at least once every nine weeks (Q9W) or at least once every ten weeks (Q10W). In selected embodiments the DLL3 ADC will be administered at least every three weeks (Q3W), at least once every four weeks (Q4W), at least once every five weeks (Q5W) or at least once every six weeks (Q6W). In certain other embodiments the DLL3 ADC will be administered at least every seven weeks (Q7W), at least once every eight weeks (Q8W), at least once every nine weeks (Q9W) or at least once every ten weeks (Q10W). In other selected embodiments the DLL3 ADC will be administered at a dose of about 0.05 mg/kg, 0.1 mg/kg, 0.2 mg/kg, 0.3 mg/kg, 0.4 mg/kg, 0.5 mg/kg, 0.6 mg/kg, 0.7 mg/kg or 0.8 mg/kg. Certain embodiments will comprise treating the patient with a single administration of the DLL3 ADC. Yet other embodiments will comprise treating the patient at specified intervals (i.e. Q2W, Q3W, Q4W, Q5W, Q6W, etc) for two cycles (×2), for three cycles (×3), for four cycles (×4), for five cycles (×5), for six cycles (×6), for seven cycles (×7), for eight cycles (×8), for nine cycles (×9) or for ten cycles (×10). Still other aspects of the invention will comprise administering the DLL3 ADC an indefinite number of cycles depending on the response of the patient and any toxicity. In other embodiments the initial DLL3 ADC treatment (of x cycles) may be completed and no further DLL3 ADC treatment is undertaken until the cancer shows signs of progressing (treatment at progression). In yet other embodiments the initial DLL3 ADC treatment (of x cycles) may be completed and then the patient is put on maintenance therapy (e.g., 0.1 mg/kg DLL3 ADC Q6W indefinitely).

Exemplary dosing regimens compatible with the instant invention are set out below in Table 3. Note that, as discussed in more detail below, the disclosed exemplary dosing regimens are compatible with using DLL3 ADCs as a single agent or in combination with other therapeutic agents or cancer treatments (e.g., surgery or beam radiation).

TABLE 3

| | | | | |
|---|---|---|---|---|
| 0.05 mg/kg Q1W × 2 | 0.05 mg/kg Q1W × 3 | 0.05 mg/kg Q1W × 4 | 0.05 mg/kg Q1W × 5 | 0.05 mg/kg Q1W × 6 |
| 0.05 mg/kg Q2W × 2 | 0.05 mg/kg Q2W × 3 | 0.05 mg/kg Q2W × 4 | 0.05 mg/kg Q2W × 5 | 0.05 mg/kg Q2W × 6 |
| 0.05 mg/kg Q3W × 2 | 0.05 mg/kg Q3W × 3 | 0.05 mg/kg Q3W × 4 | 0.05 mg/kg Q3W × 5 | 0.05 mg/kg Q3W × 6 |
| 0.05 mg/kg Q4W × 2 | 0.05 mg/kg Q4W × 3 | 0.05 mg/kg Q4W × 4 | 0.05 mg/kg Q4W × 5 | 0.05 mg/kg Q4W × 6 |
| 0.05 mg/kg Q5W × 2 | 0.05 mg/kg Q5W × 3 | 0.05 mg/kg Q5W × 4 | 0.05 mg/kg Q5W × 5 | 0.05 mg/kg Q5W × 6 |
| 0.05 mg/kg Q6W × 2 | 0.05 mg/kg Q6W × 3 | 0.05 mg/kg Q6W × 4 | 0.05 mg/kg Q6W × 5 | 0.05 mg/kg Q6W × 6 |
| 0.05 mg/kg Q7W × 2 | 0.05 mg/kg Q7W × 3 | 0.05 mg/kg Q7W × 4 | 0.05 mg/kg Q7W × 5 | 0.05 mg/kg Q7W × 6 |
| 0.05 mg/kg Q8W × 2 | 0.05 mg/kg Q8W × 3 | 0.05 mg/kg Q8W × 4 | 0.05 mg/kg Q8W × 5 | 0.05 mg/kg Q8W × 6 |
| 0.05 mg/kg Q9W × 2 | 0.05 mg/kg Q9W × 3 | 0.05 mg/kg Q9W × 4 | 0.05 mg/kg Q9W × 5 | 0.05 mg/kg Q9W × 6 |
| 0.05 mg/kg Q10W × 2 | 0.05 mg/kg Q10W × 3 | 0.05 mg/kg Q10W × 4 | 0.05 mg/kg Q10W × 5 | 0.05 mg/kg Q10W × 6 |
| 0.1 mg/kg Q1W × 2 | 0.1 mg/kg Q1W × 3 | 0.1 mg/kg Q1W × 4 | 0.1 mg/kg Q1W × 5 | 0.1 mg/kg Q1W × 6 |
| 0.1 mg/kg Q2W × 2 | 0.1 mg/kg Q2W × 3 | 0.1 mg/kg Q2W × 4 | 0.1 mg/kg Q2W × 5 | 0.1 mg/kg Q2W × 6 |
| 0.1 mg/kg Q3W × 2 | 0.1 mg/kg Q3W × 3 | 0.1 mg/kg Q3W × 4 | 0.1 mg/kg Q3W × 5 | 0.1 mg/kg Q3W × 6 |
| 0.1 mg/kg Q4W × 2 | 0.1 mg/kg Q4W × 3 | 0.1 mg/kg Q4W × 4 | 0.1 mg/kg Q4W × 5 | 0.1 mg/kg Q4W × 6 |
| 0.1 mg/kg Q5W × 2 | 0.1 mg/kg Q5W × 3 | 0.1 mg/kg Q5W × 4 | 0.1 mg/kg Q5W × 5 | 0.1 mg/kg Q5W × 6 |
| 0.1 mg/kg Q6W × 2 | 0.1 mg/kg Q6W × 3 | 0.1 mg/kg Q6W × 4 | 0.1 mg/kg Q6W × 5 | 0.1 mg/kg Q6W × 6 |
| 0.1 mg/kg Q7W × 2 | 0.1 mg/kg Q7W × 3 | 0.1 mg/kg Q7W × 4 | 0.1 mg/kg Q7W × 5 | 0.1 mg/kg Q7W × 6 |
| 0.1 mg/kg Q8W × 2 | 0.1 mg/kg Q8W × 3 | 0.1 mg/kg Q8W × 4 | 0.1 mg/kg Q8W × 5 | 0.1 mg/kg Q8W × 6 |
| 0.1 mg/kg Q9W × 2 | 0.1 mg/kg Q9W × 3 | 0.1 mg/kg Q9W × 4 | 0.1 mg/kg Q9W × 5 | 0.1 mg/kg Q9W × 6 |
| 0.1 mg/kg Q10W × 2 | 0.1 mg/kg Q10W × 3 | 0.1 mg/kg Q10W × 4 | 0.1 mg/kg Q10W × 5 | 0.1 mg/kg Q10W × 6 |
| 0.2 mg/kg Q1W × 2 | 0.2 mg/kg Q1W × 3 | 0.2 mg/kg Q1W × 4 | 0.2 mg/kg Q1W × 5 | 0.2 mg/kg Q1W × 6 |
| 0.2 mg/kg Q2W × 2 | 0.2 mg/kg Q2W × 3 | 0.2 mg/kg Q2W × 4 | 0.2 mg/kg Q2W × 5 | 0.2 mg/kg Q2W × 6 |
| 0.2 mg/kg Q3W × 2 | 0.2 mg/kg Q3W × 3 | 0.2 mg/kg Q3W × 4 | 0.2 mg/kg Q3W × 5 | 0.2 mg/kg Q3W × 6 |
| 0.2 mg/kg Q4W × 2 | 0.2 mg/kg Q4W × 3 | 0.2 mg/kg Q4W × 4 | 0.2 mg/kg Q4W × 5 | 0.2 mg/kg Q4W × 6 |
| 0.2 mg/kg Q5W × 2 | 0.2 mg/kg Q5W × 3 | 0.2 mg/kg Q5W × 4 | 0.2 mg/kg Q5W × 5 | 0.2 mg/kg Q5W × 6 |
| 0.2 mg/kg Q6W × 2 | 0.2 mg/kg Q6W × 3 | 0.2 mg/kg Q6W × 4 | 0.2 mg/kg Q6W × 5 | 0.2 mg/kg Q6W × 6 |
| 0.2 mg/kg Q7W × 2 | 0.2 mg/kg Q7W × 3 | 0.2 mg/kg Q7W × 4 | 0.2 mg/kg Q7W × 5 | 0.2 mg/kg Q7W × 6 |
| 0.2 mg/kg Q8W × 2 | 0.2 mg/kg Q8W × 3 | 0.2 mg/kg Q8W × 4 | 0.2 mg/kg Q8W × 5 | 0.2 mg/kg Q8W × 6 |
| 0.2 mg/kg Q9W × 2 | 0.2 mg/kg Q9W × 3 | 0.2 mg/kg Q9W × 4 | 0.2 mg/kg Q9W × 5 | 0.2 mg/kg Q9W × 6 |
| 0.2 mg/kg Q10W × 2 | 0.2 mg/kg Q10W × 3 | 0.2 mg/kg Q10W × 4 | 0.2 mg/kg Q10W × 5 | 0.2 mg/kg Q10W × 6 |
| 0.3 mg/kg Q1W × 2 | 0.3 mg/kg Q1W × 3 | 0.3 mg/kg Q1W × 4 | 0.3 mg/kg Q1W × 5 | 0.3 mg/kg Q1W × 6 |
| 0.3 mg/kg Q2W × 2 | 0.3 mg/kg Q2W × 3 | 0.3 mg/kg Q2W × 4 | 0.3 mg/kg Q2W × 5 | 0.3 mg/kg Q2W × 6 |
| 0.3 mg/kg Q3W × 2 | 0.3 mg/kg Q3W × 3 | 0.3 mg/kg Q3W × 4 | 0.3 mg/kg Q3W × 5 | 0.3 mg/kg Q3W × 6 |
| 0.3 mg/kg Q4W × 2 | 0.3 mg/kg Q4W × 3 | 0.3 mg/kg Q4W × 4 | 0.3 mg/kg Q4W × 5 | 0.3 mg/kg Q4W × 6 |
| 0.3 mg/kg Q5W × 2 | 0.3 mg/kg Q5W × 3 | 0.3 mg/kg Q5W × 4 | 0.3 mg/kg Q5W × 5 | 0.3 mg/kg Q5W × 6 |
| 0.3 mg/kg Q6W × 2 | 0.3 mg/kg Q6W × 3 | 0.3 mg/kg Q6W × 4 | 0.3 mg/kg Q6W × 5 | 0.3 mg/kg Q6W × 6 |
| 0.3 mg/kg Q7W × 2 | 0.3 mg/kg Q7W × 3 | 0.3 mg/kg Q7W × 4 | 0.3 mg/kg Q7W × 5 | 0.3 mg/kg Q7W × 6 |
| 0.3 mg/kg Q8W × 2 | 0.3 mg/kg Q8W × 3 | 0.3 mg/kg Q8W × 4 | 0.3 mg/kg Q8W × 5 | 0.3 mg/kg Q8W × 6 |
| 0.3 mg/kg Q9W × 2 | 0.3 mg/kg Q9W × 3 | 0.3 mg/kg Q9W × 4 | 0.3 mg/kg Q9W × 5 | 0.3 mg/kg Q9W × 6 |
| 0.3 mg/kg Q10W × 2 | 0.3 mg/kg Q10W × 3 | 0.3 mg/kg Q10W × 4 | 0.3 mg/kg Q10W × 5 | 0.3 mg/kg Q10W × 6 |
| 0.4 mg/kg Q1W × 2 | 0.4 mg/kg Q1W × 3 | 0.4 mg/kg Q1W × 4 | 0.4 mg/kg Q1W × 5 | 0.4 mg/kg Q1W × 6 |
| 0.4 mg/kg Q2W × 2 | 0.4 mg/kg Q2W × 3 | 0.4 mg/kg Q2W × 4 | 0.4 mg/kg Q2W × 5 | 0.4 mg/kg Q2W × 6 |
| 0.4 mg/kg Q3W × 2 | 0.4 mg/kg Q3W × 3 | 0.4 mg/kg Q3W × 4 | 0.4 mg/kg Q3W × 5 | 0.4 mg/kg Q3W × 6 |
| 0.4 mg/kg Q4W × 2 | 0.4 mg/kg Q4W × 3 | 0.4 mg/kg Q4W × 4 | 0.4 mg/kg Q4W × 5 | 0.4 mg/kg Q4W × 6 |
| 0.4 mg/kg Q5W × 2 | 0.4 mg/kg Q5W × 3 | 0.4 mg/kg Q5W × 4 | 0.4 mg/kg Q5W × 5 | 0.4 mg/kg Q5W × 6 |
| 0.4 mg/kg Q6W × 2 | 0.4 mg/kg Q6W × 3 | 0.4 mg/kg Q6W × 4 | 0.4 mg/kg Q6W × 5 | 0.4 mg/kg Q6W × 6 |
| 0.4 mg/kg Q7W × 2 | 0.4 mg/kg Q7W × 3 | 0.4 mg/kg Q7W × 4 | 0.4 mg/kg Q7W × 5 | 0.4 mg/kg Q7W × 6 |
| 0.4 mg/kg Q8W × 2 | 0.4 mg/kg Q8W × 3 | 0.4 mg/kg Q8W × 4 | 0.4 mg/kg Q8W × 5 | 0.4 mg/kg Q8W × 6 |
| 0.4 mg/kg Q9W × 2 | 0.4 mg/kg Q9W × 3 | 0.4 mg/kg Q9W × 4 | 0.4 mg/kg Q9W × 5 | 0.4 mg/kg Q9W × 6 |
| 0.4 mg/kg Q10W × 2 | 0.4 mg/kg Q10W × 3 | 0.4 mg/kg Q10W × 4 | 0.4 mg/kg Q10W × 5 | 0.4 mg/kg Q10W × 6 |
| 0.5 mg/kg Q1W × 2 | 0.5 mg/kg Q1W × 3 | 0.5 mg/kg Q1W × 4 | 0.5 mg/kg Q1W × 5 | 0.5 mg/kg Q1W × 6 |
| 0.5 mg/kg Q2W × 2 | 0.5 mg/kg Q2W × 3 | 0.5 mg/kg Q2W × 4 | 0.5 mg/kg Q2W × 5 | 0.5 mg/kg Q2W × 6 |
| 0.5 mg/kg Q3W × 2 | 0.5 mg/kg Q3W × 3 | 0.5 mg/kg Q3W × 4 | 0.5 mg/kg Q3W × 5 | 0.5 mg/kg Q3W × 6 |
| 0.5 mg/kg Q4W × 2 | 0.5 mg/kg Q4W × 3 | 0.5 mg/kg Q4W × 4 | 0.5 mg/kg Q4W × 5 | 0.5 mg/kg Q4W × 6 |
| 0.5 mg/kg Q5W × 2 | 0.5 mg/kg Q5W × 3 | 0.5 mg/kg Q5W × 4 | 0.5 mg/kg Q5W × 5 | 0.5 mg/kg Q5W × 6 |
| 0.5 mg/kg Q6W × 2 | 0.5 mg/kg Q6W × 3 | 0.5 mg/kg Q6W × 4 | 0.5 mg/kg Q6W × 5 | 0.5 mg/kg Q6W × 6 |
| 0.5 mg/kg Q7W × 2 | 0.5 mg/kg Q7W × 3 | 0.5 mg/kg Q7W × 4 | 0.5 mg/kg Q7W × 5 | 0.5 mg/kg Q7W × 6 |
| 0.5 mg/kg Q8W × 2 | 0.5 mg/kg Q8W × 3 | 0.5 mg/kg Q8W × 4 | 0.5 mg/kg Q8W × 5 | 0.5 mg/kg Q8W × 6 |
| 0.5 mg/kg Q9W × 2 | 0.5 mg/kg Q9W × 3 | 0.5 mg/kg Q9W × 4 | 0.5 mg/kg Q9W × 5 | 0.5 mg/kg Q9W × 6 |
| 0.5 mg/kg Q10W × 2 | 0.5 mg/kg Q10W × 3 | 0.5 mg/kg Q10W × 4 | 0.5 mg/kg Q10W × 5 | 0.5 mg/kg Q10W × 6 |

In some aspects of the invention the DLL3 ADC will comprise a PBD. In other aspects the DLL3 will comprise SC16LD6.5 (e.g., hSC16.56DL1 or ADC1). In other aspects the DLL3 ADC will comprise a site-specific ADC and, in certain preferred aspects, will comprise hSC16.56ss1PBD1 (e.g., hSC16.56ss1DL6 or ADC6). In yet other aspects the DLL3 ADC will be administered intravenously. In certain other aspects the cancer to be treated will comprise a neuroendocrine tumor. In other aspects the cancer to be treated will comprise small cell lung cancer (SCLC) or large cell neuroendocrine cancer (LCNEC). Certain embodiments will comprise using the disclosed compositions to treat frontline patients (i.e., patients that have not been treated for the particular cancer). In selected embodiments the frontline patients will exhibit extensive-stage SCLC or limited stage SCLC. In other selected aspects the cancer patients to be treated will comprise second line patients (i.e., previously treated patients). In yet other embodiments the cancer patients to be treated will comprise third line patients (i.e., patients that have been treated twice previously). In still other embodiments the cancer patients will comprise fourth line patients (i.e., patients that have been treated three times previously).

Certain preferred embodiments of the invention will comprise treating a patient with 0.2 mg/kg of DLL3 ADC every 3 weeks for 3 cycles (0.2 mg/kg Q3W×3). In selected embodiments the patient to be treated at 0.2 mg/kg Q3W×3 will be suffering from SCLC. In other embodiments the patient to be treated at 0.2 mg/kg Q3W×3 will be suffering from LCNEC. In some aspects the patient has not been treated for the cancer. In certain aspects the patient will comprise a second line patient. In yet other embodiments the patient will comprise a third line patient. In other aspects the patient will be treated at progression following the 0.2 mg/kg Q3W×3 treatment cycle. In yet other aspects the patient will be shifted to DLL3 ADC maintenance therapy following the 0.2 mg/kg Q3W×3 treatment cycle. In yet other embodiments the DLL3 ADC will comprise SC16LD6.5 (e.g., ADC1) while in still other embodiments the DLL3 ADC will comprise h16.56ss1DL6.

In such treatments (and as discussed in more detail below) the DLL3 ADC may be combined with selected anti-cancer agent(s) to improve patient response. For certain subjects the DLL3 ADC may be administered every three weeks for two or more cycles (e.g., 0.2 mg/kg Q3W×2 or 0.2 mg/kg Q3W×3, etc.) either before or after the administration of one or more anti-cancer agent(s). In selected embodiments the subject will be suffering from SCLC and the anti-cancer agents will be cisplatin, carboplatin and/or etoposide. Accordingly, one exemplary dosing regimen may comprise DLL3 ADC at 0.2 mg/kg Q3W×2 or 0.2 mg/kg Q3W×3 followed by cisplatin (e.g., at 80 mg/m²) and etoposide (e.g., at 100 mg/m²) where each may be administered at, for example, Q3W×4. Thus, in such instances one regimen would comprise DLL3 ADC at 0.2 mg/kg Q3W×2 or 0.2 mg/kg Q3W×3; CDDP and ETP at Q3W×4 where the CDDP/ETP regimen may be started one week, two weeks, three weeks, four weeks, five weeks, six weeks or later following conclusion of the DLL3 ADC cycle. In other compatible embodiments the CDDP/ETP may be given prior to treatment with the DLL3 ADC (i.e., CDDP/ETP at Q3W×4; DLL3 ADC at 0.2 mg/kg Q3W×2 or 0.2 mg/kg Q3W×3 where the DLL3 ADC regimen may be started one week, two weeks, three weeks, four weeks, five weeks, six weeks or later following completion of the CDDP/ETP cycles). In still other embodiments the CDDP/ETP regimen and the DLL3 ADC regimen (substantially as described above but optionally with a DLL3 ADC dosage of 0.1 mg/kg) may be given concurrently (i.e., they both start on week 1). In certain embodiments the subject to be treated in accordance with the aforementioned regimens will be a frontline patient. In other preferred embodiments the frontline patient will be suffering from SCLC or LCNEC. In yet other embodiments cisplatin will be administered at 60 mg/m² while in still other embodiments cisplatin/etoposide combinations may be given every 3 weeks (Platinum D1, Etoposide D1, 2, 3, every 3 weeks×4 or ×6). In still other embodiments Carboplatin (5 AUC)/Etoposide may be administered with a similar frequency to CDDP/E Q21d×4-6 cycles. In yet other embodiments the DLL3 ADCs may be administered to the patient prior to any chemotherapeutic agent (including carboplatin, cisplatin and/or etoposide) to act as a sensitizing agent and potentiate the effects of the therapeutic combination.

As discussed above, selected embodiments of the invention comprise ADCs that have improved pharmacokinetic and pharmacodynamic properties which provide for an enhanced therapeutic index and allow for optimization of dosing regimens. In this regard the disclosed ADCs will have a terminal half-life exceeding six days, a terminal half-life of greater than seven days or a terminal half-life of greater than eight days when measured as set forth herein. Still other aspects of the invention will comprise ADCs having a terminal half-life of greater than nine days, a terminal half-life of greater than ten days, a terminal half-life of greater than eleven days, a terminal half-life of greater than twelve days (each as measured in human subjects). In still other embodiments the disclosed ADCs will have a terminal half-life of greater than thirteen days, a terminal half-life of greater than about fourteen days, a terminal half-life of greater than fifteen days, a terminal half-life of greater than about sixteen days, a terminal half-life of greater than about seventeen days, a terminal half-life of greater than eighteen days, a terminal half-life of greater than about nineteen days, a terminal half-life of greater than about twenty days or a terminal half-life of greater than three weeks in human subjects. In yet other embodiments the ADCs of the invention will exhibit a terminal half-life of about six days, a terminal half-life of about seven days, a terminal half-life of about eight days, a terminal half-life of about nine days, a terminal half-life of about ten days, a terminal half-life of about eleven days, a terminal half-life of about twelve days, a terminal half-life of about thirteen days, a terminal half-live of about fourteen days, a terminal half-life of about fifteen days, a terminal half-life of about sixteen days, a terminal half-life of about seventeen days, a terminal half-life of about eighteen days, a terminal half-life of about nineteen days, a terminal half-life of about twenty days or a terminal half-live of about three weeks. Those of skill in the art will appreciate that such protracted half-lives will allow for less frequent dosing of the disclosed ADCs thereby providing the desired efficacy while exhibiting similar or reduced toxicity.

Accordingly, certain other preferred embodiments of the invention will comprise treating a patient with 0.3 mg/kg of DLL3 ADC every 6 weeks for 2 or more cycles (e.g., 0.3 mg/kg Q6W×2). As shown below in the Examples such a regimen may be particularly effective (exhibit an efficacious therapeutic index) because of the relatively long half-life of the DLL3 ADCs of the instant invention. In selected embodiments the patient to be treated at 0.3 mg/kg Q6W×2 will be suffering from SCLC. In other embodiments the patient to be treated at 0.3 mg/kg Q6W×2 will be suffering from LCNEC. Still other embodiments will comprise administering DLL3 ADCs at 0.3 mg/kg Q6W for three cycles (×3), for four cycles (×4), for five cycles (×5) for six cycles (×6) or more. In such embodiments the cycles may be continuous (every six weeks) or intermittent (skip every third cycle then resume). In some aspects the patient has not been treated for the cancer (frontline). In certain aspects the patient will comprise a second line patient. In yet other embodiments the patient will comprise a third line patient. In other aspects the patient will be treated at progression following the 0.3 mg/kg Q6W×2 treatment cycle. In yet other aspects the patient will be shifted to DLL3 ADC maintenance therapy following the 0.3 mg/kg Q6W×2 treatment cycle. In certain embodiments the DLL3 ADC will comprise SC16LD6.5 (e.g., ADC1) while in other preferred aspects the DLL3 ADC will comprise hSC16.56ss1DL6 (e.g., ADC6).

As discussed in more detail below the DLL3 ADC may be combined with selected anti-cancer agent(s) to improve patient response. For certain subjects the DLL3 ADC may be administered every six weeks for two cycles (e.g., 0.3 mg/kg Q6W×2) either before or after the administration of one or more anti-cancer agent(s). In selected embodiments the subject will be suffering from SCLC and the anti-cancer agents will be cisplatin, carboplatin and/or etoposide. Accordingly, one exemplary dosing regimen may comprise DLL3 ADC at 0.3 mg/kg Q6W×2 followed by cisplatin (e.g., at 80 mg/m²) and etoposide (e.g., at 100 mg/m²) where each are administered at, for example, Q3W×4. Thus, in such instances one cycle would comprise DLL3 ADC at 0.3 mg/kg Q6W×2; CDDP and ETP at Q3W×4 where the CDDP/ETP regimen may be started one week, two weeks, three weeks, four weeks, five weeks, six weeks or later following conclusion of the DLL3 ADC cycle. In other compatible embodiments the CDDP/ETP may be given prior to treatment with the DLL3 ADC (i.e., CDDP/ETP at Q3W×4; DLL3 ADC at 0.3 mg/kg Q6W×2 where the DLL3 ADC regimen may be started one week, two weeks, three weeks, four weeks, five weeks, six weeks or later following completion of the CDDP/ETP cycles). In still other embodiments the CDDP/ETP regimen and the DLL3 ADC regimen (substantially as described above but optionally with a DLL3 ADC dosage of 0.1 mg/kg) may be given concurrently (i.e., they both start on week 1). In certain embodiments the subject to be treated in accordance with the aforementioned regimens will be a frontline patient. In other preferred embodiments the frontline patient will be suffering from SCLC or LCNEC. In yet other embodiments cisplatin will be administered at 60 mg/m² while in still other embodiments cisplatin/etoposide combinations may be given every 3 weeks (Platinum D1, Etoposide D1, 2, 3, every 3 weeks×4 or ×6). In still other embodiments Carboplatin (5 AUC)/Etoposide may be administered with a similar frequency to CDDP/E Q21d×4-6 cycles. In yet other embodiments the DLL3 ADCs may be administered to the patient prior to any chemotherapeutic agent (including carboplatin, cisplatin and/or etoposide) to act as a sensitizing agent and potentiate the effects of the therapeutic combination.

In further embodiments the DLL3 ADCs of the instant invention may be administered at different dosages in any one cycle. For example, the drug may be administered (i.e., loaded or drug loading) at a relatively high dose (e.g., 0.5 mg/kg) followed by a lower dose of DLL3 ADC (e.g., 0.2 mg/kg) four weeks later (Q4W) as part of the same cycle. Again such cycles may be repeated (2×, 3×, etc.) or delayed until progression (treat at progression) or followed up by DLL3 ADC maintenance (e.g., 0.1 mg/kg Q4W indefinite or 0.1 mg/kg Q6W indefinite). In this regard the DLL3 antibodies or ADCs of the instant invention may be used in a maintenance therapy setting to reduce or eliminate the chance of tumor recurrence following the initial presentation of the disease. Such maintenance therapy may be used whether the first treatment was with DLL3 ADC or another anti-neoplastic agent or treatment (e.g., radiation, surgery, chemotherapy, etc.). Preferably the disorder will have been treated and the initial tumor mass eliminated, reduced or otherwise ameliorated so the patient is asymptomatic or in remission. At such time the subject may be administered pharmaceutically effective amounts of the disclosed ADCs one or more times even though there is little or no indication of disease using standard diagnostic procedures. In preferred embodiments the DLL3 ADC will comprise a PBD and in selected embodiments will comprise SC16LD6.5 or SC16.56ss1 DL6.

In certain embodiments the maintenance therapy will comprise the administration of DLL3 ADCs following chemotherapeutic treatment including standard of care (SOC) chemotherapeutic treatments. In selected aspects this maintenance therapy will comprise frontline patients initially treated with SOC chemotherapeutics such as cisplatin/etoposide which is followed by a DLL3 ADC maintenance regimen. By way of example a patient may be treated with one or more cycles of frontline chemotherapy (e.g., four cycles of platinum-based chemotherapy) followed by the administration of one or more cycles of DLL3 ADCs. In certain embodiments the DLL3 ADCs may be administered at 0.3 mg/kg Q6W omitting every third cycle. It will be appreciated that such maintenance regimens may be continued as long as the patient is responding and not exhibiting disease progression or adverse events.

In another preferred embodiment the antibodies or ADCs of the present invention may be used to prophylactically or as an adjuvant therapy to prevent or reduce the possibility of tumor metastasis following a debulking procedure. As used in the instant disclosure a “debulking procedure” means any procedure, technique or method that reduces the tumor mass or ameliorates the tumor burden or tumor proliferation. Exemplary debulking procedures include, but are not limited to, surgery, radiation treatments (i.e., beam radiation), chemotherapy, immunotherapy or ablation. At appropriate times readily determined by one skilled in the art in view of the instant disclosure the disclosed ADCs may be administered as suggested by clinical, diagnostic or theragnostic procedures to reduce tumor metastasis and the possibility of tumor recurrence.

Yet other embodiments of the invention comprise administering the disclosed antibodies or ADCs to subjects that are asymptomatic but at risk of developing cancer. That is, the antibodies or ADCs of the instant invention may be used in a truly preventative sense and given to patients that have been examined or tested and have one or more noted risk factors (e.g., genomic indications, family history, in vivo or in vitro test results, etc.) but have not developed neoplasia.

Dosages and regimens may also be determined empirically for the disclosed therapeutic compositions in individuals who have been given one or more administration(s). For example, individuals may be given incremental dosages of a therapeutic composition produced as described herein. In selected embodiments the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

C. Combination Therapies

As alluded to above combination therapies may be particularly useful in decreasing or inhibiting unwanted neoplastic cell proliferation, decreasing the occurrence of cancer, decreasing or preventing the recurrence of cancer, or decreasing or preventing the spread or metastasis of cancer. In such cases the antibodies or ADCs of the instant invention may function as sensitizing or chemosensitizing agents by removing CSCs that would otherwise prop up and perpetuate the tumor mass and thereby allow for more effective use of current standard of care debulking or anti-cancer agents. That is, the disclosed antibodies or ADCs may, in certain embodiments, provide an enhanced effect (e.g., additive or synergistic in nature) that potentiates the mode of action of another administered therapeutic agent. In the context of the instant invention “combination therapy” shall be interpreted broadly and merely refers to the administration of an anti-DLL3 antibody or ADC and one or more anti-cancer agents that include, but are not limited to, cytotoxic agents, cytostatic agents, anti-angiogenic agents, debulking agents, chemotherapeutic agents, radiotherapy and radiotherapeutic agents, targeted anti-cancer agents (including both monoclonal antibodies and small molecule entities), BRMs, therapeutic antibodies, cancer vaccines, cytokines, hormone therapies, radiation therapy and anti-metastatic agents and immunotherapeutic agents, including both specific and non-specific approaches.

There is no requirement for the combined results to be additive of the effects observed when each treatment (e.g., DLL3 ADC and an anti-cancer agent) is conducted separately. Although at least additive effects are generally desirable, any increased anti-tumor effect above one of the single therapies is beneficial. Furthermore, the invention does not require the combined treatment to exhibit synergistic effects. However, those skilled in the art will appreciate that with certain selected combinations that comprise preferred embodiments, synergism may be observed.

As such, In certain aspects the combination therapy has therapeutic synergy or improves the measurable therapeutic effects in the treatment of cancer over (i) the anti-DLL3 antibody or ADC used alone, or (ii) the therapeutic moiety used alone, or (iii) the use of the therapeutic moiety in combination with another therapeutic moiety without the addition of an anti-DLL3 antibody or ADC. The term “therapeutic synergy”, as used herein, means the combination of an anti-DLL3 antibody or ADC and one or more therapeutic moiety(ies) having a therapeutic effect greater than the additive effect of the combination of the anti-DLL3 antibody or ADC and the one or more therapeutic moiety(ies).

Desired outcomes of the disclosed combinations are quantified by comparison to a control or baseline measurement. As used herein, relative terms such as “improve,” “increase,” or “reduce” indicate values relative to a control, such as a measurement in the same individual prior to initiation of treatment described herein, or a measurement in a control individual (or multiple control individuals) in the absence of the anti-DLL3 antibodies or ADCs described herein but in the presence of other therapeutic moiety(ies) such as standard of care treatment. A representative control individual is an individual afflicted with the same form of cancer as the individual being treated, who is about the same age as the individual being treated (to ensure that the stages of the disease in the treated individual and the control individual are comparable).

Changes or improvements in response to therapy (whether additive or synergistic) may prove to be statistically significant. As used herein, the term “significance” or “significant” relates to a statistical analysis of the probability that there is a non-random association between two or more measured responses. To determine whether or not a relationship is “significant” or has “significance,” a “p-value” can be calculated. P-values that fall below a user-defined cut-off point are regarded as significant. For the purposes of the instant invention a p-value less than or equal to 0.1, less than 0.05, less than 0.01, less than 0.005, or less than 0.001 may be regarded as significant.

A synergistic therapeutic effect may be an effect of at least about two-fold greater than the therapeutic effect elicited by a single therapeutic moiety or anti-DLL3 antibody or ADC, or the sum of the therapeutic effects elicited by the anti-DLL3 antibody or ADC or the single therapeutic moiety(ies) of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single therapeutic moiety or anti-DLL3 antibody or ADC, or the sum of the therapeutic effects elicited by the anti-DLL3 antibody or ADC or the single therapeutic moiety(ies) of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

In practicing combination therapy, the anti-DLL3 antibody or ADC and therapeutic moiety(ies) may be administered to the subject simultaneously, either in a single composition, or as two or more distinct compositions using the same or different administration routes. Alternatively, treatment with the anti-DLL3 antibody or ADC may precede or follow the therapeutic moiety treatment by, e.g., intervals ranging from minutes to weeks. In one embodiment, both the therapeutic moiety and the antibody or ADC are administered within about 5 minutes to about two weeks of each other. In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the antibody and the therapeutic moiety.

The combination therapy can be administered until the condition is treated, palliated or cured on various schedules such as once, twice or three times daily, once every two days, once every three days, once weekly, once every two weeks, once every month, once every two months, once every three months, once every six months, or may be administered continuously. The antibody and therapeutic moiety(ies) may be administered on alternate days or weeks; or a sequence of anti-DLL3 antibody or ADC treatments may be given, followed by one or more treatments with the additional therapeutic moiety. In one embodiment an anti-DLL3 antibody or ADC is administered in combination with one or more therapeutic moiety(ies) for short treatment cycles. In other embodiments the combination treatment is administered for long treatment cycles or in a maintenance therapy type setting.

In selected embodiments the compounds and compositions of the present invention may be used in conjunction with checkpoint inhibitors such as PD-1 inhibitors or PD-L1 inhibitors. PD-1, together with its ligand PD-L1, are negative regulators of the antitumor T lymphocyte response. In one embodiment the combination therapy may comprise the administration of anti-DLL3 antibodies or ADCs together with an anti-PD-1 antibody (e.g. pembrolizumab, nivolumab, pidilizumab) and optionally one or more other therapeutic moiety(ies). In another embodiment the combination therapy may comprise the administration of anti-DLL3 antibodies or ADCs together with an anti-PD-L1 antibody (e.g. avelumab, atezolizumab, durvalumab) and optionally one or more other therapeutic moiety(ies). In yet another embodiment, the combination therapy may comprise the administration of anti-DLL3 antibodies or ADCs together with an anti PD-1 antibody or anti-PD-L1 administered to patients who continue progress following treatments with checkpoint inhibitors and/or targeted BRAF combination therapies (e.g. vemurafenib or dabrafinib). As discussed herein and set forth in the Examples below such therapeutic combinations may prove to be surprisingly efficacious in inhibiting tumor growth and propagation.

While not wishing to be bound by any particular theory the results set forth herein suggest that the DLL3 ADC may bind and kill DLL3 expressing tumor cells in a manner that activates and/or attracts immunocompetent cells, including T-lymphocytes, to the tumor site. Moreover, eliminating or disrupting DLL3+ cancer stem cells through administration of DLL3 ADCs (e.g., by cellular disruption) may release CSC specific antigens that focus the subject's immune response on the germinal “seed cells” of the tumor. It will be appreciated that this patient-mediated focused immune attack on cancer stem cells can provide a mechanism for enhanced elimination of tumorigenic cells and corresponding inhibition of tumor growth or maintenance. The presence of PD-1 antibodies may then act to prevent the newly activated T-lymphocytes from receiving inhibitory signals through the PD-1 receptor on their surface (e.g., by engaging PD-L1 or PD-L2 ligands) and maintain or enhance the focused immune response. Thus, treatment with the DLL3 ADC may enhance the anti-tumorigenic effects of the administered PD-1 antibodies by generating or attracting newly activated T cells to eliminate cancer stem cells at the tumor site while, at the same time or subsequently, the PD-1 antibodies may enhance the anti-tumor effect of the CSC activated T-lymphocytes thereby eliminating more tumor cells than would otherwise be possible in the absence of the checkpoint inhibitors.

It will be appreciated that such DLL3 ADC/checkpoint inhibitor (e.g., an anti-PD-1 antibody) combinations allow for dosages and corresponding dosing regimens to be attenuated to provide particularly beneficial therapeutic profiles. More specifically such combinations may allow for the effective inhibition or elimination of tumorigenic cells at lower (or more infrequent) doses of either or both drugs when compared to their use as single agents. Thus, in certain aspects of the instant invention it may be possible to use relatively lower doses (as compared to use as a single agent) of the disclosed DLL3 ADCs with an anti-PD-1 antibody and still provide a therapeutic response with less toxicity. For example, rather than using the disclosed DLL3 ADC dosing regimens of 0.2 mg/kg Q3W×3 or 0.3 mg/kg Q6W×2 as discussed above, it may be possible to obtain equivalent therapeutic results using a lower DLL3 ADC dosage (e.g., 0.1 mg/kg, 0.05 mg/kg, etc) in combination with PD-1 antibodies. Similarly, effective therapeutic results may be obtained using a DLL3 ADC/anti-PD-1 combination with reduced or attenuated regimens such as fewer DLL3 ADC cycles (e.g., 0.2 mg/kg Q3W×2) or extended time between DLL3 ADC dosing (e.g., 0.2 mg/kg Q4W×2). In each such scenario (i.e., lower doses or extended dosing) it will be appreciated that the unexpected benefits provided by the disclosed therapeutic combinations allow for the effective treatment of the patient with relatively less toxicity. This, in turn, provides healthier patients that are better able to tolerate necessary treatments and/or prolong the therapeutic regimen to increase the odds of a positive response.

In other embodiments the anti-DLL3 antibodies or ADCs may be used in combination with various approved first line cancer treatments. In selected embodiments the combination therapy comprises the use of an anti-DLL3 antibody or ADC and a cytotoxic agent such as ifosfamide, mytomycin C, vindesine, vinblastine, etoposide, ironitecan, gemcitabine, taxanes, vinorelbine, methotrexate, and pemetrexed) and optionally one or more other therapeutic moiety(ies).

In another embodiment the combination therapy comprises the use of an anti-DLL3 antibody or ADC and a platinum-based drug (e.g. carboplatin or cisplatin) and optionally one or more other therapeutic moiety(ies) (e.g. vinorelbine; gemcitabine; a taxane such as, for example, docetaxel or paclitaxel; irinotican; or pemetrexed).

In certain embodiments, for example in the treatment of BR-ERPR, BR-ER or BR-PR cancer, the combination therapy comprises the use of an anti-DLL3 antibody or ADC and one or more therapeutic moieties described as “hormone therapy”. “Hormone therapy” as used herein, refers to, e.g., tamoxifen; gonadotropin or luteinizing releasing hormone (GnRH or LHRH); everolimus and exemestane; toremifene; or aromatase inhibitors (e.g. anastrozole, letrozole, exemestane or fulvestrant).

In another embodiment, for example, in the treatment of BR-HER2, the combination therapy comprises the use of an anti-DLL3 antibody or ADC and trastuzumab or ado-trastuzumab emtansine (Kadcyla®) and optionally one or more other therapeutic moiety(ies) (e.g. pertuzumab and/or docetaxel).

In some embodiments, for example, in the treatment of metastatic breast cancer, the combination therapy comprises the use of an anti-DLL3 antibody or ADC and a taxane (e.g. docetaxel or paclitaxel) and optionally an additional therapeutic moiety(ies), for example, an anthracycline (e.g. doxorubicin or epirubicin) and/or eribulin.

In another embodiment, for example, in the treatment of metastatic or recurrent breast cancer or BRCA-mutant breast cancer, the combination therapy comprises the use of an anti-DLL3 antibody or ADC and megestrol and optionally an additional therapeutic moiety(ies).

In further embodiments, for example, in the treatment of BR-TNBC, the combination therapy comprises the use of an anti-DLL3 antibody or ADC and a poly ADP ribose polymerase (PARP) inhibitor (e.g. BMN-673, olaparib, rucaparib and veliparib) and optionally an additional therapeutic moiety(ies).

In another embodiment the combination therapy comprises the use of an anti-DLL3 antibody or ADC and a PARP inhibitor and optionally one or more other therapeutic moiety(ies).

In another embodiment, for example, in the treatment of breast cancer, the combination therapy comprises the use of an anti-DLL3 antibody or ADC and cyclophosphamide and optionally an additional therapeutic moiety(ies) (e.g. doxorubicin, a taxane, epirubicin, 5-FU and/or methotrexate.

In another embodiment combination therapy for the treatment of EGFR-positive NSCLC comprises the use of an anti-DLL3 antibody or ADC and afatinib and optionally one or more other therapeutic moiety(ies) (e.g. erlotinib and/or bevacizumab).

In another embodiment combination therapy for the treatment of EGFR-positive NSCLC comprises the use of an anti-DLL3 antibody or ADC and erlotinib and optionally one or more other therapeutic moiety(ies) (e.g. bevacizumab).

In another embodiment combination therapy for the treatment of ALK-positive NSCLC comprises the use of an anti-DLL3 antibody or ADC and ceritinib (Zykadia) and optionally one or more other therapeutic moiety(ies).

In another embodiment combination therapy for the treatment of ALK-positive NSCLC comprises the use of an anti-DLL3 antibody or ADC and crizotinib (Xalcori) and optionally one or more other therapeutic moiety(ies).

In another embodiment the combination therapy comprises the use of an anti-DLL3 antibody or ADC and bevacizumab and optionally one or more other therapeutic moiety(ies) (e.g. gemcitabine or a taxane such as, for example, docetaxel or paclitaxel; and/or a platinum analog).

In another embodiment the combination therapy comprises the use of an anti-DLL3 antibody or ADC and bevacizumab and optionally cyclophosphamide.

In a particular embodiment the combination therapy for the treatment of platinum-resistant tumors comprises the use of an anti-DLL3 antibody or ADC and doxorubicin and/or etoposide and/or gemcitabine and/or vinorelbine and/or ifosfamide and/or leucovorin-modulated 5-fluoroucil and/or bevacizumab and/or tamoxifen; and optionally one or more other therapeutic moiety(ies).

In selected embodiments the disclosed antibodies and ADCs may be used in combination with certain steroids to potentially make the course of treatment more effective and reduce side effects such as inflammation, nausea and hypersensitivity. Exemplary steroids that may be used on combination with the ADCs of the instant invention include, but are not limited to, hydrocortisone, dexamethasone, prednisone, methylprednisolone and prednisolone. In particularly preferred aspects the steroid will comprise dexamethasone. In other preferred aspects the steroid will comprise prednisone.

Combination therapy may also comprise an anti-DLL3 antibody or ADC and a chemotherapeutic moiety that is effective on a tumor (e.g. melanoma) comprising a mutated or aberrantly expressed gene or protein (e.g. BRAF V600E).

T lymphocytes (e.g., cytotoxic lymphocytes (CTL)) play an important role in host defense against malignant tumors. CTL are activated by the presentation of tumor associated antigens on antigen presenting cells. Active specific immunotherapy is a method that can be used to augment the T lymphocyte response to cancer by vaccinating a patient with peptides derived from known cancer associated antigens. In one embodiment the combination therapy may comprise an anti-DLL3 antibody or ADC and a vaccine to a cancer associated antigen (e.g. melanocyte-lineage specific antigen tyrosinase, gp100, Melan-A/MART-1 or gp75.) In other embodiments the combination therapy may comprise administration of an anti-DLL3 antibody or ADC together with in vitro expansion, activation, and adoptive reintroduction of autologous CTLs or natural killer cells. CTL activation may also be promoted by strategies that enhance tumor antigen presentation by antigen presenting cells. Granulocyte macrophage colony stimulating factor (GM-CSF) promotes the recruitment of dendritic cells and activation of dendritic cell cross-priming. In one embodiment the combination therapy may comprise the isolation of antigen presenting cells, activation of such cells with stimulatory cytokines (e.g. GM-CSF), priming with tumor-associated antigens, and then adoptive reintroduction of the antigen presenting cells into patients in combination with the use of anti-DLL3 antibodies or ADCs and optionally one or more different therapeutic moiety(ies).

In some embodiments the anti-DLL3 antibodies or ADCs may be used in combination with various first line melanoma treatments. In one embodiment the combination therapy comprises the use of an anti-DLL3 antibody or ADC and dacarbazine and optionally one or more other therapeutic moiety(ies). In further embodiments the combination therapy comprises the use of an anti-DLL3 antibody or ADC and temozolamide and optionally one or more other therapeutic moiety(ies). In another embodiment the combination therapy comprises the use of an anti-DLL3 antibody or ADC and a platinum-based therapeutic moiety (e.g. carboplatin or cisplatin) and optionally one or more other therapeutic moiety(ies). In some embodiments the combination therapy comprises the use of an anti-DLL3 antibody or ADC and a vinca alkaloid therapeutic moiety (e.g. vinblastine, vinorelbine, vincristine, or vindesine) and optionally one or more other therapeutic moiety(ies). In one embodiment the combination therapy comprises the use of an anti-DLL3 antibody or ADC and interleukin-2 and optionally one or more other therapeutic moiety(ies). In another embodiment the combination therapy comprises the use of an anti-DLL3 antibody or ADC and interferon-alpha and optionally one or more other therapeutic moiety(ies).

In other embodiments, the anti-DLL3 antibodies or ADCs may be used in combination with adjuvant melanoma treatments and/or a surgical procedure (e.g. tumor resection). In one embodiment the combination therapy comprises the use of an anti-DLL3 antibody or ADC and interferon-alpha and optionally one or more other therapeutic moiety(ies).

The invention also provides for the combination of anti-DLL3 antibodies or ADCs with radiotherapy. The term “radiotherapy”, as used herein, means, any mechanism for inducing DNA damage locally within tumor cells such as gamma-irradiation, X-rays, UV-irradiation, microwaves, electronic emissions and the like. Combination therapy using the directed delivery of radioisotopes to tumor cells is also contemplated, and may be used in combination or as a conjugate of the anti-DLL3 antibodies disclosed herein. Typically, radiation therapy is administered in pulses over a period of time from about 1 to about 2 weeks. Optionally, the radiation therapy may be administered as a single dose or as multiple, sequential doses.

In other embodiments an anti-DLL3 antibody or ADC may be used in combination with one or more of the anti-cancer agents described below.

D. Anti-Cancer Agents

The term “anti-cancer agent” as used herein is one subset of “therapeutic moieties” which, in turn, is a subset of the agents described as “pharmaceutically active moieties.” More particularly “anti-cancer agent” means any agent (or a pharmaceutically acceptable salt thereof) that can be used to treat a cell proliferative disorder such as cancer, and includes, but is not limited to, cytotoxic agents, cytostatic agents, anti-angiogenic agents, debulking agents, chemotherapeutic agents, radiotherapeutic agents, targeted anti-cancer agents, biological response modifiers, therapeutic antibodies, cancer vaccines, cytokines, hormone therapy, anti-metastatic agents and immunotherapeutic agents. Note that the foregoing classifications of anti-cancer agents are not exclusive of each other and that selected agents may fall into one or more categories. For example, a compatible anti-cancer agent may be classified as a cytotoxic agent and a chemotherapeutic agent. Accordingly, each of the foregoing terms should be construed in view of the instant disclosure and then in accordance with their use in the medical arts.

In preferred embodiments an anti-cancer agent can include any chemical agent (e.g., a chemotherapeutic agent) that inhibits or eliminates, or is designed to inhibit or eliminate, a cancerous cell or a cell likely to become cancerous or generate tumorigenic progeny (e.g., tumorigenic cells). In this regard selected chemical agents (cell-cycle dependent agents) are often directed to intracellular processes necessary for cell growth or division, and are thus particularly effective against cancerous cells, which generally grow and divide rapidly. For example, vincristine depolymerizes microtubules and thus inhibits rapidly dividing tumor cells from entering mitosis. In other cases the selected chemical agents are cell-cycle independent agents that interfere with cell survival at any point of its lifecycle and may be effective in directed therapeutics (e.g., ADCs). By way of example certain pyrrolobenzodiazepines bind to the minor groove of cellular DNA and inhibit transcription upon delivery to the nucleus. With regard to combination therapy or selection of an ADC component it will be appreciated that one skilled in the art could readily identify compatible cell-cycle dependent agents and cell-cycle independent agents in view of the instant disclosure.

In any event, and as alluded to above, it will be appreciated that selected anti-cancer agents may be administered in combination with each other (e.g., CHOP therapy) in addition to the anti-DLL3 antibodies and ADCs disclosed herein. Moreover, it will further be appreciated that in certain embodiments such anti-cancer agents may comprise conjugates and may be associated with antibodies prior to administration. In some aspects the disclosed anti-cancer agent will be linked to an anti-DLL3 antibody to provide an ADC as disclosed herein.

As used herein the term “cytotoxic agent” (or cytotoxin) generally means a substance that is toxic to cells in that it decreases or inhibits cellular function and/or causes the destruction of tumor cells. In certain embodiments the substance is a naturally occurring molecule derived from a living organism or an analog thereof (purified from natural sources or synthetically prepared). Examples of cytotoxic agents include, but are not limited to, small molecule toxins or enzymatically active toxins of bacteria (e.g., calicheamicin, Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins [PAPI, PAPII, and PAP-S], Momordica charantia inhibitor, curcin, crotin, saponaria officinalis inhibitor, mitegellin, restrictocin, phenomycin, neomycin, and the tricothecenes) or animals, (e.g., cytotoxic RNases, such as extracellular pancreatic RNases; DNase I, including fragments and/or variants thereof). Additional compatible cytotoxic agents including certain radioisotopes, maytansinoids, auristatins, dolastatins, duocarmycins, amanitins and pyrrolobenzodiazepines are set forth herein.

More generally examples of cytotoxic agents or anti-cancer agents that may be used in combination with (or conjugated to) the antibodies of the invention include, but are not limited to, alkylating agents, alkyl sulfonates, anastrozole, amanitins, aziridines, ethylenimines and methylamelamines, acetogenins, a camptothecin, BEZ-235, bortezomib, bryostatin, callystatin, CC-1065, ceritinib, crizotinib, cryptophycins, dolastatin, duocarmycin, eleutherobin, erlotinib, pancratistatin, a sarcodictyin, spongistatin, nitrogen mustards, antibiotics, enediyne dynemicin, bisphosphonates, esperamicin, chromoprotein enediyne antiobiotic chromophores, aclacinomysins, actinomycin, authramycin, azaserine, bleomycins, cactinomycin, canfosfamide, carabicin, carminomycin, carzinophilin, chromomycinis, cyclosphosphamide, dactinomycin, daunorubicin, detorubicin, 6-diazo-5-oxo-L-norleucine, doxorubicin, epirubicin, esorubicin, exemestane, fluorouracil, fulvestrant, gefitinib, idarubicin, lapatinib, letrozole, lonafarnib, marcellomycin, megestrol acetate, mitomycins, mycophenolic acid, nogalamycin, olivomycins, pazopanib, peplomycin, potfiromycin, puromycin, quelamycin, rapamycin, rodorubicin, sorafenib, streptonigrin, streptozocin, tamoxifen, tamoxifen citrate, temozolomide, tepodina, tipifarnib, tubercidin, ubenimex, vandetanib, vorozole, XL-147, zinostatin, zorubicin; anti-metabolites, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, polysaccharide complex, razoxane; rhizoxin; SF-1126, sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside; cyclophosphamide; thiotepa; taxoids, chloranbucil; gemcitabine; 6-thioguanine; mercaptopurine; methotrexate; platinum analogs, vinblastine; platinum; etoposide; ifosfamide; mitoxantrone; vincristine; vinorelbine; novantrone; teniposide; edatrexate; daunomycin; aminopterin; xeloda; ibandronate; irinotecan, topoisomerase inhibitor RFS 2000; difluorometlhylornithine; retinoids; capecitabine; combretastatin; leucovorin; oxaliplatin; XL518, inhibitors of PKC-alpha, Raf, H-Ras, EGFR and VEGF-A that reduce cell proliferation and pharmaceutically acceptable salts or solvates, acids or derivatives of any of the above. Also included in this definition are anti-hormonal agents that act to regulate or inhibit hormone action on tumors such as anti-estrogens and selective estrogen receptor antibodies, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts or solvates, acids or derivatives of any of the above.

Compatible cytotoxic agents or anti-cancer agents may also comprise commercially or clinically available compounds such as erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®). Additional commercially or clinically available anti-cancer agents comprise oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); vinorelbine (NAVELBINE®); capecitabine (XELODA®, Roche), tamoxifen (including NOLVADEX®; tamoxifen citrate, FARESTON® (toremifine citrate) MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca).

The term “pharmaceutically acceptable salt” or “salt” means organic or inorganic salts of a molecule or macromolecule. Acid addition salts can be formed with amino groups. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′ methylene bis-(2-hydroxy 3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Where multiple charged atoms are part of the pharmaceutically acceptable salt, the salt can have multiple counter ions. Hence, a pharmaceutically acceptable salt can have one or more charged atoms and/or one or more counterion.

Similarly a “Pharmaceutically acceptable solvate” or “solvate” refers to an association of one or more solvent molecules and a molecule or macromolecule. Examples of solvents that form pharmaceutically acceptable solvates include, but are not limited to, water, isopropanol, ethanol, methanol, DMSO, ethyl acetate, acetic acid, and ethanolamine.

In other embodiments the antibodies or ADCs of the instant invention may be used in combination with any one of a number of antibodies (or immunotherapeutic agents) presently in clinical trials or commercially available. The disclosed antibodies may be used in combination with an antibody selected from the group consisting of abagovomab, adecatumumab, afutuzumab, alemtuzumab, altumomab, amatuximab, anatumomab, arcitumomab, atezolizumab, avelumab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, dacetuzumab, dalotuzumab, daratumumab, detumomab, drozitumab, duligotumab, durvalumab, dusigitumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lambrolizumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nivolumab, nofetumomabn, obinutuzumab, ocaratuzumab, ofatumumab, olaratumab, olaparib, onartuzumab, oportuzumab, oregovomab, panitumumab, parsatuzumab, patritumab, pembrolizumab pemtumomab, pertuzumab, pidilizumab, pintumomab, pritumumab, racotumomab, radretumab, ramucirumab, rilotumumab, rituximab, robatumumab, satumomab, selumetinib, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, 3F8, MED10680, MDX-1105 and combinations thereof.

Other embodiments comprise the use of antibodies approved for cancer therapy including, but not limited to, rituximab, gemtuzumab ozogamcin, alemtuzumab, ibritumomab tiuxetan, tositumomab, bevacizumab, cetuximab, patitumumab, ofatumumab, ipilimumab and brentuximab vedotin. Those skilled in the art will be able to readily identify additional anti-cancer agents that are compatible with the teachings herein.

E. Radiotherapy

As briefly discussed above the present invention also provides for the combination of antibodies or ADCs with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated and the disclosed antibodies or ADCs may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### VIII. INDICATIONS

The invention provides for the use of antibodies and ADCs of the invention for the diagnosis, theragnosis, treatment and/or prophylaxis of various disorders including proliferative disorders, neoplastic, inflammatory, angiogenic and immunologic disorders and disorders caused by pathogens. In certain embodiments the diseases to be treated comprise neoplastic conditions and in certain other aspects comprise solid tumors. In other embodiments the diseases to be treated comprise hematologic malignancies. In still other embodiments the antibodies or ADCs of the invention will be used to treat tumors or tumorigenic cells expressing a DLL3 determinant. Preferably the “subject” or “patient” to be treated will be human although, as used herein, the terms are expressly held to comprise any mammalian species.

It will be appreciated that the compounds and compositions of the instant invention may be used to treat subjects at various stages of disease and at different points in their treatment cycle. Accordingly, in certain embodiments the antibodies and ADCs of the instant invention will be used as a front line therapy and administered to subjects who have not previously been treated for the cancerous condition. In other embodiments the antibodies and ADCs of the invention will be used to treat second and third line patients (i.e., those subjects that have previously been treated for the same condition one or two times respectively). Still other embodiments will comprise the treatment of fourth line or higher patients (e.g., SCLC patients) that have been treated for the same or related condition three or more times with the disclosed DLL3 ADCs or with different therapeutic agents. In other embodiments the compounds and compositions of the present invention will be used to treat subjects that have previously been treated (with antibodies or ADCs of the present invention or with other anti-cancer agents) and have relapsed or are determined to be refractory to the previous treatment. In selected embodiments the compounds and compositions of the instant invention may be used to treat subjects that have recurrent tumors.

As discussed above and shown in the appended Examples the compounds and compositions of the instant invention may be used to treat any subject suffering from a DLL3+ tumor (e.g., a tumor with a DLL3 H-score of 90 or above and/or the tumor expresses DLL3 in 20% of cells) preferably determined using IHC and/or an H-score derived as set forth herein. In this regard one embodiment of the invention comprises treatment of a patient with an H-score of at least 90 with the disclosed DLL3 ADCs. In other embodiments a patient to be treated with the DLL3 ADCs of the instant invention will have a DLL3 H-score of at least 120. In yet other embodiments a patient to be treated with the DLL3 ADCs of the instant invention will have a DLL3 H-score of at least 150 and more preferably will have a DLL3 H-score of at least 180. Still other embodiments will comprise DLL3+ tumors wherein the tumor expresses DLL3 in at least 20%, 30% 40% or at least 50% of the constituent cells as determined by staining. In certain preferred embodiments the H-score or percentage of cells stained will be measured using a sample obtained from a SCLC tumor. In other embodiments the H-score or percentage of cells stained will be measured using a sample obtained from a large cell neuroendocrine carcinoma, a glioblastoma, a melanoma or a medullary thyroid tumor. In still other embodiments the H-score or percentage of stained cells will be measured using a sample obtained from a tumor exhibiting neuroendocrine features.

In certain aspects the proliferative disorder will comprise a solid tumor including, but not limited to, adrenal, liver, kidney, bladder, breast, gastric, ovarian, cervical, uterine, esophageal, colorectal, prostate (e.g., prostate adenocarcinoma), pancreatic, lung (both small cell and non-small cell), thyroid, carcinomas, sarcomas, glioblastomas and various head and neck tumors. In other preferred embodiments, and as shown in the Examples below, the disclosed ADCs are particularly effective at treating small cell lung cancer (SCLC) and non-small cell lung cancer (NSCLC) (e.g., squamous cell non-small cell lung cancer or squamous cell small cell lung cancer). In certain embodiments the lung cancer is refractory, relapsed or resistant to a platinum based agent (e.g., carboplatin, cisplatin, oxaliplatin, topotecan) and/or a taxane (e.g., docetaxel, paclitaxel, larotaxel or cabazitaxel). In another embodiment the subject to be treated is suffering from large cell neuroendocrine carcinoma (LCNEC). In still other aspects of the invention the disclosed antibodies and ADCs may be used for the treatment of medullary thyroid cancer, glioblastoma, neuroendocrine prostate cancer, (NEPC), high-grade gastroenteropancreatic cancer (GEP) and malignant melanoma.

More generally exemplary neoplastic conditions subject to treatment in accordance with the instant invention may be benign or malignant; solid tumors or hematologic malignancies; and may be selected from the group including, but not limited to: adrenal gland tumors, AIDS-associated cancers, alveolar soft part sarcoma, astrocytic tumors, autonomic ganglia tumors, bladder cancer (squamous cell carcinoma and transitional cell carcinoma), blastocoelic disorders, bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, epithelial disorders, Ewing's tumors, extraskeletal myxoid chondrosarcoma, fibrogenesis imperfecta ossium, fibrous dysplasia of the bone, gallbladder and bile duct cancers, gastric cancer, gastrointestinal, gestational trophoblastic disease, germ cell tumors, glandular disorders, head and neck cancers, hypothalamic, intestinal cancer, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), macrophagal disorders, medulloblastoma, melanoma, meningiomas, medullary thyroid cancer, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, stromal disorders, synovial sarcoma, testicular cancer, thymic carcinoma, thymoma, thyroid metastatic cancer, and uterine cancers (carcinoma of the cervix, endometrial carcinoma, and leiomyoma).

In still other preferred embodiments the compounds or compositions will be administered to a subject suffering from melanoma. More generally the compositions and methods disclosed herein may be used to diagnose, monitor, treat or prevent melanoma. The term “melanoma”, as used herein, includes all types of melanoma including, but not limited to, primary melanoma, malignant melanoma, cutaneous melanoma, extracutaneous melanoma, superficial spreading melanoma, polypoid melanoma, melanocarcinomas, melanoepitheliomas, melanosarcomas, melanoma in situ, nodular malignant melanoma, lentigo maligna melanoma, lentiginous melanoma, lentiginous malignant melanoma, mucosal lentiginous melanoma, mucosal melanoma, acral lentiginous melanoma, soft tissue melanoma, ocular melanoma, invasive melanoma, familial atypical mole and melanoma (FAM-M) syndrome, desmoplastic malignant melanoma or uveal melanoma.

Metastatic melanoma may be derived from melanocytes, melanocytic nevi or dysplastic nevi and can evolve through different phases of tumor progression (e.g. radial growth phase or vertical growth phase. Melanoma can be caused by chromosomal abnormalities, degenerative growth and/or developmental disorders, mitogenic agents, ultraviolet radiation, viral infections, carcinogenic agents, various genetic mutations or abnormal expression of a gene.

As alluded to above the disclosed antibodies and ADCs are especially effective at treating lung cancer, including the following subtypes: small cell lung cancer and non-small cell lung cancer (e.g. squamous cell non-small cell lung cancer or squamous cell small cell lung cancer) and large cell neuroendocrine carcinoma. In selected embodiments the antibodies and ADCs can be administered to patients exhibiting limited stage disease or extensive stage disease. In other embodiments the disclosed conjugated antibodies will be administered to refractory patients (i.e., those whose disease recurs during or shortly after completing a course of initial therapy); sensitive patients (i.e., those whose relapse is longer than 2-3 months after primary therapy); or patients exhibiting resistance to a platinum based agent (e.g. carboplatin, cisplatin, oxaliplatin) and/or a taxane (e.g. docetaxel, paclitaxel, larotaxel or cabazitaxel). In certain preferred embodiments the DLL3 ADCs of the instant invention may be administered to frontline patients (i.e., those who have not been treated for lung cancer). In other embodiments the DLL3 ADCs of the instant invention may be administered to second line lung cancer patients. In still other embodiments the DLL3 ADCs of the instant invention may be administered to third or fourth line lung cancer patients.

In particularly preferred embodiments the disclosed ADCs may be used to treat small cell lung cancer. With regard to such embodiments the conjugated antibodies may be administered to patients exhibiting limited stage disease. In other embodiments the disclosed ADCs will be administered to patients exhibiting extensive stage disease. In other preferred embodiments the disclosed ADCs will be administered to refractory patients (i.e., those who recur during or shortly after completing a course of initial therapy) or recurrent small cell lung cancer patients. Still other embodiments comprise the administration of the disclosed ADCs to sensitive patients (i.e., those whose relapse is longer than 2-3 months after primary therapy for SCLC). In each case it will be appreciated that compatible ADCs may be used in combination with other anti-cancer agents (e.g., platinum based agents or taxanes or antibodies to PD-1 or PD-L1) depending the selected dosing regimen and the clinical diagnosis.

As set forth herein the disclosed ADCs may further be used to prevent, treat or diagnose tumors with neuroendocrine features or phenotypes including neuroendocrine tumors. True or canonical neuroendocrine tumors (NETs) arising from the dispersed endocrine system are relatively rare, with an incidence of 2-5 per 100,000 people, but highly aggressive. Neuroendocrine tumors occur in the kidney, genitourinary tract (bladder, prostate, ovary, cervix, and endometrium), gastrointestinal tract (colon, stomach), thyroid (medullary thyroid cancer), and lung (small cell lung carcinoma and large cell neuroendocrine carcinoma). These tumors may secrete several hormones including serotonin and/or chromogranin A that can cause debilitating symptoms known as carcinoid syndrome. Such tumors can be denoted by positive immunohistochemical markers such as neuron-specific enolase (NSE, also known as gamma enolase, gene symbol=ENO2), CD56 (or NCAM1), chromogranin A (CHGA), and synaptophysin (SYP) or by genes known to exhibit elevated expression such as ASCL1. Unfortunately traditional chemotherapies have not been particularly effective in treating NETs and liver metastasis is a common outcome.

While the disclosed ADCs may be advantageously used to treat neuroendocrine tumors they may also be used to treat, prevent or diagnose pseudo neuroendocrine tumors (pNETs) that genotypically or phenotypically mimic, resemble or exhibit common traits with canonical neuroendocrine tumors. Pseudo neuroendocrine tumors or tumors that exhibit neuroendocrine features are tumors that arise from cells of the diffuse neuroendocrine system or from cells in which a neuroendocrine differentiation cascade has been aberrantly reactivated during the oncogenic process. Such pNETs commonly share certain phenotypic or biochemical characteristics with traditionally defined neuroendocrine tumors (i.e., they exhibit neuroendocrine features), including the ability to produce subsets of biologically active amines, neurotransmitters, and peptide hormones. Histologically, such tumors (NETs and pNETs) share a common appearance often showing densely connected small cells with minimal cytoplasm of bland cytopathology and round to oval stippled nuclei. For the purposes of the instant invention commonly expressed histological markers or genetic markers that may be used to define neuroendocrine and pseudo neuroendocrine tumors include, but are not limited to, chromogranin A, CD56, synaptophysin, PGP9.5, ASCL1 and neuron-specific enolase (NSE).

Accordingly the ADCs of the instant invention may beneficially be used to treat both pseudo neuroendocrine tumors and canonical neuroendocrine tumors. In this regard the ADCs may be used as described herein to treat neuroendocrine tumors (both NET and pNET) arising in the kidney, genitourinary tract (bladder, prostate, ovary, cervix, and endometrium), gastrointestinal tract (colon, stomach), thyroid (medullary thyroid cancer), and lung (small cell lung carcinoma and large cell neuroendocrine carcinoma). Moreover, the ADCs of the instant invention may be used to treat tumors expressing one or more markers selected from the group consisting of NSE, CD56, synaptophysin, chromogranin A, ASCL1 and PGP9.5 (UCHL1). That is, the present invention may be used to treat a subject suffering from a tumor that is NSE or CD56⁺ or PGP9.5⁺ or ASCL1⁺ or SYR⁺ or CHGA⁺ or some combination thereof.

As previously alluded to the DLL3 ADCs of the invention may be used to treat SCLC patients with progressive disease after one or two treatments (i.e., second or third line SCLC patients) particularly if they exhibit the aforementioned markers.

With regard to hematologic malignancies it will be further be appreciated that the compounds and methods of the present invention may be particularly effective in treating a variety of B-cell lymphomas, including low grade/NHL follicular cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, Waldenstrom's Macroglobulinemia, lymphoplasmacytoid lymphoma (LPL), mantle cell lymphoma (MCL), follicular lymphoma (FL), diffuse large cell lymphoma (DLCL), Burkitt's lymphoma (BL), AIDS-related lymphomas, monocytic B cell lymphoma, angioimmunoblastic lymphoadenopathy, small lymphocytic, follicular, diffuse large cell, diffuse small cleaved cell, large cell immunoblastic lymphoblastoma, small, non-cleaved, Burkitt's and non-Burkitt's, follicular, predominantly large cell; follicular, predominantly small cleaved cell; and follicular, mixed small cleaved and large cell lymphomas. See, Gaidono et al., “Lymphomas”, IN CANCER: PRINCIPLES & PRACTICE OF ONCOLOGY, Vol. 2: 2131-2145 (DeVita et al., eds., 5.sup.th ed. 1997). It should be clear to those of skill in the art that these lymphomas will often have different names due to changing systems of classification, and that patients having lymphomas classified under different names may also benefit from the combined therapeutic regimens of the present invention.

### IX. ARTICLES OF MANUFACTURE

The invention includes pharmaceutical packs and kits comprising one or more containers or receptacles, wherein a container can comprise one or more doses of an antibody or ADC of the invention. Such kits or packs may be diagnostic or therapeutic in nature. In certain embodiments, the pack or kit contains a unit dosage, meaning a predetermined amount of a composition comprising, for example, an antibody or ADC of the invention, with or without one or more additional agents and optionally, one or more anti-cancer agents. In certain other embodiments, the pack or kit contains a detectable amount of an anti-DLL3 antibody or ADC, with or without an associated reporter molecule and optionally one or more additional agents for the detection, quantitation, staining and/or visualization of cancerous cells.

In any event kits of the invention will generally comprise an antibody or ADC of the invention in a suitable container or receptacle comprising a pharmaceutically acceptable formulation and, optionally, one or more anti-cancer agents in the same or different containers. The kits may also contain other pharmaceutically acceptable formulations or devices, either for diagnosis or combination therapy. Examples of diagnostic devices or instruments include those that can be used to detect, interrogate, monitor, quantify or profile cells or markers associated with proliferative disorders (for a list of such exemplary markers, see above). In some embodiments the devices may be used to detect, monitor and/or quantify circulating tumor cells either in vivo or in vitro (see, for example, WO 2012/0128801). In still other embodiments the circulating tumor cells may comprise tumorigenic cells. The kits contemplated by the invention can also contain appropriate reagents to combine the antibody or ADC of the invention with an anti-cancer agent or diagnostic agent.

When the components of the kit are provided in one or more liquid solutions, the liquid solution can be non-aqueous, though typically an aqueous solution is preferred, with a sterile aqueous solution being particularly preferred. The formulation in the kit can also be provided as dried powder(s) or in lyophilized form that can be reconstituted upon addition of an appropriate liquid. The liquid used for reconstitution can be contained in a separate container. Such liquids can comprise sterile, pharmaceutically acceptable buffer(s) or other diluent(s) such as bacteriostatic water for injection, sterile water for injection, phosphate-buffered saline, Ringer's solution or dextrose solution. Where the kit comprises the antibody or ADC of the invention in combination with additional therapeutics or agents, the solution may be pre-mixed, either in a molar equivalent combination, or with one component in excess of the other. Alternatively, the antibody or ADC of the invention and any optional anti-cancer agent or other agent can be maintained separately within distinct containers prior to administration to a patient.

In certain preferred embodiments kits of the invention will comprise vials or bottles containing a lyophilized DLL3 ADC. Preferably the lyophilized ADC will comprise a DLL3 ADC selected from the group consisting of ADC1, ADC2, ADC3, ADC4, ADC5 and ADC6 and more preferably the lyophilized DLL3 ADC will comprise hSC16.56DL1 or hSC16.56ss1DL6. In certain embodiments the lyophilized DLL3 ADC will further comprise a lyoprotectant. In other aspects the kits may optionally comprise a container comprising an aqueous solution that may be used to reconstitute the lyophilized DLL3 ADC. In still other embodiments the kits may comprise an insert or instructions indicating that the lyophilized DLL3 remains stable for 3 months, 6 months, 1 year, 18 months, 2 years, 30 months or three years at 2-8° C. (e.g., 4 CC). In selected preferred embodiments the insert or instructions will indicate that the lyophilized ADC will remain stable for two years at 2-8° C. (e.g., 4 CC). In certain preferred embodiments the aforementioned kits comprising the lyophilized DLL3 ADC will comprise a label, marker, package insert, bar code and/or reader indicating that the kit contents may be used for the treatment, prevention and/or diagnosis of cancer. In a particularly preferred aspect the label, marker, package insert, bar code and/or reader indicates that the kit contents may be used for the treatment, prevention and/or diagnosis of small cell lung cancer. In other particularly preferred aspects the label, marker, package insert, bar code and/or reader indicates that the kit contents may be used for the treatment, prevention and/or diagnosis of large cell neuroendocrine carcinoma, melanoma, thyroid cancer or glioblastoma.

More generally the kit can comprise one or multiple containers or receptacles and a label or package insert in, on or associated with the container(s), indicating that the enclosed composition is used for diagnosing or treating the disease condition of choice (e.g., cancer). Suitable containers include, for example, bottles, vials, syringes, infusion bags (IV bags), etc. The containers can be formed from a variety of materials such as glass or pharmaceutically compatible plastics. In certain embodiments the container(s) can comprise a sterile access port such as, for example, an intravenous solution bag or a vial having a stopper that can be pierced by a hypodermic injection needle.

In some embodiments the kit can contain a means by which to administer the antibody and any optional components to a patient, e.g., one or more needles or syringes (pre-filled or empty), an eye dropper, pipette, or other such like apparatus, from which the formulation may be injected or introduced into the subject or applied to a diseased area of the body. The kits of the invention will also typically include a means for containing the vials, or such like, and other components in close confinement for commercial sale, such as, e.g., blow-molded plastic containers into which the desired vials and other apparatus are placed and retained.

### X. MISCELLANEOUS

Unless otherwise defined herein, scientific and technical terms used in connection with the invention shall have the meanings that are commonly understood by those of ordinary skill in the art. Further, unless otherwise required by context, singular terms shall include pluralities and plural terms shall include the singular. In addition, ranges provided in the specification and appended claims include both end points and all points between the end points. Therefore, a range of 2.0 to 3.0 includes 2.0, 3.0, and all points between 2.0 and 3.0.

Generally, techniques of cell and tissue culture, molecular biology, immunology, microbiology, genetics and chemistry described herein are those well-known and commonly used in the art. The nomenclature used herein, in association with such techniques, is also commonly used in the art. The methods and techniques of the invention are generally performed according to conventional methods well known in the art and as described in various references that are cited throughout the present specification unless otherwise indicated.

### XI. REFERENCES

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PBD, and translations from annotated coding regions in GenBank and RefSeq) cited herein are incorporated by reference, regardless of whether the phrase “incorporated by reference” is or is not used in relation to the particular reference. The foregoing detailed description and the examples that follow have been given for clarity of understanding only. No unnecessary limitations are to be understood therefrom. The invention is not limited to the exact details shown and described. Variations obvious to one skilled in the art are included in the invention defined by the claims. Any section headings used herein are for organizational purposes only and are not to be construed as limiting the subject matter described.

### XII. SEQUENCES

Appended to the instant application are figures and a sequence listing comprising a number of nucleic acid and amino acid sequences. The following Table 4 provides a summary of the included sequences.

TABLE 4

| SEQ ID NO. | Description |
|---|---|
| 1 | DLL3 isoform 1 protein |
| 2 | DLL3 isoform 2 protein |
| 3 | Epitope protein - SC16.23 |
| 4 | Epitope protein - SC16.34 & SC 16.56 |
| 5 | Kappa constant region protein |
| 6 | IgG1 constant region protein |
| 7 | hSC16.56 full length heavy chain protein |
| 8 | hSC16.56 and hSC16.56ss1 full length light chain prot. |
| 9 | hSC16.56ss1 full length heavy chain protein |
| 10-19 | Reserved |
| 20 | SC16.3 VL DNA (aligned with encoded protein) |
| 21 | SC16.3 VL protein |
| 22 | SC16.3 VH DNA (aligned with encoded protein) |
| 23 | SC16.3 VH protein |
| 24-387 | Additional murine clones as in SEQ ID NOs: 20-23 |
| 388-407 | Humanized clones as in SEQ ID NOs: 20-23 |
| 408, 409, 410 | hSC16.13 CDRL1, CDRL2, CDRL3 |
| 411, 412, 413 | hSC16.13 CDRH1, CDRH2, CDRH3 |
| 414, 415, 416 | hSC16.15 CDRL1, CDRL2, CDRL3 |
| 417, 418, 419 | hSC16.15 CDRH1, CDRH2, CDRH3 |
| 420, 421, 422 | hSC16.25 CDRL1, CDRL2, CDRL3 |
| 423, 424, 425 | hSC16.25 CDRH1, CDRH2, CDRH3 |
| 426, 427, 428 | hSC16.34 CDRL1, CDRL2, CDRL3 |
| 429, 430, 431 | hSC16.34 CDRH1, CDRH2, CDRH3 |
| 432, 433, 434 | hSC16.56 CDRL1, CDRL2, CDRL3 |
| 435, 436, 437 | hSC16.56 CDRH1, CDRH2, CDRH3 |

As discussed in Example 2 below, Table 4 above may further be used to designate SEQ ID NOS for exemplary Kabat CDRs delineated in FIGS. 1A and 1B. More particularly FIGS. 1A and 1B denote the three Kabat CDRs of each heavy (CDRH) and light (CDRL) chain variable region sequence and Table 4 above provides for assignment of a SEQ ID designation that may be applied to each CDRL1, CDRL2 and CDRL3 of the light chain and each CDRH1, CDRH2 and CDRH3 of the heavy chain. Using this methodology each unique CDR set forth in FIGS. 1A and 1B may be assigned a sequential SEQ ID NO and can be used to provide the derived antibodies of the instant invention.

### XIII. TUMOR LISTING

PDX tumor cell types are denoted by an abbreviation followed by a number, which indicates the particular tumor cell line. The passage number of the tested sample is indicated by p0-p# appended to the sample designation where p0 is indicative of an unpassaged sample obtained directly from a patient tumor and p# is indicative of the number of times the tumor has been passaged through a mouse prior to testing. As used herein, the abbreviations of the tumor types and subtypes are shown in Table 5 as follows:

TABLE 5

| Tumor Type | Abbreviation | Tumor subtype | Abbreviation |
|---|---|---|---|
| Breast | BR | | |
| | | estrogen receptor positive and/or | BR-ERPR |
| | | progesterone receptor positive | |
| | | ERBB2/Neu positive | BR-ERBB2/Neu |
| | | HER2 positive | BR-HER2 |
| | | triple-negative | TNBC |
| | | claudin subtype of triple-negative | TNBC-CLDN |
| colorectal | CR | | |
| endometrial | EN | | |
| gastric | GA | | |
| | | diffuse adenocarcinoma | GA-Ad-Dif/Muc |
| | | intestinal adenocarcinoma | GA-Ad-Int |
| | | stromal tumors | GA-GIST |
| glioblastoma | GB | | |
| head and neck | HN | | |
| kidney | KDY | | |
| | | clear renal cell carcinoma | KDY-CC |
| | | papillary renal cell carcinoma | KDY-PAP |
| | | transitional cell or urothelial | KDY-URO |
| | | carcinoma | |
| | | unknown | KDY-UNK |
| liver | LIV | | |
| | | hepatocellular carcinoma | LIV-HCC |
| | | cholangiocarcinoma | LIV-CHOL |
| lymphoma | LN | | |
| lung | LU | | |
| | | adenocarcinoma | LU-Ad |
| | | carcinoid | LU-CAR |
| | | large cell neuroendocrine | LU-LCC |
| | | non-small cell | NSCLC |
| | | squamous cell | LU-SCC |
| | | small cell | SCLC |
| | | spindle cell | LU-SPC |
| melanoma | MEL | | |
| ovarian | OV | | |
| | | clear cell | OV-CC |
| | | endometroid | OV-END |
| | | mixed subtype | OV-MIX |
| | | malignant mixed mesodermal | OV-MMMT |
| | | mucinous | OV-MUC |
| | | neuroendocrine | OV-NET |
| | | papillary serous | OV-PS |
| | | serous | OV-S |
| | | small cell | OV-SC |
| | | transitional cell carcinoma | OV-TCC |
| pancreatic | PA | | |
| | | acinar cell carcinoma | PA-ACC |
| | | duodenal carcinoma | PA-DC |
| | | mucinous adenocarcinoma | PA-MAD |
| | | neuroendocrine | PA-NET |
| | | adenocarcinoma | PA-PAC |
| | | adenocarcinoma exocrine type | PA-PACe |
| | | ductal adenocarcinoma | PA-PDAC |
| | | ampullary adenocarcinoma | PA-AAC |
| prostate | PR | | |
| skin | SK | | |
| | | melanoma | MEL |
| | | squamous cell carcinomas | SK-SCC |

### EXAMPLES

The invention, thus generally described above, will be understood more readily by reference to the following examples, which are provided by way of illustration and are not intended to be limiting of the instant invention. The examples are not intended to represent that the experiments below are all or the only experiments performed. Unless indicated otherwise, parts are parts by weight, molecular weight is weight average molecular weight, temperature is in degrees Centigrade, and pressure is at or near atmospheric.

### Example 1

### Generation of Murine Anti-DLL3 Antibodies

Anti-DLL3 murine antibodies were produced as follows. In a first immunization campaign, three mice (one from each of the following strains: Balb/c, CD-1, FVB) were inoculated with human DLL3-fc protein (hDLL3-Fc) emulsified with an equal volume of TiterMax® or alum adjuvant. The hDLL3-Fc fusion construct was purchased from Adipogen International (Catalog No. AG-40A-0113). An initial immunization was performed with an emulsion of 10 μg hDLL3-Fc per mouse in TiterMax. Mice were then boosted biweekly with 5 μg hDLL3-Fc per mouse in alum adjuvant. The final injection prior to fusion was with 5 μg hDLL3-Fc per mouse in PBS.

In a second immunization campaign six mice (two each of the following strains: Balb/c, CD-1, FVB), were inoculated with human DLL3-His protein (hDLL3-His), emulsified with an equal volume of TiterMax® or alum adjuvant. Recombinant hDLL3-His protein was purified from the supernatants of CHO-S cells engineered to overexpress hDLL3-His. The initial immunization was with an emulsion of 10 μg hDLL3-His per mouse in TiterMax. Mice were then boosted biweekly with 5 μg hDLL3-His per mouse in alum adjuvant. The final injection was with 2×10⁵ HEK-293T cells engineered to overexpress hDLL3.

Solid-phase ELISA assays were used to screen mouse sera for mouse IgG antibodies specific for human DLL3. A positive signal above background was indicative of antibodies specific for DLL3. Briefly, 96 well plates (VWR International, Cat. #610744) were coated with recombinant DLL3-His at 0.5 μg/ml in ELISA coating buffer overnight. After washing with PBS containing 0.02% (v/v) Tween 20, the wells were blocked with 3% (w/v) BSA in PBS, 200 μL/well for 1 hour at room temperature (RT). Mouse serum was titrated (1:100, 1:200, 1:400, and 1:800) and added to the DLL3 coated plates at 50 μL/well and incubated at RT for 1 hour. The plates are washed and then incubated with 50 μL/well HRP-labeled goat anti-mouse IgG diluted 1:10,000 in 3% BSA-PBS or 2% FCS in PBS for 1 hour at RT. Again the plates were washed and 40 μL/well of a TMB substrate solution (Thermo Scientific 34028) was added for 15 minutes at RT. After developing, an equal volume of 2N H₂SO₄ was added to stop substrate development and the plates were analyzed by spectrophotometer at OD 450.

Sera-positive immunized mice were sacrificed and draining lymph nodes (popliteal, inguinal, and medial iliac) were dissected and used as a source for antibody producing cells. Cell suspensions of B cells (approximately 229×10⁶ cells from the hDLL3-Fc immunized mice, and 510×10⁶ cells from the hDLL3-His immunized mice) were fused with non-secreting P3×63Ag8.653 myeloma cells at a ratio of 1:1 by electro cell fusion using a model BTX Hybrimmune System (BTX Harvard Apparatus). Cells were re-suspended in hybridoma selection medium consisting of DMEM medium supplemented with azaserine, 15% fetal clone I serum, 10% BM Condimed (Roche Applied Sciences), 1 mM nonessential amino acids, 1 mM HEPES, 100 IU penicillin-streptomycin, and 50 μM 2-mercaptoethanol, and were cultured in four T225 flasks in 100 mL selection medium per flask. The flasks were placed in a humidified 37° C. incubator containing 5% CO₂ and 95% air for six to seven days.

On day six or seven after the fusions the hybridoma library cells were collected from the flasks and plated at one cell per well (using the FACSAria I cell sorter) in 200 μL of supplemented hybridoma selection medium (as described above) into 64 Falcon 96-well plates, and 48 96-well plates for the hDLL3-His immunization campaign. The rest of the library was stored in liquid nitrogen.

The hybridomas were cultured for 10 days and the supernatants were screened for antibodies specific to hDLL3 using flow cytometry performed as follows. 1×10⁵ per well of HEK-293T cells engineered to overexpress human DLL3, mouse DLL3 (pre-stained with dye), or cynomolgus DLL3 (pre-stained with Dylight800) were incubated for 30 minutes with 25 μL hybridoma supernatant. Cells were washed with PBS/2% FCS and then incubated with 25 μL per sample DyeLight 649 labeled goat-anti-mouse IgG, Fc fragment specific secondary diluted 1:300 in PBS/2% FCS. After a 15 minute incubation cells were washed twice with PBS/2% FCS and re-suspended in PBS/2% FCS with DAPI and analyzed by flow cytometry for fluorescence exceeding that of cells stained with an isotype control antibody. Remaining unused hybridoma library cells were frozen in liquid nitrogen for future library testing and screening.

The hDLL3-His immunization campaign yielded approximately 50 murine anti-hDLL3 antibodies and the hDLL3-Fc immunization campaign yielded approximately 90 murine anti-hDLL3 antibodies.

### Example 2

### Sequencing of Anti-DLL3 Antibodies

Based on the foregoing, a number of exemplary distinct monoclonal antibodies that bind immobilized human DLL3 or h293-hDLL3 cells with apparently high affinity were selected for sequencing and further analysis. Sequence analysis of the light chain variable regions and heavy chain variable regions from selected monoclonal antibodies generated in Example 1 confirmed that many had novel complementarity determining regions and often displayed novel VDJ arrangements.

Initially selected hybridoma cells expressing the desired antibodies were lysed in Trizol® reagent (Trizol® Plus RNA Purification System, Life Technologies) to prepare the RNA encoding the antibodies. Between 10⁴ and 10⁵ cells were re-suspended in 1 mL Trizol and shaken vigorously after addition of 200 μL chloroform. Samples were then centrifuged at 4° C. for 10 minutes and the aqueous phase was transferred to a fresh microfuge tube and an equal volume of 70% ethanol was added. The sample was loaded on an RNeasy Mini spin column, placed in a 2 mL collection tube and processed according to the manufacturer's instructions. Total RNA was extracted by elution, directly to the spin column membrane with 100 μL RNase-free water. The quality of the RNA preparations was determined by fractionating 3 μL in a 1% agarose gel before being stored at −80° C. until used.

The variable region of the Ig heavy chain of each hybridoma was amplified using a 5′ primer mix comprising 32 mouse specific leader sequence primers designed to target the complete mouse VH repertoire in combination with a 3′ mouse Cy primer specific for all mouse Ig isotypes. Similarly, a primer mix containing thirty two 5′ Vκ leader sequences designed to amplify each of the Vκ mouse families was used in combination with a single reverse primer specific to the mouse kappa constant region in order to amplify and sequence the kappa light chain. For antibodies containing a lambda light chain, amplification was performed using three 5′ VL leader sequences in combination with one reverse primer specific to the mouse lambda constant region. The VH and VL transcripatients were amplified from 100 ng total RNA using the Qiagen One Step RT-PCR kit as follows. A total of eight RT-PCR reactions were run for each hybridoma, four for the Vκ light chain and four for the Vγ heavy chain. PCR reaction mixtures included 3 μL of RNA, 0.5 μL of 100 μM of either heavy chain or kappa light chain primers (custom synthesized by Integrated Data Technologies), 5 μL of 5×RT-PCR buffer, 1 μL dNTPs, 1 μL of enzyme mix containing reverse transcriptase and DNA polymerase, and 0.4 μL of ribonuclease inhibitor RNasin (1 unit). The thermal cycler program was RT step 50° C. for 30 minutes, 95° C. for 15 minutes followed by 30 cycles of (95° C. for 30 seconds, 48° C. for 30 seconds, 72° C. for 1 minute). There was then a final incubation at 72° C. for 10 minutes.

The extracted PCR products were sequenced using the same specific variable region primers as described above for the amplification of the variable regions. To prepare the PCR products for direct DNA sequencing, they were purified using the QIAquick™ PCR Purification Kit (Qiagen) according to the manufacturer's protocol. The DNA was eluted from the spin column using 50 μL of sterile water and then sequenced directly from both strands (MCLAB).

Selected nucleotide sequences were analyzed using the IMGT sequence analysis tool (http://www.imgt.org/IMGTmedical/_sequence_analysis.html) to identify germline V, D and J gene members with the highest sequence homology. These derived sequences were compared to known germline DNA sequences of the Ig V- and J-regions by alignment of VH and VL genes to the mouse germline database using a proprietary antibody sequence database.

FIG. 1A depicts the contiguous amino acid sequences of numerous novel murine light chain variable regions from anti-DLL3 antibodies and exemplary humanized light chain variable regions derived from the variable light chains of representative murine anti-DLL3 antibodies (as per Example 3 below). FIG. 1B depicts the contiguous amino acid sequences of novel murine heavy chain variable regions from the same anti-DLL3 antibodies and humanized heavy chain variable regions derived from the same murine antibodies providing the humanized light chains (as per Example 3 below). Murine light and heavy chain variable region amino acid sequences are provided in SEQ ID NOS: 21-387, odd numbers while humanized light and heavy chain variable region amino acid sequences are provided in SEQ ID NOS: 389-407, odd numbers.

Thus, taken together FIGS. 1A and 1B provide the annotated sequences of numerous murine anti-DLL3 binding or targeting domains, termed SC16.3, SC16.4, SC16.5, SC16.7, SC16.8, SC16.10, SC16.11, SC16.13, SC16.15, SC16.18, SC16.19, SC16.20, SC16.21, SC16.22, SC16.23, SC16.25, SC16.26, SC16.29, SC16.30, SC16.31, SC16.34, SC16.35, SC16.36, SC16.38, SC16.41, SC16.42, SC16.45, SC16.47, SC16.49, SC16.50, SC16.52, SC16.55, SC16.56, SC16.57, SC16.58, SC16.61, SC16.62, SC16.63, SC16.65, SC16.67, SC16.68, SC16.72, SC16.73, SC16.78, SC16.79, SC16.80, SC16.81, SC16.84, SC16.88, SC16.101, SC16.103, SC16.104, SC16.105, SC16.106, SC16.107, SC16.108, SC16.109, SC16.110, SC16.111, SC16.113, SC16.114, SC16.115, SC16.116, SC16.117, SC16.118, SC16.120, SC16.121, SC16.122, SC16.123, SC16.124, SC16.125, SC16.126, SC16.129, SC16.130, SC16.131, SC16.132, SC16.133, SC16.134, SC16.135, SC16.136, SC16.137, SC16.138, SC16.139, SC16.140, SC16.141, SC16.142, SC16.143, SC16.144, SC16.147, SC16.148, SC16.149 and SC16.150 and humanized antibodies, termed hSC16.13, hSC16.15, hSC16.25, hSC16.34 and hSC16.56.

In particular aspects of the invention the ADC binding domain binds specifically to hDLL3 and comprises or competes for binding with an antibody comprising: a light chain variable region (VL) of SEQ ID NO: 21 and a heavy chain variable region (VH) of SEQ ID NO: 23; or a VL of SEQ ID NO: 25 and a VH of SEQ ID NO: 27; or a VL of SEQ ID NO: 29 and a VH of SEQ ID NO: 31; or a VL of SEQ ID NO: 33 and a VH of SEQ ID NO: 35; or a VL of SEQ ID NO: 37 and a VH of SEQ ID NO: 39; or a VL of SEQ ID NO: 41 and a VH of SEQ ID NO: 43; or a VL of SEQ ID NO: 45 and a VH of SEQ ID NO: 47; or a VL of SEQ ID NO: 49 and a VH of SEQ ID NO: 51; or a VL of SEQ ID NO: 53 and a VH of SEQ ID NO: 55; or a VL of SEQ ID NO: 57 and a VH of SEQ ID NO: 59; or a VL of SEQ ID NO: 61 and a VH of SEQ ID NO: 63; or a VL of SEQ ID NO: 65 and a VH of SEQ ID NO: 67; or a VL of SEQ ID NO: 69 and a VH of SEQ ID NO: 71; or a VL of SEQ ID NO: 73 and a VH of SEQ ID NO: 75; or a VL of SEQ ID NO: 77 and a VH of SEQ ID NO: 79; or a VL of SEQ ID NO: 81 and a VH of SEQ ID NO: 83; or a VL of SEQ ID NO: 85 and a VH of SEQ ID NO: 87; or a VL of SEQ ID NO: 89 and a VH of SEQ ID NO: 91; or a VL of SEQ ID NO: 93 and a VH of SEQ ID NO: 95; or a VL of SEQ ID NO: 97 and a VH of SEQ ID NO: 99; or a VL of SEQ ID NO: 101 and a VH of SEQ ID NO: 103; or a VL of SEQ ID NO: 105 and a VH of SEQ ID NO: 107; or a VL of SEQ ID NO: 109 and a VH of SEQ ID NO: 111; or a VL of SEQ ID NO: 113 and a VH of SEQ ID NO: 115; or a VL of SEQ ID NO: 117 and a VH of SEQ ID NO: 119; or a VL of SEQ ID NO: 121 and a VH of SEQ ID NO: 123; or a VL of SEQ ID NO: 125 and a VH of SEQ ID NO: 127; or a VL of SEQ ID NO: 129 and a VH of SEQ ID NO: 131; or a VL of SEQ ID NO: 133 and a VH of SEQ ID NO: 135; or a VL of SEQ ID NO: 137 and a VH of SEQ ID NO: 139; or a VL of SEQ ID NO: 141 and a VH of SEQ ID NO: 143; or a VL of SEQ ID NO: 145 and a VH of SEQ ID NO: 147; or a VL of SEQ ID NO: 149 and a VH of SEQ ID NO: 151; or a VL of SEQ ID NO: 153 and a VH of SEQ ID NO: 155; or a VL of SEQ ID NO: 157 and a VH of SEQ ID NO: 159; or a VL of SEQ ID NO: 161 and a VH of SEQ ID NO: 163; or a VL of SEQ ID NO: 165 and a VH of SEQ ID NO: 167; or a VL of SEQ ID NO: 169 and a VH of SEQ ID NO: 171; or a VL of SEQ ID NO: 173 and a VH of SEQ ID NO: 175; or a VL of SEQ ID NO: 177 and a VH of SEQ ID NO: 179; or a VL of SEQ ID NO: 181 and a VH of SEQ ID NO: 183; or a VL of SEQ ID NO: 185 and a VH of SEQ ID NO: 187; or a VL of SEQ ID NO: 189 and a VH of SEQ ID NO: 191; or a VL of SEQ ID NO: 193 and a VH of SEQ ID NO: 195; or a VL of SEQ ID NO: 197 and a VH of SEQ ID NO: 199; or a VL of SEQ ID NO: 201 and a VH of SEQ ID NO: 203; or a VL of SEQ ID NO: 205 and a VH of SEQ ID NO: 207; or a VL of SEQ ID NO: 209 and a VH of SEQ ID NO: 211; or a VL of SEQ ID NO: 213 and a VH of SEQ ID NO: 215; or a VL of SEQ ID NO: 217 and a VH of SEQ ID NO: 219; or a VL of SEQ ID NO: 221 and a VH of SEQ ID NO: 223; or a VL of SEQ ID NO: 225 and a VH of SEQ ID NO: 227; or a VL of SEQ ID NO: 229 and a VH of SEQ ID NO: 231; or a VL of SEQ ID NO: 233 and a VH of SEQ ID NO: 235; or a VL of SEQ ID NO: 237 and a VH of SEQ ID NO: 239; or a VL of SEQ ID NO: 241 and a VH of SEQ ID NO: 243; or a VL of SEQ ID NO: 245 and a VH of SEQ ID NO: 247; or a VL of SEQ ID NO: 249 and a VH of SEQ ID NO: 251; or a VL of SEQ ID NO: 253 and a VH of SEQ ID NO: 255; or a VL of SEQ ID NO: 257 and a VH of SEQ ID NO: 259; or a VL of SEQ ID NO: 261 and a VH of SEQ ID NO: 263; or a VL of SEQ ID NO: 265 and a VH of SEQ ID NO: 267; or a VL of SEQ ID NO: 269 and a VH of SEQ ID NO: 271; or a VL of SEQ ID NO: 273 and a VH of SEQ ID NO: 275; or a VL of SEQ ID NO: 277 and a VH of SEQ ID NO: 279; or a VL of SEQ ID NO: 281 and a VH of SEQ ID NO: 283; or a VL of SEQ ID NO: 285 and a VH of SEQ ID NO: 287; or a VL of SEQ ID NO: 289 and a VH of SEQ ID NO: 291; or a VL of SEQ ID NO: 293 and a VH of SEQ ID NO: 295; or a VL of SEQ ID NO: 297 and a VH of SEQ ID NO: 299; or a VL of SEQ ID NO: 301 and a VH of SEQ ID NO: 303; or a VL of SEQ ID NO: 305 and a VH of SEQ ID NO: 307; or a VL of SEQ ID NO: 309 and a VH of SEQ ID NO: 311; or a VL of SEQ ID NO: 313 and a VH of SEQ ID NO: 315; or a VL of SEQ ID NO: 317 and a VH of SEQ ID NO: 319; or a VL of SEQ ID NO: 321 and a VH of SEQ ID NO: 323; or a VL of SEQ ID NO: 325 and a VH of SEQ ID NO: 327; or a VL of SEQ ID NO: 329 and a VH of SEQ ID NO: 331; or a VL of SEQ ID NO: 333 and a VH of SEQ ID NO: 335; or a VL of SEQ ID NO: 337 and a VH of SEQ ID NO: 339; or a VL of SEQ ID NO: 341 and a VH of SEQ ID NO: 343; or a VL of SEQ ID NO: 345 and a VH of SEQ ID NO: 347; or a VL of SEQ ID NO: 349 and a VH of SEQ ID NO: 351; or a VL of SEQ ID NO: 353 and a VH of SEQ ID NO: 355; or a VL of SEQ ID NO: 357 and a VH of SEQ ID NO: 359; or a VL of SEQ ID NO: 361 and a VH of SEQ ID NO: 363; or a VL of SEQ ID NO: 365 and a VH of SEQ ID NO: 367; or a VL of SEQ ID NO: 369 and a VH of SEQ ID NO: 371; or a VL of SEQ ID NO: 373 and a VH of SEQ ID NO: 375; or a VL of SEQ ID NO: 377 and a VH of SEQ ID NO: 379; or a VL of SEQ ID NO: 381 and a VH of SEQ ID NO: 383; or a VL of SEQ ID NO: 385 and a VH of SEQ ID NO: 387; or a VL of SEQ ID NO: 389 and a VH of SEQ ID NO: 391; or a VL of SEQ ID NO: 393 and a VH of SEQ ID NO: 395; or a VL of SEQ ID NO: 397 and a VH of SEQ ID NO: 399; or a VL of SEQ ID NO: 401 and a VH of SEQ ID NO: 403; or a VL of SEQ ID NO: 405 and a VH of SEQ ID NO: 407.

For the purposes of the instant application the SEQ ID NOS of each particular antibody are sequential odd numbers. Thus the monoclonal anti-DLL3 antibody, SC16.3, comprises amino acid SEQ ID NOS: 21 and 23 for the light and heavy chain variable regions respectively; SC16.4 comprises SEQ ID NOS: 25 and 27; SC16.5 comprises SEQ ID NOS: 29 and 31, and so on. The corresponding nucleic acid sequence for each antibody amino acid sequence is included in the appended sequence listing and has the SEQ ID NO immediately preceding the corresponding amino acid SEQ ID NO. Thus, for example, the SEQ ID NOS of the VL and VH of the SC16.3 antibody are 21 and 23, respectively, and the SEQ ID NOS of the nucleic acid sequences encoding the VL and VH of the SC16.3 antibody are SEQ ID NOS: 20 and 22, respectively. The CDRs in FIGS. 1A and 1B are defined as per Kabat et al. (supra) using a proprietary version of the Abysis database.

### Example 3

### Generation of Chimeric and Humanized Anti-DLL3 Antibodies

To provide a benchmark for humanized binding domains compatible with the instant invention chimeric anti-DLL3 antibodies were generated using art-recognized techniques as follows. Total RNA was extracted from the hybridomas and amplified as set forth in Example 1. Data regarding V, D and J gene segments of the VH and VL chains of the murine antibodies were obtained from the derived nucleic acid sequences. Primer sets specific to the leader sequence of the VH and VL chain of the antibody were designed using the following restriction sites: AgeI and XhoI for the VH fragments, and XmaI and DraIII for the VL fragments. PCR products were purified with a QIAquick PCR purification kit (Qiagen), followed by digestion with restriction enzymes AgeI and XhoI for the V_{H} fragments and XmaI and DraIII for the VL fragments. The VL and VH digested PCR products were purified and ligated into kappa CL (SEQ ID NO: 5) human light chain constant region expression vector or IgG1 (SEQ ID NO: 6) human heavy chain constant region expression vector, respectively.

Ligation reactions were performed in a total volume of 10 μL with 200U T4-DNA Ligase (New England Biolabs), 7.5 μL of digested and purified gene-specific PCR product and 25 ng linearized vector DNA. Competent E. coli DH10B bacteria (Life Technologies) were transformed via heat shock at 42° C. with 3 μL ligation product and plated onto plates with ampicillin at a concentration of 100 μg/mL. Following purification and digestion of the amplified ligation products, the V_{H} fragment was cloned into the AgeI-XhoI restriction sites of the pEE6.4HulgG1 expression vector (Lonza) and the VL fragment was cloned into the XmaI-DraIII restriction sites of the pEE12.4Hu-Kappa expression vector (Lonza Ltd.).

Chimeric antibodies were expressed by co-transfection of HEK-293T cells with pEE6.4HulgG1 and pEE12.4Hu-Kappa expression vectors. Prior to transfection the HEK-293T cells were cultured in 150 mm plates under standard conditions in Dulbecco's Modified Eagle's Medium (DMEM) supplemented with 10% heat inactivated FCS, 100 μg/mL streptomycin and 100 U/mL penicillin G. For transient transfections cells were grown to 80% confluency. 12.5 μg each of pEE6.4HulgG1 and pEE12.4Hu-Kappa vector DNA were added to 50 μL HEK-293T transfection reagent in 1.5 mL Opti-MEM. The mix was incubated for 30 minutes at room temperature and plated. Supernatants were harvested three to six days after transfection. Culture supernatants containing recombinant chimeric antibodies were cleared from cell debris by centrifugation at 800×g for 10 minutes and stored at 4° C. Recombinant chimeric antibodies were purified by Protein A affinity chromatography.

The same murine anti-DLL3 antibodies (e.g. SC16.13, SC16.15, SC16.25, SC16.34 and SC16.56) were also used to derive CDR-grafted or humanized binding domains. The murine antibodies were humanized using a proprietary computer-aided CDR-grafting method (Abysis Database, UCL Business) and standard molecular engineering techniques as follows. Human framework regions of the variable regions were designed based on the highest homology between the framework sequences and CDR canonical structures of human germline antibody sequences and the framework sequences and CDRs of the relevant mouse antibodies. For the purpose of the analysis the assignment of amino acids to each of the CDR domains was done in accordance with Kabat et al. Once the variable regions were selected, they were generated from synthetic gene segments (Integrated DNA Technologies). Humanized antibodies were cloned and expressed using the molecular methods described above for chimeric antibodies.

The genetic composition for the selected human acceptor variable regions are shown in Table 6 immediately below for each of the humanized antibodies. The sequences depicted in Table 6 correspond to the contiguous variable region sequences set forth in SEQ ID NOS: 389 and 391 (hSC16.13), SEQ ID NOS: 393 and 395 (hSC16.15), SEQ ID NOS: 397 and 399 (hSC16.25), SEQ ID NOS: 401 and 403 (hSC16.34) and SEQ ID NOS: 405 and 407 (hSC16.56). Table 6 shows that no framework changes or back mutations were necessary to maintain the favorable binding properties of the selected antibodies.

TABLE 6

| | | | human | FW | | human | FW |
|---|---|---|---|---|---|---|---|
| mAb | human VH | human DH | JH | changes | human VK | JK | changes |
| hSC16.13 | IGHV2- | IGHD1-1 | JH6 | None | IGKV1- | JK1 | None |
| | 5*01 | | | | 39*01 | | |
| hSC16.15 | IGHV1- | IGHD2-2 | JH4 | None | IGKV1- | JK4 | None |
| | 46*01 | | | | 13*02 | | |
| hSC16.25 | IGHV2- | IGHD3-16 | JH6 | None | IGKV6- | JK2 | None |
| | 5*01 | | | | 21*01 | | |
| hSC16.34 | IGHV1- | IGHD3-22 | JH4 | None | IGKV1- | JK1 | None |
| | 3*02 | | | | 27*01 | | |
| hSC16.56 | IGHV1- | IGHD2-21 | JH4 | None | IGKV3- | JK2 | None |
| | 18*01 | | | | 15*01 | | |

Although no residues were altered in the framework regions, in one of the humanized clones (hSC16.13) mutations were introduced into heavy chain CDR2 to address stability concerns. The binding affinity of the antibody with the modified CDR was checked to ensure that it was equivalent to either the corresponding chimeric or murine antibody.

Following humanization the resulting VL and VH chain amino acid sequences were analyzed to determine their homology with regard to the murine donor and human acceptor light and heavy chain variable regions. The results shown in Table 7, immediately below, reveal that the humanized constructs consistently exhibited a higher homology with respect to the human acceptor sequences than with the murine donor sequences. The murine heavy and light chain variable regions show a similar overall percentage homology to a closest match of human germline genes (85%-93%) compared with the homology of the humanized antibodies and the donor hybridoma protein sequences (74%-83%).

TABLE 7

| | Homology to Human | Homology to Murine Parent |
|---|---|---|
| mAb | (CDR acceptor) | (CDR donor) |
| hSC16.13 HC | 93% | 81% |
| hSC16.13 LC | 87% | 77% |
| hSC16.15 HC | 85% | 83% |
| hSC16.15 LC | 85% | 83% |
| hSC16.25 HC | 91% | 83% |
| hSC16.25 LC | 85% | 79% |
| hSC16.34 HC | 87% | 79% |
| hSC16.34 LC | 85% | 81% |
| hSC16.56 HC | 87% | 74% |
| hSC16.56 LC | 87% | 76% |

As with the chimeric antibodies the humanized VL and VH digested PCR products were purified and ligated into kappa CL (SEQ ID NO: 5) human light chain constant region expression vector or IgG1 (SEQ ID NO: 6) human heavy chain constant region expression vector, respectively. Following expression each of the derived humanized constructs were analyzed using surface plasmon resonance, to determine if the CDR grafting process had appreciably altered their apparent affinity for DLL3 protein. The humanized constructs were compared with chimeric antibodies comprising the murine parent (or donor) heavy and light chain variable domains and a human constant region substantially equivalent to that used in the humanized constructs. The humanized anti-DLL3 antibodies exhibited binding characteristics roughly comparable to those shown by the chimeric parent antibodies (data not shown).

### Example 4

### Generation of Site Specific Antibodies

An engineered human IgG1/kappa anti-DLL3 site-specific antibody was constructed comprising a native light chain (LC) constant region and heavy chain (HC) constant region, wherein cysteine 220 (C220) in the upper hinge region of the HC, which forms an interchain disulfide bond with cysteine 214 (C214) in the LC, was substituted with serine (C220S). When assembled the HCs and LCs form an antibody comprising two free cysteines that are suitable for conjugation to a therapeutic agent. Unless otherwise noted, all numbering of constant region residues is in accordance with the EU numbering scheme as set forth in Kabat et al.

The engineered antibodies were generated as follows. An expression vector encoding the full-length humanized anti-DLL3 antibody hSC16.56 was used as a template for PCR amplification and site directed mutagenesis. Site directed mutagenesis was performed using the Quickchange® system (Agilent Technologies) according to the manufacturer's instructions.

The vector encoding the mutant C220S heavy chain of hSC16.56 was co-transfected with the native full-length kappa light chain in CHO-S cells and expressed using a mammalian transient expression system. The engineered anti-DLL3 site-specific antibody containing the C220S mutant was termed hSC16.56ss1 (SEQ ID NOS: 8 and 9). Once expressed the engineered anti-DLL3 antibodies were characterized by SDS-PAGE to confirm that the correct mutants had been generated. SDS-PAGE was conducted on a pre-cast 10% Tris-Glycine mini gel from life technologies in the presence and absence of a reducing agent such as DTT (dithiothreitol). Following electrophoresis, the gels were stained with a colloidal Coomassie solution. Under reducing conditions, two bands corresponding to the free LCs and free HCs, were observed (data not shown). This pattern is typical of IgG molecules in reducing conditions. Under non-reducing conditions, the band patterns were different from native IgG molecules, indicative of the absence of a disulfide bond between the HC and LC. A band around 98 kD corresponding to the HC-HC dimer was observed. In addition, a faint band corresponding to the free LC and a predominant band around 48 kD that corresponded to a LC-LC dimer was observed. The formation of some amount of LC-LC species is expected due to the free cysteines on the C-terminus of each LC.

### Example 5

### Domain and Epitope Mapping of Anti-DLL3 Antibodies

In order to characterize and position the epitopes that the disclosed anti-DLL3 antibodies bind to, domain-level epitope mapping was performed using a modification of the protocol described by Cochran et al., 2004 (supra). Individual domains of DLL3 comprising specific amino acid sequences were expressed on the surface of yeast, and binding by each anti-DLL3 antibody was determined through flow cytometry.

Yeast display plasmid constructs were created for the expression of the following constructs: DLL3 extracellular domain (amino acids 27-466); DLL1-DLL3 chimera, which consists of the N-terminal region and DSL domain of DLL1 (amino acids 22-225) fused to EGF-like domains 1 through 6 of DLL3 (amino acids 220-466); DLL3-DLL1 chimera, which consists of the N-terminal region and DSL domain of DLL3 (amino acids 27-214) fused to EGF-like domains 1 through 8 of DLL1 (amino acids 222-518); EGF1 (amino acids 215-249); EGF2 (amino acids 274-310); EGF1 and EGF2 (amino acids 215-310); EGF3 (amino acids 312-351); EGF4 (amino acids 353-389); EGF5 (amino acids 391-427); and EGF6 (amino acids 429-465). For domain information see generally UniProtKB/Swiss-Prot database entry Q9NYJ7. Note that the amino acid numbering references an unprocessed DLL3 protein with a leader sequence included in the sequence set forth in SEQ ID NO. 1.) For analysis of the N-terminal region or the EGF domains as a whole, chimeras with the family member DLL1 (DLL1-DLL3 and DLL3-DLL1) were used as opposed to fragments to minimize potential problems with protein folding. Domain-mapped antibodies had previously been shown not to cross-react with DLL1 indicating that any binding to these constructs was occurring through association with the DLL3 portion of the construct. These plasmids were transformed into yeast, which were then grown and induced as described in Cochran et al.

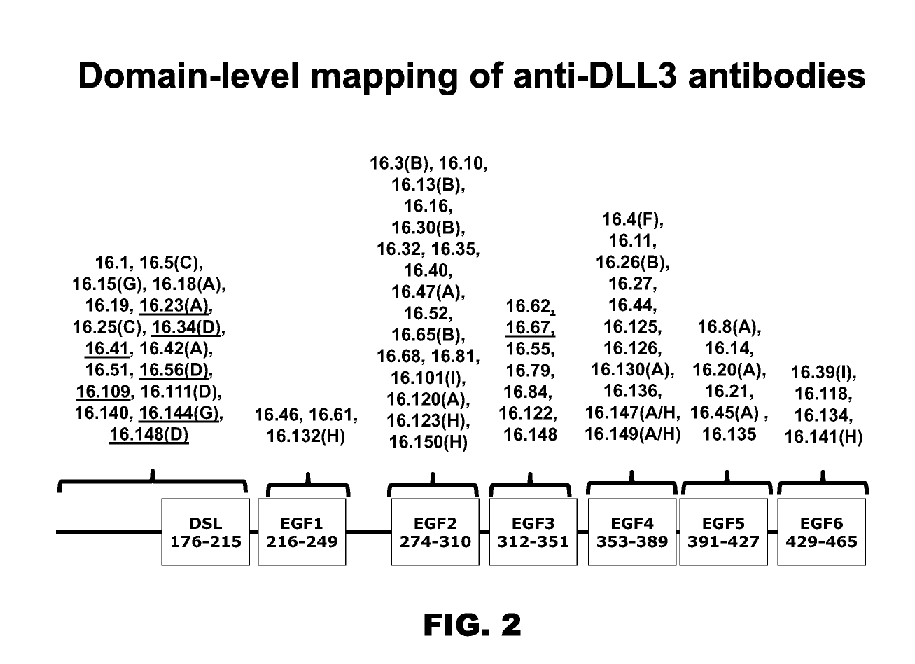

To test for binding to a particular construct, 200,000 induced yeast cells expressing the desired construct were washed twice in PBS+1 mg/mL BSA (PBSA), and incubated in 50 μL of PBSA with biotinylated anti-HA clone 3F10 (Roche Diagnostics) at 0.1 μg/mL and either 50 nM purified antibody or 1:2 dilution of unpurified supernatant from hybridomas cultured for 7 days. Cells were incubated for 90 minutes on ice, followed by two washes in PBSA. Cells were then incubated in 50 μL PBSA with the appropriate secondary antibodies: for murine antibodies, Alexa 488 conjugated streptavidin, and Alexa 647 conjugated goat anti mouse (Life Technologies) were added at 1 μg/mL each; and for humanized or chimeric antibodies, Alexa 647 conjugated streptavidin (Life Technologies) and R-phycoerythrin conjugated goat anti human (Jackson Immunoresearch) were added at 1 μg/mL each. After a twenty minute incubation on ice, cells were washed twice with PBSA and analyzed on a FACS Canto II. Antibodies that bound to DLL3-DLL1 chimera were designated as binding to the N-terminal region+DSL. Antibodies that bound specifically to an epitope present on a particular EGF-like domain were designated as binding to its respective domain (FIG. 2.)

In order to classify an epitope as conformational (e.g., discontinuous) or linear, yeast displaying the DLL3 ECD was heat treated for 30 minutes at 80° C. to denature the DLL3 ECD, and then washed twice in ice-cold PBSA. The ability of anti-DLL3 antibodies to bind the denatured yeast was tested by FACS using the same staining protocol as described above. Antibodies that bound to both the denatured and native yeast were classified as binding to a linear epitope, whereas antibodies that bound native yeast but not denatured yeast were classified as conformationally specific.

A schematic summary of the domain-level epitope mapping data of the antibodies tested is presented in FIG. 2, with antibodies binding a linear epitope underlined and, where determined, the corresponding bin noted in parenthesis. A review of FIG. 2 shows that the majority of anti-DLL3 antibodies tended to map to epitopes found either in the N-terminal/DSL region of DLL3 or EGF2. FIG. 2 presents similar data in a tabular form on bin determination and domain mapping for various anti-DLL3 antibodies.

Fine epitope mapping was further performed on selected antibodies using one of two methods. The first method employed the Ph.D.-12 phage display peptide library kit (New England Biolabs) which was used in accordance with the manufacturer's instructions. The antibody for epitope mapping was coated overnight at 50 μg/mL in 3 mL 0.1 M sodium bicarbonate solution, pH 8, onto a Nunc MaxiSorp tube (Nunc). The tube was blocked with 3% BSA solution in bicarbonate solution. Then, 10¹¹ input phage in PBS+0.1% Tween-20 was allowed to bind, followed by ten consecutive washes with 0.1% Tween-20 to wash away non-binding phage. Remaining phage were eluted with 1 mL 0.2 M glycine for 10 minutes at room temperature with gentle agitation, followed by neutralization with 150 μL 1M Tris-HCl pH 9. Eluted phage were amplified and panned again with 10¹¹ input phage, using 0.5% Tween-20 during the wash steps to increase selection stringency. DNA from 24 plaques of the eluted phage from the second round was isolated using the Qiaprep M13 Spin kit (Qiagen) and sequenced. Binding of clonal phage was confirmed using an ELISA assay, where the mapped antibody or a control antibody was coated onto an ELISA plate, blocked, and exposed to each phage clone. Phage binding was detected using horseradish peroxidase conjugated anti-M13 antibody (GE Healthcare), and the 1-Step Turbo TMB ELISA solution (Pierce). Phage peptide sequences from specifically binding phage were aligned using Vector NTI (Life Technologies) against the antigen ECD peptide sequence to determine the epitope of binding.

Alternatively, a yeast display method (Chao et al., 2007, PMID: 17406305) was used to map the epitopes of selected antibodies. Libraries of DLL3 ECD mutants were generated with error prone PCR using nucleotide analogues 8-oxo-2′deoxyguanosine-5′-triphosphate and 2′-deoxy-p-nucleoside-5′triphosphate (TriLink Bio) for a target mutagenesis rate of one amino acid mutation per clone. These were transformed into a yeast display format. Using the technique described above for domain-level mapping, the library was stained for HA and antibody binding at 50 nM. Using a FACS Aria (BD), clones that exhibited a loss of binding compared to wild type DLL3 ECD were sorted. These clones were re-grown, and subjected to another round of FACS sorting for loss of binding to the target antibody. Using the Zymoprep Yeast Plasmid Miniprep kit (Zymo Research), individual ECD clones were isolated and sequenced. Where necessary, mutations were reformatted as single-mutant ECD clones using the Quikchange site directed mutagenesis kit (Agilent).

Individual ECD clones were next screened to determine whether loss of binding was due to a mutation in the epitope, or a mutation that caused misfolding. Mutations that involved cysteine, proline, and stop codons were automatically discarded due to the high likelihood of a misfolding mutation. Remaining ECD clones were then screened for binding to a non-competing, conformationally specific antibody. ECD clones that lost binding to non-competing, conformationally specific antibodies were concluded to contain misfolding mutations, whereas ECD clones that retained equivalent binding to wild type DLL3 ECD were concluded to be properly folded. Mutations in the ECD clones in the latter group were concluded to be in the epitope.

A summary of selected antibodies with their derived epitopes comprising amino acid residues that are involved in antibody binding are listed in Table 8 below. Antibodies SC16.34 and SC16.56 interact with common amino acid residues which is consistent with the binning information and domain mapping results shown in FIG. 2. Moreover, SC16.23 was found to interact with a distinct contiguous epitope and was found not to bin with SC16.34 or SC16.56. Note that for the purposes of the appended sequence listing SEQ ID NO: 4 comprises a placeholder amino acid at position 204.

TABLE 8

| Antibody Clone | Epitope | SEQ ID NO: |
|---|---|---|
| SC16.23 | Q93, P94, G95, A96, P97 | 3 |
| SC16.34 | G203, R205, P206 | 4 |
| SC16.56 | G203, R205, P206 | 4 |

### Example 6

### Conjugation of Anti-DLL3 Antibodies to Pyrrolobenzodiazepines (PBDs)

A humanized anti-DLL3 antibody (hSC16.56) and a humanized site-specific anti-DLL3 antibody (hSC16.56ss1) were conjugated to pyrrolobenzodiazepine drug linkers (DL1 and DL6 respectively, each comprising PBD1) via a terminal maleimido moiety with a free sulfhydryl group to create the ADCs termed hSC16.56DL1 and hSC16.56ss1DL6. hSC16.56PDL1 (i.e., SC16LD6.5) and hSC16.56ss1 DL6 were made under GMP conditions as it was intended for use in clinical trials.

The humanized DLL3 (hSC16.56) antibody drug conjugates (ADCs) were prepared in two distinct stages; a reduction step and a conjugation step to conjugate PBD1 (DL1) to hSC16.56. The ADCs were then processed through Cation Exchange (CEX) Chromatography, followed by diafiltration and formulation steps to produce the drug substance. The process is described in detail below.

The antibodies were adjusted to pH 7.5 with the addition of 200 mM Tris Base, 32 mM EDTA pH 8.5. Cysteine bonds of the pH adjusted DLL3 antibodies were then partially reduced with a pre-determined molar addition of mol tris(2-carboxyethyl)-phosphine (TCEP) per mol antibody for 90 min. at 20° C. The resulting partially reduced preparations were then conjugated to PBD1 (as set forth above) via a maleimide linker for a minimum of 30 minutes at 20° C. The reaction was then quenched with the addition of excess N-acetyl cysteine (NAC) compared to linker-drug using a 10 mM stock solution prepared in water. After a minimum quench time of 20 minutes the pH was adjusted to 5.5 with the addition of 0.5 M acetic acid. Preparations of the ADCs were then processed through Cation Exchange (CEX) Chromatography in a bind and elute mode with a step elution to remove aggregates formed during the conjugation step. CEX purified ADCs were then buffer exchanged into diafiltration buffer by diafiltration using a 30 kDa membrane. The dialfiltered anti-DLL3 ADC was then formulated with sucrose and polysorbate-20 to the target final concentration to produce drug substance.

The site specific humanized anti-DLL3 (hSC16.56ss1) ADCs were conjugated to DL6 using a modified partial reduction process. In this respect the desired product is an ADC that is maximally conjugated on the unpaired cysteine (C214) on each LC constant region and that minimizes ADCs having a drug to antibody ratio (DAR) which is greater than 2 (DAR>2) while maximizing ADCs having a DAR of 2 (DAR=2). In order to further improve the specificity of the conjugation, the antibodies were selectively reduced using a process comprising a stabilizing agent (e.g. L-arginine) and a mild reducing agent (e.g. glutathione) prior to conjugation with the linker-drug. The ADCs were then processed through preparative Hydrophobic Interaction Chromatography (HIC), followed by a diafiltration and formulation step to produce the drug substance. The process is described in detail below.

The site-specific antibody constructs were partially reduced in a buffer containing 1M L-arginine/5 mM EDTA with a pre-determined concentration of reduced glutathione (GSH), pH 8.0 for a minimum of two hours at room temperature. All preparations were then buffer exchanged into a 20 mM Tris/3.2 mM EDTA, pH 7.0 buffer using a 30 kDa membrane to remove the reducing buffer. The resulting partially reduced preparations were then conjugated to PBD1 (DL6 as set forth above) via a maleimide linker for a minimum of 30 mins. at 20° C. The reaction was then quenched with the addition of excess NAC compared to linker-drug using a 10 mM stock solution prepared in water. After a minimum quench time of 20 minutes the pH was adjusted to 6.0 with the addition of 0.5 M acetic acid. The pH adjusted ADCs were then processed through preparative Hydropobic Interaction Chromatography (HIC) (Butyl Sepharose FF) in a bind and elute mode with a step elution to further purify the DAR 2 species. The purified ADCs were then buffer exchanged into diafiltration buffer by diafiltration using a 30 kDa membrane. The dialfiltered anti-DLL3 ADC was then formulated with sucrose and polysorbate-20 to the target final concentration to produce the site-specific drug substance.

### Example 7

### Immunohistochemistry

Immunohistochemistry (IHC) was performed on primary human tumor tissue sections to assess the expression and location of DLL3 in tumor cells.

In order to identify an IHC-compatible anti-DLL3 antibody, IHC was performed on HEK293T parental cell pellets or DLL3-expressing HEK293T cell pellets using numerous anti-DLL3 antibodies of the invention. Several murine anti-DLL3 antibodies (including SC16.65) were able to specifically detect DLL3-overexpressing HEK293T cell pellets more effectively than other anti-DLL3 antibodies of the invention that were tested (data not shown). The ability of these antibodies to specifically detect DLL3 was confirmed by a competition experiment in which the relevant anti-DLL3 antibody was mixed with a 5× molar ratio excess of hDLL3-His protein and then incubated with DLL3-expressing HEK293T formalin fixed and paraffin embedded (FFPE) sections. The absence of positive staining demonstrated that the hDLL3-His protein interfered with the binding of the anti-DLL3 antibody to the DLL3-overexpressing HEK293T cells (data not shown).

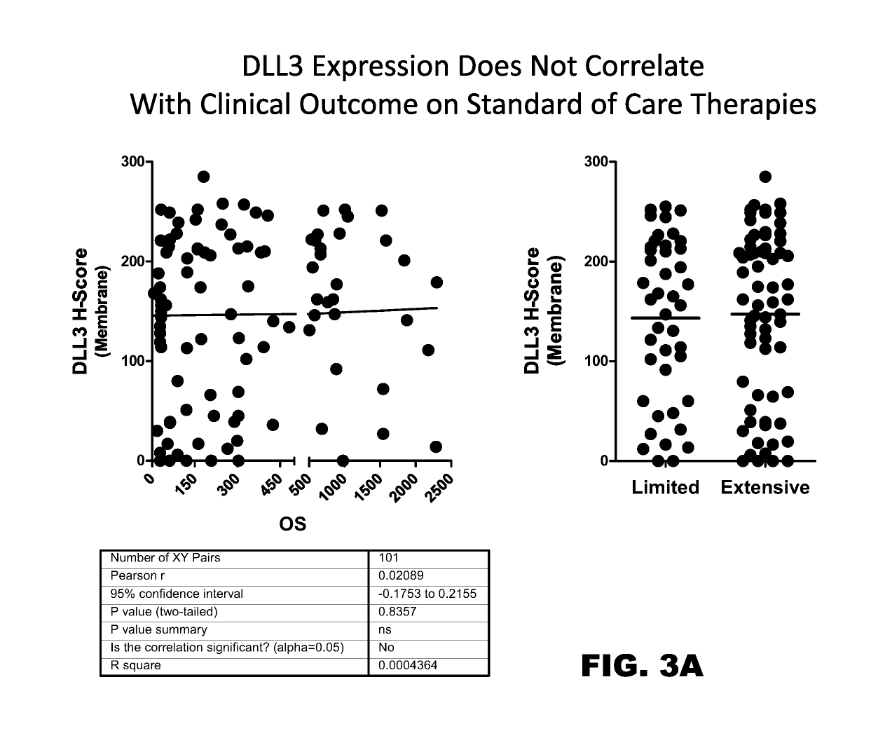

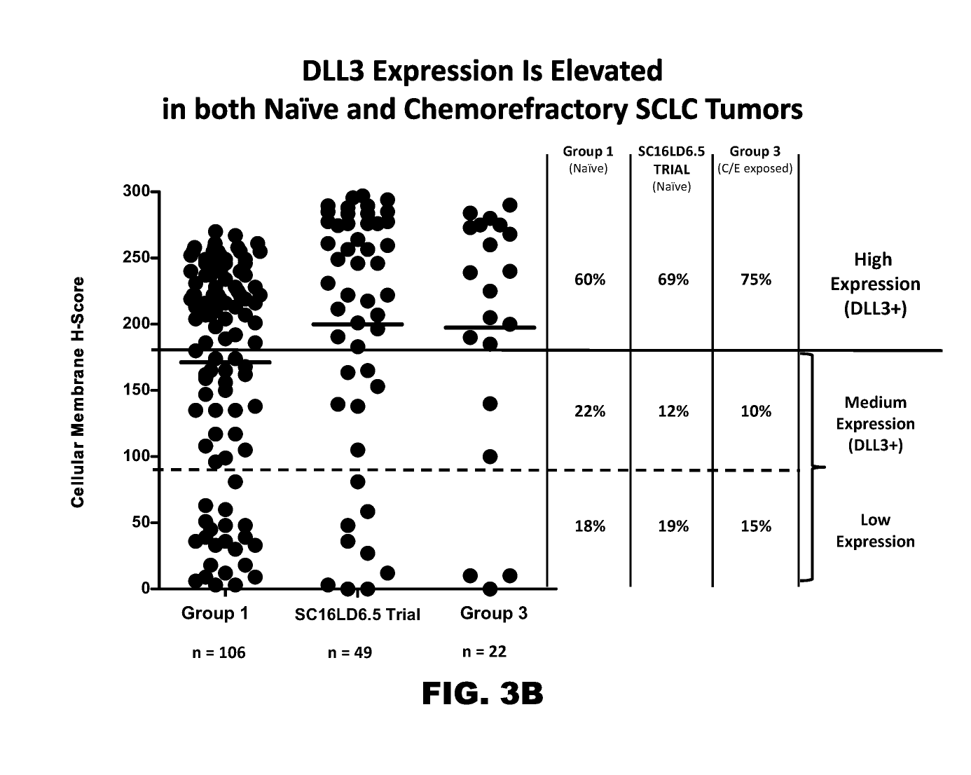

IHC was performed, as described below, on formalin fixed and paraffin embedded (FFPE) tissues as is standard in the art. Planar sections of tissues were cut and mounted on glass microscope slides. After xylene de-paraffinization 5 μm sections were pre-treated with Antigen Retrieval Solution (Dako) for 20 mins. at 99° C., cooled to 75° C. and then treated with 0.3% hydrogen peroxide in PBS followed by Avidin/Biotin Blocking Solution (Vector Laboratories). FFPE slides were then blocked with 10% horse serum in 3% BSA in PBS buffer and incubated with a primary anti-DLL3 antibody of the invention, diluted to 10 μg/ml in 3% BSA/PBS, for 30 mins. at room temperature. The FFPE slides were then incubated with biotin-conjugated horse anti-mouse antibody (Vector Laboratories), diluted to 2.5 μg/ml in 3% BSA/PBS, for 30 mins. at room temperature followed by incubation in streptavidin-HRP (ABC Elite Kit; Vector Laboratories). FFPE slides of primary human tumors were then incubated in biotinyl tyramide followed by incubation in streptavidin-HRP following manufacturers' instruction from the TSA amplification kit (TSA Amplification Kit; Perkin Elmer). Chromogenic detection was developed with 3,3′-diaminobenzidine (Thermo Scientific) for 5 mins. at room temperature and tissues were counterstained with Meyer's hematoxylin (IHC World), washed with alcohol and immersed in xylene. PDX tumors did not receive the TSA amplification. Sections were then viewed by brightfield microscopy and DLL3 membranous expression on tumor epithelium was noted by H-score. The H-score is obtained by the formula: 3×percentage of strongly staining membranous surface+2×percentage of moderately staining membranous surface+percentage of weakly staining membranous surface, giving a range of 0 to 300. Results of the studies are shown in FIGS. 3A and 3B.

FIGS. 3A and 3B graphically depict the expression of DLL3 protein vs overall survival (OS) and in limited vs extensive SCLC tumors (FIG. 3A) or in naïve and chemorefractory SCLC tumors. An examination of FIG. 3A shows that DLL3 expression is not indicative of overall survival (in patients treated with prior art standard of care agents) and that both limited SCLC tumors and extensive SCLC tumors express varying levels of DLL3. Unlike melanoma, where DLL3 expression has been inversely correlated with patient survival, DLL3 expression in tumors exhibiting neuroendocrine features does not appear indicative of mortality with regard to standard of care therapies. Similarly FIG. 3B demonstrates that DLL3 expression levels are approximately the same both naïve and with standard of care treated tumors. FIG. 3B further shows empirically derived DLL3 H-scores of approximately 90 (dashed line) and 180 (solid line) on a 300 point scale. Other DLL3 H-scores between 90 and 180 (e.g., 120) were also derived (not shown). These H-scores was examined in accordance with the Phase I trial described in Example 8 and were found to be indicative of patients that may respond favorably to treatment with the DLL3 ADCs of the instant invention. Accordingly in one embodiment a patient to be treated with the DLL3 ADCs of the instant invention will have a DLL3 H-score of at least 90. In other embodiments a patient to be treated with the DLL3 ADCs of the instant invention will have a DLL3 H-score of at least 120. In yet other embodiments a patient to be treated with the DLL3 ADCs of the instant invention will have a DLL3 H-score of at least 150 and more preferably will have a DLL3 H-score of at least 180. For the purposes of the instant disclosure any tumor exhibiting a DLL3 H-score of 90 or greater (or, as discussed above, where ≥10% of the constituent cells express DLL3) shall be considered DLL3+ and potentially treatable with the disclosed compounds and compositions.

Note that in other studies an H-score with a 200 point scale was also developed. In such 200 H-score scales an H-score of 120 is approximately equivalent to an H-score of 180 on a 300 H-score scale. In both cases (e.g., 120/200 or 180/300) such H-scores may be classified as biomarker positive (BM+) or DLL3+ (i.e., they are both above an H-score of 90 on a 300 point scale and/or ≥10% of the constituent cells express DLL3) and are suggestive of patients that may respond favorably to the treatment methods of the instant invention.

### Example 8

### SC16.56PBD1 (SC16LD6.5) Study Overview and Results

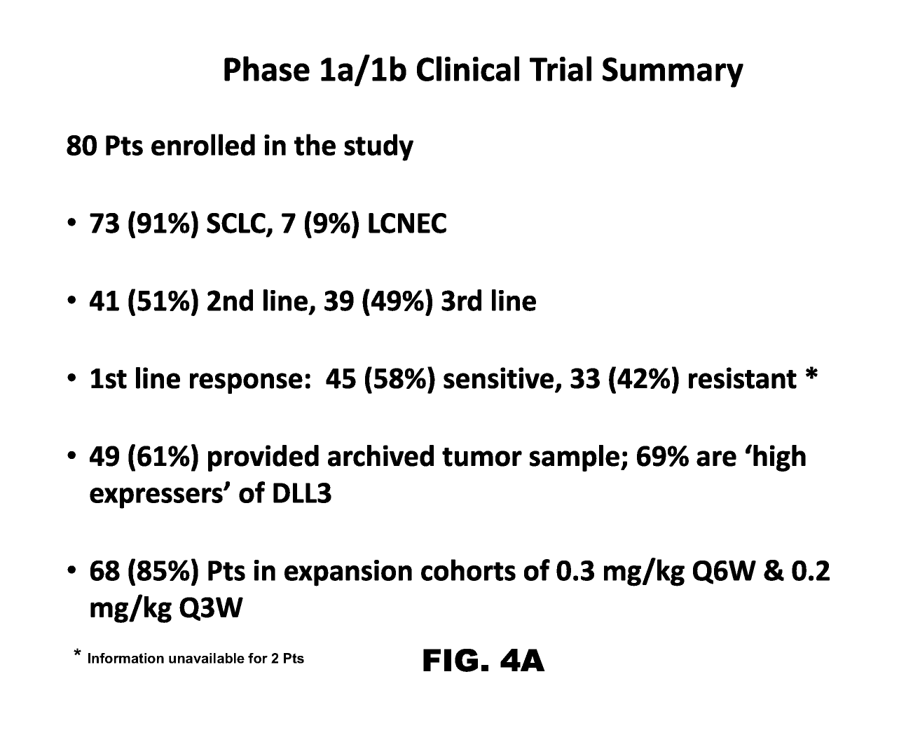

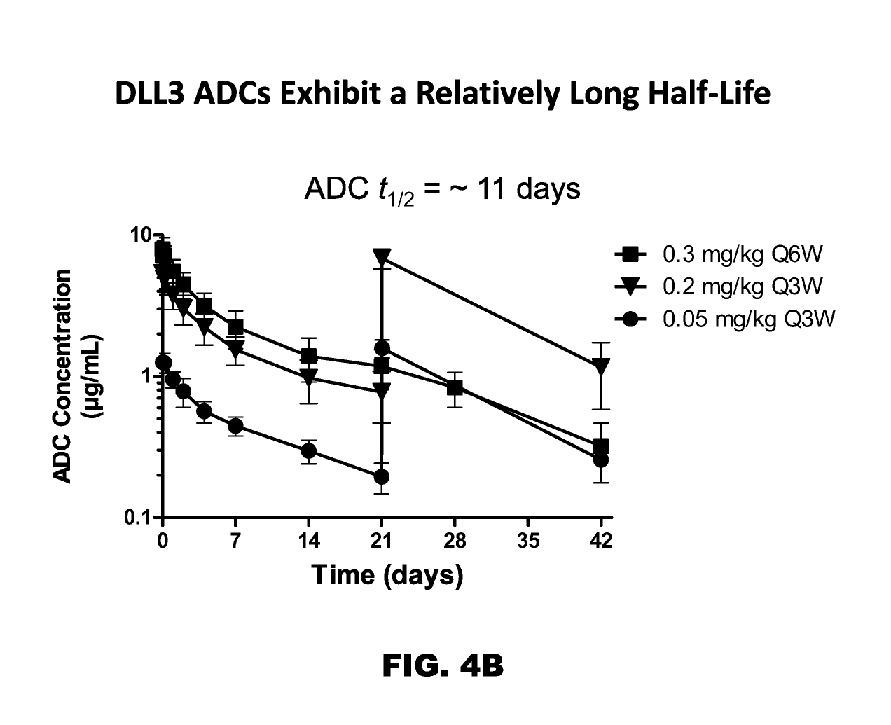

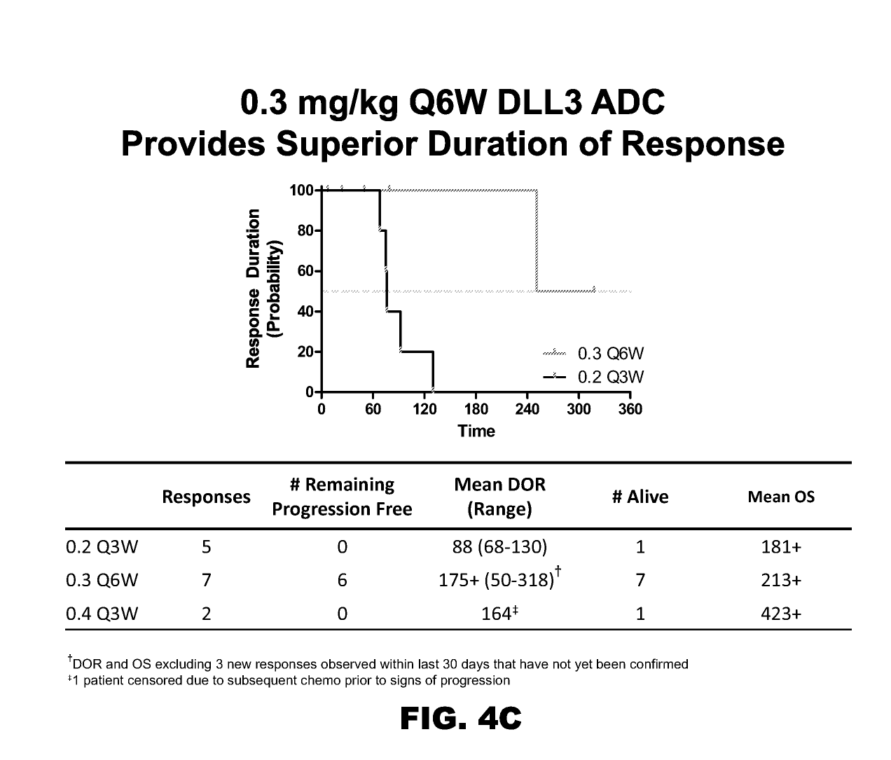

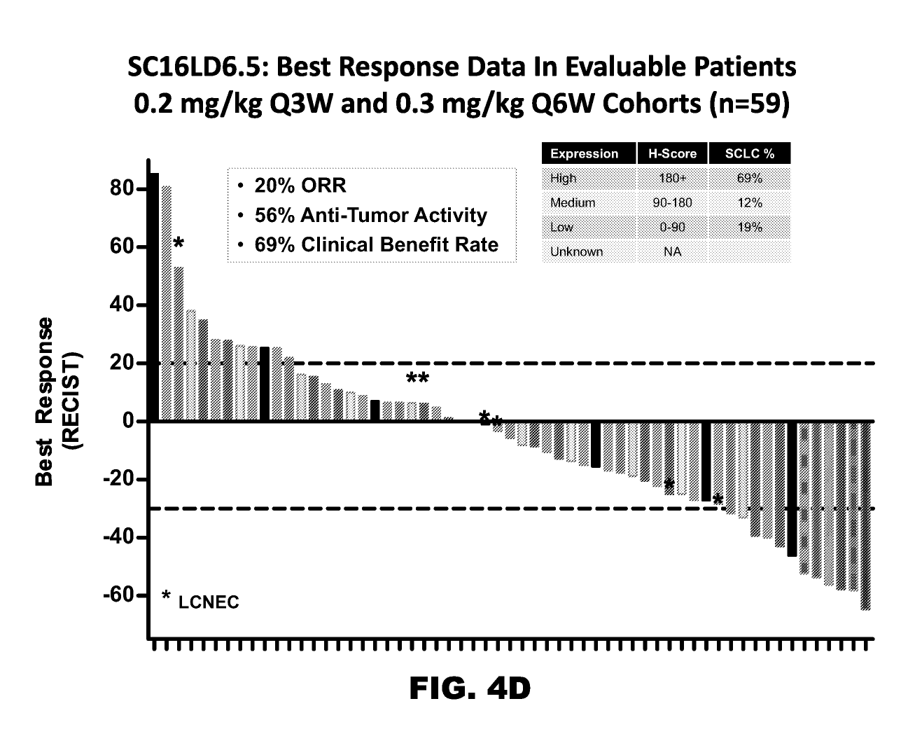

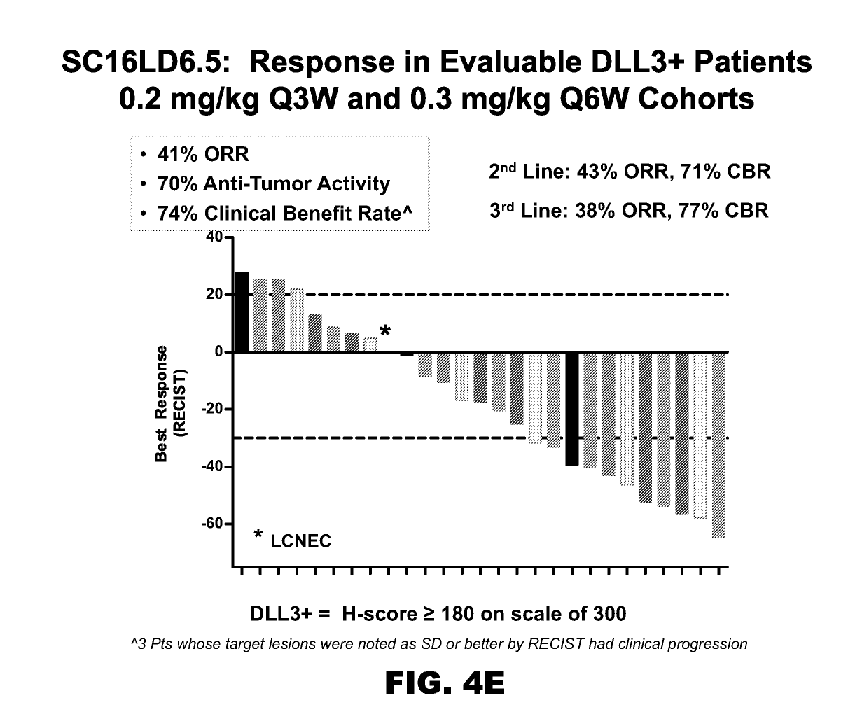

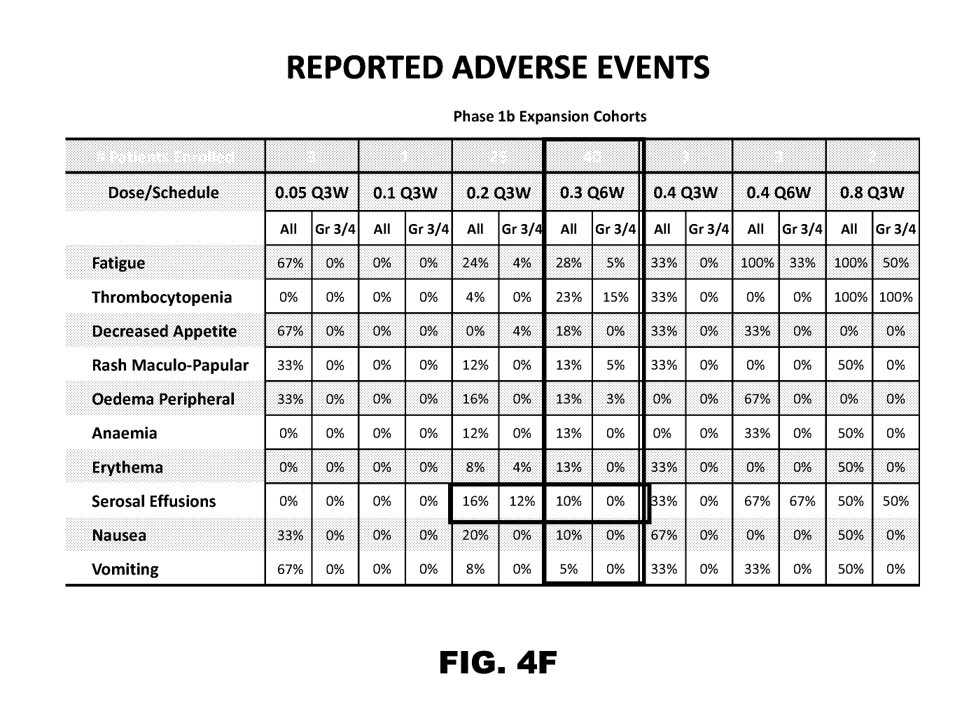

A Phase I clinical study was conducted to explore the possibility of using the disclosed DLL3-targeted ADCs to treat patients with recurrent small cell lung cancer (SCLC) and large cell neuroendocrine carcinoma (LCNEC). A summary of the clinical trial is provided in FIG. 4A.

Background:

Rovalpituzumab tesirine (i.e. Rova-T, SC16LD6.5, hSC16.56PBD1 or hSC16.56DL1 described herein) is a Delta-like protein 3 (DLL3) targeted antibody-drug conjugate (ADC) comprised of a humanized monoclonal antibody, dipeptide linker, and pyrrolobenzodiazepine (PBD) dimer toxin with a drug-to-antibody ratio of 2. SC16LD6.5 may be prepared substantially as set forth in Example 6 under GMP conditions. DLL3 is highly expressed in human neuroendocrine tumors and their tumor-initiating cells, including approximately two-thirds of SCLC and LCNEC tumors. DLL3 protein is not expressed at detectable levels in normal tissues. Rovalpituzumab tesirine induced tumor regression and prolonged time to progression significantly outperforming cisplatin/etoposide in DLL3-expressing SCLC and LCNEC patient-derived xenograft (PDX) tumor models. Based on this promising activity, a first-in-human phase I trial (NCT01901653) in patients with recurrent SCLC was initiated and preliminary results are reported below.

Methods:

SCLC patients with progressive disease after 1 or 2 previous lines of therapy (i.e., second or third line patients) received escalating doses of rovalpituzumab tesirine as a single agent once every 3 weeks (Q3W) in 1-3 pt cohorts until dose limiting toxicities (DLTs) were observed. The doses were 0.05, 0.1, 0.2, 0.4 and 0.8 mg/kg Q3W. Midway through accrual, pharmacokinetic data (FIG. 4B and as discussed in more detail in Example 9 below) revealed a longer than expected ADC half-life of −11 days, prompting evaluation of a Q6W schedule. As described in the previous Examples a DLL3 antibody was developed and utilized to assess antigen expression in archived tumor specimens. For the purposes of this study biomarker positive (BM+) tumors were defined by IHC membrane-associated H-Scores 120 (on a 200 H-score scale).

Results:

79 patients were treated: 34 Q3W and 45 Q6W; 33F/46M; median age, 61 years (44-81). Acute and chronic DLTs of thrombocytopenia and serosal effusions (according to a data monitoring committee comprised of experts mistakenly annotated by investigators in the Phase I trial as capillary leak syndrome, “CLS”) were observed at 0.8 and 0.4 mg/kg Q3W, respectively. Maximum tolerated doses (MTD) of 0.2 mg/kg Q3W×3 cycles and 0.3 mg/kg Q6W×2 cycles were further evaluated in expansion cohorts. The most common (≥20%) treatment emergent AEs of any grade among all patients were fatigue (47%), dyspnea (24%), nausea (24%), and decreased appetite (22%). The most common related treatment emergent SAEs were serosal effusions and thrombocytopenia, reported in 11 (14%) and 3 (4%) patients respectively (FIG. 4F).

Of 49 archived tumor specimens received from enrolled patients, 34 (69%) were DLL3 BM+. Among the 27 confirmed DLL3 BM+ patients treated at the MTDs, 11 patients (41%) had partial response (PR) and 12 patients (44%) achieved stable disease (SD) for a combined clinical benefit rate (CBR) of 85% (FIG. 4E). In all evaluable patients treated at the MTD without regard for DLL3 biomarker status (n=59), the ORR was 20% (n=12 PR) and SD 59% (n=35), for a CBR of 80% (FIG. 4D). Notably, all patients with PRs that were treated at the MTD, and those having the most durable clinical benefit (up to 569 days OS), were BM+. Similar response rates were observed among patients sensitive and refractory to first-line therapy and in the third-line setting where no standard-of-care currently exists.

Interestingly, despite similar overall exposure to the ADC, the 0.3 mg/kg Q6W×2 showed a superior duration of response when compared with the 0.2 mg/kg Q3W×3 cohort (FIG. 4C), averaging >175 days vs. 88 days, respectively. FIG. 4C further shows that the same 0.3 mg/kg Q6W×2 cohort had a better mean OS than the 0.2 mg/kg Q3W×3 cohort.

Conclusions:

Rovalpituzumab tesirine (Rova-T), a first-in-class DLL3-targeted ADC, has manageable toxicity and demonstrated significant anti-tumor activity (41% ORR and 80% CBR) as a single agent in second- and third-line patients with recurrent DLL3 BM+ SCLC.

### Example 9

### SC16.56PBD1 (SC16LD6.5) Pharmacokinetics

As alluded to in the previous Example the pharmacokinetics (PK) of SC16LD6.5 was assessed in the Phase 1a/1b clinical trial described above. The concentration of SC16LD6.5 in human serum was assessed following administration of a pre-specified dose of SC16LD6.5 to patients with SCLC or LCNEC. Blood samples for serum levels were collected from all subjects entered into study immediately prior to their first dose of SC16LD6.5 and at multiple time points on Day 1 (Day 1 being the day of infusion). Additional blood for serum measurements were drawn once each on Days 2, 3, 5, 8, and 15 of Cycle 1. In cycles 2 and 3, blood was taken immediately pre (trough) and post (peak) infusion on Day 1. At or near estimated steady-state (Cycle 4), additional samples were collected on Day 1, 8 and 15. Following blood collection, specimens were processed to serum and frozen.

The serum concentration of SC16LD6.5 was measured in an enzyme linked immunosorbent assay (ELISA) with chemilumenescent detection on a mesoscale discovery (MSD) platform. This assay employed a pair of anti-idiopathic antibodies that specifically capture and detect SC16LD6.5 with one or more conjugated toxins. This assay cannot distinguish the number of toxin conjugates on SC16LD6.5. The concentration-time profile was generated based on these measured concentrations (FIG. 4B).

Based on the serum concentration of SC16LD6.5, the PK of SC16LD6.5 was estimated by noncompartmental analysis with the software package, Phoenix WinNonlin. PK was linear with dose-proportional increases in exposure. The terminal half-life was estimated by log-linear regression of the terminal phase of the concentration-time profile to determine the terminal rate coefficient (lambda). The quotient of the natural log of 2 and lambda was calculated to estimate the terminal half-life. The terminal half-life of SC16LD6.5 was estimated to be approximately 10.1 days following administration of 0.2 mg/kg of SC16LD6.5 every 3 weeks and approximately 12.1 days following administration of 0.3 mg/kg of SC16LD6.5 every 6 weeks. This estimated half-life exceeds that of other antibody-drug conjugates such as brentuximab vedotin and ado-trastuzumab emtansine. Such an unexpectedly long half-life allows for the achievement of a robust therapeutic index using the novel dosing regimens (e.g., 0.2 mg/kg Q3W×3 and 0.3 mg/kg Q6W×2).

### Example 10

### Anti-DLL3 ADC Lyophilization

In order to provide options for long-term storage and subsequent commercial activities, exemplary DLL3-ADCs of the instant invention were lyophilized. The lyophilized samples, stored in pharmaceutically compatible vials, were then used to conduct stability studies to determine the applicability of the technique to ADCs comprising PBDs. As the PBD drug linker conjugates are large and relatively hydrophobic, the ability to provide such lyophilized compositions that exhibit long-term stability was unclear.

For this study SC16LD6.5 antibody-drug conjugate preparations (prepared substantially as set forth in Example 6 above) were formulated at 10 mg/mL SC16LD6.5, 175 mM sucrose, 20 mM L-histidine hydrochloride (pH 6.0), and 0.4 mg/mL polysorbate 20. This exemplary formulation was chosen as suitable for long-term storage as a frozen liquid at the intended conditions of <−70° C. for Phase I/II clinical trials and potentially stable in lyophilized form under appropriate conditions. A lyophilized dosage form (30 mg/vial, approximately 3 mL in a 10-mL glass vial) intended for storage under refrigerated conditions (2-8° C.) using the identical formulation was developed for later clinical trials and potential commercial use.

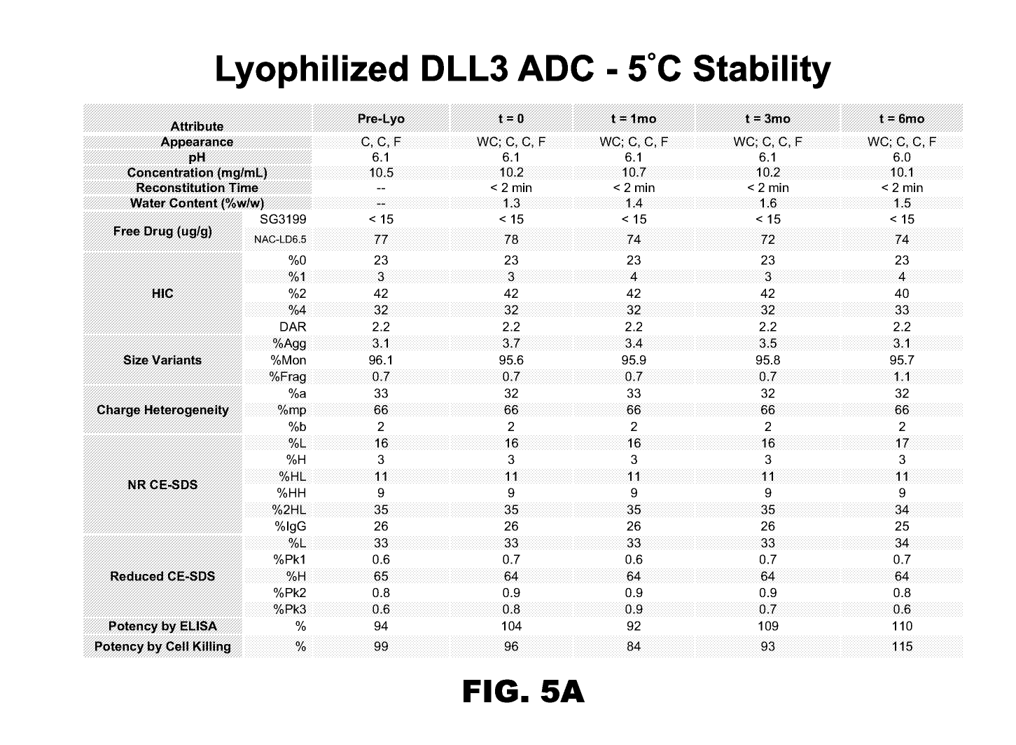

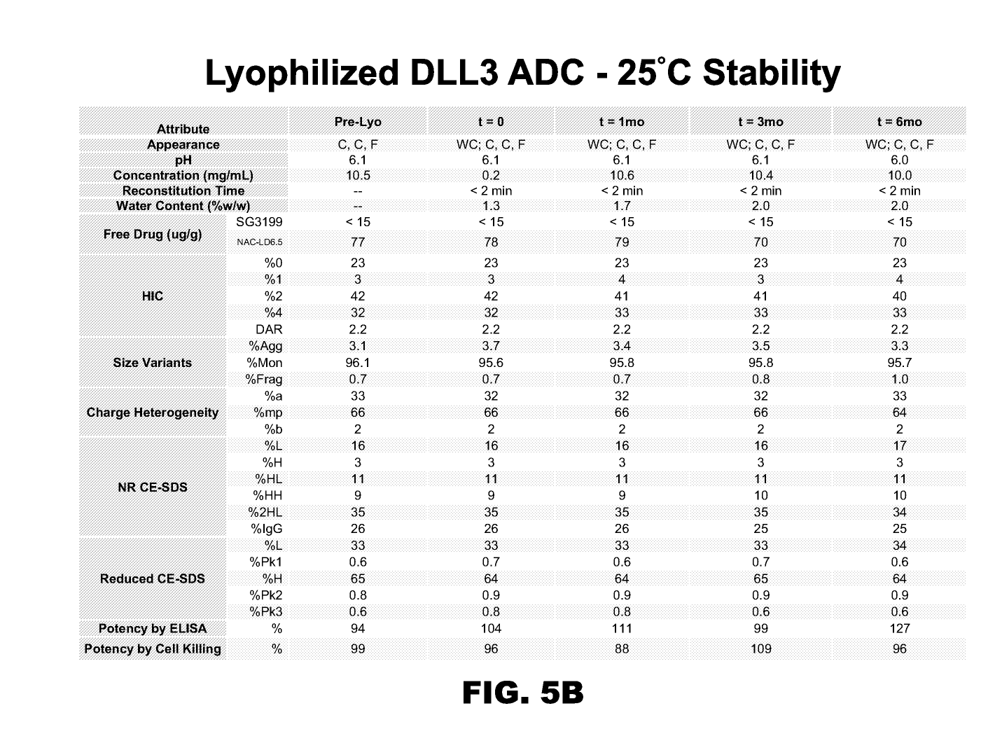

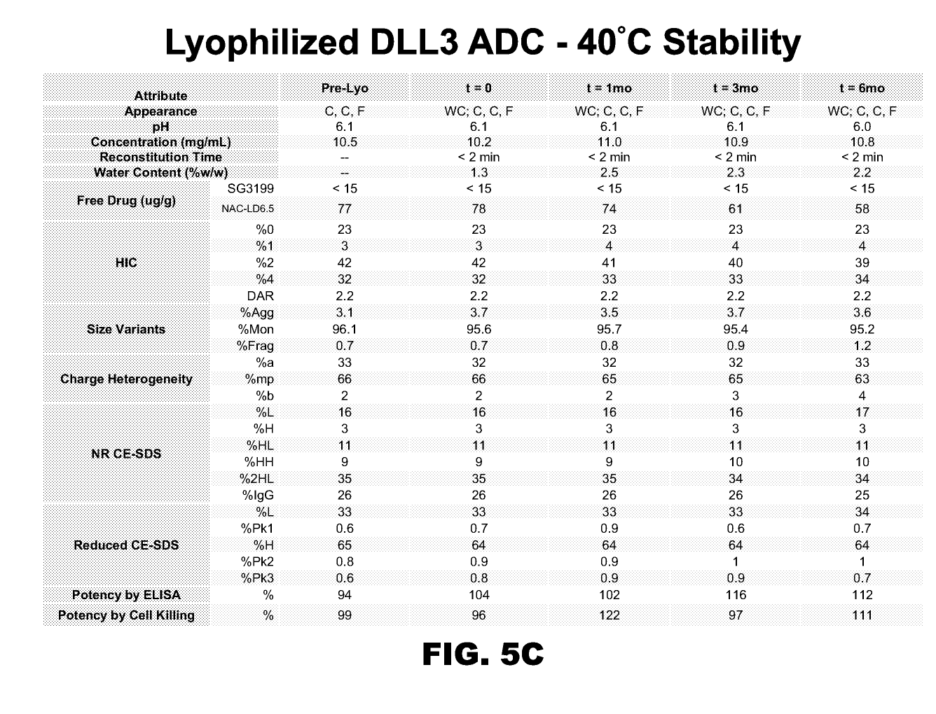

The lyophilization process and conditions were derived using a development-scale lyophilizer. The process includes the following steps: freeze ramp, freeze hold, primary drying, and secondary drying. Using this process, a batch of lyophilized vials was produced in the equipment intended for production of clinical material. Following lyophilization, stoppered vials containing the powdered DLL3-ADCs were stored at different temperatures (5° C., 25° C. and 40° C.) for a prolonged period. At predetermined times selected vials were reconstituted and the resulting formulation analyzed for any material deviation from the starting formulation. Results of the analysis for time-points up to six months are shown in FIGS. 5A-5C.

A review of the data demonstrates that the lyophilized ADCs showed little degradation at any of the chosen temperatures. Accordingly lyophilized vials produced at the development-scale were demonstrated to be stable under real-time, stressed, and accelerated storage conditions.

### Example 11

### Combinations of Anti-DLL3 ADCs and Anti-PD-1 Antibodies Inhibit Small Cell Lung Cancer Tumor Growth In Vivo

As discussed above the DLL3 ADCs of the instant invention may be combined with anti-PD-1 antibodies to effectively treat various DLL3 expressing tumors. In order to demonstrate the therapeutic ability of the instant invention hSC16.56DL1 (Rova-T) was tested in combination with anti-PD-1 antibodies that inhibit the interaction between PD-1 and its ligand PDL1 in syngeneic in vivo models of small cell lung cancer (SCLC).

Rova-T is a DLL3-specific antibody-drug conjugate that, as demonstrated in this application, has shown activity against SCLC in a clinical setting. Antibodies directed against PD-1, which block the interaction between PD-1, which is mainly expressed on activated T cells and its ligands PD-L1 and PD-L2 have shown clinical utility in a number of cancer indications. Currently, the anti-PD-1 antibody drugs Opdivo® (nivolumab) and Keytruda® (pembrolizumab) are approved for use in certain patients with melanoma, non-small cell lung cancer (NSCLC), head and neck squamous cell carcinoma and Hodgkin's lymphoma but some activity has also been demonstrated in a subset of patients with SCLC. To determine whether Rova-T and anti PD-1 antibodies have inhibitive, neutral, additive or synergistic effects when used in combination their activities as single agents and in combination were tested in animal models of SCLC.

Since the mechanism of action of anti-PD-1 antibodies requires an immunocompetent host the DLL3 expressing mouse cell line KP1 in conjunction with immune compatible syngeneic mice was used. The KP1 cell line has been derived from spontaneous small cell lung cancer tumors found in the lungs of mice lacking the RB1 and TP53 genes (PMID: 21983857). Because Rova-T is capable of binding the human and murine DLL3 protein with comparable affinities and in vitro cytotoxic activity towards DLL3 expressing cells, the drug can be used in mouse models. But, as neither nivolumab nor pembrolizumab sufficiently interact with murine PD-1, an anti-mouse PD-1 surrogate antibody clone was selected. In this regard anti-mouse PD-1 antibody clone EH12.2H7 from BioLegend (San Diego, USA) has been shown to inhibit the interaction between PD-1 and PD-L1 similar to the anti-human antibodies used in clinical settings and to mimic their T cell enhancing and anti-cancer activities.

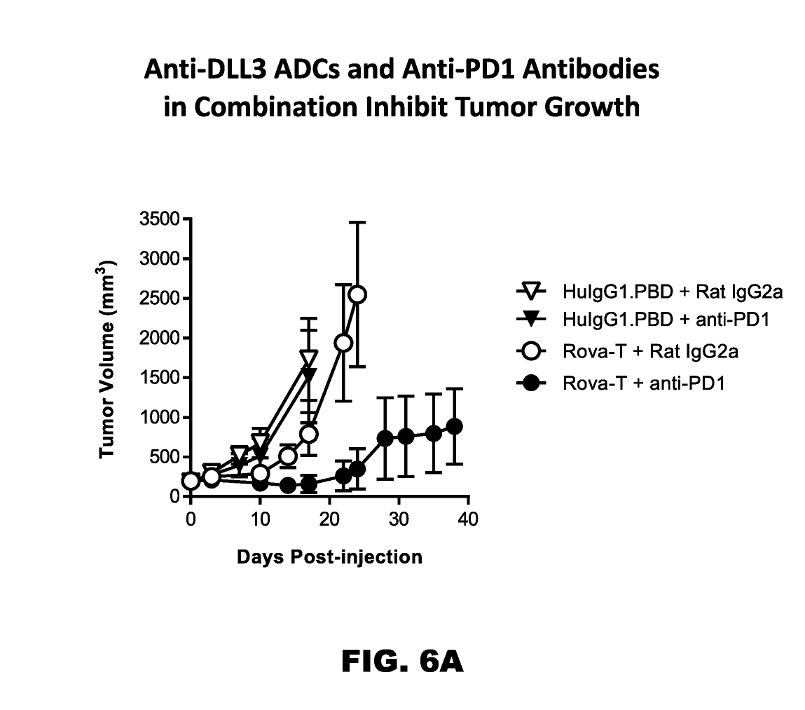

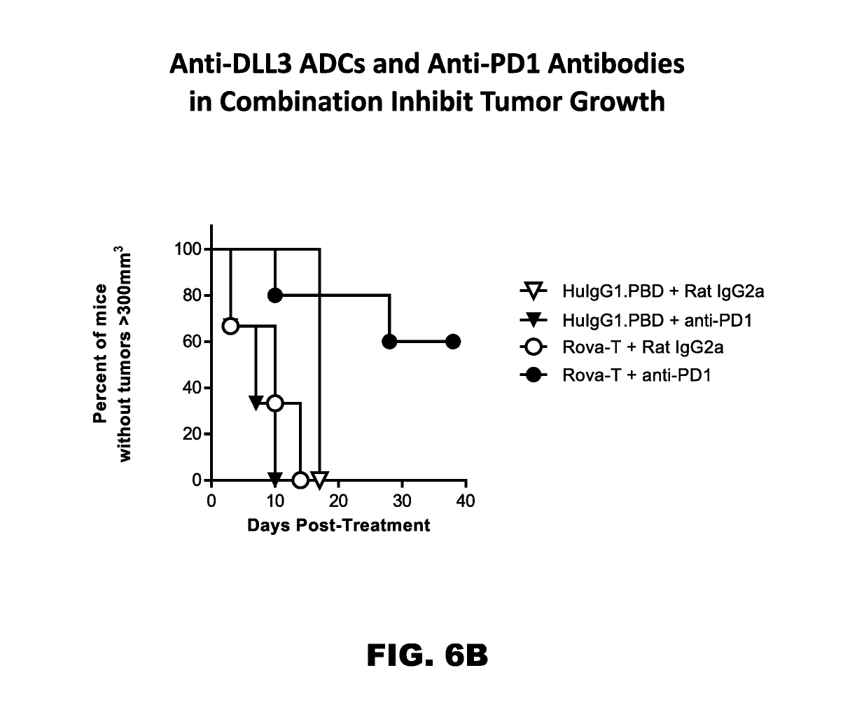

KP1 cells were grown as subcutaneous tumors in the flanks of B6129SF1/J mice (The Jackson Laboratory #101043, USA). Tumor volumes were measured twice weekly with calipers. When average tumor volumes were approximately 200 mm³, mice were randomized into cohorts of 5 mice each. By intraperitoneal injection, mice were treated with either SC16LD6.5 (“Rova-T”, 0.1 mg/kg, treatment Day 1 only) or HulgG1.DL1 (“HulgG1.PBD”, 0.5 mg/kg, treatment Day 1 only) and the same mice were also treated with either anti-mouse PD-1 antibody (“anti-PD-1”, 200 ug/mouse, treatment Days 1, 3, 7; BioLegend, USA) or Rat IgG2a antibody (“Rat IgG2a”, 200 ug/mouse, treatment days 1,3,7; BioLegend, USA). Tumor volumes continued to be measured twice weekly and mice were euthanized when tumor volumes exceeded 1500 mm³. The results of the study are shown in FIGS. 6A and 6B appended hereto.

As shown in FIG. 6A the combination of the anti-PD-1 antibody and the HuIgG1.PBD control does not significantly inhibit KP1 tumor growth. Similarly combinations of the Rat IgG2a (isotype control for anti-PD-1) with the HuIgG1.PBD control or Rova-T fail to substantially inhibit the growth of KP1 tumors in vivo, (FIG. 6A) for an appreciable time. While the delay was not extensive it will be appreciated that at these dosages Rova-T, essentially acting as a single agent, apparently showed some inhibition of tumor growth.

Likewise, as shown in FIG. 6B, the combination control treatments HulgG1.PBD+Rat IgG2a and HulgG1.PBD+anti-PD-1 did not substantially prolong the time before KP1 tumor volumes grew to exceed 300 mm³. Moreover, at the doses shown FIG. 6B the Rova-T+isotype control (Rat IgG2a) combination does not significantly inhibit KP1 tumor growth compared to treatment with HulgG1.PBD. While Rova-T (hSC16.56DL1) essentially acting as the sole active agent completely eliminates KP1 tumors at a dose of 0.5 mg/kg (data not shown), the relatively low dose of 0.1 mg/kg shows little effect against this tumor cell line.

In sharp contrast with the control combinations, treatment with Rova-T and with an anti-PD-1 antibody in combination produced a significant inhibition of KP1 tumor growth, in vivo, compared to treatment with either of the two active agents alone (FIGS. 6A and 6B). As shown in both FIG. 6A and FIG. 6B mean tumor growth is severely delayed. While all mice from combination control treatment groups had tumors larger than 300 mm³ after 17 days, only two of five (40%) combination group mice had tumors larger than 300 mm³ (FIG. 6B). Similarly FIG. 6A shows that the mean tumor volume in the mice treated with Rova-T and an anti-PD-1 antibody remained below 1000 mm³ for over twice as long as the Rova-T+isotype control treated mice. Moreover, the median percent tumor growth inhibition (the percent by which the smallest measured post-treatment tumor volume was smaller than the starting tumor volume) for the group treated with Rova-T+anti-PD-1 was 69%, compared to 0% for “HulgG1.PBD+Rat IgG2a”, 0% for “HuIgG1.PBD+anti-PD-1”, and 0% for “Rova-T+Rat IgG2a” treatment groups. This tumor growth inhibition data strongly suggests that the combination of a DLL3 ADC and an anti-PD-1 antibody may be synergistic in nature.

In any event the data set forth in this Example clearly show that the combination of DLL3 ADCs with anti-PD-1 antibodies demonstrate at least additive, if not synergistic, effects against SCLC by reducing tumor volumes and facilitating the long lasting survival of tumor bearing mice. The observed effect is specific and dependent on DLL3 recognition by Rova-T as treatment with an isotype PBD ADC in combination with PD-1 antibodies shows no improved efficacy towards the vehicle control. The measured effect is also, at least in part, due to PD-1 binding and inhibition as an isotype control Ab in combination with Rova-T fails to show the same results.

In summary, the data presented show that targeted cell killing of DLL3 expressing cancer by Rova-T may further enhance the effect of immunotherapy, including immunotherapy comprising the use of PD-1 and/or PD-L1 antibodies.

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PBD, and translations from annotated coding regions in GenBank and RefSeq cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method of treating a subject having a tumor exhibiting a DLL3 H-score of at least 90 on a 300 point scale and/or ≥10% positively stained DLL3 cells comprising the step of administering a DLL3 ADC.

2. The method of claim 1 wherein the DLL3 ADC comprises cytotoxin selected from the group consisting of PBDs, calicheamicins, auristatins, maytansinoids and duocarmycins.

3. The method of claim 2 wherein the cytotoxin comprises a PBD.

4. The method of claim 3 wherein the PBD comprises PBD1.

5. The method of any one of claims 1-4 comprising a DLL3 ADC that binds to a tumor initiating cell expressing DLL3.

6. The method of any one of claims 1-5 wherein the DLL3 ADC comprises an antibody which is a chimeric, CDR grafted, human or humanized antibody, or a fragment thereof.

7. The method of claim 6 wherein the antibody is an internalizing antibody.

8. The method of any one of claims 1-7 wherein the tumor exhibits a DLL3 H-score of at least 120 on a 300 point scale.

9. The method of any one of claims 1-8 wherein the tumor exhibits a DLL3 H-score of at least 180 on a 300 point scale.

10. The method of any one of claims 1-9 wherein the tumor comprises a neuroendocrine tumor.

11. The method of any one of claims 1-10 wherein the tumor comprises a small cell lung cancer (SCLC) tumor.

12. The method of any one of claims 1-10 wherein the tumor comprises a large cell neuroendocrine cancer (LCNEC) tumor.

13. The method of any one of claims 1-12 wherein the tumor comprises a medullary thyroid cancer tumor.

14. The method of any one of claims 1-13 wherein the subject is treated with a DLL3 ADC regimen comprising a 0.2 mg/kg Q3W×3 dosing regimen.

15. The method of any one of claims 1-13 wherein the subject is treated with a DLL3 ADC regimen comprising a 0.3 mg/kg Q6W×2 dosing regimen.

16. The method of claim 14 or 15 wherein the subject is treated at progression following the DLL3 ADC regimen.

17. The method of claim 14 or 15 wherein the subject is shifted to a DLL3 ADC maintenance therapy following the DLL3 ADC regimen.

18. The method of any one of claims 1-17 wherein the subject is a front line patient.

19. The method of any one of claims 1-17 wherein the subject is a second line patient.

20. The method of any one of claims 1-17 wherein the subject is a third line patient.

21. The method of any one of claims 1-20 wherein the DLL3 ADC comprises SC16LD.5.

22. The method of any one of claims 1-20 wherein the DLL3 ADC comprises hSC16.56ss1DL6.

23. A method of treating a subject having a tumor comprising the steps of:
obtaining a sample of the tumor;
interrogating the tumor sample to calculate a DLL3 H-score and/or determine the percentage of positively stained DLL3 cells
treating the patient with a DLL3 ADC when the calculated DLL3 H-score is at least 90 on a 300 point scale and/or the positively stained DLL3 cells comprise ≥10% of the tumor cells.

24. The method of claim 23 wherein the interrogation step comprises immunohistochemistry.

25. A lyophilized composition comprising the antibody drug conjugate (ADC) of the formula Ab-[L-D]n or a pharmaceutically acceptable salt thereof wherein:
Ab comprises an anti-DLL3 antibody;
L comprises an optional linker;
D comprises a drug; and
n is an integer from 1 to 20.

26. The lyophilized composition of claim 25 wherein D comprises a PBD.

27. The lyophilized composition of claim 25 further comprising a pharmaceutically acceptable sugar.

28. The lyophilized composition of claim 26 wherein the ADC comprises SC16LD6.5.

29. The lyophilized composition of claim 26 wherein the ADC comprises hSC16.56ss1DL6.

30. An article of manufacture useful for diagnosing or treating DLL3 associated disorders comprising the composition of claim 25.

31. A method for treating cancer comprising the steps of:
reconstituting the lyophilized composition of claim 25 to provide a liquid pharmaceutical composition; and
administering the liquid pharmaceutical composition to a subject in need thereof.

32. The method of claim 31 wherein the cancer comprises a tumor exhibiting neuroendocrine features.

33. The method of claim 32, wherein the cancer comprises a neuroendocrine tumor

34. The method of claim 30 wherein the cancer comprises small cell lung cancer.

35. The method of claim 30 wherein the cancer comprises large cell neuroendocrine cancer.

36. An article of manufacture useful for diagnosing or treating DLL3 associated disorders comprising a receptacle comprising a lyophilized DLL3 ADC and associated with instructional materials for using said article of manufacture to treat or diagnose the DLL3 associated disorder.

37. The article of manufacture of claim 36 wherein said DLL3 ADC comprises a PBD.

38. A method of treating a subject having a tumor comprising the step of administering a DLL3 ADC having a terminal half-life of greater than about six days.

39. The method of claim 38 wherein said DLL3 ADC has a terminal half-life of greater than about seven days.

40. The method of claim 38 wherein said DLL3 ADC has a terminal half-life of greater than about eight days.

41. The method of claim 38 wherein said DLL3 ADC has a terminal half-life of greater than about nine days.

42. The method of claim 38 wherein said DLL3 ADC has a terminal half-life of greater than about 10 days.

43. The method of any one of claims 38-42 wherein the DLL3 ADC comprises cytotoxin selected from the group consisting of PBDs, calicheamicins, auristatins, maytansinoids and duocarmycins.

44. The method of claim 43 wherein the cytotoxin comprises a PBD.

45. The method of claim 44 wherein the PBD comprises PBD1.

46. The method of any one of claims 38-45 wherein the DLL3 ADC comprises an antibody which is a chimeric, CDR grafted, human or humanized antibody, or a fragment thereof.

47. The method of claim 46 wherein the antibody is an internalizing antibody.

48. The method of any one of claims 38-45 wherein the tumor comprises a tumor exhibiting neuroendocrine features.

49. The method of claim 46 wherein the tumor comprises a neuroendocrine tumor.

50. The method of any one of claims 38-47 wherein the tumor comprises a small cell lung cancer (SCLC) tumor.

51. The method of any one of claims 38-47 wherein the tumor comprises a large cell neuroendocrine cancer (LCNEC) tumor.

52. The method of any one of claims 38-51 wherein the subject is treated with a DLL3 ADC regimen comprising a 0.2 mg/kg Q3W dosing regimen.

53. The method of claim 52 wherein the subject is treated with a DLL3 ADC regimen comprising a 0.2 mg/kg Q3W×3 dosing regimen.

54. The method of any one of claims 38-51 wherein the subject is treated with a DLL3 ADC regimen comprising a 0.3 mg/kg Q6W dosing regimen.

55. The method of claim 54 wherein the subject is treated with a DLL3 ADC regimen comprising a 0.3 mg/kg Q6W×2 dosing regimen.

56. The method of claims 52 to 55 wherein the subject is treated at progression following the DLL3 ADC regimen.

57. The method of claims 52 to 55 wherein the subject is shifted to a DLL3 ADC maintenance therapy following the DLL3 ADC regimen.

58. The method of any one of claims 38-57 wherein the subject is a front line patient.

59. The method of any one of claims 38-57 wherein the subject is a second line patient.

60. The method of any one of claims 38-57 wherein the subject is a third line patient.

61. The method of any one of claims 38-60 wherein the DLL3 ADC comprises SC16LD.5.

62. The method of any one of claims 38-60 wherein the DLL3 ADC comprises hSC16.56ss1DL6.

63. A method of reducing the frequency of cancer stem cells in a subject in need thereof comprising the step of administering a DLL3 ADC having a terminal half-life of greater than about six days.

64. A DLL3 ADC comprising the following formula: 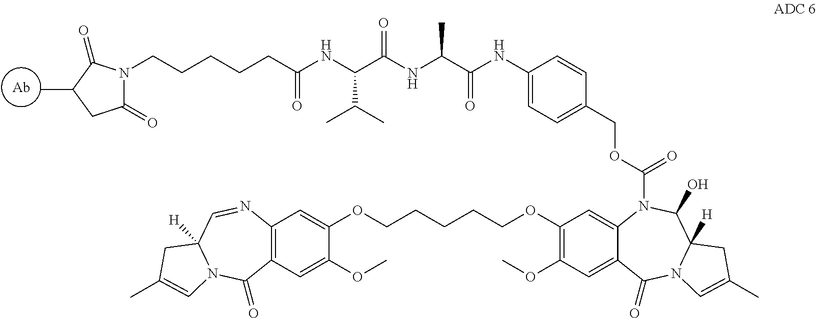
wherein Ab comprises an anti-DLL3 antibody or immunoreactive fragment thereof.

65. The DLL3 ADC of claim 64 wherein the anti-DLL3 antibody comprises a site-specific antibody.

66. The DLL3 ADC of claim 64 wherein the anti-DLL3 antibody is hSC16.56ss1.

67. A method of reducing the frequency of cancer stem cells in a subject in need thereof comprising the steps of administering a DLL3 ADC and an anti-PD-1 antibody.

68. The method of claim 67 wherein the DLL3 ADC comprises SC16LD5

69. The method of claim 67 wherein the DLL3 ADC comprises hSC16.56551DL6.

70. A method of reducing the frequency of cancer stem cells in a subject in need thereof comprising the steps of administering a DLL3 ADC and an anti-PD-L1 antibody.

71. The method of claim 70 wherein the DLL3 ADC comprises SC16LD5

72. The method of claim 70 wherein the DLL3 ADC comprises hSC16.56ss1DL6.

73. A method of treating cancer in a subject in need thereof comprising the steps of administering a DLL3 ADC and an anti-PD-1 antibody.

74. The method of claim 73 wherein the DLL3 ADC comprises SC16LD5

75. The method of claim 73 wherein the DLL3 ADC comprises hSC16.56ss1DL6.

76. A method of treating cancer in a subject in need thereof comprising the steps of administering a DLL3 ADC and an anti-PD-L1 antibody.

77. The method of claim 76 wherein the DLL3 ADC comprises SC16LD5

78. The method of claim 76 wherein the DLL3 ADC comprises hSC16.56ss1DL6.
